(19) United States
(12) Patent Application Publication
(10) Pub. No.: US 2025/0188470 A1
(43) Pub. Date: Jun. 12, 2025

(54) MULTIPLEXING TARGETING LIGANDS THROUGH CLICK CHEMISTRY AT THE ANOMERIC SITE OF SUGARS

(71) Applicant: ALNYLAM PHARMACEUTICALS, INC., Cambridge, MA (US)

(72) Inventors: Muthiah MANOHARAN, Cambridge, MA (US); Dhrubajyoti DATTA, Cambridge, MA (US)

(73) Assignee: ALNYLAM PHARMACEUTICALS, INC., Cambridge, MA (US)

(21) Appl. No.: 18/577,948

(22) PCT Filed: Jul. 15, 2022
(86) PCT No.: PCT/US2022/037262

§ 371 Date: Jan. 9, 2024

Related U.S. Application Data
(60) Provisional application No. 63/236,029, filed on Aug. 23, 2021.
(60) Provisional application No. 63/222,090, filed on Jul. 15, 2021.

(51) Int. Cl. C12N 15/113

(52) U.S. Cl. CPC C12N 15/1137; C12N 15/113; C12Y 115/01001; C12N 2310/11; C12N 2310/31; C12N 2310/321; C12N 2310/3231; C12N 2310/336; C12N 2310/351; C12N 2310/51

(57) ABSTRACT

The present disclosure relates to monomers and methods for conjugating one or more ligands to oligonucleotides by Click chemistry through attachment of azido group or triazolyl moiety at the anomeric site of pentose or hexose sugars. Another aspect the invention relates to a method of modulating the expression of a target gene in a cell, comprising administering to said cell an oligonucleotide and/or dsRNA molecule described herein.

### CROSS-REFERENCE TO RELATED APPLICATIONS

This application is a 35 U.S.C. § 371 National Phase Entry Application of International Application No. PCT/US2022/037262 filed Jul. 15, 2022, which designates the U.S. and claims benefit under claims benefit under 35 U.S.C. § 119(e) of U.S. Provisional Application No. 63/236,029 filed on Aug. 23, 2021 and U.S. Provisional Application No. 63/222,090 filed on Jul. 15, 2021, the contents of all of which are incorporated herein by reference in their entireties.

### SEQUENCE LISTING

The instant application contains a Sequence Listing which has been submitted electronically in XIL format and is hereby incorporated by reference in its entirety. Said XIL copy, created on Sep. 2, 2022, is named 051058-190520WOPT.xml and is 107,000 bytes in size.

### TECHNICAL FIELD

The present disclosure relates generally to monomers and methods for conjugating one or more ligands to oligonucleotides by azide alkyne cycloadditions (AAC or “Click”) chemistry at the anomeric site of sugars, such as pentose sugars or hexose sugars.

### BACKGROUND

There is a need in the art for monomers and methods for conjugating ligands to oligonucleotides. The present disclosure addresses these needs.

### SUMMARY

In one aspect, provided herein is a compound of Formula (III):

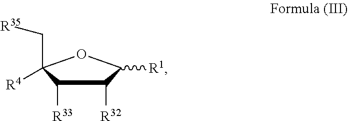

wherein:
R¹ is N₃ or

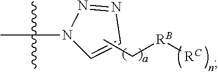

wherein:
a is 0 or 1;
n is 1, 2, 3, 4, or 5;
R^{B} is O, N, S, a heteroalkyl, a branched alkyl, a cycloalkyl, heterocyclyl, aryl or heteroaryl;
each R^{C} independently is

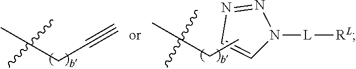

wherein:
each b′ is independently 0 or 1;
each L independently is absent or linker;
each R^{L} is a ligand, (e.g., selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, and polyethylene glycols (PEGs));
R³² is hydrogen, hydroxy, halogen, protected hydroxy, phosphate group, reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R³³ is hydrogen, hydroxy, halogen, protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support, and optionally, only one of R³² and R³³ is a phosphate group, a reactive phosphorous group, a solid support, a linker, or a linker covalently bonded (e.g., —C(O)CH₂CH₂C(O)—) to a solid support; R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R³² taken together are 4′-C(R¹⁰R¹¹)ᵥ—Y-2′ or 4′-Y—C(R¹⁰R¹¹)ᵥ-2′; Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—.
   R¹⁰ and R¹¹ independently are H, optionally substituted C₁-C₆alkyl, optionally substituted C₂-C₆alkenyl or optionally substituted C₂-C₆alkynyl;
   R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
   v is 1, 2 or 3;
or R⁴ and R³³ taken together with the atoms to which they are attached form an optionally substituted C₃₋₈cycloalkyl, optionally substituted C₃₋₈cycloalkenyl, or optionally substituted 3-8 membered heterocyclyl;
R³⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10); and
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In some embodiments of any one of the aspects described herein, a compound of Formula (III) is of Formula (IIIa):

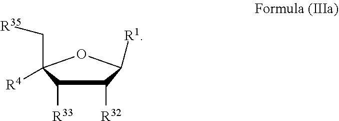

In some embodiments of any one of the aspects described herein, a compound of Formula (III) is of Formula (IIIb):

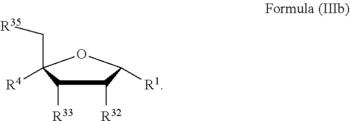

In another aspect, provided herein is a compound of Formula (IIIc):

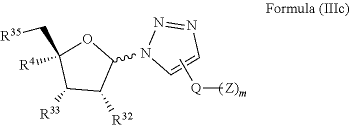

wherein
R³² is hydrogen, hydroxy, halogen, protected hydroxy, phosphate group, reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R³³ is hydrogen, hydroxy, halogen, protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support, and optionally, only one of R³² and R³³ is a phosphate group, a reactive phosphorous group, a solid support, a linker, or a linker covalently bonded (e.g., —C(O)CH₂CH₂C(O)—) to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R³² taken together are 4′-C(R¹⁰R¹¹)ᵥ—Y-2′ or 4′-Y—C(R¹⁰R¹¹)ᵥ-2′; Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)_.
   R¹⁰ and R¹¹ independently are H, optionally substituted C₁-C₆alkyl, optionally substituted C₂-C₆alkenyl or optionally substituted C₂-C₆alkynyl;
   R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
   v is 1, 2 or 3;
or R⁴ and R³³ taken together with the atoms to which they are attached form an optionally substituted C₃₋₈cycloalkyl, optionally substituted C₃₋₈cycloalkenyl, or optionally substituted 3-8 membered heterocyclyl;
R³⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10);
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl; and
Q, Z, and m are defined as one of sets (i), (ii) or (iii), wherein
   (i) Q is optionally substituted aryl (e.g., phenyl) or optionally substituted heteroaryl;
      m is an integer selected from 1 to the maximum number of substituents for Q (e.g., when Q is phenyl, then m is 1, 2, 3, 4 or 5, such as 1, 2 or 3; or 1 or 2);
      and each Z is —Z¹, —Z², or —C(R^{C})₃, wherein
         R^{C} is aryl (e.g., phenyl or naphthyl) or heteroaryl, each substituted with one or more Z¹ or Z² groups (e.g., 1, 2, or 3);
      each Z¹ is selected from the group consisting of

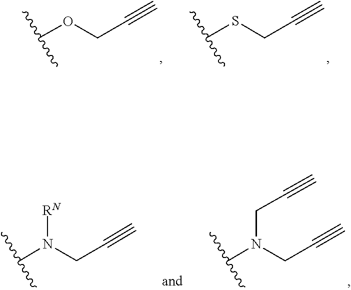

wherein R^{N} is hydrogen or C₁₋₆ alkyl; and
Z² is

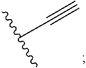 [SMILES: *C#C]

(ii) m is 1;
   Q is —CH₂O—, —CH₂S—, or —CH₂N(R^{N})—, wherein the N, O, or S is bonded to Z;
   and Z is

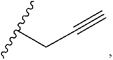 [SMILES: *CC#C]

—(CH₂)₀₋₁—Y—(Z³)ₚ, —C(H)(CH₂Z¹)₂, —CH₂C(H)(CH₂Z¹)₂, or —CH₂C(CH₂Z¹)₃, wherein
Y is optionally substituted aryl or optionally substituted heteroaryl;
each Z³ is Z¹ or Z²; and
p is an integer selected from 1 to the maximum number of substituents for Y (e.g., when Y is phenyl, then p is 1, 2, 3, 4 or 5, such as 1, 2, or 3; or 1 or 2);
or
   (iii) Q is —CH₂N—;
      m is 2;
      and each Z is

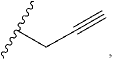 [SMILES: *CC#C]

—(CH₂)₀₋₁—Y—(Z³)ₚ, or —CH₂C(CH₂Z¹)₃.

In some embodiments of any one of the aspects described, R³⁵ is a protected hydroxy (e.g., 4,4′-dimethoxytrityl-protected) or a phosphate group and R³³ is hydroxy or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite).

In some embodiments of any one of the aspects described herein, R³² is hydrogen, hydroxy, halogen, protected hydroxy, optionally substituted C₁₋₃₀ alkyl, optionally substituted C₂₋₃₀alkenyl, optionally substituted C₂₋₃₀alkynyl, optionally substituted C₁₋₃₀alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹).

In some embodiments of any one of the aspects described herein, R³² is hydrogen, hydroxy, fluoro, chloro, methoxy, ethoxy, 2-methoxyethyl, or C₆₋₂₄ alkyl (e.g., n-C₆₋₂₄ alkyl).

In some embodiments of compounds of Formula (IIIc), each Z is selected independently from the group consisting of:

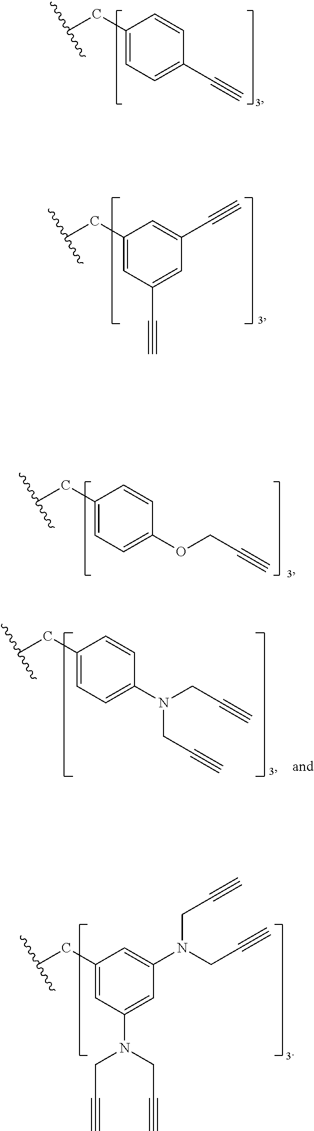

In another aspect, provided herein is a compound of Formula (IV):

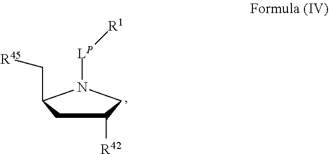

wherein:
L^{P} is absent or a linker;
R¹ is N₃ or

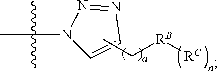

wherein:
a is 0 or 1;
n is 1, 2, 3, 4, or 5;
R^{B} is O, N, S, a heteroalkyl, a branched alkyl, a cycloalkyl, heterocyclyl, aryl or heteroaryl;
each R^{C} independently is

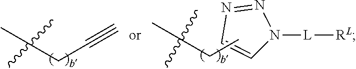

wherein:
each b′ is independently 0 or 1;
each L independently is absent or linker;
each R^{L} is a ligand, (e.g., selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, and polyethylene glycols (PEGs));
R⁴² is hydroxy, halogen, protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R⁴⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10); and
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In another aspect, provided herein is a compound of Formula (IVb):

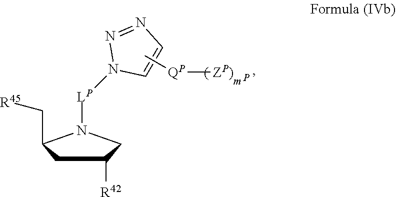

wherein:
L^{P} is absent or a linker;
R⁴² is hydroxy, halogen protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R⁴⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10);
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl; and
Q^{P}, Z^{P}, and m^{P} are defined as one of sets (i), (ii) or (iii), wherein
   (i) Q^{P} is optionally substituted aryl (e.g., phenyl) or optionally substituted heteroaryl;
      m^{P} is an integer selected from 1 to the maximum number of substituents for Q^{P} (e.g., when Q^{P} is phenyl, then m is 1, 2, 3, 4 or 5, such as 1, 2 or 3; or 1 or 2);
      and each Z^{P} is —Z^{P1}, —Z^{P2}, or —C(R^{PC})₃, wherein
         R^{PC} is aryl (e.g., phenyl or naphthyl) or heteroaryl, each substituted with one or more Z^{P1} or Z^{P2} groups (e.g., 1, 2, or 3);
         each Z^{P} is selected from the group consisting of

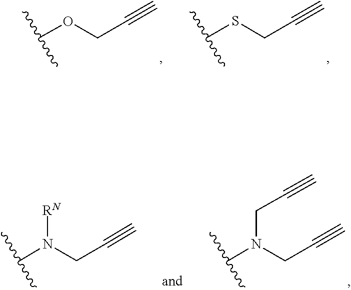

wherein R^{N} is hydrogen or C₁₋₆ alkyl; and
Z^{P2} is

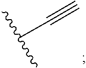 [SMILES: *C#C]

(ii) m^{P} is 1;
   Q^{P} is —CH₂O—, —CH₂S—, or —CH₂N(R^{N})—, wherein the N, O, or S is bonded to Z^{P};
   and Z^{P} is

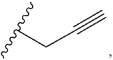 [SMILES: *CC#C]

—(CH₂)₀₋₁—Y—(Z^{P3})ₚₚ, —C(H)(CH₂Z^{P1})₂, —CH₂C(H)(CH₂Z^{P1})₂, or —CH₂C(CH₂Z^{P1})₃, wherein
Y^{P} is optionally substituted aryl or optionally substituted heteroaryl;
each Z^{P3} is Z^{P1} or Z^{P2}; and
pp is an integer selected from 1 to the maximum number of substituents for Y^{P} (e.g., when Y^{P} is phenyl, then pp is 1, 2, 3, 4 or 5, such as 1, 2, or 3; or 1 or 2);
or
   (iii) Q^{P} is —CH₂N—;
      m^{P} is 2;
      and each Z^{P} is

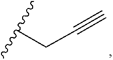 [SMILES: *CC#C]

—(CH₂)₀₋₁—Y—(Z^{P3})ₚₚ, or —CH₂C(CH₂Z^{P1})₃.

In some embodiments of compounds of Formula (IVb), each Z^{P} is selected independently from the group consisting of:

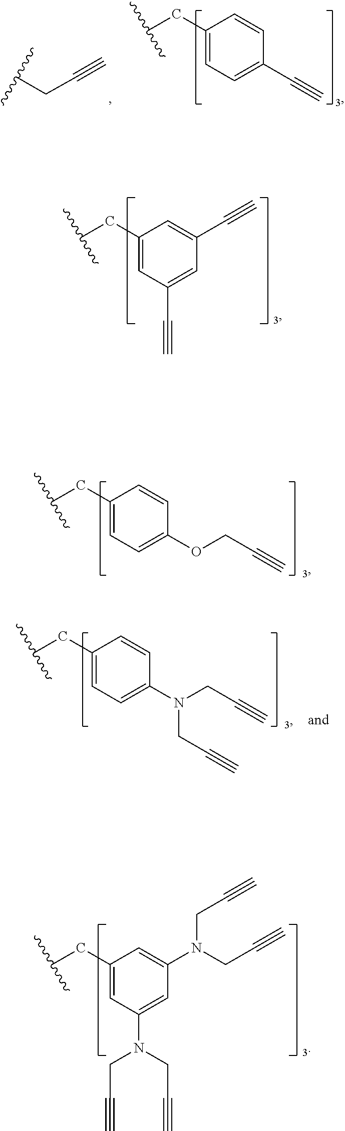

In some embodiments of any one of the aspects described, R⁴² is a hydroxy, protected hydroxy or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite).

In some embodiments of any one of the aspects described herein, R⁴⁵ is a hydroxy, protected hydroxy, vinylphosphonate group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidate, or alkylphosphonate. For example, R⁴⁵ is a hydroxy, protected hydroxy, or vinylphosphonate group.

In some embodiments of any one of the aspects described herein, R⁴² is a hydroxy, protected hydroxy or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite), and R⁴⁵ is a hydroxy, protected hydroxy, vinylphosphonate group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidate, or alkylphosphonate. For example, R⁴² is a hydroxy, protected hydroxy or a reactive phosphorous group, and R⁴⁵ is a hydroxy, protected hydroxy, or vinylphosphonate group

In some embodiments of any one of the aspects described herein, R⁴² is a reactive phosphorous group and R⁴⁵ is a protected hydroxy.

In another aspect, provided herein is a compound of Formula VI, VII, VIII or IX:

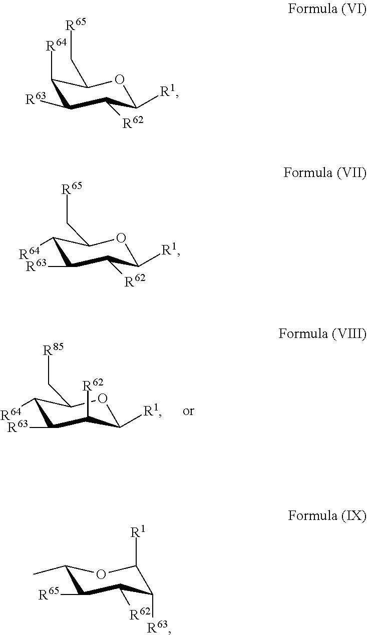

wherein:
R¹ is N₃ or

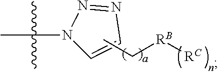

wherein:
a is 0 or 1;
n is 1, 2, 3, 4, or 5;
R^{B} is O, N, S, a heteroalkyl, a branched alkyl, a cycloalkyl, heterocyclyl, aryl or heteroaryl;
each R^{C} independently is

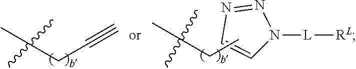

wherein:
each b′ is independently 0 or 1;
each L independently is absent or linker;
each R^{L} is a ligand, (e.g., selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, and polyethylene glycols (PEGs));
R⁶² is hydroxy, protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R⁶³ and R⁶⁴ independently are hydrogen, hydroxy, halogen, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), or —O-lipid;
R⁶⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10); and
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

In some embodiments of any one of the aspects described herein, at least one of R⁶², R⁶³, R⁶⁴ and R⁶⁵ is not a hydroxyl. For example, at least two of R⁶², R⁶³, R⁶⁴ and R⁶⁵ are not hydroxyl at the same time. In some embodiments, R⁶² and R⁶³ are not hydroxyl at the same time. In some embodiments, R⁶² and R⁶⁴ are not hydroxyl at the same time. In some embodiments, R⁶² and R⁶⁵ are not hydroxyl at the same time. In some embodiments, R⁶³ and R⁶⁴ are not hydroxyl at the same time. In some embodiments, R⁶³ and R⁶⁵ are not hydroxyl at the same time. In some embodiments, R⁶⁵ are not hydroxyl at the same time. In some embodiments, at least three of R⁶², R⁶³, R⁶⁴ and R⁶⁵ are not a hydroxyl at the same time. In some embodiments, all four of R⁶², R⁶³, R⁶⁴ and R⁶⁵ are not a hydroxyl at the same time.

In another aspect, provided herein is a compound of Formula VIb, VIIb, VIIIb or IXb.

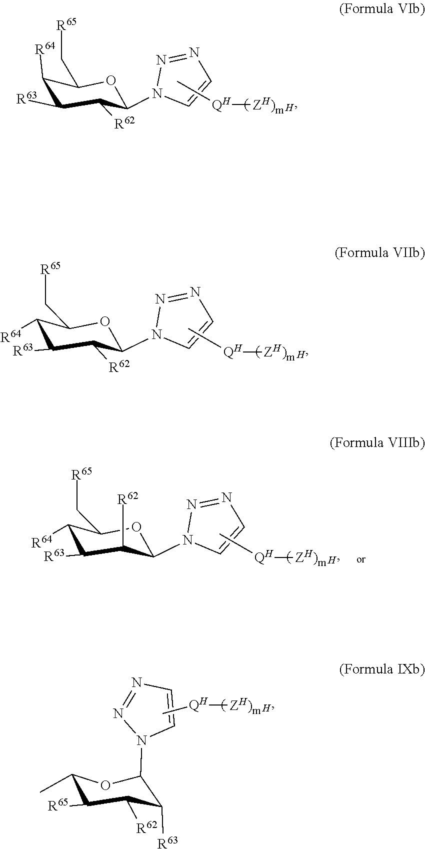

wherein:
R⁶² is hydroxy, protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R⁶³ and R⁶⁴ independently are hydrogen, hydroxy, halogen, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), or —O-lipid;
R⁶⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10); and
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl; and
Q^{H}, Z^{H}, and m^{H} are defined as one of sets (i), (ii) or (iii), wherein
   (i) Q^{H} is optionally substituted aryl (e.g., phenyl) or optionally substituted heteroaryl;
      m^{H} is an integer selected from 1 to the maximum number of substituents for Q^{H} (e.g., when Q^{H} is phenyl, then m is 1, 2, 3, 4 or 5, such as 1, 2 or 3; or 1 or 2);
      and each Z^{H} is —Z^{H1}, —Z^{H2}, or —C(R^{HC})₃, wherein
         R^{H}C is aryl (e.g., phenyl or naphthyl) or heteroaryl, each substituted with one or more Z^{H1} or Z^{H2} groups (e.g., 1, 2, or 3);
         each Z^{H1} is selected from the group consisting of

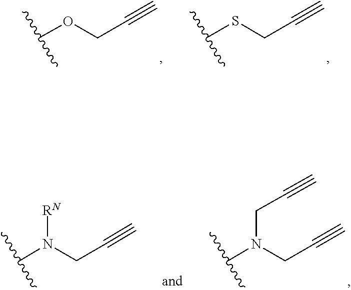

wherein R^{N} is hydrogen or C₁₋₆ alkyl; and
Z^{H2} is

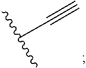 [SMILES: *C#C]

(ii) m^{H} is 1;
   Q^{H} is —CH₂O—, —CH₂S—, or —CH₂N(R^{N})—, wherein the N, O, or S is bonded to Z^{H};
   and Z^{H} is

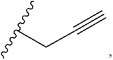 [SMILES: *CC#C]

—(CH₂)₀₋₁—Y—(Z^{H3})ₕₚ, —C(H)(CH₂Z^{P1})₂, —CH₂C(H)(CH₂Z^{H1})₂, or —CH₂C(CH₂Z^{H1})₃, wherein
Y^{H} is optionally substituted aryl or optionally substituted heteroaryl;
each Z^{H3} is Z^{H1} or Z^{H2}; and
hp is an integer selected from 1 to the maximum number of substituents for Y^{H} (e.g., when Y^{H} is phenyl, then hp is 1, 2, 3, 4 or 5, such as 1, 2, or 3; or 1 or 2);
or
   (iii) Q^{H} is —CH₂N—;
      m^{H} is 2
      and each Z^{H} is

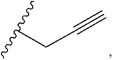 [SMILES: *CC#C]

—(CH₂)₀₋₁—Y—(Z^{H3})ₕₚ, or —CH₂C(CH₂Z^{H1})₃.

In some embodiments of compounds of VIb, VIIb, VIIIb or, each Z^{H} is selected independently from the group consisting of:

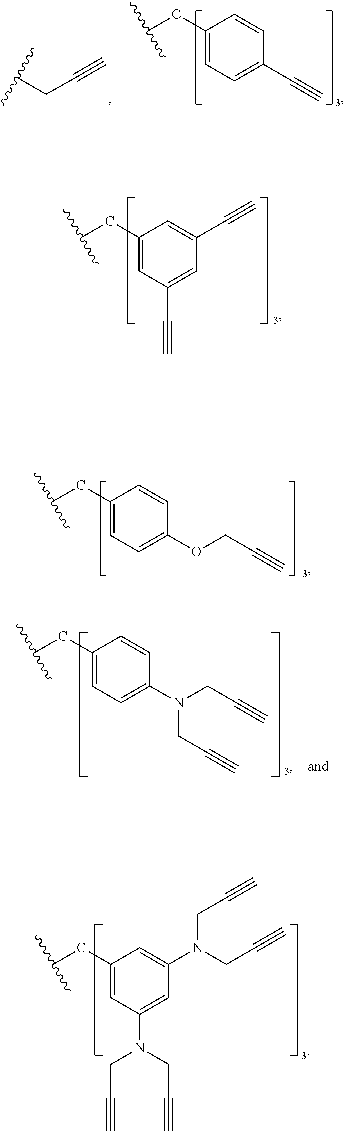

In some embodiments of any one of the aspects described, R⁶² is a hydroxy, protected hydroxy or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite).

In some embodiments of any one of the aspects described herein, R⁶⁵ is a hydroxy, protected hydroxy, vinylphosphonate group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidate, or alkylphosphonate. For example, R⁶⁵ is a hydroxy, protected hydroxy, or vinylphosphonate group.

In some embodiments of any one of the aspects described herein, R⁶² is a hydroxy, protected hydroxy or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite), and R⁶⁵ is a hydroxy, protected hydroxy, vinylphosphonate group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidate, or alkylphosphonate. For example, R⁶² is a hydroxy, protected hydroxy or a reactive phosphorous group, and R⁶⁵ is a hydroxy, protected hydroxy, or vinylphosphonate group

In some embodiments of any one of the aspects described herein, R⁶² is a reactive phosphorous group and R⁶⁵ is a protected hydroxy.

In some embodiments of any one of the aspects described herein, R⁶² is a hydroxy, protected hydroxy or a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite); R⁶³ and R⁶⁴ independently are hydrogen, hydroxy, halogen, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), or —O-lipid; and R⁶⁵ is a hydroxy, protected hydroxy, vinylphosphonate group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidate, or alkylphosphonate. For example, R⁶² is a hydroxy, protected hydroxy or a reactive phosphorous group; R⁶³ and R⁶⁴ independently are hydroxy, protected hydroxy, optionally substituted C₁₋₃₀ alkoxy (e.g., methoxy), alkoxyalkylamine, amino, alkylamino, dialkylamino, or —O-lipid; and R⁶⁵ is a hydroxy, protected hydroxy, or vinylphosphonate group

In some embodiments of any one of the aspects described herein, R⁶² is a reactive phosphorous group; R⁶³ and R⁶⁴ independently are hydroxy, protected hydroxy, optionally substituted C₁₋₃₀ alkoxy (e.g., methoxy), alkoxyalkylamine, amino, alkylamino, dialkylamino, or —O-lipid; and R⁶⁵ is a protected hydroxy.

The compunds of Formulae (III), (IIIa), (IIIb), (IIIc), (IV), (IVb), (VI)-(IX) and (VIb)-(IXb) are useful in the synthesis oligonucleotides. Accordingly, in another aspect, provided herein is an oligonucleotide prepared using a compound of Formula (III), (IIIa), (IIIb), (IIIc), (IV), (IVb), (VI), (VIb), (VII), (VIIb), (VIII), (VIIIb), (IX), or (IXb). For example, an oligonucleotide comprising nucleoside of Formula (I):

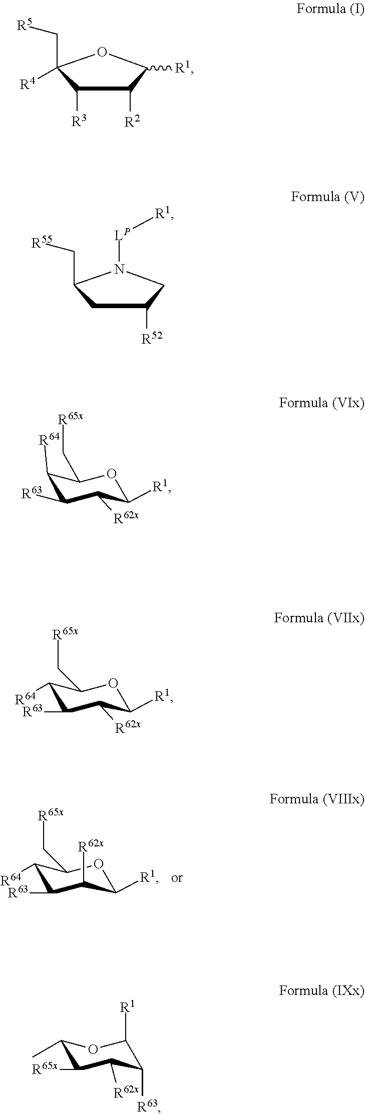

wherein:
L^{P} is absent or a linker;
R¹ is N₃ or

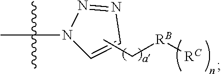

wherein:
a′ is 0 or 1;
n is 1, 2, 3, 4, or 5;
R^{B} is O, N, S, a heteroalkyl, a cycloalkyl, heterocyclyl, aryl or heteroaryl;
each R^{C} independently is

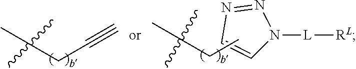

wherein:
   each b′ is independently 0 or 1;
   each L independently is absent or linker;
   each R^{L} is selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, and polyethylene glycols (PEGs);
R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a bond to an internucleotide linkage to a subsequent nucleotide, a 3′-oligonuclotide capping group, a ligand, a linker covalently bonded to one or more ligands, a solid support, a linker or a linker covalently bonded a solid support;
R³, R⁵² and R^{62x} are independently a bond to an internucleotide linkage to a subsequent nucleotide, hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a 3′-oligonuclotide capping group, a ligand, a linker covalently bonded to one or more ligands, a solid support, a linker or a linker covalently bonded to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R² taken together are 4′-C(R¹⁰R¹¹)ᵥ—Y-2′ or 4′-Y—C(R¹⁰R¹¹)ᵥ-2′;
   Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R^{a13})C(O)—, or —C(O)N(R¹²)—.
   R¹⁰ and R¹¹ independently are H, optionally substituted C₁-C₆alkyl, optionally substituted C₂-C₆alkenyl or optionally substituted C₂-C₆alkynyl;
   R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group;
   v is 1, 2 or 3;
or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl;
R⁵, R⁵⁵ and R^{65x} independently represent a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate;
phosphoramidates ((HO)₂(O)P—NH-5′, (HO)(NH₂)(O)P—O-5′), alkylphosphonates [(R^{P})(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, R^{P1} is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
   X is O or S;
   a and b are each independently 1-10;
R⁶³ and R⁶⁴ independently are hydrogen, hydroxy, halogen, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), or —O-lipid;
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl,
provided that,
(i) no more than one of R² and R³ is a bond to an internucleotide linkage to a subsequent nucleotide;
(ii) when both of R² and R³ are not a bond to an internucleotide linkage to a subsequent nucleotide, then R⁵ is a bond to an internucleotide linkage to a preceding nucleotide;
(iii) at least one of R⁵² and R⁵⁵ is a bond to an internucleotide linkage; and
(vi) at least one of R^{62x} and R^{65x} is a bond to an internucleotide linkage.

In the oligonucleotide comprising a nucleoside of Formula (I), at least one of R², R³ and R⁵ is a bond to a internucleotide linkage.

In the oligonucleotide comprising a nucleoside of Formula (V), at least one of R⁴² and R⁴⁵ is a bond to a internucleotide linkage.

In the oligonucleotide comprising a nucleoside of Formula (VIx), (VIIx), (VIIIx) or (IXx), at least one of R^{62x} and R^{65x} is a bond to a internucleotide linkage.

In some embodiments of any one of the aspects described herein, a nucleoside of Formula (I) is of Formula (Ia):

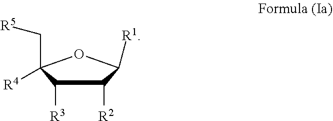

In some embodiments of any one of the aspects described herein, a nucleoside of Formula (I) is of Formula (Ib):

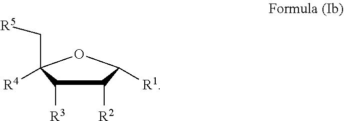

In yet another aspect, provided herein is a double-stranded nucleic acid comprising a first strand and a second strand complementary to the first strand, and wherein at least one of the first and second strand is an oligonucleotide comprising a nucleotide of Formula (I) described herein.

In another aspect, provided herein is a method for inhibiting or reducing the expression of a target gene in a subject. The method comprises administering to the subject: (i) a double-stranded RNA described herein, wherein one of the strands of the dsRNA is complementary to a target gene; and/or (ii) an oligonucleotide described herein, wherein the oligonucleotide is complementary to a target gene.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIGS. 1-3 are schematics for synthesis of multivalent conjugates via CuAAC click chemistry on a pentose sugar. Only β-isomers shown for clarity.

FIG. 4 shows exemplary azide comprising ligands amenable to conjugation by Click chemistry.

FIG. 5 is a schematic showing conjugation via various Click chemistries

FIGS. 6A-6C show various parameters for multiplex ligand conjugation through 1′ Click chemistry—α and β anomers (FIG. 6A), valency (FIG. 6B) and regioisomers of triazoles (FIG. 6C).

FIG. 7 is a schematic representation of the diverse regiochemistry possibilities with a single ligand R.

FIGS. 8A and 8B show some exemplary ligands that are amenable to the invention.

FIG. 9 shows various possible geometries for a single construct. Only β-isomers shown for clarity.

FIG. 10 shows exemplary building blocks.

FIGS. 11-13 are synthetic scheme for synthesis of exemplary building blocks.

FIG. 14 is a ¹H NMR showing 1′-Deoxy Sugar Anomers: α-configuration assignment.

FIG. 15 is a ¹H NMR showing 1′-Deoxy Sugar Anomers: β-configuration assignment.

FIG. 16 is a synthesis scheme showing the synthesis of monovalent and trivalent GalNAc azides.

FIGS. 17-19 are synthesis schemes showing solution chemistry of conjugate building blocks for oligonucleotide synthesis—multiplexing lipid ligands (FIG. 17), multiplexing lipids (FIG. 18) and multiplexing polyamines (FIG. 19).

FIG. 20 depicts exemplary dsRNAs with an exemplary ligand, GalNAc.

FIG. 21 depicts another exemplary dsRNA, where the highlighted (Uhd) nucleoside within a control sense strand is replaced by, for example, the nucleoside structure of one of the boxed nucleotide monomers, “F” refers to a 2′-deoxy-2′-fluoro modified nucleotide, and “OMe” refers to a 2′-methoxy modified nucleotide.

FIG. 22 depicts some exemplary azido-sugar building blocks.

FIG. 23 depicts some exemplary azido-proline building blocks.

FIG. 24 depicts representative multivalent alkynes which are either prepared (18)²³ or commercially available (15-17).

FIG. 25 depicts some exemplary amidites derived from CuAAC between sugar building blocks and multivalent alkynes: ready for click chemistry on solid supports

FIG. 26 depicts some exemplary CPGs derived from CuAAC between sugar building blocks and multivalent alkynes: ready for click chemistry on solid supports.

FIG. 27 depicts some exemplary amidites derived from RuAAC between sugar building blocks and multivalent alkynes: ready for click chemistry on solid supports

FIG. 28 depicts some exemplary CPGs derived from RuAAC between sugar building blocks and multivalent alkynes: ready for click chemistry on solid supports.

FIG. 29 depicts some exemplary products derived from CuAAC between alkyne monomers shown in FIG. 25 and various azides (FIGS. 8A and 8B). All triazoles are 1,4-regioisomers.

FIG. 30 depicts some exemplary products derived from RuAAC between alkyne monomers shown in FIG. 28 and various azides (FIGS. 8A and 8B). All triazoles are 1,5-regioisomers.

FIG. 31 depicts some exemplary products derived from RuAAC between alkyne monomers shown in FIG. 25 and various azides (FIGS. 8A and 8B). Combination of 1,4- and 1,5-regioisomers.

FIG. 32 depicts some exemplary products derived from CuAAC between alkyne monomers shown in FIG. 28 and various azides (FIGS. 8A and 8B). Combination of 1,4- and 1,5-regioisomers.

FIG. 33 depicts some exemplary compounds derived from CuAAC between GalNA-azides 3/4 (shown in FIG. 22) and mono-, bi-, tri-valent alkyne building blocks (FIG. 24).

FIG. 34 depicts some exemplary compounds derived from CuAAC between FuNA-azides 6 (shown in FIG. 22) and mono-, bi-, tri-valent alkyne building blocks (FIG. 24).

FIG. 35 depicts some exemplary compounds derived from CuAAC between GluNA-azides 7/8 (shown in FIG. 22) and mono-, bi-, tri-valent alkyne building blocks (FIG. 24).

FIG. 36 depicts some exemplary compounds derived from CuAAC between ManNA-azides 10/11 (shown in FIG. 22) and mono-, bi-, tri-valent alkyne building blocks (FIG. 24).

FIG. 37A depicts immobilized Cu(I) ion on a solid support.

FIG. 37B depicts immobilized Ru(III) ion on a polymer support.

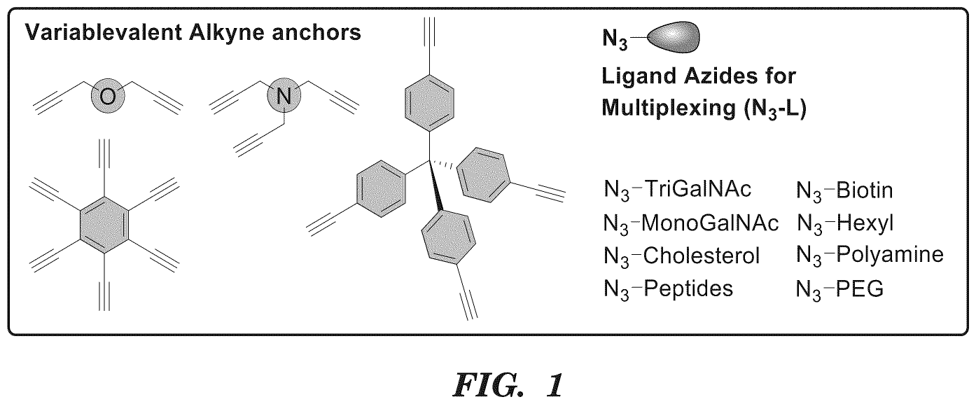

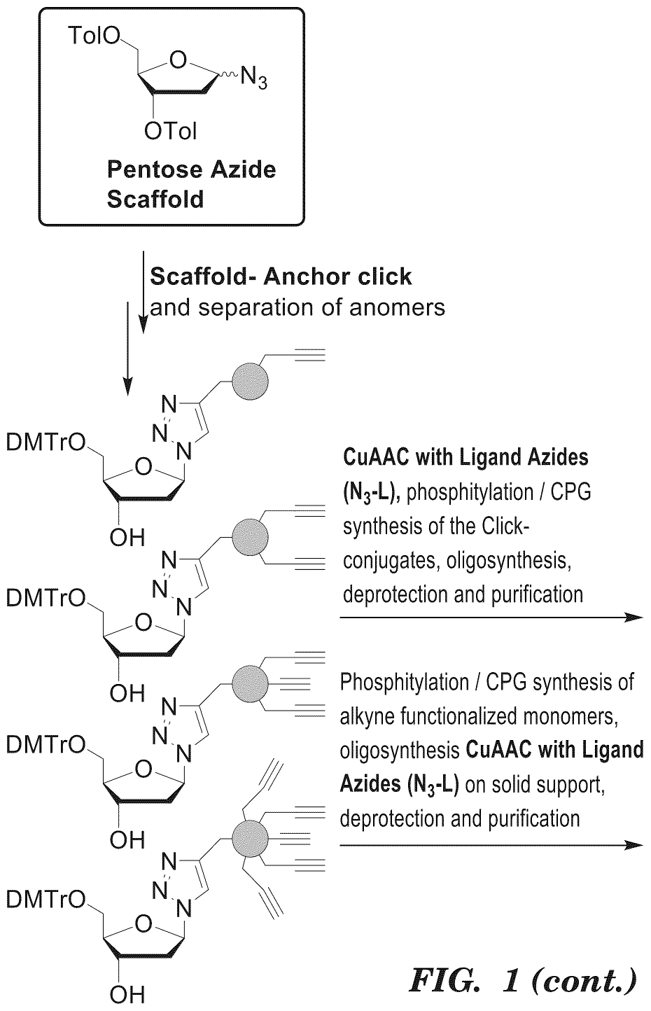

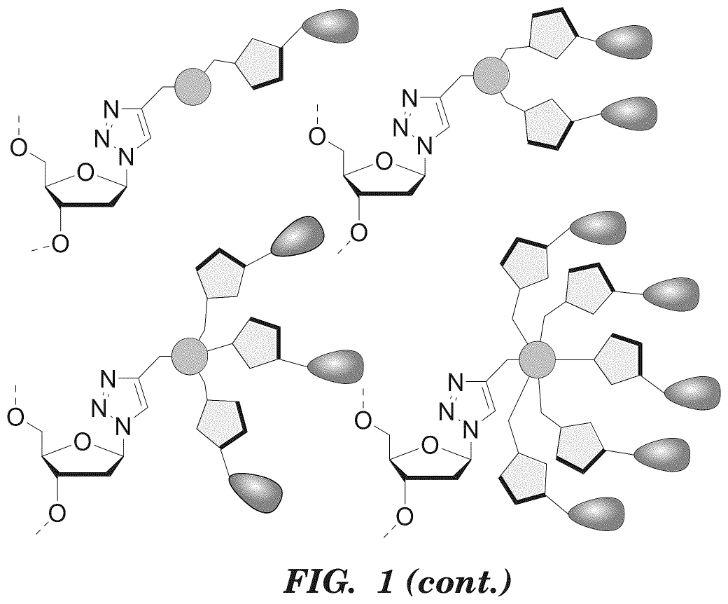

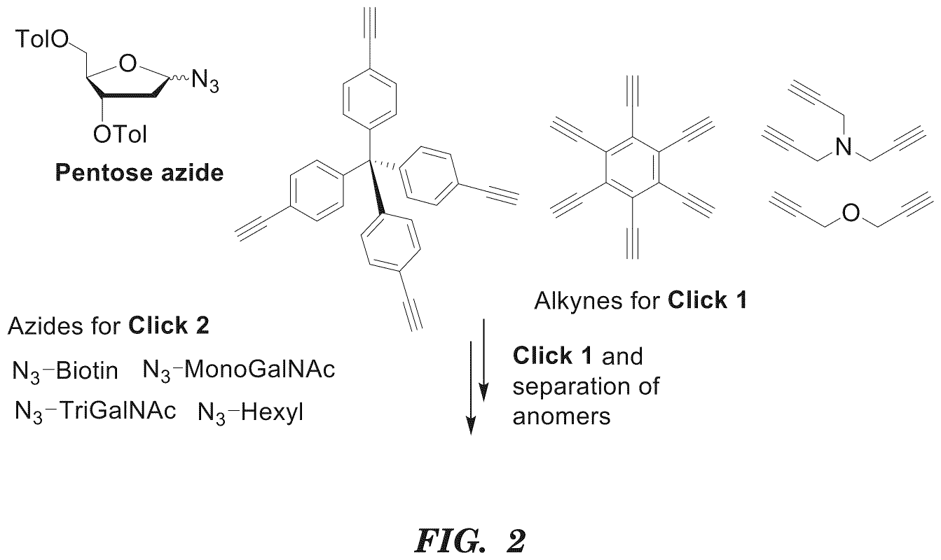

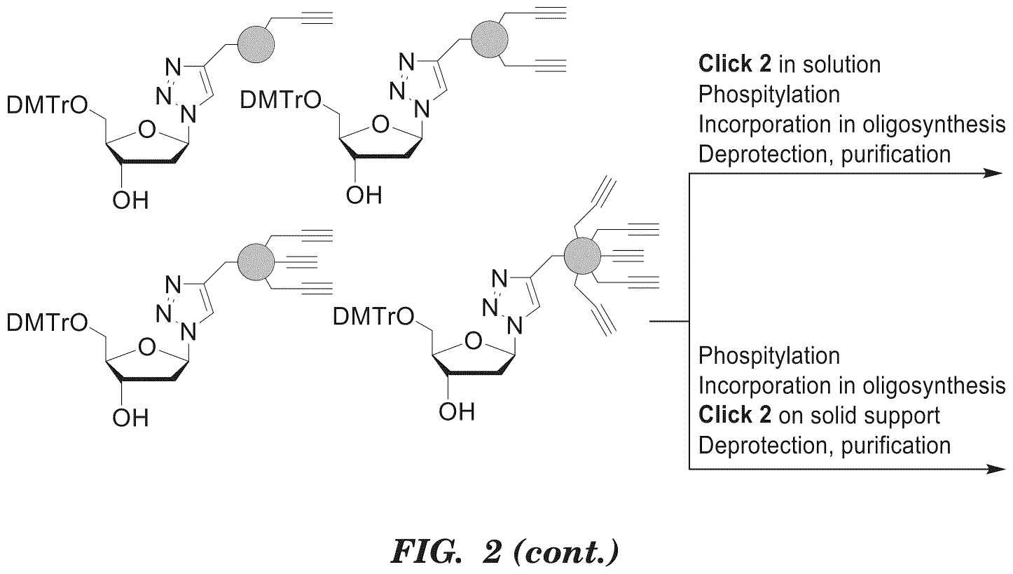

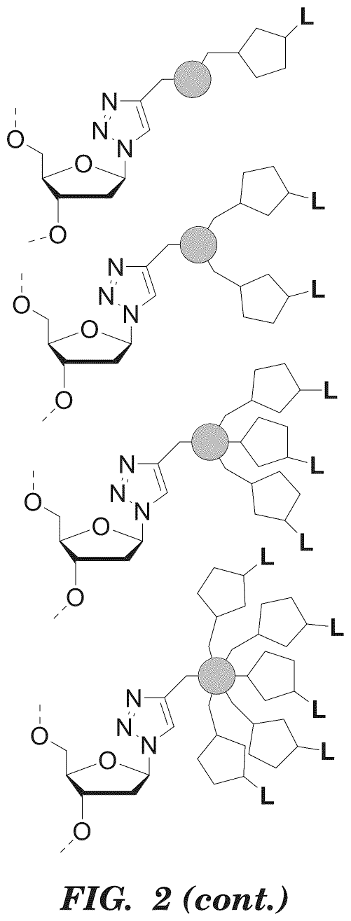

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

In one aspect, provided herein is a compound of Formula (III):

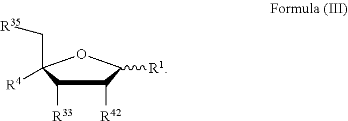

In another aspect provided herein is a compound of Formula (IIIc):

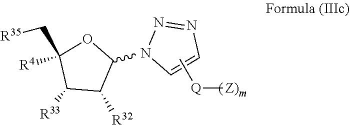

In another aspect, provided herein is an oligonucleotide comprising nucleoside of Formula (I):

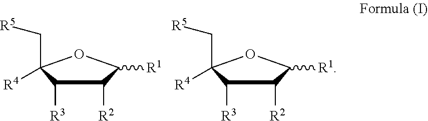

In the various aspects described herein, R¹ can be N₃ or

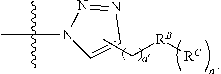

In some embodiment so the various aspects described herein, R¹ is

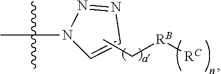

where a′ can be 0 or 1. In some embodiments, a′ is 0. In some other embodiments, a′ is 1.

It is noted that the —(CH₂)ₐ·R^{B}(R^{C})ₙ group can be attached to the triazole group at the 4- or 5-position. Accordingly, in some embodiments of any one of the aspects, R¹ is

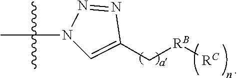

In some other embodiments of any one of the aspects, R¹ is

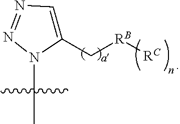

In the various aspects described herein, R^{B} can be O, N, S, heteroalkyl, a a cycloalkyl, heterocyclyl, aryl or heteroaryl. In some embodiments of any one of the aspects, R^{B} is O, N, heteroalkyl or aryl. For example, R^{B} can be O, N, C(CH₂O—)₄ or benzyl. In some embodiments, R^{B} is O. In some other embodiments, R^{B} is N. In yet some other embodiments, R^{B} is C(CH₂O—)₄. In still some other embodiments, R^{B} is benzyl. In some embodiments, R^{B} is

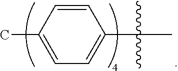

In the various aspects described herein, n can be 1, 2, 3, 4 or 5. In some embodiments of any one of the aspects described herein, n is 1. In some other embodiments of any one of the aspects described herein, n is 2. In yet some other embodiments of any one of the aspects described herein, n is 3. In still some other embodiments of any one of the aspects described herein, n is 4. In still yet some other embodiments of any one of the aspects described herein, n is 5.

In some embodiments of any one of the aspects described herein n is 1 and R^{B} is O.

In some embodiments of any one of the aspects described herein n is 2 and R^{B} is N.

In some embodiments of any one of the aspects described herein n is 3 and R^{B} is C(CH₂O—)₄.

In some embodiments of any one of the aspects described herein n is 5 and R^{B} is benzyl.

In some embodiments, R^{B} is phenyl.

In the various aspects described herein, each R^{C} independently can be

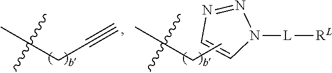

or or -LR^{L}, where each b′ can be independently 0 or 1. In some embodiments, b′ is 0. In one other embodiments b′ is 1.

In some embodiments, R^{C} is

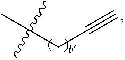

where b′ is 0 or 1.

In some embodiments, R^{C} is

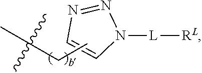

wherein b′ is 0 or 1. It is noted that the triazole group of each R^{C} can be attached to R^{B} via the 4- or 5-position of the triazole. Accordingly, R^{C} can be

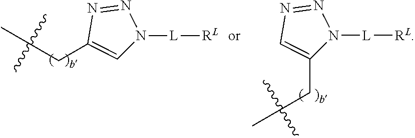

In some embodiments, b′ is 0. Accordingly, in some embodiments of any one of the aspects described herein, each R^{C} is —CH₂C≡C

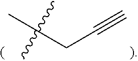 [SMILES: *CC#C]

In some other embodiments of any one of the aspects described herein, each R^{C} is

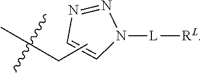

It is noted that the triazole group of each R^{C} can be attached to R^{B} via the 4- or 5-position of the triazole. Accordingly, in some embodiments of any one of the aspects, R^{C} is

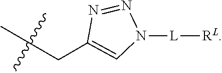

In some other embodiments of any one of the aspects, R^{C} is

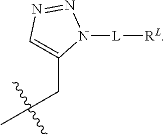

### R^{L}

Embodiments of the various aspects described herein include the group R^{L}. Each R^{L} can be independently selected from the groups consisting of H, carbohydrates, lipids, vitamins, peptides, proteins, lipoproteins, peptidomimetics, polyamines, nucelsides and nucleotides, oligonucleotides, detectable labels, diagnostic agents (e.g., bitoin), fluorescent dyes, polyethylene glycols (PEGs), antibodies, antibody fragments (e.g., nanobodies).

In some embodiments of any one of the aspects described herein, R^{L} is a ligand. Without wishing to be bound by a theory, ligands modify one or more properties of the attached molecule (e.g., the oligonucleotide described herein) including but not limited to pharmacodynamic, pharmacokinetic, binding, absorption, cellular distribution, cellular uptake, charge and clearance. Ligands are routinely used in the chemical arts and are linked directly or via an optional linking moiety or linking group to a parent compound. A preferred list of ligands includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred ligands amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]2, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

(SEQ ID NO. 9)
AALEALAEALEALAEALEALAEAAAAGGC (GALA);
(SEQ ID NO. 10)
AALAEALAEALAEALAEALAEALAAAAGGC (EALA);
(SEQ ID NO. 11)
ALEALAEALEALAEA;
(SEQ ID NO. 12)
GLFEAIEGFIENGWEGMIWDYG (INF-7);
(SEQ ID NO. 13)
GLFGAIAGFIENGWEGMIDGWYG (Inf HA-2);
(SEQ ID NO. 14)
GLFEAIEGFIENGWEGMIDGWYGCGLFEAIEGFIENGWEGMID GWYGC (diINF-7);
(SEQ ID NO. 15)
GLFEAIEGFIENGWEGMIDGGCGLFEAIEGFIENGWEGMIDGGC (diINF-3);
(SEQ ID NO. 16)
GLFGALAEALAEALAEHLAEALAEALEALAAGGSC (GLF);
(SEQ ID NO. 17)
GLFEAIEGFIENGWEGLAEALAEALEALAAGGSC (GALA-INF3);
(SEQ ID NO.18)
GLF EAI EGFI ENGW EGnI DG K GLF EAI EGFI ENGW EGnI
DG (INF-5, n is norleucine);
(SEQ ID NO. 19)
LFEALLELLESLWELLLEA (JTS-1);
(SEQ ID NO. 20)
GLFKALLKLLKSLWKLLLKA (ppTG1);
(SEQ ID NO. 21)
GLFRALLRLLRSLWRLLLRA (ppTG20);
(SEQ ID NO. 22)
WEAKLAKALAKALAKHLAKALAKALKACEA (KALA);
(SEQ ID NO. 23)
GLFFEAIAEFIEGGWEGLIEGC (HA);
(SEQ ID NO. 24)
GIGAVLKVLTTGLPALISWIKRKRQQ (Melittin);
(SEQ ID NO. 25)
H₅WYG;
and
(SEQ ID NO. 26)
CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also refered to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to,

(SEQ ID NO. 27)
RQIKIWFQNRRMKWKK (penetratin);
(SEQ ID NO. 28)
GRKKRRQRRRPPQC (Tat fragment 48-60);
(SEQ ID NO. 29)
GALFLGWLGAAGSTMGAWSQPKKKRKV (signal sequence based peptide);
(SEQ ID NO. 30)
LLIILRRRIRKQAHAHSK (PVEC);
(SEQ ID NO. 31)
GWTLNSAGYLLKINLKALAALAKKIL (transportan);
(SEQ ID NO. 32)
KLALKLALKALKAALKLA (amphiphilic model peptide);
(SEQ ID NO. 33)
RRRRRRRRR (Arg9);
(SEQ ID NO. 34)
KFFKFFKFFK (Bacterial cell wall permeating peptide);
(SEQ ID NO. 35)
LLGDFFRKSKEKIGKEFKRIVQRIKDFLRNL VPRTES (LL-37);
(SEQ ID NO. 36)
SWLSKTAKKLENSAKKRISEGIAIAIQGGPR (cecropin P1);
(SEQ ID NO. 37)
ACYCRIPACIAGERRYGTCIYQGRLWAFCC (α-defensin);
(SEQ ID NO. 38)
DHYNCVSSGGQCLYSACPIFTKIQGTCYRGKAKCCK (β-defensin);
(SEQ ID NO. 39)
RRRPRPPYLPRPRPPPFFPPRLPPRIPPGFPPRFPPRFPGKR-NH2 (PR-39);
(SEQ ID NO. 40)
ILPWKWPWWPWRR-NH2 (indolicidin);
(SEQ ID NO. 41)
AAVALLPAVLLALLAP (RFGF);
(SEQ ID NO. 42)
AALLPVLLAAP (RFGF analogue);
and
(SEQ ID NO. 43)
RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc2 and GalNAc3; D-mannose, multivalent mannose, multivalent lactose, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of oligonucleotides described herein. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2,4,6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleoside linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, scuh as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In some embodiments of any one of the aspects, the ligand has a structure shown in any of Formula (IV)-(VII):

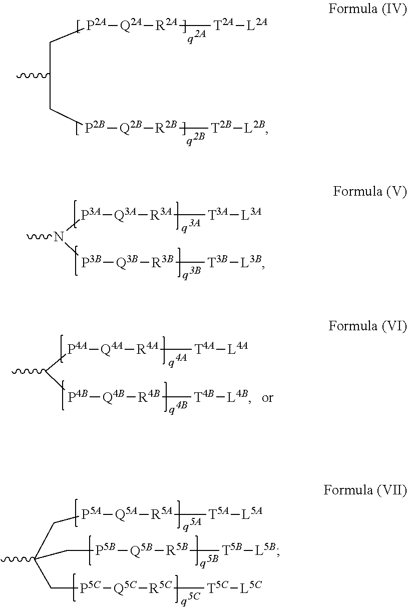

wherein:
   q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{5A}, T^{5B}, T^{5C} are each independently for each occurrence absent, CO, NH, O, S, OC(O), NHC(O), CH₂, CH₂NH or CH₂O;
   Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
   R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

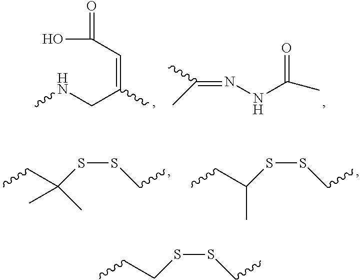

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and
R^{a} is H or amino acid side chain.

In some embodiments of any one of the aspects, the ligand is of Formula (VII):

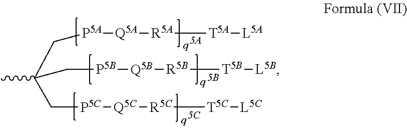

wherein L^{5A}, L^{5B} and L^{5C} represent a monosaccharide, such as GalNAc derivative.

Exemplary ligands include, but are not limited to, the following:

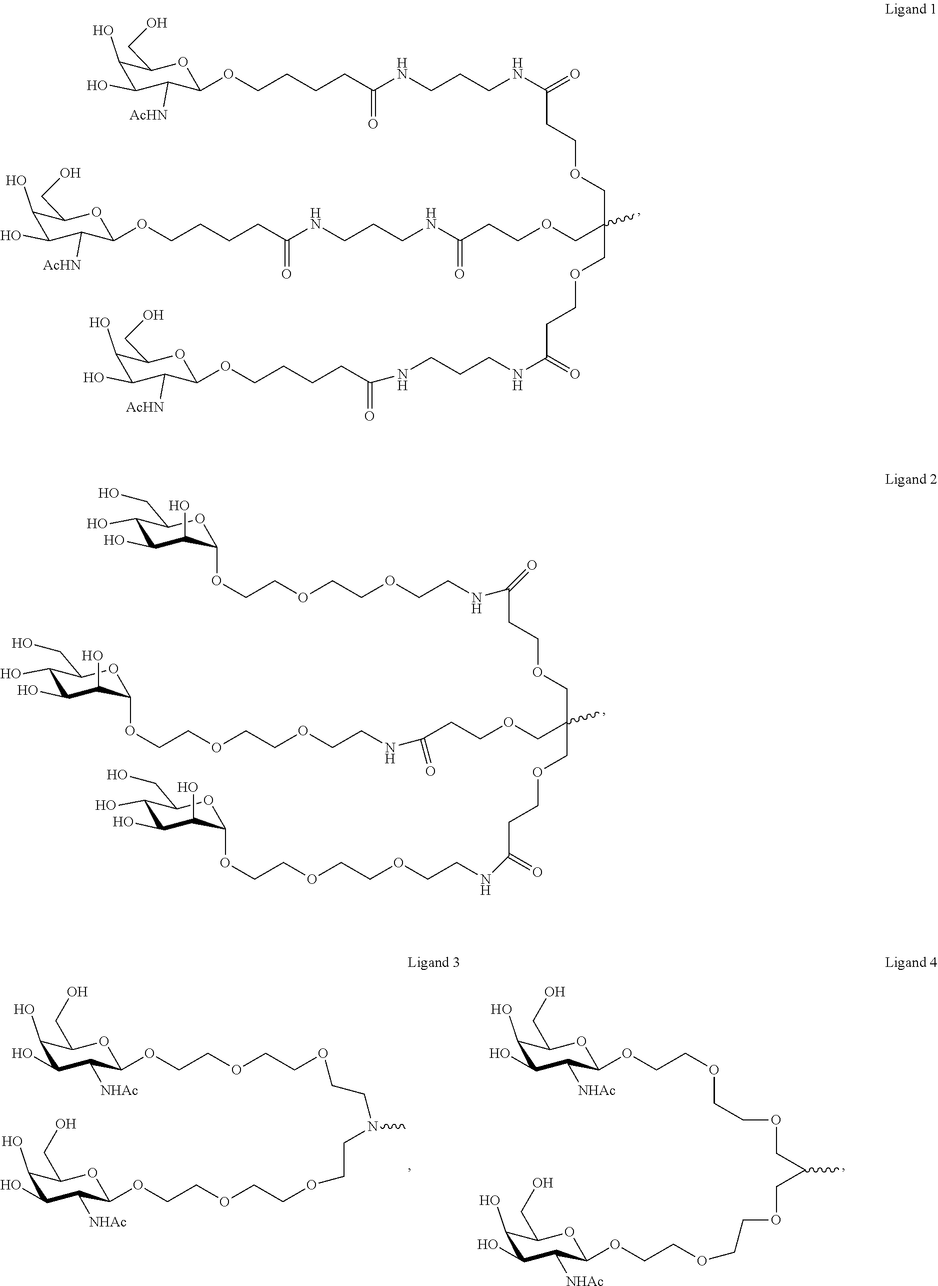

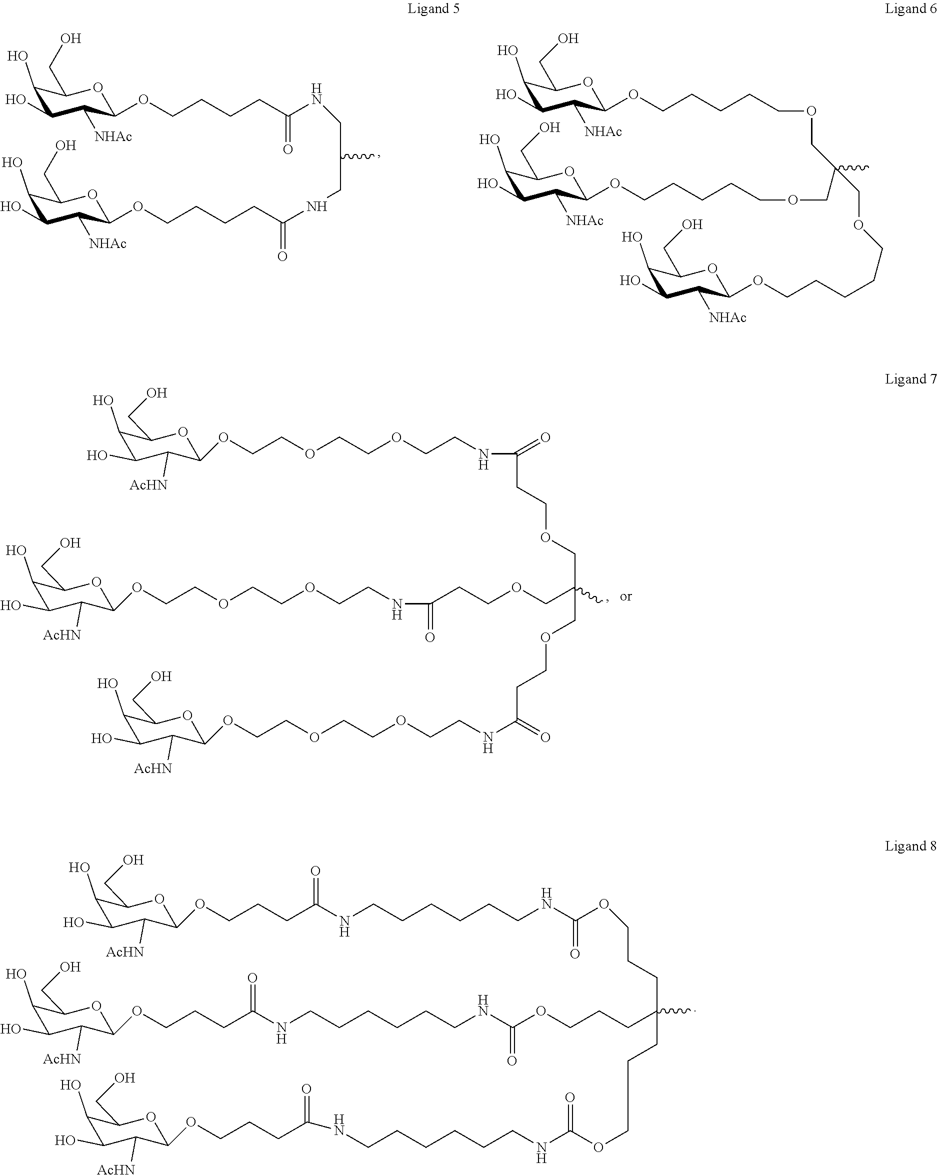

In some embodiments of any one of the aspects described herein, the ligand is a ligand described in U.S. Pat. No. 5,994,517 or U.S. Pat. No. 6,906,182, content of each of which is incorporated herein by reference in its entirety.

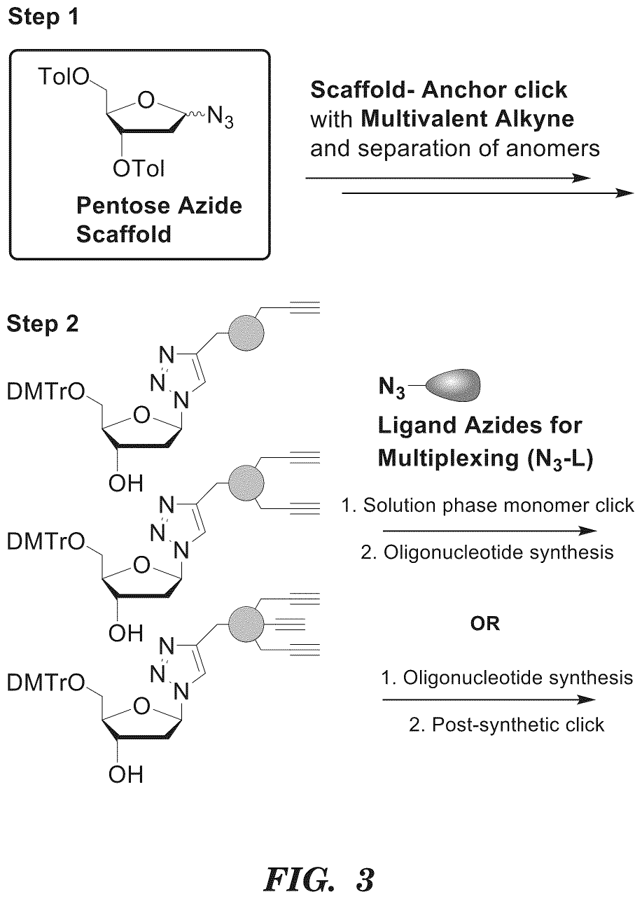

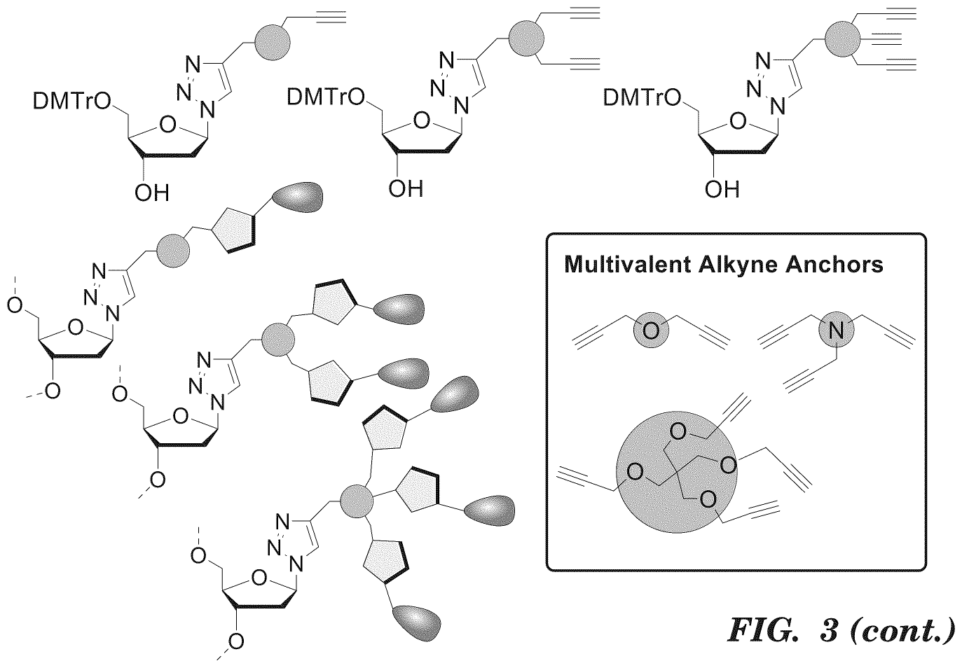

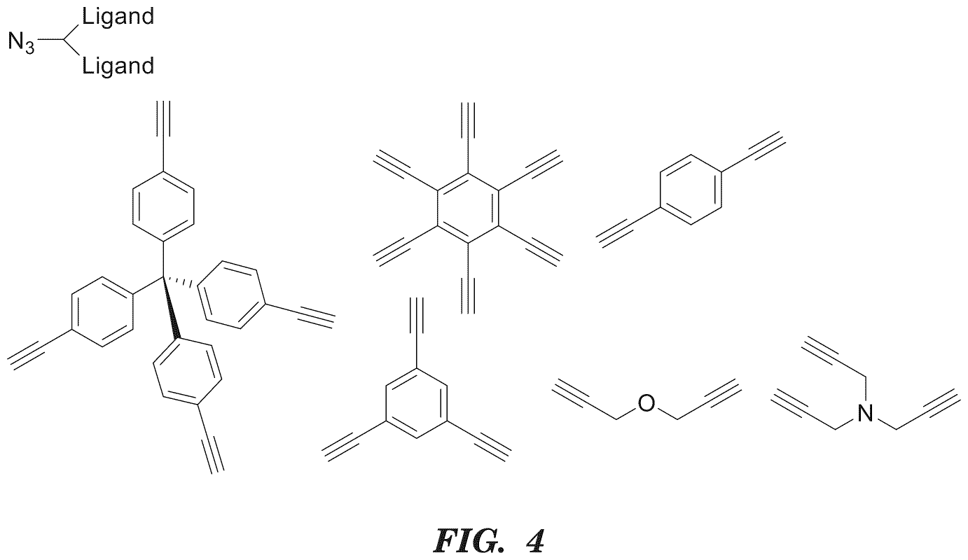

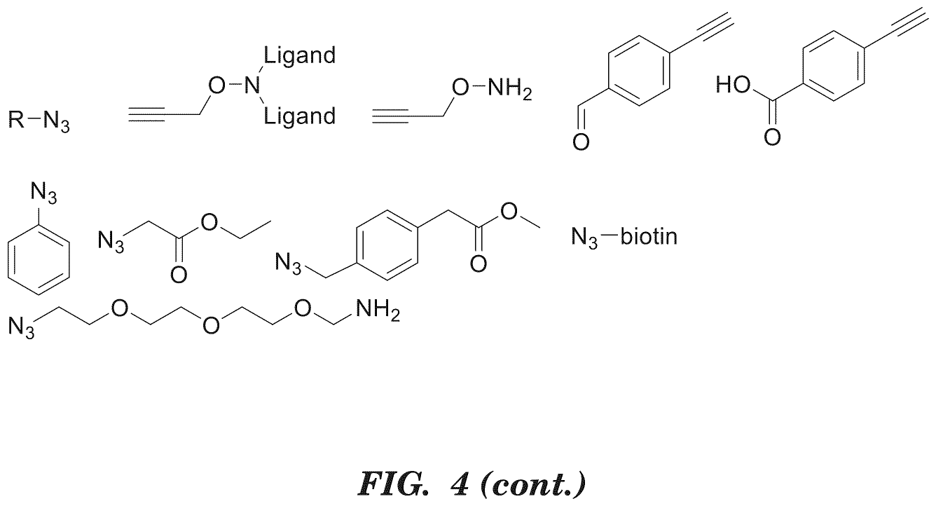

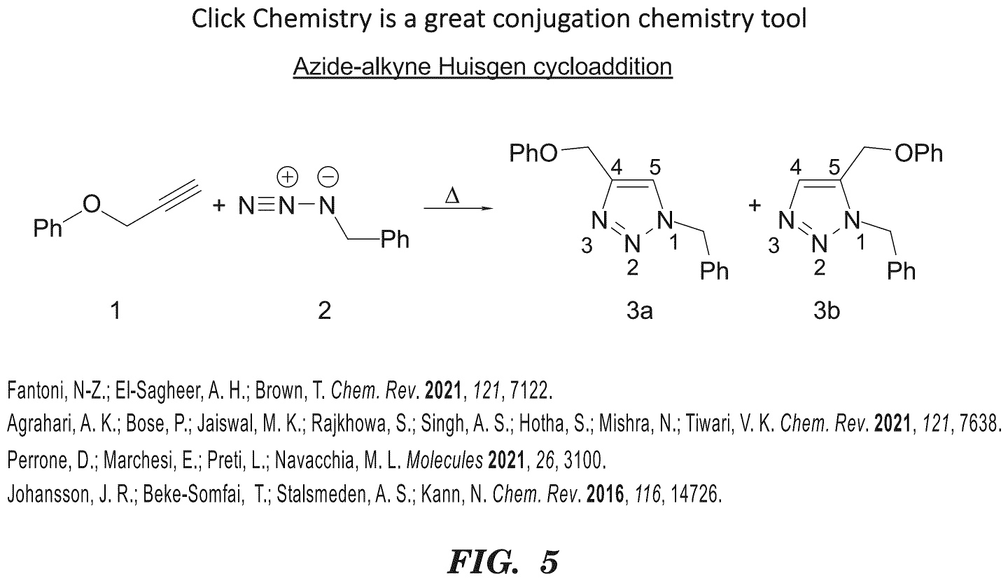

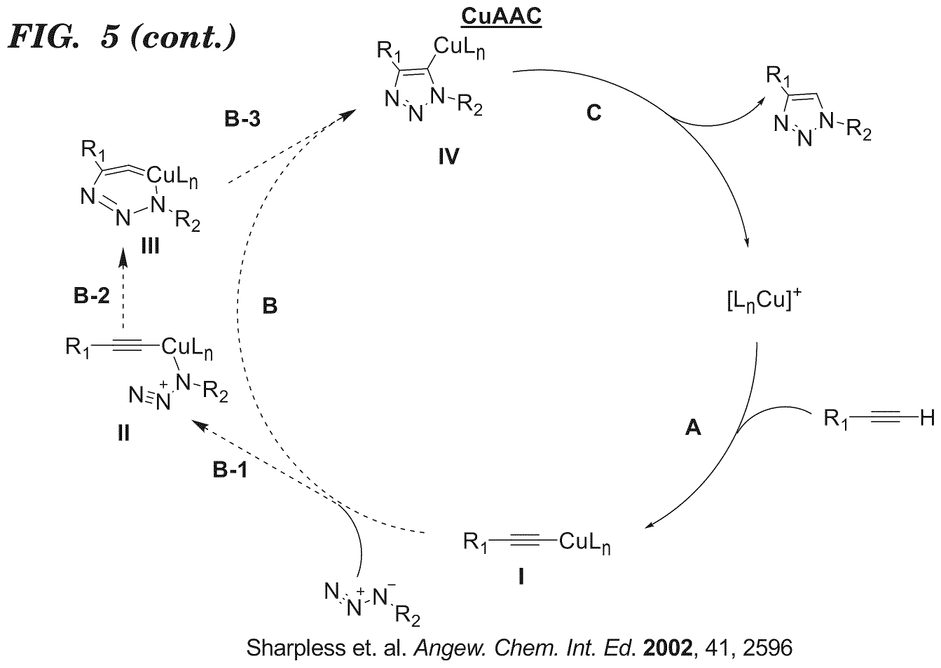

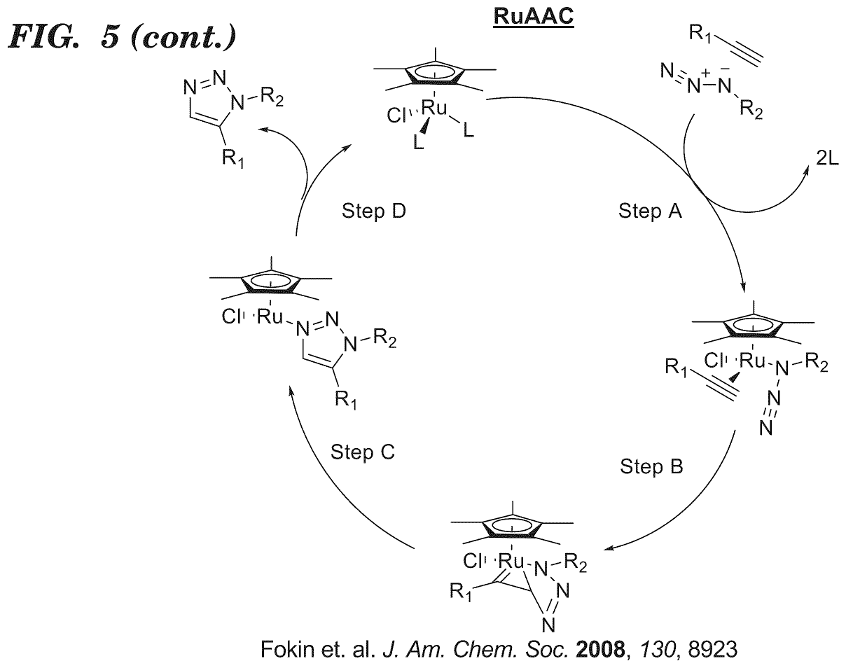

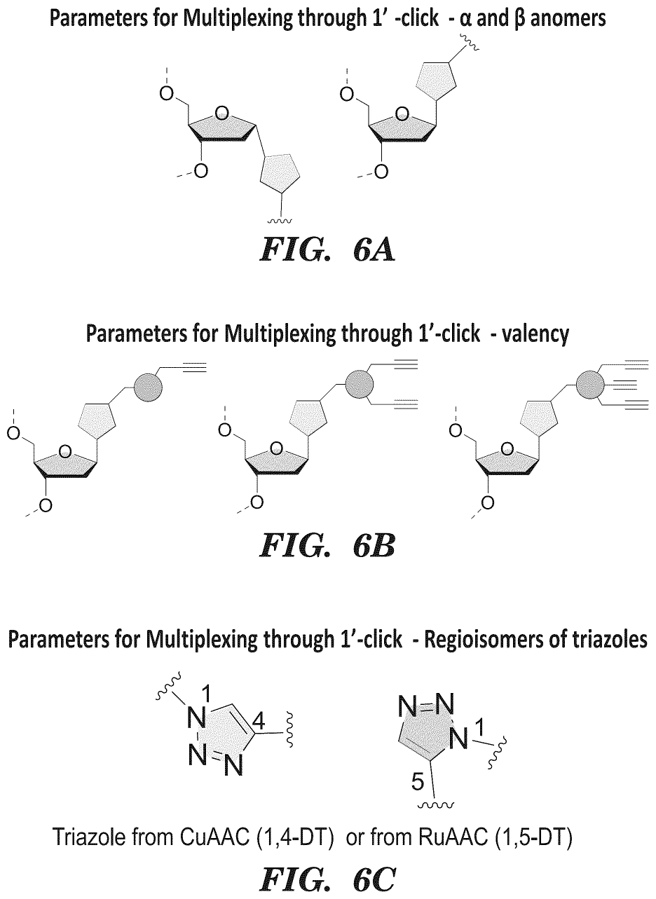

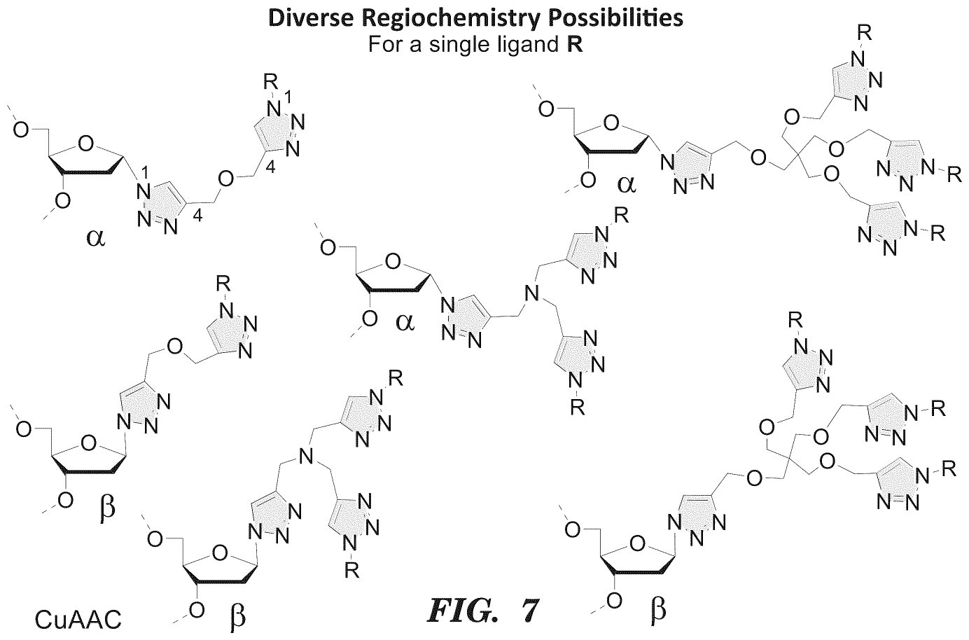

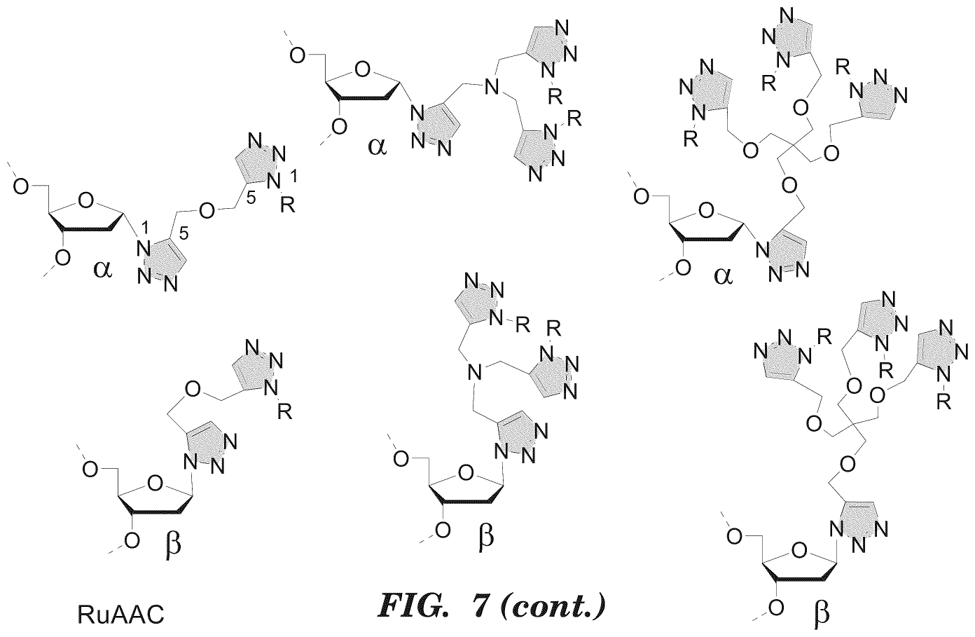

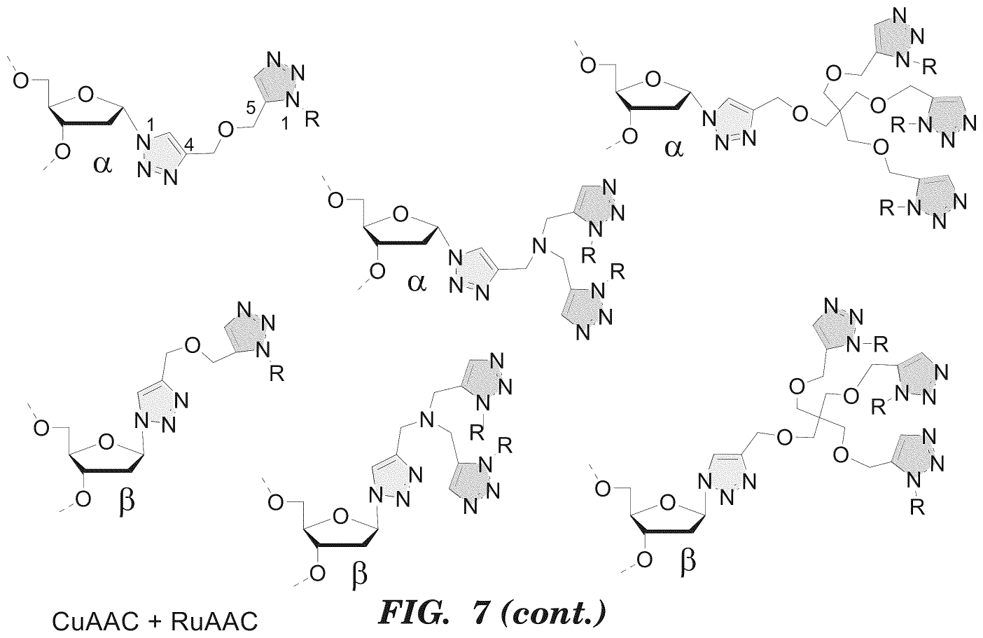

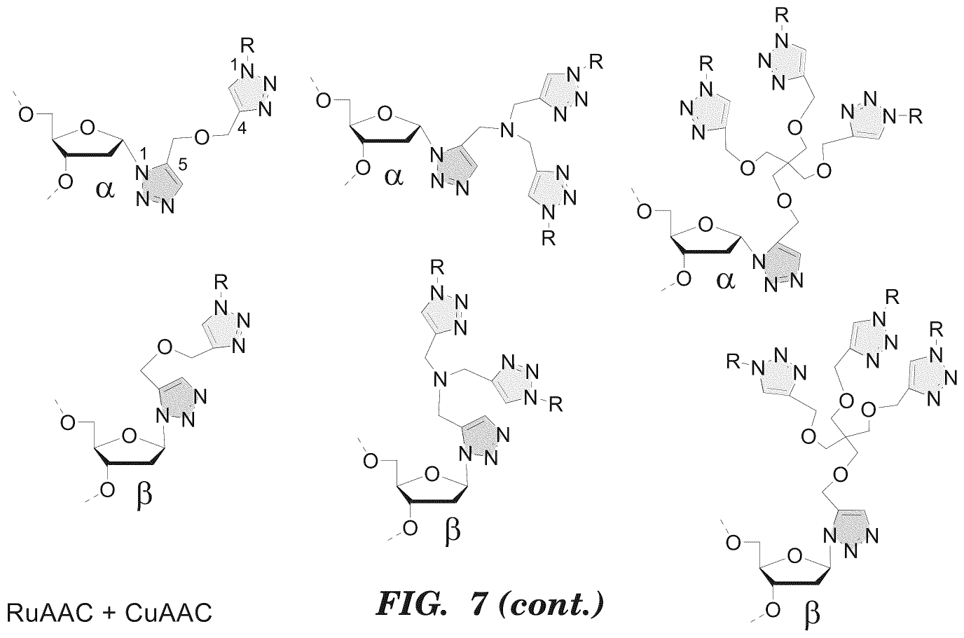

In some embodiments, the ligand can be a tri-antennary ligand described in FIG. 3 of U.S. Pat. No. 6,906,182. For example, the ligand is selected from the following tri-antennary ligands:

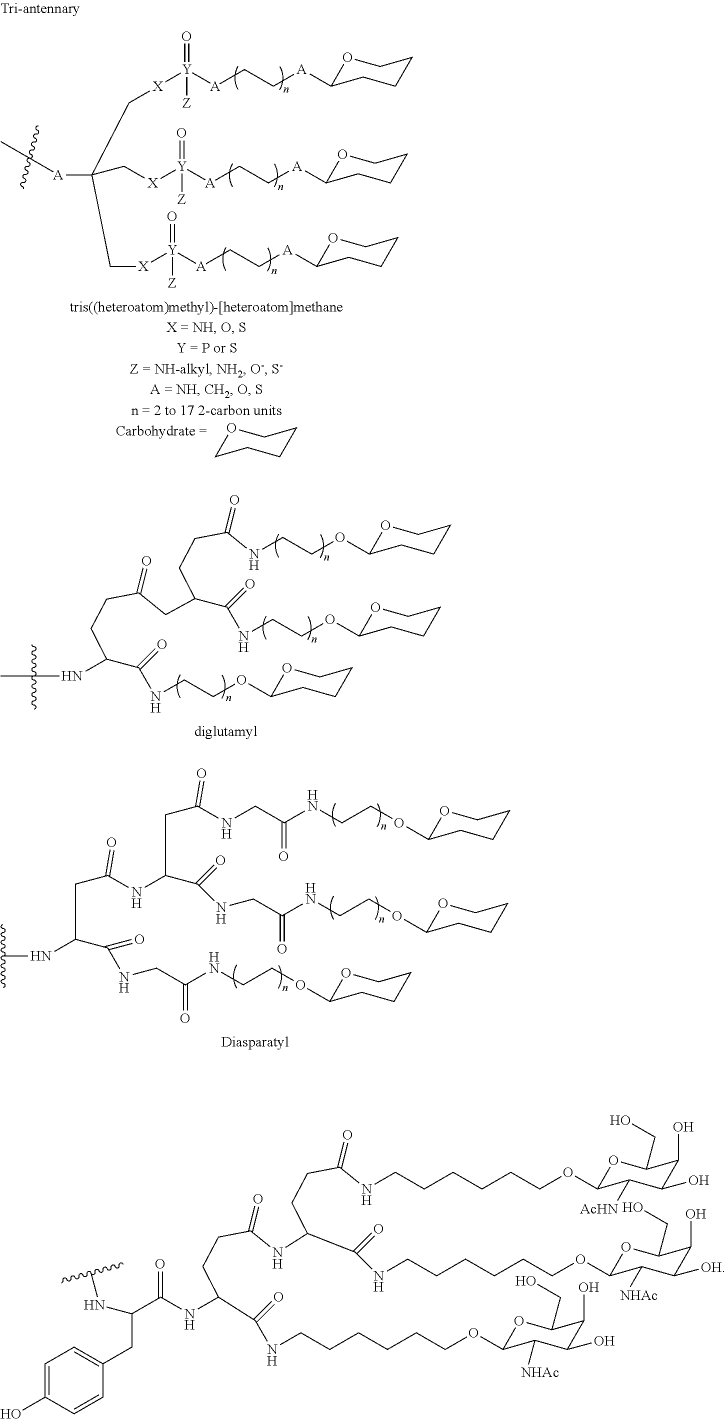

In some embodiments of any one of the aspects described herein, R^{L} is a ligand. It is noted that when more than one R^{L} are present, they can be same or different. Accordingly, in some embodiments of any one of the aspects described herein, all R^{L} are same. In some other embodiments of any one of the aspects described herein, R^{L} are different.

### R²

In some embodiments of any one of the aspects described herein, R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R² is hydrogen, hydroxy, protected hydroxy, halogen, optionally substituted C₁₋₃₀ alkoxy, alkoxyalkyl (e.g., methoxyethyl), alkoxyalkylamine, alkoxyoxycarboxylate, amino, alkylamino, or dialkylamino.

In some embodiments of any one of the aspect, R² is hydrogen, hydroxy, protected hydroxy, halogen, optionally substituted C₁₋₃₀ alkoxy, or alkoxyalkyl (e.g., methoxyethyl. In some embodiments of any one of the aspects, R² is hydrogen, hydroxy, protected hydroxy, fluoro or methoxy.

In some embodiments of any one of the aspects R² is halogen. For example, R² can be fluoro, chloro, bromo or iodo. In some embodiments of any one of the aspects described herein, R² is fluoro.

In some embodiments of any one of the aspects described herein, R² and R⁴

In some embodiments of any one of the aspects described herein, R² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects, v is 1. In some other embodiments of any one of the aspects, v is 2.

In some embodiments, Y is O. For example, R² and R⁴ taken together are 4′-C(R¹⁰R¹¹)ᵥ—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃.

In some embodiments of any one of the aspects, R¹⁰ and R¹¹ attached to the same C are the same. For example, R¹⁰ and R¹¹ attached to the same C are H.

In some embodiments of any one of the aspects, R² and R⁴ taken together are 4′-CH₂—O-2′, 4′-CH(CH₃)—O-2′,4′-CH(CH₂OCH₃)—O-2′, or 4′-CH₂CH₂—O-2′. For example, R² and R⁴ taken together are 4′-CH₂CH₂—O-2′.

In some embodiments of any one of the aspects described herein, R² is a bond to an internucleotide linkage to a subsequent nucleotide. It is noted that only one of R² and R³ can be a bond to an internucleotide linkage to a subsequent nucleotide.

### R³

In some embodiments of any one of the aspects described herein, R³ can be a bond to an internucleotide linkage to a subsequent nucleotide, hydroxy, protected hydroxy, optionally substituted C₁₋₃₀ alkoxy, halogen, alkoxyalkyl (e.g., methoxyethyl), amino, alkylamino, dialkylamino, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support.

In some embodiments of any one of the aspects described herein, R³ is a bond to an internucleotide linkage to a subsequent nucleotide, hydroxy, protected hydroxy, optionally substituted C₁₋₃₀ alkoxy, a 3′-oligonuclotide capping group (e.g., an inverted nucleotide or an inverted abasic nucleotide), a solid support, or a linker covalently bonded (e.g., —C(O)CH₂CH₂C(O)—) to a solid support. For example, R³ is a bond to an internucleotide linkage to a subsequent nucleotide, hydroxy, a solid support, or a linker covalently bonded (e.g., —C(O)CH₂CH₂C(O)—) to a solid support. In some embodiments of any one of the aspects described herein, R³ is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R³ is a bond to an internucleotide linkage to a subsequent nucleotide.

In some embodiments of any one of the aspects described herein, R³ is a solid support, or a linker covalently bonded to a solid support.

In some embodiments of any one of the aspects described herein, R³ is hydroxyl.

In some embodiments of any one of the aspected described herein, R³ and R⁴ taken together with the atoms to which they are attached form an optionally substituted C₃-8cycloalkyl, optionally substituted C₃₋₈cycloalkenyl, or optionally substituted 3-8 membered heterocyclyl.

### R⁴

In some embodiments of any one of the aspects described herein, R⁴ can be hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ can be hydrogen, optionally substituted C₁₋₆alkyl or optionally substituted C₁₋₆alkoxy.

In some embodiments of any one of the aspects described herein, R⁴ is H.

### R⁵

In some embodiments of any one of the aspects described herein, R⁵ can be a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10).

In some embodiments of any one of the aspects described herein, R⁵ can be a bond to an internucleotide linkage to a preceding nucleotide, hydroxy, protected hydroxy, optionally substituted C₂₋₃₀alkenyl, optionally substituted C₂₋₃₀alkynyl, optionally substituted C₁₋₃₀ alkoxy, vinylphosphonate (VP) group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidates, or alkylphosphonates.

In some embodiments of any one of the aspects described herein, R⁵ is a bond to an internucleotide linkage to a preceding nucleotide, hydroxy, protected hydroxy, optionally substituted C₂₋₃₀alkenyl, optionally substituted C₁₋₃₀ alkoxy or a vinylphosphonate (VP) group.

In some embodiments of any one of the aspects described herein, R⁵ is a bond to an internucleotide linkage to a preceding nucleotide.

In some embodiments of any one of the aspects described herein, R⁵ is a hydroxyl or protected hydroxyl.

In some embodiments of any one of the aspects described herein, R⁵ is optionally substituted C₂₋₃₀alkenyl or optionally substituted C₁₋₃₀ alkoxy.

In some embodiments of any one of the aspects described herein, R⁵ is a vinylphosphonate group.

In some embodiments of any one of the aspects descried herein, R⁵ can be —CH(R⁵¹)—X⁵—R⁵², where X⁵ is absent, a bond or O; R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of any one of the aspects described herein, X⁵ is O or a bond. For example, X⁵ is O. In some other embodiments of any one of the aspects described herein, X⁵ is absent, i.e., R⁵ is —CH(R⁵¹)R⁵².

In some embodiments of the various aspects described herein, R⁵ can be —CH(R⁵¹)—R⁵² or —C(R⁵¹)═CHR⁵², where R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of the various aspects described herein, R⁵ is —CH(R⁵¹)—X⁵—R⁵². For example, R⁵ is —CH(R⁵¹)—X⁵—R⁵² and where R⁵¹ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ (CH₂)ₚ—OH, CH₂—[CH(OH)]ₘ (CH₂)ₚ NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁₋₆alkoxy.

In some embodiments of the various aspects described herein, R⁵ is —CH(R⁵¹)—O—R⁵², where R⁵¹ is H or C₁-C₃₀alkyl optionally substituted with 1, 2, 3, 4 or 5 substituents independently selected from 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ (CH₂)ₚ—OH, CH₂—[CH(OH)]ₘ (CH₂)ₚ NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of any one of the aspects described herein, R⁵ is —C(R⁵¹)═CHR⁵². It is noted that the double bond in —C(R⁵¹)═CHR⁵² can be in the cis or trans configuration. Accordingly, in some embodiments of any one of the aspects, R⁵ is —C(R⁵¹)═CHR⁵² and wherein the double bond is in the cis configuration. In some other embodiments of any one of the aspects, R⁵ is —C(R⁵¹)═CHR⁵² and wherein the double bond is in the trans configuration. In some embodiments of any one of the aspects described herein, R⁵ is —CH═CHR⁵².

In some embodiments of any one of the aspects described herein, R⁵² is a bond to an internucleoside linkage to the preceding nucleotide.

In embodiments of the various aspects described herein, R⁵ is optionally substituted C₁₋₆alkyl-R⁵³, optionally substituted —C₂₋₆alkenyl-R⁵³, or optionally substituted —C₂₋₆alkynyl-R⁵³. In embodiments of the various aspects described herein, R⁵³ can be —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; where R⁵⁴ is hydrogen or oxygen protecting group; R⁵ is hydrogen or sulfur protecting group; each R⁵⁶ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁷ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is hydrogen.

In some other embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, or —SP(S)(SR⁵⁷)(OR⁵⁶) is not hydrogen. For example, at least one at least one R⁵⁶ in P(O)(OR⁵⁶)₂, —P(S)(OR⁵⁶)₂, —P(S)(SR⁵⁷)(OR⁵⁶)—OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁶ is H and at least one R⁵⁶ is other than H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶).

In some embodiments of any one of the aspects, all R⁵⁶ are H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, all R⁵⁶ are other than H in in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is H.

In some embodiments of any one of the aspects, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is other than H. For example, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁷ is H and at least one R⁵⁷ is other than H in —P(S)(SR⁵⁷)₂, —OP(S)(SR⁵⁷)₂ and —SP(S)(SR⁵⁷)₂.

In some embodiments, all R⁵⁷ are H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments, all R⁵⁷ are other than H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments of any one of the aspects described herein, R⁵ is optionally substituted —C₂₋₆alkenyl-R⁵³. For example, R⁵ is —C₂₋₆alkenyl-R⁵³, where C₂₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ (CH₂)ₚ—OH, CH₂—[CH(OH)]ₘ (CH₂)ₚ NH₂ or CH₂-aryl-alkoxy, where “m” and “p” are independently 1, 2, 3, 4, 5 or 6; and R⁵³ is —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects, R⁵ is —CH═CHR⁵³. It is noted that a double bond in the optionally substituted —C₂₋₆alkenyl-R⁵³ can be in the cis or trans configuration. Accordingly, in some embodiments of any one of the aspects, R⁵ is —CH═CHR⁵³ and wherein the double bond is in the cis configuration. In some other embodiments of any one of the aspects, R⁵ is —CH═CHR⁵³ and wherein the double bond is in the trans configuration.

In some embodiments of any one of the aspects, R⁵ is —CH═CH—P(O)(OR⁵⁶)₂, —CH═CH—P(S)(OR⁵⁶)₂, —CH═CH—P(S)(SR⁵⁷)(OR⁵⁶), —CH═CH—P(S)(SR⁵⁷)₂, —CH═CH—OP(O)(OR⁵⁶)₂, —CH═CH—OP(S)(OR⁵⁶)₂, —CH═CH—OP(S)(SR⁵⁷)(OR⁵⁶), —CH═CH—OP(S)(SR⁵⁷)₂, —CH═CH—SP(O)(OR⁵⁶)₂, —CH═CH—SP(S)(OR⁵⁶)₂, —CH═CH—SP(S)(SR⁵⁷)(OR⁵⁶), or —CH═CH—SP(S)(SR⁵⁷)₂. For example, R⁵ is —CH═CH—P(O)(OR⁵⁶)₂.

In some embodiments, of any one of the aspects, R⁵⁴ is hydrogen or an oxygen protecting group. For example, R⁵⁴ is hydrogen or 4,4′-dimethoxytrityl (DMT). In some preferred embodiments, R⁵⁴ is H.

In some embodiments of any one of the aspects described herein, R⁵ is optionally substituted —C₁₋₆alkenyl-R⁵³. For example, R⁵ is —C₁₋₆alkenyl-R⁵³, where C₁₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ (CH₂)ₚ—OH, CH₂—[CH(OH)]ₘ (CH₂)ₚ NH₂ or CH₂-aryl-alkoxy, where “m” and “p” are independently 1, 2, 3, 4, 5 or 6; and R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects described herein, R⁵ can be —CH(R⁵⁸)—R⁵³, where R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; and R⁵⁸ is H, optionally substituted C₁₋₃₀alkyl, optionally substituted C₂₋₃₀alkenyl, or optionally substituted C₂₋₃₀alkynyl.

In some embodiments of any one of the aspects described herein, R⁵⁸ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. In one non-limiting example, R⁵⁸ is H. In some other non-limiting examples, R⁵⁸ is C₁-C₃₀alkyl optionally substituted with a substituent selected from NH₂, OH, C(O)NH₂, COOH, halo, SH, and C₁-C₆alkoxy.

In some embodiments of any one of the aspects described herein, R⁵ is —CH(R⁵⁸)—O—R⁵⁹, where R⁵⁹ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂. For example, R⁵ is —CH(R⁵⁸)—O—R⁵⁹, where R⁵⁸ is H or optionally substituted C₁-C₃₀alkyl and R⁵⁹ is H or —P(O)(OR⁵⁶)₂.

In some embodiments of any one of the aspects described herein, R⁵ is —CH(R⁵⁸)—S—R⁶⁰, where R⁶⁰ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂.

### R³²

In some embodiments of any one of the aspects described herein, R³² is hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁶, a lipid, a linker covalently attached to a lipid, a ligand, a linker covalently attached to a ligand, a solid support, a linker covalently attached to a solid support, or a reactive phosphorus group.

R³²² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R³²⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R³² is R³² is hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴ cyano, alkyl-thio-alkyl, thioalkoxy, cycloalkyl, aryl, heteroaryl, —NH(CH₂CH₂NH)ₛCH₂CH₂—R³²⁵, NHC(O)R³²⁴.

In some embodiments of any one of the aspects described herein, R³² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R³² is hydrogen, hydroxy, protected hydroxy, halogen, optionally substituted C₁₋₃₀ alkoxy, alkoxyalkyl (e.g., methoxyethyl), alkoxyalkylamine, alkoxyoxycarboxylate, amino, alkylamino, or dialkylamino.

In some embodiments of any one of the aspect, R³² is hydrogen, hydroxy, protected hydroxy, halogen, optionally substituted C₁₋₃₀ alkoxy, or alkoxyalkyl (e.g., methoxyethyl. In some embodiments of any one of the aspects, R³² is hydrogen, hydroxy, protected hydroxy, fluoro or methoxy.

In some embodiments of any one of the aspects R³² is halogen. For example, R³² can be fluoro, chloro, bromo or iodo. In some embodiments of any one of the aspects described herein, R³² is fluoro.

In some embodiments of any one of the aspects described herein, R³² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group. In some embodiments of any one of the aspects, v is 1. In some other embodiments of any one of the aspects, v is 2. In some embodiments, Y is O. For example, R³² and R⁴ taken together are 4′-C(R¹⁰R¹¹)ᵥ—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ (CH₂)ₚ—OH, CH₂—[CH(OH)]ₘ (CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃. In some embodiments of any one of the aspects, R¹⁰ and R¹¹ attached to the same C are the same. For example, R¹⁰ and R¹¹ attached to the same C are H.

In some embodiments of any one of the aspects, R³² and R⁴ taken together are 4′-CH₂—O-2′, 4′-CH(CH₃)—O-2′, 4′-CH(CH₂OCH₃)—O-2′, or 4′-CH₂CH₂—O-2′. For example, R³² and R⁴ taken together are 4′-CH₂CH₂—O-2′.

In some embodiments of any one of the aspects described herein, R³² is a reactive phosphorus group.

Without wishing to be bound by a theory, reactive phosphorus groups are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. Reactive phosphorous group in the form of phosphoramidites (P^{III} chemistry) as reactive phosphites are a preferred reactive phosphorous group for solid phase oligonucleotide synthesis. The intermediate phosphite compounds are subsequently oxidized to the Pv state using known methods to yield phosphodiester or phosphorothioate internucleoside linkages.

In some embodiments of any one of the aspects described herein, the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}. For example, the reactive phosphorous group is —OP(OR^{P})(N(R^{P2})₂).

In some embodiments of any one of the aspects, R^{P} is an optionally substituted C₁₋₆ alkyl. For example, R^{P} is a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)N₁₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In some embodiments, R^{P} is a C₁₋₆alkyl, optionally substituted with a CN or —SC(O)Ph. For example, R^{P} is cyanoethyl (—CH₂CH₂CN).

In the reactive phosphorous groups, each R^{P2} is independently optionally substituted C₁₋₆alkyl. For example, each R^{P2} can be independently selected from methyl, ethyl, propyl, isopropyl, n-butyl, iso-butyl, pentyl or hexyl. It is noted that when two or more R^{P2} groups are present in the reactive phosphorous group, they can be same or different. Thus, in some none-limiting examples, when two or more R^{P2} groups are present, the R^{P2} groups are different. In some other non-limiting examples, when two or more R^{P2} groups are present, the R^{P2} groups are same. In some embodiments of any one of the aspects, each R^{P2} is isopropyl.

In some embodiments of any one of the aspects, both R^{P2} taken together with the nitrogen atom to which they are attached form an optionally substituted 3-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In some embodiments of any one of the aspects, R^{P} and one of R^{P2} taken together with the atoms to which they are attached form an optionally substituted 4-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In the reactive phosphorous groups, each R^{P3} is independently optionally substituted C₁₋₆alkyl. For example, R^{P3} can be a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{P3} is methyl, ethyl, propyl, isopropyl, n-butyl, iso-butyl, pentyl or hexyl, each of which can be optionally substituted with a NH₂, OH, C(O)N₁₂, COOH, halo, SH, or C₁-C₆alkoxy.

In some embodiments of any one of the aspects, the reactive phosphorous group is —OP(OR^{P})(N(R^{P2})₂). For example, the reactive phosphorous group is —OP(OR^{P})(N(R^{P2})₂), where R^{P} is cyanoethyl (—CH₂CH₂CN) and each R^{P2} is isopropyl.

In some embodiments of any one of the aspects described herein, R³² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

In some embodiments of any one of the aspects, R³² is —OP(OR^{P}) (N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, each R^{P2} is independently optionally substituted C₁₋₆alkyl; and each R^{P3} is independently optionally substituted C₁₋₆ alkyl.

In some embodiments of any one of the aspects, R³² is —OP(OR^{P})(N(R^{P2})₂). For example, the R³² is —OP(OR^{P})(N(R^{P2})₂), where R^{P} is cyanoethyl (—CH₂CH₂CN) and each R^{P2} is isopropyl.

In some embodiments of any one of the aspects descried herein, R³² is a solid support or a linker covalently attached to a solid support. For example, R³² is —OC(O)CH₂CH₂C(O)NH—Z, where Z is a solid support. In some embodiments, R³² is —OC(O)CH₂CH₂CO₂H.

In some embodiments of any one of the aspects, when R³² is —OR³²², R³²² can be hydrogen or a hydroxyl protecting group.

When R³² is —SR³²³, R³²³ can be hydrogen or a sulfur protecting group. Accordingly, in some embodiments of any one of the aspects, R³²³ is hydrogen.

When R³² is —O(CH₂CH₂O)ᵣCH₂CH₂OR³²⁴, r can be 1-50; R³²⁴ is independently for each occurrence H, C₁-C₃₀alkyl, cyclyl, heterocyclyl, aryl, heteroaryl, aralkyl, sugar or R³²⁵; and R³²⁵ is independently for each occurrence amino (NH₂), alkylamino, dialkylamino, arylamino, diarylamino, heteroarylamino, or diheteroaryl amino.

When R³² is —NH(CH₂CH₂NH)ₛCH₂CH₂—R³²⁵, s can be 1-50 and R³²⁵ can be independently for each occurrence amino (NH₂), alkylamino, dialkylamino, arylamino, diarylamino, heteroarylamino, or diheteroaryl amino.

In some embodiments of any one of the aspects described herein, R³² is hydrogen, halogen, —OR³²², or optionally substituted C₁-C₃₀alkoxy. For example, R³² is halogen, —OR³²², or optionally substituted C₁-C₃₀alkoxy. In some embodiments of any one of the aspects described herein, R³² is F, OH or optionally substituted C₁-C₃₀alkoxy.

In some embodiments of any one of the aspects described herein, R³² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R³² is C₁-C₃₀alkoxy optionally substituted with a NH₂, OH, C(O)NH₂, COOH, halo, SH, or C₁-C₆alkoxy. In some embodiments of any one of the aspects described herein, R³² is —O(CH₂)ₜCH₃, where t is 1-21. For example, t is 14, 15, 16, 17 or 18. In one non-limiting example, t is 16.

In some embodiments of any one of the aspects, R³² is —O(CH₂)uR³²⁷, where u is 2-10; R³²⁷ is C₁-C₆alkoxy, amino (NH₂), CO₂H, OH or halo. For example, R³²⁷ is —CH₃ or NH₂. Accordingly, in some embodiments of any one of the aspects described herein, R³² is —O(CH₂)ᵤ—OMe or R³² is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R³² is a C₁-C₆haloalkyl. For example, R³² is a C₁-C₄haloalkyl. In some embodiments of any one of the aspects described herein, R³² is —CF₃, —CF₂CF₃, —CF₂CF₂CF₃ or —CF₂(CF₃)₂.

In some embodiments of any one of the aspects described herein, R³² is —OCH(CH₂OR³²⁸)CH₂OR³²⁹, where R³²⁸ and R³²⁹ independently are H, optionally substituted C₁-C₃₀alkyl, optionally substituted C₂-C₃₀alkenyl or optionally substituted C₂-C₃₀alkynyl. For example, R³²⁸ and R³²⁹ independently are optionally substituted C₁-C₃₀alkyl. In some embodiments of any one of the aspects described herein, R³² is —CH₂C(O)NHR³²¹⁰ where R³²¹⁰ is H, optionally substituted C₁-C₃₀alkyl, optionally substituted C₂-C₃₀alkenyl or optionally substituted C₂-C₃₀alkynyl. For example, R³²¹⁰ is H or optionally substituted C₁-C₃₀alkyl. In some embodiments, R³²¹⁰ is optionally substituted C₁-C₆alkyl.

### R³³

In some embodiments of any one of the aspects described herein, R³³ is hydrogen, halogen, —OR³³², —SR³³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³³⁴, cyano, alkyl-thio-alkyl, thioalkoxy, cycloalkyl, aryl, heteroaryl, —NH(CH₂CH₂NH)ₛCH₂CH₂—R³³⁵, NHC(O)R³³⁶, a lipid, a linker covalently attached to a lipid, a ligand, a linker covalently attached to a ligand, a solid support, a linker covalently attached to a solid support, or a reactive phosphorus group.

R³³² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³³³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³³⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³³⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R³³⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R³³ is a reactive phosphorus group. For example, R³³ is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR)(NR^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

In some embodiments of any one of the aspects, R³³ is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, each R^{P2} is independently optionally substituted C₁₋₆alkyl; and each R^{P3} is independently optionally substituted C₁₋₆ alkyl.

In some embodiments of any one of the aspects, R³³ is —OP(OR^{P})(N(R^{P2})₂). For example, the R³³ is —OP(OR^{P})(N(R^{P2})₂), where R^{P} is cyanoethyl (—CH₂CH₂CN) and each R^{P2} is isopropyl.

Optionally, only one of R³² and R³³ is a reactive phosphorous group.

In some embodiments of any one of the aspects descried herein, R³³ is a solid support or a linker covalently attached to a solid support. For example, R³³ is —OC(O)CH₂CH₂C(O)NH—Z, where Z is a solid support.

Optionally, only one of R³² and R³³ is a solid support or a linker covalently attached to a solid support.

In some embodiments of any one of the aspects, when R³³ is —OR³³², R³³² can be hydrogen or a hydroxyl protecting group. For example, R³³² can be hydrogen in some embodiments of any one of the aspects described herein. In some embodiments, R³³ is —OC(O)CH₂CH₂CO₂H.

When R³³ is —SR³³, R³³³ can be hydrogen or a sulfur protecting group. Accordingly, in some embodiments of any one of the aspects, R³³³ is hydrogen.

When R³³ is —O(CH₂CH₂O)ᵣCH₂CH₂OR³³⁴, r can be 1-50; R³³⁴ is independently for each occurrence H, C₁-C₃₀alkyl, cyclyl, heterocyclyl, aryl, heteroaryl, aralkyl, sugar or R³³⁵; and R³³⁵ is independently for each occurrence amino (NH₂), alkylamino, dialkylamino, arylamino, diarylamino, heteroarylamino, or diheteroaryl amino.

When R³³ is —NH(CH₂CH₂NH)ₛCH₂CH₂—R³³⁵, s can be 1-50 and R³³⁵ can be independently for each occurrence amino (NH₂), alkylamino, dialkylamino, arylamino, diarylamino, heteroarylamino, or diheteroaryl amino.

In some embodiments of any one of the aspects described herein, R³³ is hydrogen, halogen, —OR³³², or optionally substituted C₁-C₃₀alkoxy. For example, R³³ is halogen, —OR³³², or optionally substituted C₁-C₃₀alkoxy. In some embodiments of any one of the aspects described herein, R³³ is F, OH or optionally substituted C₁-C₃₀alkoxy.

In some embodiments of any one of the aspects described herein, R³³ is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R³³ is C₁-C₃₀alkoxy optionally substituted with a NH₂, OH, C(O)NH₂, COOH, halo, SH, or C₁-C₆alkoxy. In some embodiments of any one of the aspects described herein, R³³ is —O(CH₂)tCH3, where t is 1-21. For example, t is 14, 15, 16, 17 or 18. In one non-limiting example, t is 16.

In some embodiments of any one of the aspects, R³³ is —O(CH₂)uR³³⁷, where u is 2-10; R³³⁷ is C₁-C₆alkoxy, amino (NH₂), CO₂H, OH or halo. For example, R³³⁷ is —CH₃ or NH₂. Accordingly, in some embodiments of any one of the aspects described herein, R³³ is —O(CH₂)ᵤ—OMe or R³³ is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R³³ is a C₁-C₆haloalkyl. For example, R³³ is a C₁-C₄haloalkyl. In some embodiments of any one of the aspects described herein, R³³ is —CF₃, —CF₂CF₃, —CF₂CF₂CF₃ or —CF₂(CF₃)₂.

In some embodiments of any one of the aspects described herein, R³³ is —OCH(CH₂OR³³⁸)CH₂OR³³⁹, where R³³⁸ and R³³⁹ independently are H, optionally substituted C₁-C₃₀alkyl, optionally substituted C₂-C₃₀alkenyl or optionally substituted C₂-C₃₀alkynyl. For example, R³³⁸ and R³³⁹ independently are optionally substituted C₁-C₃₀alkyl.

In some embodiments of any one of the aspects described herein, R³³ is —CH₂C(O)NHR³³¹⁰, where R³³¹⁰ is H, optionally substituted C₁-C₃₀alkyl, optionally substituted C₂-C₃₀alkenyl or optionally substituted C₂-C₃₀alkynyl. For example, R³³¹⁰ is H or optionally substituted C₁-C₃₀alkyl. In some embodiments, R³¹⁰ is optionally substituted C₁-C₆alkyl.

In some embodiments of any one of the aspected described herein, R³³ and R⁴ taken together with the atoms to which they are attached form an optionally substituted C₃-8cycloalkyl, optionally substituted C₃₋₈cycloalkenyl, or optionally substituted 3-8 membered heterocyclyl.

### R³⁵

In some embodiments of the various aspects described herein, R³⁵ is R⁵⁵¹, optionally substituted C₁₋₆alkyl-R⁵⁵¹, optionally substituted —C₂₋₆alkenyl-R⁵⁵¹, or optionally substituted —C₂₋₆alkynyl-R⁵⁵¹, where R⁵⁵¹ can be —OR⁵⁵², —SR⁵, hydrogen, a phosphorous group, a solid support or a linker to a solid support. When R⁵⁵¹ is —OR⁵⁵², R⁵⁵² can be H or a hydroxyl protecting group. Similarly, when R⁵⁵¹ is —SR⁵⁵³, R⁵⁵³ can be H or a sulfur protecting group.

In some embodiments of any one of the aspects described herein, R³⁵ is —OR⁵⁵² or —SR⁵⁵³.

In some embodiments of any one of the aspects described herein, R⁵⁵² is a hydroxyl protecting group. Exemplary hydroxyl protecting groups for R⁵⁵² include, but are not limited to, benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In some embodiments of any one of the aspects described herein, R³⁵ is —OR⁵⁵² and R⁵⁵² is 4,4′-dimethoxytrityl (DMT), e.g., R³⁵ is —O-DMT.

In some embodiments of any one of the aspects described herein, R³⁵ is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R³⁵ is —CH(R⁵⁵⁴)—R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R³⁵ is —CH(R⁵⁵⁴)—O—R⁵⁵², where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R³⁵ is optionally substituted C₁₋₆alkyl-R⁵⁵¹ or optionally substituted —C₂₋₆alkenyl-R⁵⁵¹,

In some embodiments of any one of the aspects described herein, R³⁵ is —C(R⁵⁵⁴)═CR⁵⁵¹. It is noted that the double bond in —C(R⁵⁵⁴)═CHR⁵⁵¹ can be in the cis or trans configuration. Accordingly, in some embodiments of any one of the aspects, R^{d} is —C(R⁵⁵⁴)═CHR⁵⁵¹ and wherein the double bond is in the cis configuration. In some other embodiments of any one of the aspects, R^{d} is —C(R⁵⁵⁴)═CHR⁵⁵¹ and wherein the double bond is in the trans configuration.

In some embodiments of any one of the aspects described herein, R³⁵ is —CH═CHR⁵⁵¹.

In some embodiments of any one of the aspects, when R³⁵ is —C(R⁵⁵⁴)═CHR⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁—C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵⁵¹ is a phosphorous group. For example, R³⁵ is —CH═CHR⁵¹.

In some embodiments of any one of the aspects described herein, R⁵⁵¹ is a reactive phosphorous group.

In some embodiments of any one of the aspects, R³⁵ is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵⁵⁶)(OR⁵⁵), or —CH═CH═SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is hydrogen.

In some other embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, or —SP(S)(SR⁵⁵⁶)(OR⁵⁵⁵) is not hydrogen. For example, at least one at least one R⁵⁵⁵ in P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁵⁵ is H and at least one R⁵⁵⁵ is other than H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵).

In some embodiments of any one of the aspects, all R⁵⁵⁵ are H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, all R⁵⁵⁵ are other than H in in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR55), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is H.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is other than H. For example, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁵⁶ is H and at least one R⁵⁵⁶ is other than H in —P(S)(SR⁵⁵⁶)₂, —OP(S)(SR⁵⁵⁶)₂ and —SP(S)(SR⁵⁵⁶)₂.

In some embodiments, all R⁵⁵⁶ are H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments, all R⁵⁵⁶ are other than H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments of any one of the aspects, R³⁵ is —CH═CH—P(O)(OR⁵⁵⁵)₂, where each R⁵⁵⁵ is H or an oxygen protecting group.

In some embodiments of any one of the aspects, R³³ is a reactive phosphorous group, a solid support, a linker to a solid support, and R³⁵ is a protected hydroxyl.

In some other embodiments of any one of the aspects, R³² is a reactive phosphorous group, a solid support, a linker to a solid support, and R³⁵ is a protected hydroxyl.

### R⁴²

In some embodiments of any one of the aspects described herein, R⁴² is halogen, —OR⁴²², —SR⁴²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR⁴²⁴, cyano, alkyl-thio-alkyl, thioalkoxy, cycloalkyl, aryl, heteroaryl, —NH(CH₂CH₂NH)ₛCH₂CH₂—R⁴²⁵, NHC(O)R⁴²⁶, a lipid, a linker covalently attached to a lipid, a ligand, a linker covalently attached to a ligand, a solid support, a linker covalently attached to a solid support, or a reactive phosphorus group.

R⁴²² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁴²³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁴²⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁴²⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R⁴²⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R⁴² is a reactive phosphorus group. For example, R⁴² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(NR^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

In some embodiments of any one of the aspects, R⁴² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, each R^{P2} is independently optionally substituted C₁₋₆alkyl; and each R^{P3} is independently optionally substituted C₁₋₆ alkyl.

In some embodiments of any one of the aspects, R⁴² is —OP(OR^{P})(N(R^{P2})₂). For example, the R⁴² is —OP(OR^{P})(N(R^{P2})₂), where R^{P} is cyanoethyl (—CH₂CH₂CN) and each R^{P2} is isopropyl.

In some embodiments of any one of the aspects descried herein, R⁴² is a solid support or a linker covalently attached to a solid support. For example, R⁴² is —OC(O)CH₂CH₂C(O)NH—Z, where Z is a solid support.

In some embodiments of any one of the aspects, when R⁴² is —OR⁴²², R⁴²² can be hydrogen or a hydroxyl protecting group. For example, R⁴²² can be hydrogen in some embodiments of any one of the aspects described herein. In some embodiments, R⁴² is —OC(O)CH₂CH₂CO₂H.

When R⁴² is —SR⁴²³, R⁴²³ can be hydrogen or a sulfur protecting group. Accordingly, in some embodiments of any one of the aspects, R⁴²³ is hydrogen.

When R⁴² is —O(CH₂CH₂O)ᵣCH₂CH₂OR⁴²⁴, r can be 1-50; R⁴²⁴ is independently for each occurrence H, C₁-C₃₀alkyl, cyclyl, heterocyclyl, aryl, heteroaryl, aralkyl, sugar or R⁴²⁵; and R⁴²⁵ is independently for each occurrence amino (NH₂), alkylamino, dialkylamino, arylamino, diarylamino, heteroarylamino, or diheteroaryl amino.

When R⁴² is —NH(CH₂CH₂NH)ₛCH₂CH₂—R⁴²⁵, s can be 1-50 and R⁴²⁵ can be independently for each occurrence amino (NH₂), alkylamino, dialkylamino, arylamino, diarylamino, heteroarylamino, or diheteroaryl amino.

In some embodiments of any one of the aspects described herein, R⁴² is-OR⁴²², or optionally substituted C₁-C₃₀alkoxy. For example, R⁴² is halogen, —OR⁴²², or optionally substituted C₁-C₃₀alkoxy. In some embodiments of any one of the aspects described herein, R⁴² is F, OH or optionally substituted C₁-C₃₀alkoxy.

In some embodiments of any one of the aspects described herein, R⁴² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R⁴² is C₁-C₃₀alkoxy optionally substituted with a NH₂, OH, C(O)NH₂, COOH, halo, SH, or C₁-C₆alkoxy. In some embodiments of any one of the aspects described herein, R⁴² is —O(CH₂)tCH3, where t is 1-21. For example, t is 14, 15, 16, 17 or 18. In one non-limiting example, t is 16.

In some embodiments of any one of the aspects, R⁴² is —O(CH₂)uR⁴²⁷, where u is 2-10; R⁴²⁷ is C₁-C₆alkoxy, amino (NH₂), CO₂H, OH or halo. For example, R⁴²⁷ is —CH₃ or NH₂. Accordingly, in some embodiments of any one of the aspects described herein, R⁴² is —O(CH₂)ᵤ—OMe or R⁴² is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R⁴² is a C₁-C₆haloalkyl. For example, R⁴² is a C₁-C₄haloalkyl. In some embodiments of any one of the aspects described herein, R⁴² is —CF₃, —CF₂CF₃, —CF₂CF₂CF₃ or —CF₂(CF₃)₂.

In some embodiments of any one of the aspects described herein, R⁴² is —OCH(CH₂OR⁴²⁸)CH₂OR⁴²⁹, where R⁴²⁸ and R⁴²⁹ independently are H, optionally substituted C₁-C₃₀alkyl, optionally substituted C₂-C₃₀alkenyl or optionally substituted C₂-C₃₀alkynyl. For example, R⁴²⁸ and R⁴²⁹ independently are optionally substituted C₁-C₃₀alkyl. In some embodiments of any one of the aspects described herein, R⁴² is —CH₂C(O)NHR⁴²¹⁰ where R⁴²¹⁰ is H, optionally substituted C₁-C₃₀alkyl, optionally substituted C₂-C₃₀alkenyl or optionally substituted C₂-C₃₀alkynyl. For example, R⁴²¹⁰ is H or optionally substituted C₁-C₃₀alkyl. In some embodiments, R⁴²¹⁰ is optionally substituted C₁-C₆alkyl.

In some embodiments of any one of the aspected described herein, R⁴² and R⁴ taken together with the atoms to which they are attached form an optionally substituted C₃-8cycloalkyl, optionally substituted C₃₋₈cycloalkenyl, or optionally substituted 3-8 membered heterocyclyl.

### R⁴⁵

In some embodiments of the various aspects described herein, R⁴⁵ is R⁵⁵¹, optionally substituted C₁₋₆alkyl-R⁵⁵¹, optionally substituted —C₂₋₆alkenyl-R⁵⁵¹, or optionally substituted —C₂₋₆alkynyl-R⁵⁵¹, where R⁵⁵¹ can be —OR⁵⁵², —SR⁵, hydrogen, a phosphorous group, a solid support or a linker to a solid support. When R⁵⁵¹ is —OR⁵⁵², R⁵⁵² can be H or a hydroxyl protecting group. Similarly, when R⁵⁵¹ is —SR⁵⁵³, R⁵⁵³ can be H or a sulfur protecting group.

In some embodiments of any one of the aspects described herein, R⁴⁵ is —OR⁵⁵² or —SR⁵⁵³.

In some embodiments of any one of the aspects described herein, R⁵⁵² is a hydroxyl protecting group. Exemplary hydroxyl protecting groups for R⁵⁵² include, but are not limited to, benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In some embodiments of any one of the aspects described herein, R⁴⁵ is —OR⁵⁵² and R⁵⁵² is 4,4′-dimethoxytrityl (DMT), e.g., R⁴⁵ is —O-DMT.

In some embodiments of any one of the aspects described herein, R⁴⁵ is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R⁴⁵ is —CH(R⁵⁵⁴)—R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R⁴⁵ is —CH(R⁵⁵⁴)—O—R⁵⁵², where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈ alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R⁴⁵ is optionally substituted C₁₋₆alkyl-R⁵⁵¹ or optionally substituted —C₂₋₆alkenyl-R⁵⁵¹,

In some embodiments of any one of the aspects described herein, R⁴⁵ is —C(R⁵⁵⁴)=CRM⁵⁵¹. It is noted that the double bond in —C(R⁵⁵⁴)═CHR⁵⁵¹ can be in the cis or trans configuration. Accordingly, in some embodiments of any one of the aspects, R^{d} is —C(R⁵⁵⁴)═CHR⁵⁵¹ and wherein the double bond is in the cis configuration. In some other embodiments of any one of the aspects, R^{d} is —C(R⁵⁵⁴)═CHR⁵⁵¹ and wherein the double bond is in the trans configuration.

In some embodiments of any one of the aspects described herein, R⁴⁵ is —CH═CHR⁵⁵¹.

In some embodiments of any one of the aspects, when R⁴⁵ is —C(R⁵⁵⁴)═CHR⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵⁵¹ is a phosphorous group. For example, R⁴⁵ is —CH═CHR⁵⁵¹.

In some embodiments of any one of the aspects described herein, R⁵⁵¹ is a reactive phosphorous group.

In some embodiments of any one of the aspects, R⁴⁵ is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵⁵⁶)(OR⁵⁵⁵), or —CH═CH—SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is hydrogen.

In some other embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, or —SP(S)(SR⁵⁵⁶)(OR⁵⁵⁵) is not hydrogen. For example, at least one at least one R⁵⁵⁵ in P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁵⁵ is H and at least one R⁵⁵⁵ is other than H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵).

In some embodiments of any one of the aspects, all R⁵⁵⁵ are H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, all R⁵⁵⁵ are other than H in in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR55), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is H.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is other than H. For example, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁵⁶ is H and at least one R⁵⁵⁶ is other than H in —P(S)(SR⁵⁵⁶)₂, —OP(S)(SR⁵⁵⁶)₂ and —SP(S)(SR⁵⁵⁶)₂.

In some embodiments, all R⁵⁵⁶ are H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments, all R⁵⁵⁶ are other than H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments of any one of the aspects, R⁴⁵ is —CH═CH—P(O)(OR⁵⁵⁵)₂, where each R⁵⁵⁵ is H or an oxygen protecting group.

In some embodiments of any one of the aspects, R⁴² is a reactive phosphorous group, a solid support, a linker to a solid support, and R⁴⁵ is a protected hydroxyl. R⁵²

In some embodiments of any one of the aspects described herein, R⁵² can be a bond to an internucleotide linkage to a subsequent nucleotide, hydroxy, protected hydroxy, optionally substituted C₁₋₃₀ alkoxy, halogen, alkoxyalkyl (e.g., methoxyethyl), amino, alkylamino, dialkylamino, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support.

In some embodiments of any one of the aspects described herein, R⁵² is a bond to an internucleotide linkage to a subsequent nucleotide, hydroxy, protected hydroxy, optionally substituted C₁₋₃₀ alkoxy, a 3′-oligonuclotide capping group (e.g., an inverted nucleotide or an inverted abasic nucleotide), a solid support, or a linker covalently bonded (e.g., —C(O)CH₂CH₂C(O)—) to a solid support. For example, R⁵² is a bond to an internucleotide linkage to a subsequent nucleotide, hydroxy, a solid support, or a linker covalently bonded (e.g., —C(O)CH₂CH₂C(O)—) to a solid support. In some embodiments of any one of the aspects described herein, R⁵² is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R⁵² is a bond to an internucleotide linkage to a subsequent nucleotide.

In some embodiments of any one of the aspects described herein, R⁵² is a solid support, or a linker covalently bonded to a solid support.

In some embodiments of any one of the aspects described herein, R⁵² is hydroxyl.

### R⁵⁵

In some embodiments of any one of the aspects described herein, R⁵⁵ can be a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10).

In some embodiments of any one of the aspects described herein, R⁵⁵ can be a bond to an internucleotide linkage to a preceding nucleotide, hydroxy, protected hydroxy, optionally substituted C₂₋₃₀alkenyl, optionally substituted C₂₋₃₀alkynyl, optionally substituted C₁₋₃₀ alkoxy, vinylphosphonate (VP) group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidates, or alkylphosphonates.

In some embodiments of any one of the aspects described herein, R⁵⁵ is a bond to an internucleotide linkage to a preceding nucleotide, hydroxy, protected hydroxy, optionally substituted C₂₋₃₀alkenyl, optionally substituted C₁₋₃₀ alkoxy or a vinylphosphonate (VP) group.

In some embodiments of any one of the aspects described herein, R⁵⁵ is a bond to an internucleotide linkage to a preceding nucleotide.

In some embodiments of any one of the aspects described herein, R⁵⁵ is a hydroxyl or protected hydroxyl.

In some embodiments of any one of the aspects described herein, R⁵⁵ is optionally substituted C₂₋₃₀alkenyl or optionally substituted C₁₋₃₀ alkoxy.

In some embodiments of any one of the aspects described herein, R⁵⁵ is a vinylphosphonate group.

In some embodiments of any one of the aspects descried herein, R⁵⁵ can be —CH(R⁵¹)—X⁵—R⁵², where X⁵ is absent, a bond or O; R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of the various aspects described herein, R⁵⁵ can be —CH(R⁵¹)—R⁵² or —C(R⁵¹)═CHR⁵², where R⁵¹ is hydrogen, optionally substituted C₁₋₃₀alkyl, optionally substituted —C₂₋₃₀alkenyl, or optionally substituted —C₂₋₃₀alkynyl, and R⁵² is a bond to an internucleoside linkage to the preceding nucleotide.

In some embodiments of the various aspects described herein, R⁵⁵ is —CH(R⁵¹)—X⁵—R⁵². For example, R⁵⁵ is —CH(R⁵¹)—X⁵—R⁵² and where R⁵¹ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁₋₆alkoxy.

In some embodiments of the various aspects described herein, R⁵⁵ is —CH(R⁵¹)—O—R⁵², where R⁵¹ is H or C₁-C₃₀alkyl optionally substituted with 1, 2, 3, 4 or 5 substituents independently selected from 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂—C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R⁵¹ is H. In some other non-limiting examples, R⁵¹ is C₁-C₃₀alkyl optionally substituted with a NH₂, OH, C(O)NH₂, COOH, halo, SH, or C₁-C₆alkoxy.

In some embodiments of any one of the aspects described herein, R⁵⁵ is —C(R⁵¹)═CHR⁵². It is noted that the double bond in —C(R⁵¹)═CHR⁵² can be in the cis or trans configuration. Accordingly, in some embodiments of any one of the aspects, R⁵⁵ is —C(R⁵¹)═CHR⁵² and wherein the double bond is in the cis configuration. In some other embodiments of any one of the aspects, R⁵⁵ is —C(R⁵¹)═CHR⁵² and wherein the double bond is in the trans configuration. In some embodiments of any one of the aspects described herein, R⁵⁵ is —CH═CHR⁵².

In some embodiments of any one of the aspects described herein, R⁵² is a bond to an internucleoside linkage to the preceding nucleotide.

In embodiments of the various aspects described herein, R⁵⁵ is optionally substituted C₁₋₆alkyl-R⁵³, optionally substituted —C₂₋₆alkenyl-R⁵³, or optionally substituted —C₂₋₆alkynyl-R⁵³. In embodiments of the various aspects described herein, R⁵³ can be —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; where R⁵⁴ is hydrogen or oxygen protecting group; R⁵ is hydrogen or sulfur protecting group; each R⁵⁶ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁷ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is hydrogen.

In some other embodiments of any one of the aspects, at least one R⁵⁶ in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, or —SP(S)(SR⁵⁷)(OR⁵⁶) is not hydrogen. For example, at least one at least one R⁵⁶ in P(O)(OR⁵⁶)₂, —P(S)(OR⁵⁶)₂, —P(S)(SR⁵⁷)(OR⁵⁶)—OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁶ is H and at least one R⁵⁶ is other than H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), SP(O)(OR⁵⁶)₂, —SP(S)(OR⁵⁶)₂, and —SP(S)(SR⁵⁷)(OR⁵⁶).

In some embodiments of any one of the aspects, all R⁵⁶ are H in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, all R⁵⁶ are other than H in in —P(O)(OR⁵⁶)₂, —P(S)(OR⁵⁶)₂, —P(S)(SR⁵⁷)(OR⁵⁶), —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), and —SP(S)(SR⁵⁷)₂.

In some embodiments of any one of the aspects, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is H.

In some embodiments of any one of the aspects, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is other than H. For example, at least one R⁵⁷ in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁷ is H and at least one R⁵⁷ is other than H in —P(S)(SR⁵⁷)₂, —OP(S)(SR⁵⁷)₂ and —SP(S)(SR⁵⁷)₂.

In some embodiments, all R⁵⁷ are H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments, all R⁵⁷ are other than H in —P(S)(SR⁵⁷)(OR⁵⁶), —P(S)(SR⁵⁷)₂, —OP(S)(OR⁵⁶)₂, —OP(S)(SR⁵⁷)(OR⁵⁶), —OP(S)(SR⁵⁷)₂, —SP(S)(SR⁵⁷)(OR⁵⁶), and —SP(S)(SR⁵⁷)₂.

In some embodiments of any one of the aspects described herein, R⁵⁵ is optionally substituted —C₂₋₆alkenyl-R⁵³. For example, R⁵⁵ is —C₂₋₆alkenyl-R⁵³, where C₂₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6; and R⁵³ is —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects, R⁵⁵ is —CH═CHR⁵³. It is noted that a double bond in the optionally substituted —C₂₋₆alkenyl-R⁵³ can be in the cis or trans configuration. Accordingly, in some embodiments of any one of the aspects, R⁵⁵ is —CH═CHR⁵³ and wherein the double bond is in the cis configuration. In some other embodiments of any one of the aspects, R⁵⁵ is —CH═CHR⁵³ and wherein the double bond is in the trans configuration.

In some embodiments of any one of the aspects, R⁵⁵ is —CH═CH—P(O)(OR⁵⁶)₂, —CH═CH—P(S)(OR⁵⁶)₂, —CH═CH—P(S)(SR⁵⁷)(OR⁵⁶), —CH═CH—P(S)(SR⁵⁷)₂, —CH═CH—OP(O)(OR⁵⁶)₂, —CH═CH—OP(S)(OR⁵⁶)₂, —CH═CH—OP(S)(SR⁵⁷)(OR⁵⁶), —CH═CH—OP(S)(SR⁵⁷)₂, —CH═CH—SP(O)(OR⁵⁶)₂, —CH═CH—SP(S)(OR⁵⁶)₂, —CH═CH—SP(S)(SR⁵⁷)(OR⁵⁶), or —CH═CH—SP(S)(SR⁵⁷)₂. For example, R⁵⁵ is —CH═CH—P(O)(OR⁵⁶)₂.

In some embodiments, of any one of the aspects, R⁵⁴ is hydrogen or an oxygen protecting group. For example, R⁵⁴ is hydrogen or 4,4′-dimethoxytrityl (DMT). In some preferred embodiments, R⁵⁴ is H.

In some embodiments of any one of the aspects described herein, R⁵⁵ is optionally substituted —C₁₋₆alkenyl-R⁵³. For example, R⁵⁵ is —C₁₋₆alkenyl-R⁵³, where C₁₋₆alkenyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)N₁₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6; and R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂.

In some embodiments of any one of the aspects described herein, R⁵⁵ can be —CH(R⁵⁸)—R⁵³, where R⁵³ is —OR⁵⁴, —SR⁵⁵, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂, —OP(S)(OR⁵⁶)₂, —OP(S)(SR⁵⁷)(OR⁵⁶), —OP(S)(SR⁵⁷)₂, —SP(O)(OR⁵⁶)₂, —SP(S)(OR⁵⁶)₂, —SP(S)(SR⁵⁷)(OR⁵⁶), or —SP(S)(SR⁵⁷)₂; and R⁵⁸ is H, optionally substituted C₁₋₃₀alkyl, optionally substituted C₂₋₃₀alkenyl, or optionally substituted C₂₋₃₀alkynyl.

In some embodiments of any one of the aspects described herein, R⁵⁸ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. In one non-limiting example, R⁵⁸ is H. In some other non-limiting examples, R⁵⁸ is C₁-C₃₀alkyl optionally substituted with a substituent selected from NH₂, OH, C(O)NH₂, COOH, halo, SH, and C₁-C₆alkoxy.

In some embodiments of any one of the aspects described herein, R⁵⁵ is —CH(R⁵⁸)—O—R⁵⁹, where R⁵⁹ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂. For example, R⁵⁵ is —CH(R⁵⁸)—O—R⁵⁹, where R⁵⁸ is H or optionally substituted C₁-C₃₀alkyl and R⁵⁹ is H or —P(O)(OR⁵⁶)₂.

In some embodiments of any one of the aspects described herein, R⁵⁵ is —CH(R⁵⁸)—S—R⁶⁰, where R⁶⁰ is H, —P(O)(OR⁵⁶)₂, —P(S)(OR⁵⁶)₂, —P(S)(SR⁵⁷)(OR⁵⁶), —P(S)(SR⁵⁷)₂, —OP(O)(OR⁵⁶)₂.

### R⁶²

In some embodiments of any one of the aspects described herein, R⁶² is —OR⁶²², —SR⁶²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR⁶²⁴, cyano, alkyl-thio-alkyl, thioalkoxy, cycloalkyl, aryl, heteroaryl, —NH(CH₂CH₂NH)ₛCH₂CH₂—R⁶²⁵, NHC(O)R⁶²⁶, a lipid, a linker covalently attached to a lipid, a ligand, a linker covalently attached to a ligand, a solid support, a linker covalently attached to a solid support, or a reactive phosphorus group.

R⁶²² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶²³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶²⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶²⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R⁶²⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R⁶² is a reactive phosphorus group. For example, R⁶² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR)(NR^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}.

In some embodiments of any one of the aspects, R⁶² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, each R^{P2} is independently optionally substituted C₁₋₆alkyl; and each R^{P3} is independently optionally substituted C₁₋₆ alkyl.

In some embodiments of any one of the aspects, R⁶² is —OP(OR^{P})(N(R^{P2})₂). For example, the R⁶² is —OP(OR^{P})(N(R^{P2})₂), where R^{P} is cyanoethyl (—CH₂CH₂CN) and each R^{P2} is isopropyl.

In some embodiments of any one of the aspects descried herein, R⁶² is a solid support or a linker covalently attached to a solid support. For example, R⁶² is —OC(O)CH₂CH₂C(O)NH—Z, where Z is a solid support.

In some embodiments of any one of the aspects, when R⁶² is —OR⁶²², R⁶²² can be hydrogen or a hydroxyl protecting group. For example, R⁶²² can be hydrogen in some embodiments of any one of the aspects described herein. In some embodiments, R⁶² is —OC(O)CH₂CH₂CO₂H.

When R⁶² is —SR⁶²³, R⁶²³ can be hydrogen or a sulfur protecting group. Accordingly, in some embodiments of any one of the aspects, R⁶²³ is hydrogen.

When R⁶² is —O(CH₂CH₂O)ᵣCH₂CH₂OR⁶²⁴, r can be 1-50; R⁶²⁴ is independently for each occurrence H, C₁-C₃₀alkyl, cyclyl, heterocyclyl, aryl, heteroaryl, aralkyl, sugar or R⁶²⁵; and R⁶²⁵ is independently for each occurrence amino (NH₂), alkylamino, dialkylamino, arylamino, diarylamino, heteroarylamino, or diheteroaryl amino.

When R⁶² is —NH(CH₂CH₂NH)ₛCH₂CH₂—R⁶²⁵, s can be 1-50 and R⁶²⁵ can be independently for each occurrence amino (NH₂), alkylamino, dialkylamino, arylamino, diarylamino, heteroarylamino, or diheteroaryl amino.

In some embodiments of any one of the aspects described herein, R⁶² is-OR⁶²², or optionally substituted C₁-C₃₀alkoxy. For example, R⁶² is halogen, —OR⁶²², or optionally substituted C₁-C₃₀alkoxy. In some embodiments of any one of the aspects described herein, R⁶² is F, OH or optionally substituted C₁-C₃₀alkoxy.

In some embodiments of any one of the aspects described herein, R⁶² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R⁶² is C₁-C₃₀alkoxy optionally substituted with a NH₂, OH, C(O)NH₂, COOH, halo, SH, or C₁-C₆alkoxy. In some embodiments of any one of the aspects described herein, R⁶² is —O(CH₂)tCH3, where t is 1-21. For example, t is 14, 15, 16, 17 or 18. In one non-limiting example, t is 16.

In some embodiments of any one of the aspects, R⁶² is —O(CH₂)uR⁶²⁷, where u is 2-10; R⁶²⁷ is C₁-C₆alkoxy, amino (NH₂), CO₂H, OH or halo. For example, R⁶²⁷ is —CH₃ or NH₂. Accordingly, in some embodiments of any one of the aspects described herein, R⁶² is —O(CH₂)ᵤ—OMe or R⁶² is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R⁶² is a C₁-C₆haloalkyl. For example, R⁶² is a C₁-C₄haloalkyl. In some embodiments of any one of the aspects described herein, R⁶² is —CF₃, —CF₂CF₃, —CF₂CF₂CF₃ or —CF₂(CF₃)₂.

In some embodiments of any one of the aspects described herein, R⁶² is —OCH(CH₂OR⁶²⁸)CH₂OR⁶²⁹, where R⁶²⁸ and R⁶²⁹ independently are H, optionally substituted C₁-C₃₀alkyl, optionally substituted C₂-C₃₀alkenyl or optionally substituted C₂-C₃₀alkynyl. For example, R⁶²⁸ and R⁶²⁹ independently are optionally substituted C₁-C₃₀alkyl.

In some embodiments of any one of the aspects described herein, R⁶² is —CH₂C(O)NHR⁶²¹⁰, where R⁶²¹⁰ is H, optionally substituted C₁-C₃₀alkyl, optionally substituted C₂-C₃₀alkenyl or optionally substituted C₂-C₃₀alkynyl. For example, R⁶²¹⁰ is H or optionally substituted C₁-C₃₀alkyl. In some embodiments, R⁶²¹⁰ is optionally substituted C₁-C₆alkyl.

### R⁶³

In some embodiments of any one of the aspects described herein, R⁶³ is hydrogen, halogen, —OR⁶³², —SR⁶³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR⁶³⁴, cyano, alkyl-thio-alkyl, thioalkoxy, cycloalkyl, aryl, heteroaryl, —NH(CH₂CH₂NH)ₛCH₂CH₂—R⁶³⁵, NHC(O)R⁶³⁶, a lipid, a linker covalently attached to a lipid, a ligand, a linker covalently attached to a ligand, a solid support, a linker covalently attached to a solid support, or a reactive phosphorus group.

R⁶³² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶³³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶³⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶³⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R⁶³⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R⁶³ is R⁶³ is hydrogen, halogen, —OR⁶³², —SR⁶³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR⁶³⁴, cyano, alkyl-thio-alkyl, thioalkoxy, cycloalkyl, aryl, heteroaryl, —NH(CH₂CH₂NH)ₛCH₂CH₂—R⁶³⁵, NHC(O)R⁶³⁴.

In some embodiments of any one of the aspects described herein, R⁶³ is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R⁶³ is hydrogen, hydroxy, protected hydroxy, halogen, optionally substituted C₁₋₃₀ alkoxy, alkoxyalkyl (e.g., methoxyethyl), alkoxyalkylamine, alkoxyoxycarboxylate, amino, alkylamino, or dialkylamino.

In some embodiments of any one of the aspect, R⁶³ is hydrogen, hydroxy, protected hydroxy, halogen, optionally substituted C₁₋₃₀ alkoxy, or alkoxyalkyl (e.g., methoxyethyl).

In some embodiments of any one of the aspects R⁶³ is halogen. For example, R⁶³ can be fluoro, chloro, bromo or iodo. In some embodiments of any one of the aspects described herein, R⁶³ is fluoro.

In some embodiments of any one of the aspects, when R⁶³ is —OR⁶³², R⁶³² can be hydrogen or a hydroxyl protecting group.

When R⁶³ is —SR⁶³³, R⁶³³ can be hydrogen or a sulfur protecting group. Accordingly, in some embodiments of any one of the aspects, R⁶³³ is hydrogen.

When R⁶³ is —O(CH₂CH₂O)ᵣCH₂CH₂OR⁶³⁴, r can be 1-50; R⁶³⁴ is independently for each occurrence H, C₁-C₃₀alkyl, cyclyl, heterocyclyl, aryl, heteroaryl, aralkyl, sugar or R⁶³⁵; and R⁶³⁵ is independently for each occurrence amino (NH₂), alkylamino, dialkylamino, arylamino, diarylamino, heteroarylamino, or diheteroaryl amino.

When R⁶³ is —NH(CH₂CH₂NH)ₛCH₂CH₂—R⁶³⁵, s can be 1-50 and R⁶³⁵ can be independently for each occurrence amino (NH₂), alkylamino, dialkylamino, arylamino, diarylamino, heteroarylamino, or diheteroaryl amino.

In some embodiments of any one of the aspects described herein, R⁶³ is hydrogen, halogen, —OR⁶³², or optionally substituted C₁-C₃₀alkoxy. For example, R⁶³ is halogen, —OR⁶³², or optionally substituted C₁-C₃₀alkoxy. In some embodiments of any one of the aspects described herein, R⁶³ is F, OH or optionally substituted C₁-C₃₀alkoxy.

In some embodiments of any one of the aspects described herein, R⁶³ is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R⁶³ is C₁-C₃₀alkoxy optionally substituted with a NH₂, OH, C(O)NH₂, COOH, halo, SH, or C₁-C₆alkoxy. In some embodiments of any one of the aspects described herein, R⁶³ is —O(CH₂)tCH3, where t is 1-21. For example, t is 14, 15, 16, 17 or 18. In one non-limiting example, t is 16.

In some embodiments of any one of the aspects, R⁶³ is —O(CH₂)uR⁶³⁷, where u is 2-10; R⁶³⁷ is C₁-C₆alkoxy, amino (NH₂), CO₂H, OH or halo. For example, R⁶³⁷ is —CH₃ or NH₂. Accordingly, in some embodiments of any one of the aspects described herein, R⁶³ is —O(CH₂)ᵤ—OMe or R⁶³ is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R⁶³ is a C₁-C₆haloalkyl. For example, R⁶³ is a C₁-C₄haloalkyl. In some embodiments of any one of the aspects described herein, R⁶³ is —CF₃, —CF₂CF₃, —CF₂CF₂CF₃ or —CF₂(CF₃)₂.

In some embodiments of any one of the aspects described herein, R⁶³ is —OCH(CH₂OR⁶³⁸)CH₂OR⁶³⁹, where R⁶³⁸ and R⁶³⁹ independently are H, optionally substituted C₁-C₃₀alkyl, optionally substituted C₂-C₃₀alkenyl or optionally substituted C₂-C₃₀alkynyl. For example, R⁶³⁸ and R⁶³⁹ independently are optionally substituted C₁-C₃₀alkyl.

In some embodiments of any one of the aspects described herein, R⁶³ is —CH₂C(O)NHR⁶³¹⁰, where R⁶³¹⁰ is H, optionally substituted C₁-C₃₀alkyl, optionally substituted C₂-C₃₀alkenyl or optionally substituted C₂-C₃₀alkynyl. For example, R⁶³¹⁰ is H or optionally substituted C₁-C₃₀alkyl. In some embodiments, R⁶³¹⁰ is optionally substituted C₁-C₆alkyl.

In some embodiments of any one of the aspects described herein, R⁶³ is hydrogen, fluoro, —O-MOE, —O-alkyl (e.g., methoxy or —O—C₁₆aliphatic), —O-alkene, —O-alkyne, —O-lipid, —O-branched lipid or aminoalkyl.

### R⁶⁴

In some embodiments of any one of the aspects described herein, R⁶⁴ is hydrogen, halogen, —OR⁶⁴², —SR⁶⁴³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR⁶⁴⁴, cyano, alkyl-thio-alkyl, thioalkoxy, cycloalkyl, aryl, heteroaryl, —NH(CH₂CH₂NH)ₛCH₂CH₂—R⁶⁴⁵, NHC(O)R⁶⁴⁶, a lipid, a linker covalently attached to a lipid, a ligand, a linker covalently attached to a ligand, a solid support, a linker covalently attached to a solid support, or a reactive phosphorus group.

R⁶⁴² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶⁴³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶⁴⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R⁶⁴⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R⁶⁴⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R⁶⁴ is R⁶⁴ is hydrogen, halogen, —OR⁶⁴², —SR⁶⁴³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR⁶⁴⁴, cyano, alkyl-thio-alkyl, thioalkoxy, cycloalkyl, aryl, heteroaryl, —NH(CH₂CH₂NH)ₛCH₂CH₂—R⁶⁴⁵, NHC(O)R⁶⁴⁴.

In some embodiments of any one of the aspects described herein, R⁶⁴ is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R⁶⁴ is hydrogen, hydroxy, protected hydroxy, halogen, optionally substituted C₁₋₃₀ alkoxy, alkoxyalkyl (e.g., methoxyethyl), alkoxyalkylamine, alkoxyoxycarboxylate, amino, alkylamino, or dialkylamino.

In some embodiments of any one of the aspect, R⁶⁴ is hydrogen, hydroxy, protected hydroxy, halogen, optionally substituted C₁₋₃₀ alkoxy, or alkoxyalkyl (e.g., methoxyethyl).

In some embodiments of any one of the aspects R⁶⁴ is halogen. For example, R⁶⁴ can be fluoro, chloro, bromo or iodo. In some embodiments of any one of the aspects described herein, R⁶⁴ is fluoro.

In some embodiments of any one of the aspects, when R⁶⁴ is —OR⁶⁴², R⁶⁴² can be hydrogen or a hydroxyl protecting group.

When R⁶⁴ is —SR⁶⁴³, R⁶⁴³ can be hydrogen or a sulfur protecting group. Accordingly, in some embodiments of any one of the aspects, R⁶⁴³ is hydrogen.

When R⁶⁴ is —O(CH₂CH₂O)ᵣCH₂CH₂OR⁶⁴⁴, r can be 1-50; R⁶⁴⁴ is independently for each occurrence H, C₁-C₃₀alkyl, cyclyl, heterocyclyl, aryl, heteroaryl, aralkyl, sugar or R⁶⁴⁵; and R⁶⁴⁵ is independently for each occurrence amino (NH₂), alkylamino, dialkylamino, arylamino, diarylamino, heteroarylamino, or diheteroaryl amino.

When R⁶⁴ is —NH(CH₂CH₂NH)ₛCH₂CH₂—R⁶⁴⁵, s can be 1-50 and R⁶⁴⁵ can be independently for each occurrence amino (NH₂), alkylamino, dialkylamino, arylamino, diarylamino, heteroarylamino, or diheteroaryl amino.

In some embodiments of any one of the aspects described herein, R⁶⁴ is hydrogen, halogen, —OR⁶⁴², or optionally substituted C₁-C₃₀alkoxy. For example, R⁶⁴ is halogen, —OR⁶⁴², or optionally substituted C₁-C₃₀alkoxy. In some embodiments of any one of the aspects described herein, R⁶⁴ is F, OH or optionally substituted C₁-C₃₀alkoxy.

In some embodiments of any one of the aspects described herein, R⁶⁴ is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ NH₂ or CH₂-aryl-alkoxy, where “m” and “p” are independently 1, 2, 3, 4, 5 or 6. For example, R⁶4 is C₁-C₃₀alkoxy optionally substituted with a NH₂, OH, C(O)NH₂, COOH, halo, SH, or C₁-C₆alkoxy. In some embodiments of any one of the aspects described herein, R⁶⁴ is —O(CH₂)tCH3, where t is 1-21. For example, t is 14, 15, 16, 17 or 18. In one non-limiting example, t is 16.

In some embodiments of any one of the aspects, R⁶⁴ is —O(CH₂)uR⁶⁴⁷, where u is 2-10; R⁶⁴⁷ is C₁-C₆alkoxy, amino (NH₂), CO₂H, OH or halo. For example, R⁶⁴⁷ is —CH₃ or NH₂. Accordingly, in some embodiments of any one of the aspects described herein, R⁶⁴ is —O(CH₂)ᵤ—OMe or R⁶⁴ is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R⁶⁴ is a C₁-C₆haloalkyl. For example, R⁶⁴ is a C₁-C₄haloalkyl. In some embodiments of any one of the aspects described herein, R⁶⁴ is —CF₃, —CF₂CF₃, —CF₂CF₂CF₃ or —CF₂(CF₃)₂.

In some embodiments of any one of the aspects described herein, R⁶⁴ is —OCH(CH₂OR⁶⁴⁸)CH₂OR⁶⁴⁹, where R⁶⁴⁸ and R⁶⁴⁹ independently are H, optionally substituted C₁-C₃₀alkyl, optionally substituted C₂-C₃₀alkenyl or optionally substituted C₂-C₃₀alkynyl. For example, R⁶⁴⁸ and R⁶⁴⁹ independently are optionally substituted C₁-C₃₀alkyl.

In some embodiments of any one of the aspects described herein, R⁶⁴ is —CH₂C(O)NHR⁶⁴¹⁰, where R⁶⁴¹⁰ is H, optionally substituted C₁-C₃₀alkyl, optionally substituted C₂-C₃₀alkenyl or optionally substituted C₂-C₃₀alkynyl. For example, R⁶⁴¹⁰ is H or optionally substituted C₁-C₃₀alkyl. In some embodiments, R⁶⁴¹⁰ is optionally substituted C₁-C₆alkyl.

In some embodiments of any one of the aspects described herein, R⁶³ is hydrogen, fluoro, —O-MOE, —O-alkyl (e.g., methoxy or —O—C₁₆aliphatic), —O-alkene, —O-alkyne, —O-lipid, —O-branched lipid or aminoalkyl.

In some embodiments of any one of the aspects described herein, one of R⁶³ and R⁶⁴ is hydroxyl and the other is hydrogen, methoxy, fluoro, —O-MOE, —O-alkyl, —O-alkene, —O-alkyne, —O—C₁₆, —O-lipid, —O-branched lipid or aminoalkyl.

### R⁶⁵

In some embodiments of the various aspects described herein, R⁶⁵ is R⁶⁵¹ optionally substituted C₁₋₆alkyl-R⁶⁵¹, optionally substituted —C₂₋₆alkenyl-R⁶⁵¹, or optionally substituted —C₂₋₆alkynyl-R⁶⁵¹, where R⁶⁵¹ can be —OR⁶⁵², —SR⁶⁵³, hydrogen, a phosphorous group, a solid support or a linker to a solid support. When R⁶⁵¹ is —OR⁶⁵², R⁶⁵² can be H or a hydroxyl protecting group. Similarly, when R⁶⁵¹ is —SR⁶⁵³, R⁶⁵³ can be H or a sulfur protecting group.

In some embodiments of any one of the aspects described herein, R⁶⁵ is —OR⁶⁵² or —SR⁶⁵³.

In some embodiments of any one of the aspects described herein, R⁶⁵² is a hydroxyl protecting group. Exemplary hydroxyl protecting groups for R⁶⁵² include, but are not limited to, benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In some embodiments of any one of the aspects described herein, R⁶⁵ is —OR⁶⁵² and R⁶⁵² is 4,4′-dimethoxytrityl (DMT), e.g., R⁶⁵ is —O-DMT.

In some embodiments of any one of the aspects described herein, R⁶⁵ is —CH(R⁶⁵⁴)—R⁶⁵¹, where R⁶⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R⁶⁵ is —CH(R⁶⁵⁴)—R⁶⁵¹, R⁶⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁶⁵⁴ is H. In some other non-limiting examples, R⁶⁵⁴ is C₁-C₃₀alkyl optionally substituted with a NH₂, OH, C(O)NH₂, COOH, halo, SH, or C₁-C₆alkoxy.

In some embodiments of the various aspects described herein, R⁶⁵ is —CH(R⁶⁵⁴)—O—R⁶⁵², where R⁶⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈ alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁶⁵⁴ is H. In some other non-limiting examples, R⁶⁵⁴ is C₁-C₃₀alkyl optionally substituted with a NH₂, OH, C(O)NH₂, COOH, halo, SH, or C₁-C₆alkoxy.

In some embodiments of the various aspects described herein, R⁶⁵ is optionally substituted C₁₋₆alkyl-R⁶⁵¹ or optionally substituted —C₂₋₆alkenyl-R⁶⁵¹,

In some embodiments of any one of the aspects described herein, R⁶⁵ is —C(R⁶⁵⁴)=CHR⁶⁵¹. It is noted that the double bond in —C(R⁶⁵⁴)=CR⁶⁵¹ can be in the cis or trans configuration. Accordingly, in some embodiments of any one of the aspects, R^{d} is —C(R⁶⁵⁴)=CHR⁶⁵¹ and wherein the double bond is in the cis configuration. In some other embodiments of any one of the aspects, R^{d} is —C(R⁶⁵⁴)=CR⁶⁵¹ and wherein the double bond is in the trans configuration.

In some embodiments of any one of the aspects described herein, R⁶⁵ is —CH═CHR⁶⁵¹.

In some embodiments of any one of the aspects, when R⁶⁵ is —C(R⁶⁵⁴)=CHR⁶⁵¹, R⁶⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁶⁵¹ is a phosphorous group. For example, R⁶⁵ is —CH═CHR⁶⁵¹.

In some embodiments of any one of the aspects described herein, R⁶⁵¹ is a reactive phosphorous group.

In some embodiments of any one of the aspects, R⁶⁵ is —CH═CH—P(O)(OR⁶⁵⁵)₂, —CH═CH—P(S)(OR⁶⁵⁵)₂, —CH═CH—P(S)(SR⁶⁵⁶)(OR⁶⁵⁵), —CH═CH—P(S)(SR⁶⁵⁶)₂, —CH═CH—OP(O)(OR⁶⁵⁵)₂, —CH═CH—OP(S)(OR⁶⁵⁵)₂, —CH═CH—OP(S)(SR⁶⁵⁶)(OR⁶⁵⁵), —CH═CH—OP(S)(SR⁶⁵⁶)₂, —CH═CH—SP(O)(OR⁶⁵⁵)₂, —CH═CH—SP(S)(OR⁶⁵⁵)₂, —CH═CH—SP(S)(SR⁶⁵⁶)(OR655) or —CH═CH—SP(S)(SR⁶⁵⁶)₂, where each R⁶⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁶⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁶⁵⁵ in —P(O)(OR⁶⁵⁵)₂, —P(S)(OR⁶⁵⁵)₂, —P(S)(SR⁶⁵⁶)(OR⁶⁵⁵), —OP(O)(OR⁶⁵⁵)₂, —OP(S)(OR⁶⁵⁵)₂, —OP(S)(SR⁶⁵⁶)(OR⁶⁵⁵), SP(O)(OR⁶⁵⁵)₂, —SP(S)(OR⁶⁵⁵)₂, and —SP(S)(SR⁶⁵⁶)(OR⁶⁵⁵) is hydrogen.

In some other embodiments of any one of the aspects, at least one R⁶⁵⁵ in —P(O)(OR⁶⁵⁵)₂, —P(S)(OR⁶⁵⁵)₂, —P(S)(SR⁶⁵⁶)(OR⁶⁵⁵), —OP(O)(OR⁶⁵⁵)₂, —OP(S)(OR⁶⁵⁵)₂, —OP(S)(SR⁶⁵⁶)(OR⁶⁵⁵), SP(O)(OR⁶⁵⁵)₂, —SP(S)(OR⁶⁵⁵)₂, or —SP(S)(SR⁶⁵⁶)(OR⁶⁵⁵) is not hydrogen. For example, at least one at least one R⁶⁵⁵ in P(O)(OR⁶⁵⁵)₂, —P(S)(OR⁶⁵⁵)₂, —P(S)(SR⁶⁵⁶)(OR⁶⁵⁵), —OP(O)(OR⁶⁵⁵)₂, —OP(S)(OR⁶⁵⁵)₂, —OP(S)(SR⁶⁵⁶)(OR⁶⁵⁵), SP(O)(OR⁶⁵⁵)₂, —SP(S)(OR⁶⁵⁵)₂, and —SP(S)(SR⁶⁵⁶)(OR⁶⁵⁵) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁶⁵⁵ is H and at least one R⁶⁵⁵ is other than H in —P(O)(OR⁶⁵⁵)₂, —P(S)(OR⁶⁵⁵)₂, —P(S)(SR⁶⁵⁶)(OR⁶⁵⁵)—OP(O)(OR⁶⁵⁵)₂, —OP(S)(OR⁶⁵⁵)₂, —OP(S)(SR⁶⁵⁶)(OR⁶⁵⁵), SP(O)(OR⁶⁵⁵)₂, —SP(S)(OR⁶⁵⁵)₂, and —SP(S)(SR⁶⁵⁶)(OR⁶⁵⁵).

In some embodiments of any one of the aspects, all R⁶⁵⁵ are H in —P(O)(OR⁶⁵⁵)₂, —P(S)(OR⁶⁵⁵)₂, —P(S)(SR⁶⁵⁶)(OR⁶⁵⁵), —OP(O)(OR⁶⁵⁵)₂, —OP(S)(OR⁶⁵⁵)₂, —OP(S)(SR⁶⁵⁶)(OR⁶⁵⁵), —OP(S)(SR⁶⁵⁶)₂, —SP(O)(OR⁶⁵⁵)₂, —SP(S)(OR⁶⁵⁵)₂, —SP(S)(SR⁶⁵⁶)(OR⁶⁵⁵), and —SP(S)(SR⁶⁵⁶)₂.

In some embodiments of any one of the aspects, all R⁶⁵⁵ are other than H in in —P(O)(OR⁶⁵⁵)₂, —P(S)(OR⁶⁵⁵)₂, —P(S)(SR⁶⁵⁶)(OR⁶⁵⁵), —OP(O)(OR⁶⁵⁵)₂, —OP(S)(OR⁶⁵⁵)₂, —OP(S)(SR⁶⁵⁶)(OR655), —OP(S)(SR⁶⁵⁶)₂, —SP(O)(OR⁶⁵⁵)₂, —SP(S)(OR⁶⁵⁵)₂, —SP(S)(SR⁶⁵⁶)(OR⁶⁵⁵), and —SP(S)(SR⁶⁵⁶)₂.

In some embodiments of any one of the aspects, at least one R⁶⁵⁶ in —P(S)(SR⁶⁵⁶)(OR⁶⁵⁵), —P(S)(SR⁶⁵⁶)₂, —OP(S)(OR⁶⁵⁵)₂, —OP(S)(SR⁶⁵⁶)(OR⁶⁵⁵), —OP(S)(SR⁶⁵⁶)₂, —SP(S)(SR⁶⁵⁶)(OR⁶⁵⁵), and —SP(S)(SR⁶⁵⁶)₂ is H.

In some embodiments of any one of the aspects, at least one R⁶⁵⁶ in —P(S)(SR⁶⁵⁶)(OR⁶⁵⁵), —P(S)(SR⁶⁵⁶)₂, —OP(S)(OR⁶⁵⁵)₂, —OP(S)(SR⁶⁵⁶)(OR⁶⁵⁵), —OP(S)(SR⁶⁵⁶)₂, —SP(S)(SR⁶⁵⁶)(OR⁶⁵⁵), and —SP(S)(SR⁶⁵⁶)₂ is other than H. For example, at least one R⁶⁵⁶ in —P(S)(SR⁶⁵⁶)(OR⁶⁵⁵), —P(S)(SR⁶⁵⁶)₂, —OP(S)(OR⁶⁵⁵)₂, —OP(S)(SR⁶⁵⁶)(OR⁶⁵⁵), —OP(S)(SR⁶⁵⁶)₂, —SP(S)(SR⁶⁵⁶)(OR⁶⁵⁵), and —SP(S)(SR⁶⁵⁶)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁶⁵⁶ is H and at least one R⁶⁵⁶ is other than H in —P(S)(SR⁶⁵⁶)₂, —OP(S)(SR⁶⁵⁶)₂ and —SP(S)(SR⁶⁵⁶)₂.

In some embodiments, all R⁶⁵⁶ are H in —P(S)(SR⁶⁵⁶)(OR⁶⁵⁵), —P(S)(SR⁶⁵⁶)₂, —OP(S)(OR⁶⁵⁵)₂, —OP(S)(SR⁶⁵⁶)(OR⁶⁵⁵), —OP(S)(SR⁶⁵⁶)₂, —SP(S)(SR⁶⁵⁶)(OR⁶⁵⁵), and —SP(S)(SR⁶⁵⁶)₂.

In some embodiments, all R⁶⁵⁶ are other than H in —P(S)(SR⁶⁵⁶)(OR⁶⁵⁵)—P(S)(SR⁶⁵⁶)₂, —OP(S)(OR⁶⁵⁵)₂, —OP(S)(SR⁶⁵⁶)(OR⁶⁵⁵), —OP(S)(SR⁶⁵⁶)₂, —SP(S)(SR⁶⁵⁶)(OR⁶⁵⁵), and —SP(S)(SR⁶⁵⁶)₂.

In some embodiments of any one of the aspects, R⁶⁵ is —CH═CH—P(O)(OR⁶⁵⁵)₂, where each R⁶⁵⁵ is H or an oxygen protecting group.

In some embodiments of any one of the aspects, R⁶³ is a reactive phosphorous group, a solid support, a linker to a solid support, and R⁶⁵ is a protected hydroxyl.

### R^{62x}

In some embodiments of any one of the aspects described herein, R^{62x} can be a bond to an internucleotide linkage to a subsequent nucleotide, hydroxy, protected hydroxy, optionally substituted C₁₋₃₀ alkoxy, halogen, alkoxyalkyl (e.g., methoxyethyl), amino, alkylamino, dialkylamino, a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, or a linker covalently bonded (e.g., —C(O)CH₂CH₂C(O)—) to a solid support.

In some embodiments of any one of the aspects described herein, R^{62x} is a bond to an internucleotide linkage to a subsequent nucleotide, hydroxy, protected hydroxy, optionally substituted C₁₋₃₀ alkoxy, a 3′-oligonuclotide capping group (e.g., an inverted nucleotide or an inverted abasic nucleotide), a solid support, or a linker covalently bonded (e.g., —C(O)CH₂CH₂C(O)—) to a solid support. For example, R^{62x} is a bond to an internucleotide linkage to a subsequent nucleotide, hydroxy, a solid support, or a linker covalently bonded (e.g., —C(O)CH₂CH₂C(O)—) to a solid support. In some embodiments of any one of the aspects described herein, R⁵² is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R^{62x} is a bond to an internucleotide linkage to a subsequent nucleotide.

In some embodiments of any one of the aspects described herein, R^{62x} is a solid support, or a linker covalently bonded to a solid support.

In some embodiments of any one of the aspects described herein, R^{62x} is hydroxyl.

### R^{65x}

In some embodiments of any one of the aspects described herein, R^{65x} can be a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10).

In some embodiments of any one of the aspects described herein, R^{65x} can be a bond to an internucleotide linkage to a preceding nucleotide, hydroxy, protected hydroxy, optionally substituted C₂₋₃₀alkenyl, optionally substituted C₂₋₃₀alkynyl, optionally substituted C₁₋₃₀ alkoxy, vinylphosphonate (VP) group, monophosphate, diphosphate, triphosphate, monothiophosphate (phosphorothioate), monodithiophosphate (phosphorodithioate), phosphorothiolate, alpha-thiotriphosphate, beta-thiotriphosphate, gamma-thiotriphosphate, phosphoramidates, or alkylphosphonates.

In some embodiments of any one of the aspects described herein, R^{65x} is a bond to an internucleotide linkage to a preceding nucleotide, hydroxy, protected hydroxy, optionally substituted C₂₋₃₀alkenyl, optionally substituted C₁₋₃₀ alkoxy or a vinylphosphonate (VP) group.

In some embodiments of any one of the aspects described herein, R^{65x} is a bond to an internucleotide linkage to a preceding nucleotide.

In some embodiments of any one of the aspects described herein, R^{65x} is a hydroxyl or protected hydroxyl.

In some embodiments of any one of the aspects described herein, R^{65x} is optionally substituted C₂₋₃₀alkenyl or optionally substituted C₁₋₃₀ alkoxy.

In some embodiments of any one of the aspects described herein, R^{65x} is a vinylphosphonate group.

### L

In embodiments of the various aspects described herein, L is a linker.

As used herein, the term “linker” means an organic moiety that connects two parts of a compound. Linkers typically comprise a direct bond or an atom such as oxygen or sulfur, a unit such as NR¹, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R^{r} is hydrogen, acyl, aliphatic or substituted aliphatic.

In some embodiments, the linker is a cleavable linker. Cleavable linkers are those that rely on processes inside a target cell to liberate the two parts the linker is holding together, as reduction in the cytoplasm, exposure to acidic conditions in a lysosome or endosome, or cleavage by specific enzymes (e.g. proteases) within the cell. As such, cleavable linkers allow the two parts to be released in their original forn after internalization and processing inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates).

Generally, the cleavable linker comprises at least one cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

One class of cleavable linking groups is redox cleavable linking groups, which may be used in the dsRNA molecule according to the present invention that are cleaved upon reduction or oxidation. An example of reductively cleavable linking group is a disulfide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups, which may be used in the dsRNA molecule according to the present invention, are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, hydrogen, C1-C20 alkyl, C1-C20 haloalkyl, C6-C10 aryl, C7-C12 aralkyl. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid cleavable linking groups, which may be used in the dsRNA molecule according to the present invention, are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups, which may be used in the dsRNA molecule according to the present invention, are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-based cleavable linking groups, which may be used in the dsRNA molecule according to the present invention, are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and R^{B} are the R groups of the two adjacent amino acids.

In some embodiments of any one of the aspects, L is a bond.

In some embodiments of any one of the aspects, L is absent, e.g., R⁶ or R⁷ is —R^{L}.

### L^{P}

In some embodiments of any one of the aspects, L^{P} is a linker. For example, L^{P} can be a bond.

In some embodiments of any one of the aspects described herein, L^{P} is an optionally substituted C₁-C₂₀alkylene, (e.g., —(CH₂)b-, where b is 1, 2, 3, 4, 5, 6, 7, 8, 9, 10, 11, 12, 13, 14, 14, 15, 16, 17, 18, 19 or 20), or optionally substituted C₂-C₂₀alkynylene, and where the backbone of the alkylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic. For example, L^{P} is an optionally substituted C₁-C₆alkylene.

In some embodiments of any one of the aspects, L^{P} is an optionally substituted C₁-C₂₀alkylene, where the backbone of the alkylene is interrupted with a heteroaryl (e.g., triazole) or NHC(O).

In some embodiments of any one of the aspects, L^{P} is optionally substituted C₂-C₂₀alkylene. For example, L^{P} is —(CH₂)₃—, —(CH₂)₅—, —(CH₂)₇—, —(CH₂)₉—, —(CH₂)₁₀—, —(CH₂)₁₁—, —(CH₂)₁₂—, —(CH₂)₁₃—, —(CH₂)₁₅—, or —(CH₂)₁₇—.

In some embodiments of any one of the aspects, L^{P} is a polyethylene glycol. For Example, L^{P} is —(CH₂CH₂O)_{L}—O—CH₂—, where L′ is an integer selected from 1 to 25. In some embodiments, L′ is an integer selected from 1 to 10. For example, L′ is 1, 2, 3, 4, 5 or 6.

In some embodiments of any one of the aspects, L^{P} is absent.

### Internucleoside Linkages

As used herein, “internucleoside linkage” refers to a covalent linkage between adjacent nucleosides. The two main classes of internucleoside linkages are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH2-N(CH3)-O—CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Modified internucleoside linkages, compared to natural phosphodiester linkages, can be used to alter, typically increase, nuclease resistance of the oligonucleotide compound. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the internucleoside linkage can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphodiester internucleoside linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral. In other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. The non-bridging oxygens can be independently any one of O, S, Se, B, C, H, N, or OR (R is alkyl or aryl).

A phosphodiester internucleoside linkage can also be modified by replacement of bridging oxygen, (i.e. oxygen that links the phosphate to the sugar of the nucleosides), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′—S—CH₂—O-5′), formacetal (3′—O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′—O—C5′), thioethers (C3′—S—C5′), thioacetamido (C3′—N(H)—C(═O)—CH₂—S—C5′, C3′—O—P(O)—O—SS—C5′, C3′-CH₂—NH—NH—C5′, 3′—NHP(O)(OCH₃)—O-5′ and 3′—NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester internucleoside linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80% o, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Additional exemplary non-phosphorus containing internucleoside linking groups are described in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, content of each of which is incorporated herein by reference.

In some embodiments of any one of the aspects, the oligonucleotides described herein comprise one or more neutral internucleoside linkages that are non-ionic. Suitable neutral internucleoside linkages include, but are not limited to, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′); nonionic linkages containing siloxane (dialkylsiloxane), carboxylate ester, carboxamide, sulfide, sulfonate ester and/or amides (See for example: Carbohydrate Modifications in Antisense Research; Y. S. Sanghvi and P. D. Cook Eds. ACS Symposium Series 580; Chapters 3 and 4, (pp. 40-65)); and nonionic linkages containing mixed N, O, S and CH₂ component parts.

In one embodiment, the non-phosphodiester backbone linkage is selected from the group consisting of phosphorothioate, phosphorodithioate, alkyl-phosphonate and phosphoramidate backbone linkages.

In some embodiments of any one of the aspects described herein, the internucleoside linkage is

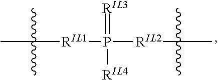

where R^{IL1} and R^{IL2} are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and R^{IL3} and R^{IL4} are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), alkyl or aryl. It is understood that one of R^{IL1} and R^{IL2} is replacing the oxygen linked to 5′ carbon of a first nucleoside sugar and the other of R^{IL1} and R^{IL2} is replacing the oxygen linked to 3′ (or 2′) carbon of a second nucleoside sugar.

In some embodiments of any one of the aspects, R^{IL1}, R^{IL2}, R^{IL3} and R^{IL4} all are O.

In some embodiments, R^{IL1} and R^{IL2} are O and at least one of R^{IL3} and R^{IL4} is other than O. For example, one of R^{IL3} and R^{IL4} is S and the other is O or both of R^{IL3} and R^{IL4} are S.

In some embodiments of any one of the aspects described herein, one of R³ or R⁵ is a bond to a modified internucleoside linkage, e.g., an internucleoside linkage of structure:

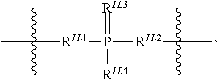

where at least one of R^{IL1}, R^{IL2}, R^{IL3} and R^{IL4} is not O. For example, at least one of R^{IL3} and R^{IL4} is S.

In some embodiments of any one of the aspects described herein, both of R³ and R⁵ are a bond to a modified internucleoside linkage.

In some embodiments of any one of the aspects described herein R³ is a bond to phosphodiester internucleoside linkage.

In some embodiments of any one of the aspects described herein R⁵ is a bond to phosphodiester internucleoside linkage.

In some embodiments of any one of the aspects described herein, R³ is a bond to a modified internucleoside linkage and R⁵ is a bond to phosphodiester internucleoside linkage.

In some embodiments of any one of the aspects described herein, R⁵ is a bond to a modified internucleoside linkage and R³ is a bond to phosphodiester internucleoside linkage.

In some embodiments of any one of the aspects, the oligonucleotide can comprise one or more, e.g., 1, 2, 3, 4, 5, 6, 7, 8 or more modified internucleoside linkages. For example, the oligonucleotide can comprise 1, 2, 3, 4, 5 or 6 modified internucleoside linkages. For example, the oligonucleotide comprises 1, 2, 3 or 4 modified internucleoside linkages. In some embodiments, the oligonucleotide comprises at least two modified internucleoside linkages between the first five nucleotides counting from the 5′-end of the oligonucleotide and further comprises at least two modified internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments of any one of the aspects, the modified internucleoside linkage is a phosphorothioate. Accordingly, in some embodiments of any one of the aspects, the oligonucleotide comprises one or more, e.g., 1, 2, 3, 4, 5, 6, 7, 8 or more phosphorothioate internucleoside linkages. For example, the oligonucleotide comprises 1, 2, 3, 4, 5 or 6 phosphorothioate internucleoside linkages. For example, the oligonucleotide comprises 1, 2, 3 or 4 phosphorothioate internucleoside linkages. In some embodiments, the oligonucleotide comprises at least two phosphorothioate internucleoside linkages between the first five nucleotides counting from the 5′-end of the oligonucleotide and further comprises at least two phosphorothioate internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

### Oxygen Protecting Groups

Some embodiments of the various aspects described herein include an oxygen protecting group (also referred to as an hydroxyl protecting group herein). Oxygen protecting groups include, but are not limited to, —R^{OP1}, —N(R^{OP2})₂, —C(═O)SR^{OP1}, —C(═O) R^{OP1}, —CO: R^{OP1}, —C(═O)N(R^{OP2})₂, —C(═NR^{OP2})R^{OP1}, —C(═NR^{OP2})OR^{OP1}, —C(═NR^{OP2})N(R^{OP2})₂, —S(═O)R^{OP1}, —SO⁺₂R^{OP1}, —Si(R^{OP1})₃, —P(R^{OP3})₂, —P(R^{OP3})⁺₃X⁻, —P(OR^{OP3})₂, —P(OR^{OP3})₃X⁻, —P(═O)(R^{OP1})₂, —P(═O)(OR^{OP3})₂, and —P(═O)(N(R^{OP2})₂)₂; wherein each X⁻ is a counterion; each R^{OP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃-10 carbocyclyl, 3-14 membered heterocyclyl, C₆₋₁₄ aryl, or 5-14 membered heteroaryl, or two R^{OP1} groups are joined to form a 3-14 membered heterocyclyl or 5-14 membered heteroaryl ring; each R^{OP2} is hydrogen, —OH, —OR^{OP1}, —N(R^{OP3})₂, —CN, —C(═O)R^{OP1}, —C(═O)N(R^{OP3})₂, —CO₂R^{OP1}, —SO₂R^{OP1}, —C(═NR^{OP3})OR^{OP1}, —C(═NR^{OP3})N(R^{OP3})₂, —SO₂N(R^{OP3})₂, —SO₂R^{OP3}, —SO₂OR^{OP3}, —SOR^{OP1}, —C(═S)N(R^{OP3})₂, —C(═O)SR^{OP3}, —C(═S)SR^{OP3}, —P(═O)(R^{OP1})₂, —P(═O)(OR^{OP3})₂, —P(═O)(N(R^{OP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃-10 carbocyclyl, 3-14 membered heterocyclyl, C₆₋₁₄ aryl, and 5-14 membered heteroaryl, or two R^{OP2} groups are joined to form a 3-14 membered heterocyclyl or 5-14 membered heteroaryl ring; and each R^{OP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃-10 carbocyclyl, 3-14 membered heterocyclyl, C₆₋₁₄ aryl, and 5-14 membered heteroaryl, or two R^{OP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{OP1}, R^{OP2} and R^{OP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₅)alkyl, O(C₁-C₈)alkyl (i.e., C₁-C₈alkoxy), O(C₁-C₅) haloalkyl, (C₂-C₅) alkenyl, (C₂-C₅) alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Oxygen protecting groups are well known in the art and include those described in detail in Greene's Protecting Groups in Organic Synthesis, P. G. M. Wuts, 5th Edition, John Wiley & Sons, 2014, incorporated herein by reference.

Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy) methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4˜ methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a, 4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyI) ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl) diphenylmethyl, 4,4′,4″-tris (4,5-dichlorophthalimidophenyl)methyl, 4,4′,4″-tris (levulinoyloxyphenyl)methyl, 4,4′,4″-tris(benzoyloxyphenyl)methyl, 3-(imidazol-1-yl) bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), adamantoate, crotonate, 4, methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuSP3inoate, (E)-2-methyl-2-butenoate, (methoxyacyl)benzoate, α-naphthoate, nitrate, alkylN,N,N′,N′-tetramethylphosphorodiamidate, alkyl N-phenylcarbamate, borate,dimethylphosphinothioyl, alkyl 2,4-dinitrophenylsulfenate, sulfate, methanesulfonate (mesylate), benzylsulfonate, and tosylate (Ts).

In some embodiments of any one of the aspects described herein, oxygen protecting group is benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl) xanthine-9-yl (MOX). In certain embodiments, the hydroxyl protecting group is selected from acetyl, benzyl, t-butyldimethylsilyl, t-butyldiphenylsilyl and dimethoxytrityl wherein a more preferred hydroxyl protecting group is 4,4′-dimethoxytrityl.

The terms “protected hydroxyl” and “protected hydroxy” as used herein mean a group of the formula-ORPro, wherein RPro is an oxygen protecting group as defined herein.

### Nitrogen Protecting Groups

Some embodiments of the various aspects described herein include a nitrogen protecting group (also referred to as an amino protecting group herein). Nitrogen protecting groups include, but are not limited to, —OH, ˜OR^{NP1}, —N(R^{NP2})₂, —C(═O)R^{NP1}, —C(═O)N(R^{NP2})₂, —CO₂R^{NP1}, —SO₂R^{NP1}—C(═NR^{NP2})R^{NP1}, —C(═NR^{NP2}) OR^{NP1}, C(═NR^{NP2})N(R^{NP2})₂, —SO₂N(R^{NP2})₂, —SO₂R^{NP2}, —SO₂OR^{NP2}, —SOR^{NP1}, —C(═S)N(R^{NP2})₂, —C(═O)SR^{NP2}, —C(═S)SR^{NP2}, C₁₋₁₀ alkyl (e.g., aralkyl, heteroaralkyl), C₂₋₁₀ alkenyl, C₂₋₁₀ alkynyl, C₃-10 carbocyclyl, 3-14 membered heterocyclyl, C₆₋₁₄ aryl, and 5-14 membered heteroaryl groups, where each R^{NP1} is independently C₁₋₁₀ alkyl, C₁₋₁₀ perhaloalkyl, C₂₋₁₀ alkenyl, C₁₋₁₀ alkynyl, heteroC₂₋₁₀ alkyl, heteroC₂₋₁₀alkenyl, heteroC₂₋₁₀alkynyl, C₃-10 carbocyclyl, 3-14 membered heterocyclyl, C₆₋₁₄ aryl, or 5-14 membered heteroaryl, or two R^{NP1} groups are joined to form a 3-14 membered heterocyclyl or 5-14 membered heteroaryl ring; and each R^{NP2} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₁₋₁₀ alkenyl, heteroC₂₋₁₀ alkynyl, C₃-10 carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring, and wherein each alkyl, alkenyl, alkynyl, carbocyclyl, heterocyclyl, aralkyl, aryl, and heteroaryl of R^{NP1} and R^{NP2} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N [(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₅) haloalkyl, (C₂-C₈) alkenyl, (C₂—C) alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Nitrogen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary amide (e.g., —C(═O)R^{NP1}) nitrogen protecting groups include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o˜ nitophenylacetamide, o-nitrophenoxyacetamide, acetoacetamide, (N′-dithiobenzyloxy acylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Exemplary carbamate (e.g., —C(═O)OR^{NP1}) nitrogen protecting groups include, but are not limited to, methyl carbamate, ethyl carbamate, 9-fluorenylmethyl carbamate (Fmoc), 9-(2-sulfo) fluorenylmethyl carbamate, 9. (2,7-dibromo) fluoroenylmethyl carbamate, 2,7-di-t-butyl-[9-(10,10-dioxo-10, 10, 10, 10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenyly) ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido) ethyl carbamate, t-butyl carbamate (BOC or Boc), I-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl) methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, 0. (N,N-dimethylcarboxamido) benzyl carbamate, 1,1-dimethyl-3-(N,N-dimethylcarboxamido) propyl carbamate, 1,1-dimethylpropynyl carbamate, di (2-pyridyl) methyl carbamate, 2-furanylmethyl carbamate, 2-iodoethyl carbamate, isoborynl carbamate, isobutyl carbamate, isonicotinyl carbamate, p-(p′-methoxyphenylazo) benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo) benzyl carbamate, 2,4,6-tri-t-butylphenyl carbamate, 4-(trimethylammonium) benzyl carbamate, and 2,4,6-trimethylbenzyl carbamate.

Exemplary sulfonamide (e.g., —S(═O)₂R^{NP1}) nitrogen protecting groups include, but are not limited to, such as p-toluenesulfonamide (Ts), benzenesulfonamide, 2,3,6, -trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamido.

Additional exemplary nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuNP2inimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl) amine, quaternary ammonium salts, N-benzylamine, N-di (4-methoxyphenyl)methylamine, N-5-dibenzosuberylamine, N-triphenylmethylamine (Tr), N-[(4, methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-bonzylideneamine, N-p-methoxybenzylideneamine, N-diphenylmethyleneamine, N-[(2-pyridyl) mesityl]methyleneamine, N—(N′,N′-dimethylaminomethylene) amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylidencamine, N-(5,5-dimethyl-3-oxo-1-cyclohexenyl) amine, N-borane and N-diphenylborinic acid derivative, N-[phenyl(pentNP1cylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfonamide, pentachlorobenzenesulfonamide, 2-nitro-4-methoxybenzenesulfonamide, triphenylmethylsulfenamide, and 3-nitropyridinesulfenamide (Npys).

### Sulfur Protecting Groups

Some embodiments of the various aspects described herein include sulfur protecting group (also referred to as a thiol protecting group herein). Sulfur protecting groups include, but are not limited to, —R^{SP1}, —N(R^{SP2})₂, —C(═O)SR^{SP1}, —C(═O) R^{SP1}, —CO₂R^{SP1}, —C(O)N(R^{SP2})₂, —C(═NR^{SP2})R^{SP1}, —C(═N^{RS2})OR^{SP1}, —C(═NR^{SP2})N(R^{SP2})₂, —S(═O) R^{SP1}, SO₂R^{SP1}, —Si (R^{SP1})₃, —P(R^{SP3})₂, —P(R^{SP3})⁺₃X⁻, —P(OR^{SP3})₂, —P(OR^{SP3})₃X⁻, —P(═O)(R^{SP1})₂, —P(═O)(OR^{SP3})₂, and —P(═O)(N(R^{SP2})₂)₂, wherein

X⁻ is a counterion; each R^{SP1} is independently C₁₋₁₀ alkyl, C₁₋₁₀ perbaloalkyl, C₂₋₁₀ alkenyl, C₁₋₁₀ alkynyl, heteroC₁₋₁₀ alkyl, heteroC₂₋₁₀alkenyl, heteroC₂₋₁₀alkynyl, C₃-10 carbocyclyl, 3-14 membered heterocyclyl, C₆₋₁₄ aryl, or 5-14 membered heteroaryl, or two R^{SP1} groups are joined to form a 3-14 membered heterocyclyl or 5-14 membered heteroaryl ring; each R^{SP2} is hydrogen, OH, OR^{SP1}, —N(R^{SP3})₂, —CN, —C(O)R^{SP1}, C(═O)N(R^{SP3})₂, —CO₂R^{SP1}, —SO₂R^{SP1}, C(═NR^{SP3})OR^{SP1}, C(═NR^{SP3})N(R^{SP3})₂, —SO₂N(R^{SP3})₂, —SO₂R^{SP3}, —SO₂OR^{SP3}, —SO₂R^{SP1}, —C(═S)N(R^{SP3})₂, C(═O)SR^{SP3}, —C(═S)SR^{SP3}, —P(═O) (R^{SP1})₂, —P(═O)(OR^{SP3})₂, —P(═O)(N(R^{SP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₁₋₁₀alkenyl, heteroC₁₋₁₀alkynyl, C₃-10 carbocyclyl, 3-14 membered heterocyclyl, C₆₋₁₄ aryl, and 5-14 membered heteroaryl, or two R^{SP2} groups are joined to form a 3-14 membered heterocyclyl or 5-14 membered heteroaryl ring; and each R^{SP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃-10 carbocyclyl, 3-14 membered heterocyclyl, C₆₋₁₄ aryl, and S-14 membered heteroaryl, or two R^{SP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{SP1}, R^{SP2} and R^{SP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N [(C₁-C₄)alkyl]₂, C(O)NH₂, COOH, COOMe, acetyl, (C₁-C₈)alkyl, O(C₁-C₅)alkyl (i.e., C₁-C₈alkoxy), O(C₁-C₈) haloalkyl, (C₂-C₅) alkenyl, (C₂-C₈) alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Sulfur protecting groups are well known in the art and include those described in detail in Greene's Protecting Groups in Organic Synthesis, P. G. M. Wuts, 5th Edition, John Wiley & Sons, 2014, incorporated herein by reference.

It is noted that the nucleoside of Formula (I) can be located anywhere in the oligonucleotide. In some embodiments, the nucleoside of Formula (I) is present at the 5′- or 3′-terminus of the oligonucleotide. In some embodiments, the nucleoside of Formula (I) is present at an internal position of the oliogunculeotide.

In some embodiments of any one of the aspects described herein, the oligonucleotide further comprises, i.e., in addition to a nucleotiside of Formula (I), a nucleoside with a modified sugar. By a “modified sugar” is meant a sugar or moiety other than 2′-deoxy (i.e, 2′-H) or 2′-OH ribose sugar. Some exemplary nucleotides comprising a modified sugar are 2′-F ribose, 2′-OMe ribose, 2′-O,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxypropyl (glycol nucleic acid, GNA). It is noted that the nucleoside with the modified sugar can be present at any position of the oligonucleotide.

In some embodiments, the oligonucleotide further comprises at least one, e.g., 1, 2, 3, 4, 5, 6, 7, 8, 9, 10 or more 2′-fluoro (2′-F) nucleotides. For example, the oligonucleotide can comprise 1, 2, 3, 4, 5, 6, 7, 8, 9 or 10 2′-F nucleotides. It is noted that the 2′-F nucleotides can be present at any position of the oligonucleotide.

In some embodiments, the oligonucleotide comprises, e.g., solely comprises 2′-nucleosides of Formula (I) and 2′-F nucleosides.

In some embodiments, the oligonucleotide further comprises at least one, e.g., 1, 2, 3, 4, 5, 6, 7, 8, 9, 10 or more 2′-OMe nucleotides. For example, the oligonucleotide can comprise 1, 2, 3, 4, 5, 6, 7, 8, 9 or 10 2′-OMe nucleotides. It is noted that the 2′-OMe nucleotides can be present at any position of the oligonucleotide.

In some embodiments, the oligonucleotide comprises, e.g., solely comprises solely comprises solely comprises 2′-nucleosides of Formula (I) and 2′-OMe nucleosides. In some other embodiments, the oligonucleotide comprises, e.g., solely comprises solely comprises 2′-nucleosides of Formula (I), 2′-OMe nucleosides and 2′-F nucleosides.

In some embodiments, the oligonucleotide further comprises at least one, e.g., 1, 2, 3, 4, 5, 6, 7, 8, 9, 10 or more 2′-deoxy, e.g., 2′-H nucleotides. For example, the oligonucleotide can comprise 1, 2, 3, 4, 5, 6, 7, 8, 9 or 10 of 2′-deoxy, e.g., 2′-H nucleotides. It is noted that the 2′-deoxy, e.g., 2′-H nucleotides can be present at any position of the oligonucleotide. For example, the oligonucleotide can comprise a 2′-deoxy, e.g., 2′-H nucleotide at 1, 2, 3, 4, 5 or 6 of positions 2, 5, 7, 12, 14 and 16, counting from 5′-end of the oligonucleotide. In some embodiments, the oligonucleotide comprises a 2′-deoxy nucleotide at positions 5 and 7, counting from 5′-end of the oligonucleotide.

In some embodiments, the oligonucleotide comprises, e.g., solely comprises solely comprises nucleosides of Formula (I)) and 2′-deoxy (2′-H) nucleotides. In some embodiments, the oligonucleotide comprises, e.g., solely comprises nucleosides of Formula (I), 2′-OMe nucleosides, and 2′-deoxy (2′-H) nucleotides. In some embodiments, the oligonucleotide comprises, e.g., solely comprises nucleosides of Formula (I), 2′-F nucleosides and 2′-deoxy (2′-H) nucleotides. In some embodiments, the oligonucleotide comprises, e.g., solely comprises nucleosides of Formula (I), 2′-OMe nucleosides, 2′-F nucleosides and 2′-deoxy (2′-H) nucleotides.

In some embodiments of any one of the aspects described herein, the oligonucleotide further comprises, i.e., in addition to a nucleotide of Formula (I), a non-natural nucleobase. In some embodiments, the oligonucleotide can comprise one or more, e.g., 1, 2, 3, 4, 5, 6, 7, 8, 9, 10 or more nucleotides comprising an independently selected non-natural nucleobase. When present, a nucleotide comprising a non-natural nucleobase can be present anywhere in the oligonucleotide.

By a “non-natural nucleobase” is meant a nucleobase other than adenine, guanine, cytosine, uracil, or thymine. Exemplary non-natural nucleobases include, but are not limited to, inosine, xanthine, hypoxanthine, nubularine, isoguanisine, tubercidine, and substituted or modified analogs of adenine, guanine, cytosine and uracil, such as 2-aminoadenine and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613, content of all which is incorporated herein by reference.

In some embodiments, the non-natural nucleobase can be selected from the group consisting of inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

In some embodiments, a non-natural nucleobase is a modified nucleobase, i.e., the nucleobase comprises a nucleobase modification described herein, e.g., the nucleobase is a substituted or modified analog of any of the natural nucleobases. Examples of the nucleobase modifications include, but not limited to: C-5 pyrimidine with an alkyl group or aminoalkyls and other cationic groups such as guanidinium and amidine functionalities, N²— and N⁶— with an alkyl group or aminoalkyls and other cationic groups such as guanidinium and amidine functionalities of purines, G-clamps, guanidinium G-clamps, and pseudouridine known in the art.

In some embodiments of any one of the aspects, the non-natural nucleobase is a universal nucleobase. As used herein, a universal nucleobase is any modified or unmodified natural or non-natural nucleobase that can base pair with all of adenine, cytosine, guanine and uracil without substantially affecting the melting behavior, recognition by intracellular enzymes or activity of the oligonucleotide comprising the universal nucleobase. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof.

In some embodiments of any one of the aspects described herein, the non-matural nucleobase is a protected nucleobase. As used herein, a “protected nucleobase” referes to a nucleobase comprising a nitrogen protecting group, and/or an oxygen protecting group, and/or a sulfur protecting group.

In some embodiments of any one of the aspects described herein, the non-natural nucleobase is a modified, protected or substituted analogs of a nucleobase selected from adenine, cytosine, guanine, thymine, and uracil.

In some embodiments, the oligonucleotide further comprises a solid support linked thereto.

The oligonucleotides described herein can range from few nucleotides (e.g., 2, 3, 4, 5, 6, 7, 8, 9 or 10 nucleotides) in length to hundreds of nucleotides in length. For example, the oligonucleotide can be from 5 nucleotides to 100 nucleotides in length. In some embodiments, the oligonucleotide is from 10 nucleotides to 50 nucleotides in length. For example, the oligonucleotide is between 15 and 35, more generally between 18 and 25, yet more generally between 19 and 24, and most generally between 19 and 21 base pairs in length. In some embodiments, longer oligonucleotides of between 25 and 30 nucleotides in length are preferred. In some embodiments, shorter oligonucleotides of between 10 and 15 nucleotides in length are preferred. In another embodiment, the oligonucleotide is at least 21 nucleotides in length.

In some embodiments, the oligonucleotide described herein comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, the oligonucleotide described herein comprises a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, the oligonucleotide described herein comprises a 5′-block is an Sp block wherein each sugar moiety comprises a 2′-fluoro modification. In some embodiments, a 5′-block is an Sp block wherein each of internucleotidic linkage is a modified internucleotidic linkage and each sugar moiety comprises a 2′-fluoro modification. In some embodiments, a 5′-block is an Sp block wherein each of internucleoside linkage is a phosphorothioate linkage and each sugar moiety comprises a 2′-fluoro modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-fluoro modification. In some embodiments, a 3′-block is an Sp block wherein each of internucleotidic linkage is a modified internucleotidic linkage and each sugar moiety comprises a 2′-fluoro modification. In some embodiments, a 3′-block is an Sp block wherein each of internucleotidic linkage is a phosphorothioate linkage and each sugar moiety comprises a 2′-fluoro modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, oligonucleotide described herein comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

In some embodiments of any one of the aspects described herein, the oligonucleotides described herein are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (e.g., R^{P}(OH)(O)—O-5′-, R=alkyl, e.g., methyl, ethyl, isopropyl, propyl, etc.), 5′-alkenylphosphonates (i.e. vinyl, substituted vinyl, e.g., OH)₂(O)P-5′—CH═ or (OH)₂(O)P-5′—CH₂—), 5′-alkyletherphosphonates (e.g., R(OH)(O)P—O-5′, R=alkylether, e.g., methoxymethyl (MeOCH2-), ethoxymethyl, etc.) Other exemplary 5′-modifications include where Z is optionally substituted alkyl at least once, e.g., ((HO)2(X)P—O[—(CH₂)ₐ—O—P(X)(OH)—O]_{b}-5′, ((HO)2(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃— and/or Se.

In some embodiments of any one of the aspects described herein, the oligonucleotide comprises a 5′-vinylphosphonate group. For example, the oligonucleotide comprises a 5′-E-vinyl phosphonate group. In some other non-limiting example, the oligonucleotide comprises a 5′-Z-vinylphosphonate group.

In some embodiments of any one of the aspects, the oligonucleotide described herein comprises a 5′-morpholino, a 5′-dimethylamino, a 5′-deoxy, an inverted abasic, or an inverted abasic locked nucleic acid modification at the 5′-end.

In some embodiments of any one of the aspects, the oligonucleotide described herein can comprise a thermally destabilizing modification. For example, the oligonucleotide can comprise at least one thermally destabilizing modification of the duplex within the first 9 nucleotide positions, counting from the 5′-end of the oligonucleotide. In some embodiments, the thermally destabilizing modification is located at position 2, 3, 4, 5, 6, 7, 8 or 9, counting from the 5′-end of the antisense strand. In some embodiments, thermally destabilizing modification is located in positions 2-9, or preferably positions 4-8, counting from the 5′-end of the oligonucleotide. In some further embodiments, the thermally destabilizing modification is located at position 5, 6, 7 or 8, counting from the 5′-end of the oligonucleotide. In still some further embodiments, the thermally destabilizing modification is located at position 7, counting from the 5′-end of the oligonucleotide.

The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s). In some embodiments, the thermally destabilizing modification is located at position 2, 3, 4, 5, 6, 7, 8 or 9, counting from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA). For example, the thermally destabilizing modifications can include, but are not limited to, mUNA and GNA building blocks as follows:

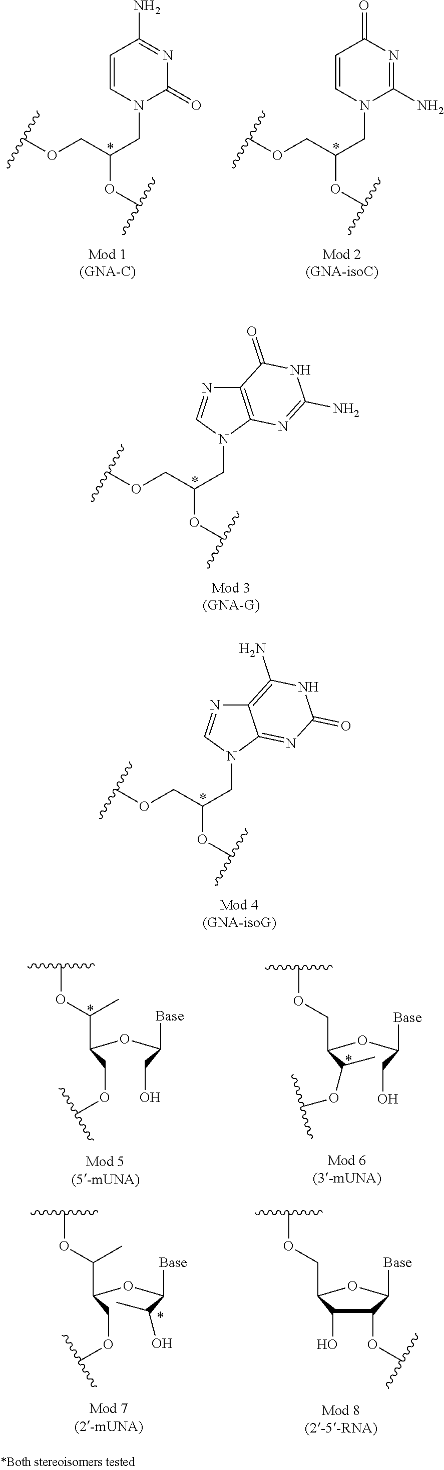

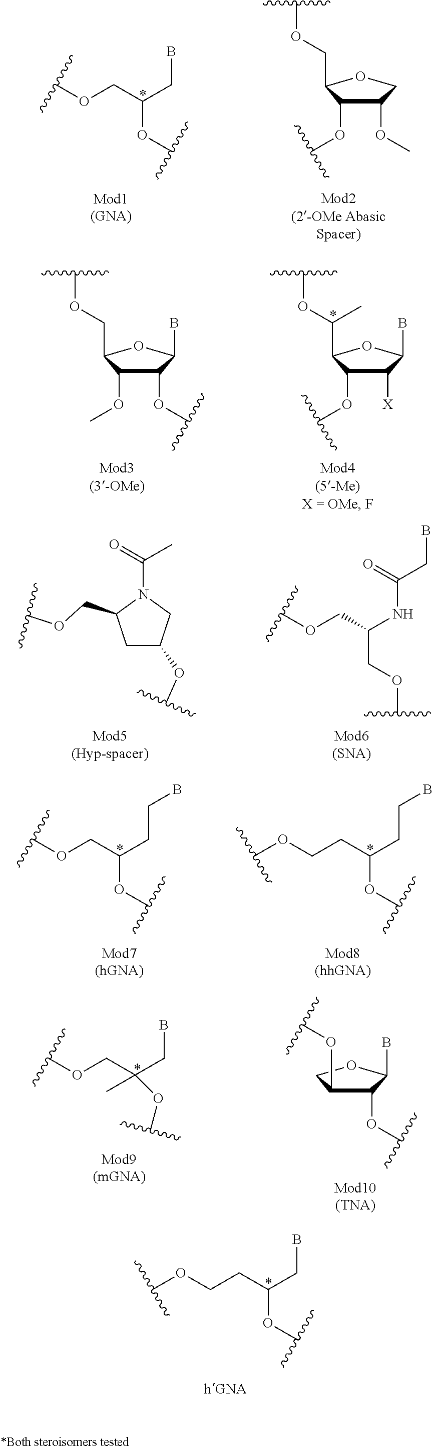

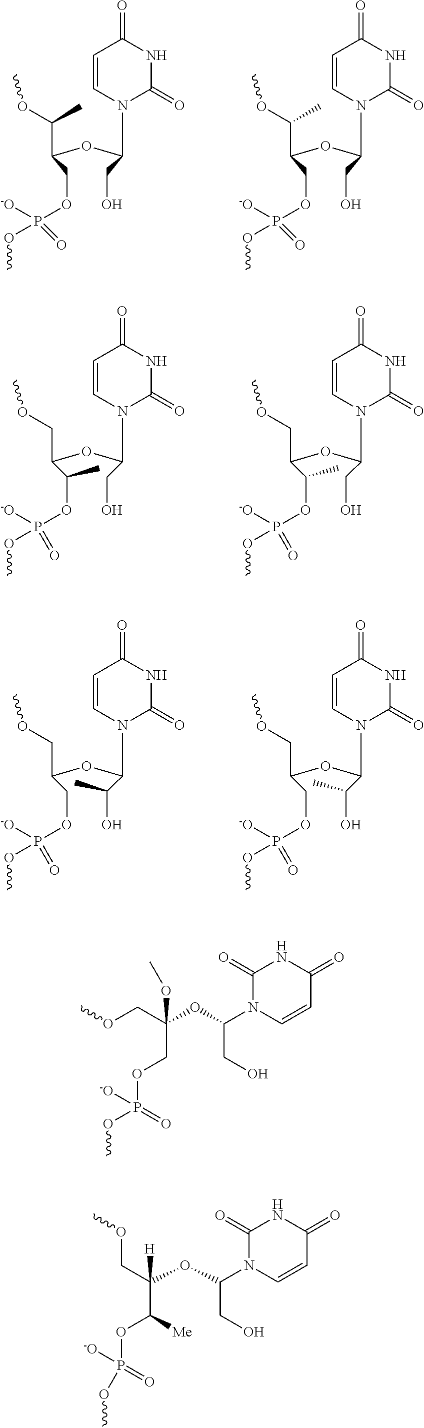

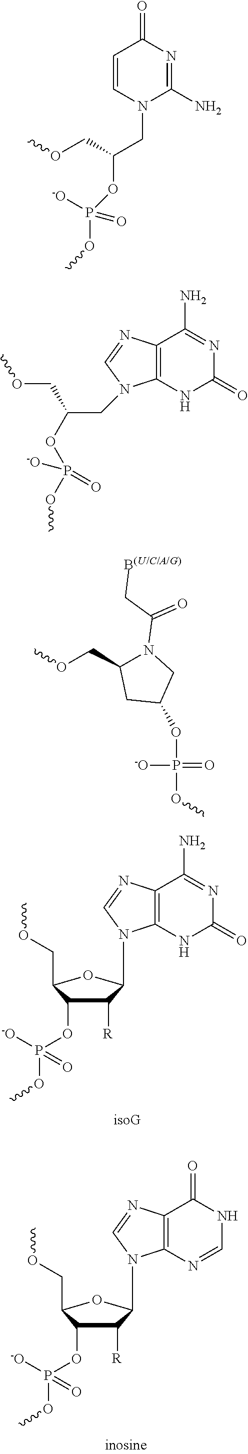

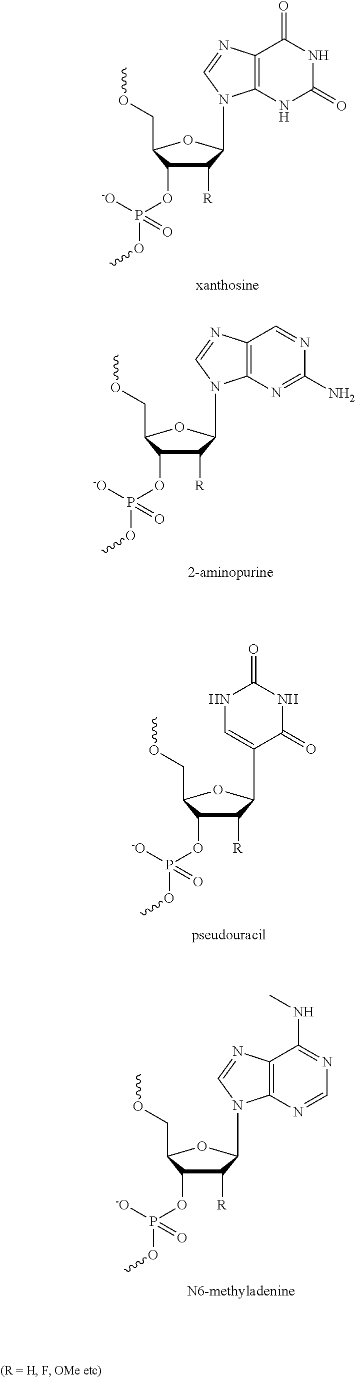

In some embodiments, the destabilizing modification is selected from the group consisting of GNA-isoC, GNA-isoG, 5′-mUNA, 4′-mUNA, 3′-mUNA, and 2′-mUNA.

In some embodiments, the destabilizing modification mUNA is selected from the group consisting of

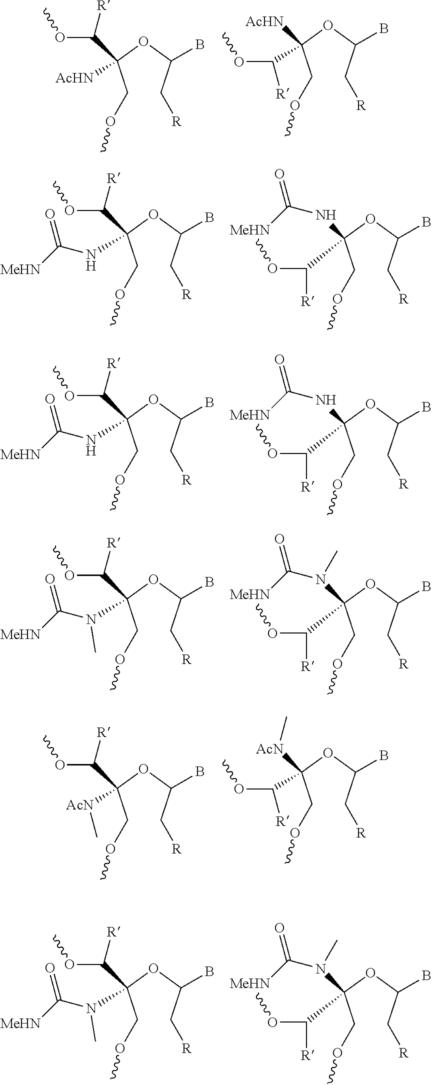

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C₅-modified pyrimidines; C₂-modified purines; N8-modified purines; 7-deazapurines, phenoxazine; G-clamp; non-canonical mono, bi and tricyclic heterocycles; and Stereochemistry is R or S and combination of R and S for the unspecified chiral centers.

In some embodiments, the destabilizing modification mUNA is selected from the group consisting of

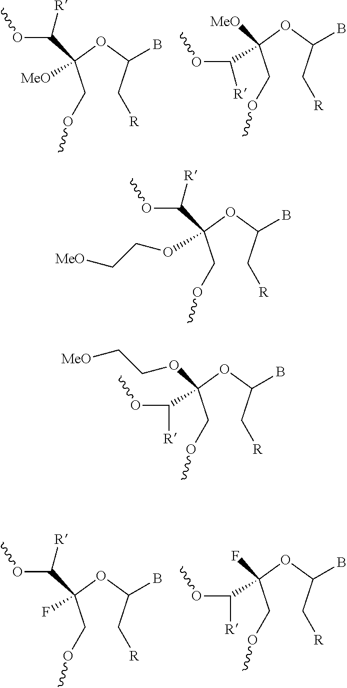

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C₅-modified pyrimidines; C₂-modified purines; N8-modified purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

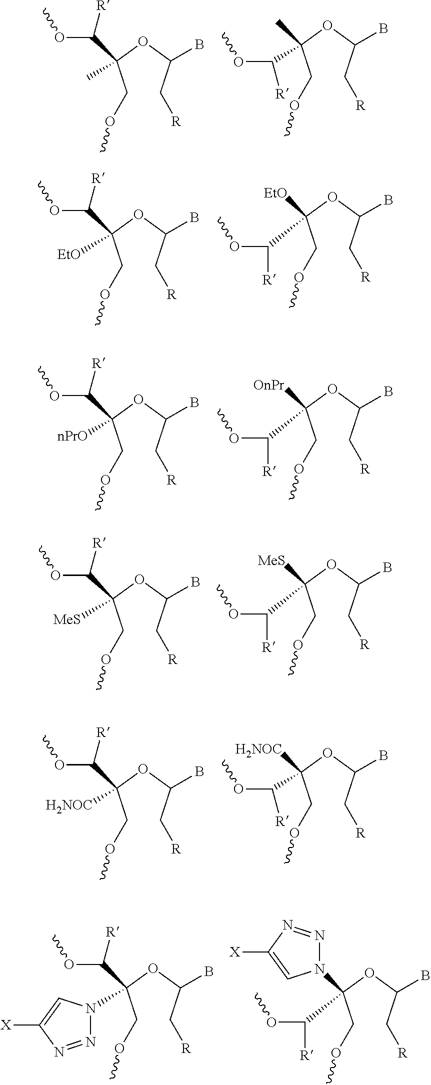

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N₆-alkyl-A; O6-alkyl-G; 7-deazapurines; and Stereochemistry is R or S and combination of R and S for the unspecified chiral centers.

In some embodiments, the destabilizing modification mUNA is selected from the group consisting of

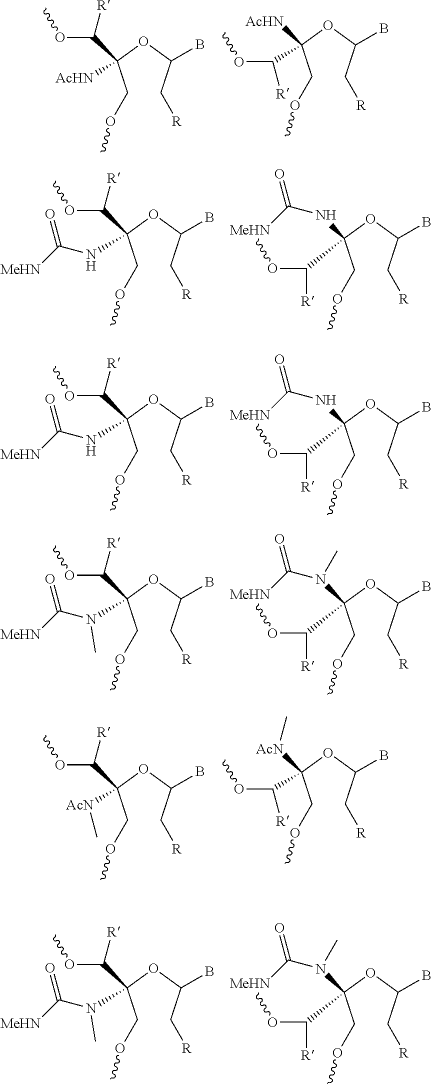

R=H, OH; OMe; Cl, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C₅-modified pyrimidines; C₂-modified purines; N8-modified purines; 7-deazapurines, phenoxazine; G-clamp; non-canonical mono, bi and tricyclic heterocycles; and Stereochemistry is R or S and combination of R and S for the unspecified chiral centers

In some embodiments, the destabilizing modification mUNA is selected from the group consisting of

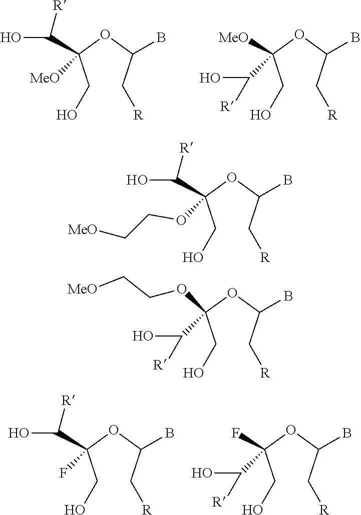

R=H, OH; OMe; Cl, F; OH; O—(CH₂)₂OMe; SMe, NMe₂; NI2; Me; CCH (alkyne), 0-nPr;
O-alkyl; O-alkylamino;
R′=H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N₆-alkyl-A; O6-alkyl-G; 2-thiouridine; 4-thiouridine; C₅-modified pyrimidines; C₂-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the modification mUNA is selected from the group consisting of

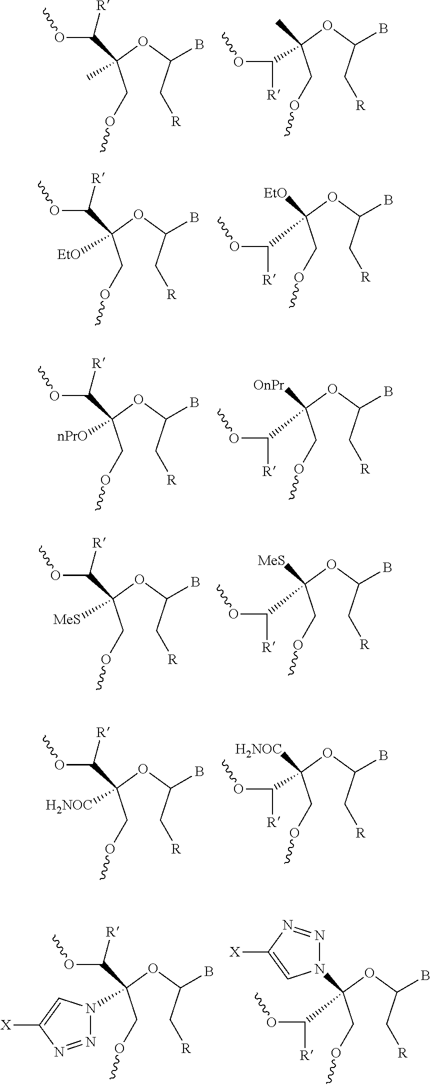

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N₆-alkyl-A; O6-alkyl-G; 7-deazapurines; and
Stereochemistry is R or S and combination of R and S for the unspecified chiral centers

Exemplary abasic modifications include, but are not limited to the following:

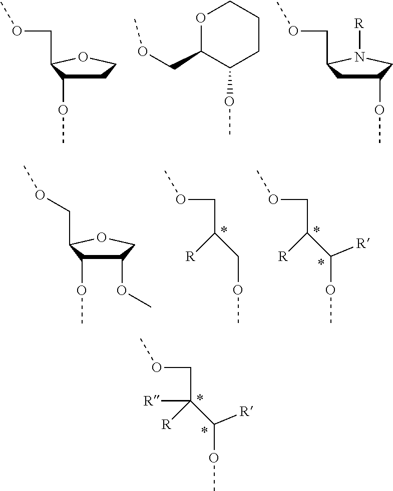

### Wherein R=H, Me, Et or OMe; R′=H, Me, Et or OMe; R″=H, Me, Et or OMe

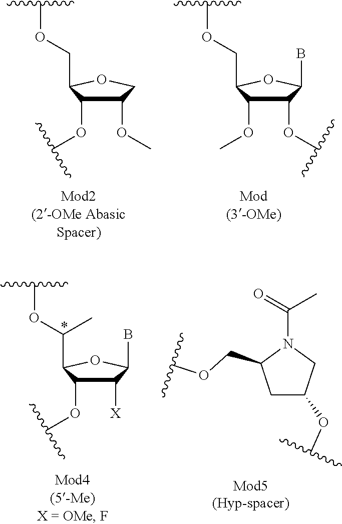

wherein B is a modified or unmodified nucleobase and the asterisk on each structure represents either R, S or racemic.

Exemplified sugar modifications include, but are not limited to the following:

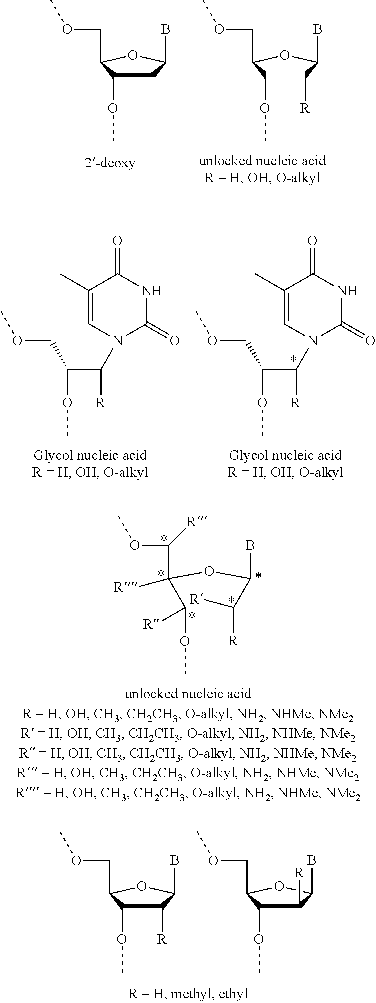

wherein B is a modified or unmodified nucleobase and the asterisk on each structure represents either R, S or racemic.

In some embodiments the thermally destabilizing modification of the duplex is selected from the mUNA and GNA building blocks described in Examples 1-3 herein. In some embodiments, the destabilizing modification is selected from the group consisting of GNA-isoC, GNA-isoG, 5′-mUNA, 4′-mUNA, 3′-mUNA, and 2′-mUNA. In some further embodiments of this, the dsRNA molecule further comprises at least one thermally destabilizing modification selected from the group consisting of GNA, 2′-OMe, 3′-OMe, 5′-Me, Hy p-spacer, SNA, hGNA, hhGNA, mGNA, TNA and h′GNA (Mod A-Mod K).

The term “acyclic nucleotide” refers to any nucleotide having an acyclic ribose sugar, for example, where any of bonds between the ribose carbons (e.g., C1′—C2′, C2′—C3′, C3′—C4′, C4′—O4′, or C1′—O4′) is absent and/or at least one of ribose carbons or oxygen (e.g., C1′, C2′, C3′, C4′ or O4′) are independently or in combination absent from the nucleotide. In some embodiments, acyclic nucleotide is

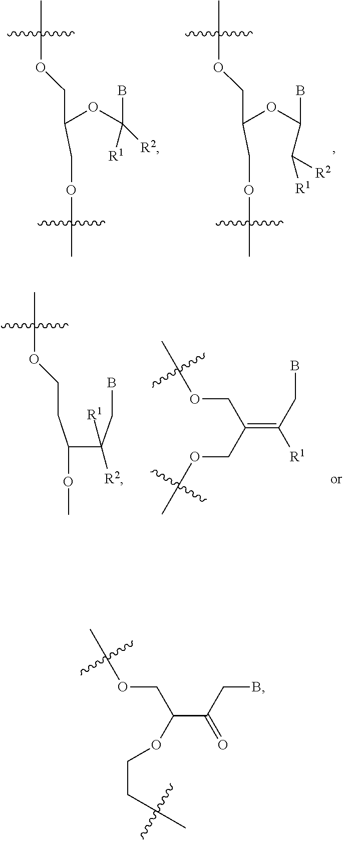

wherein B is a modified or unmodified nucleobase, R1 and R2 independently are H, halogen, OR3, or alkyl; and R3 is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′—C4′ being removed (i.e. the covalent carbon-oxygen-carbon bond between the C1′ and C4′ carbons). In another example, the C2′—C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

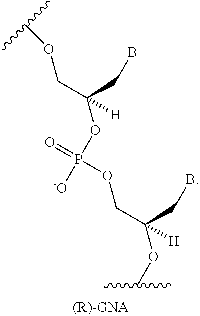

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W—C H-bonding to complementary base on the target mRNA, such as:

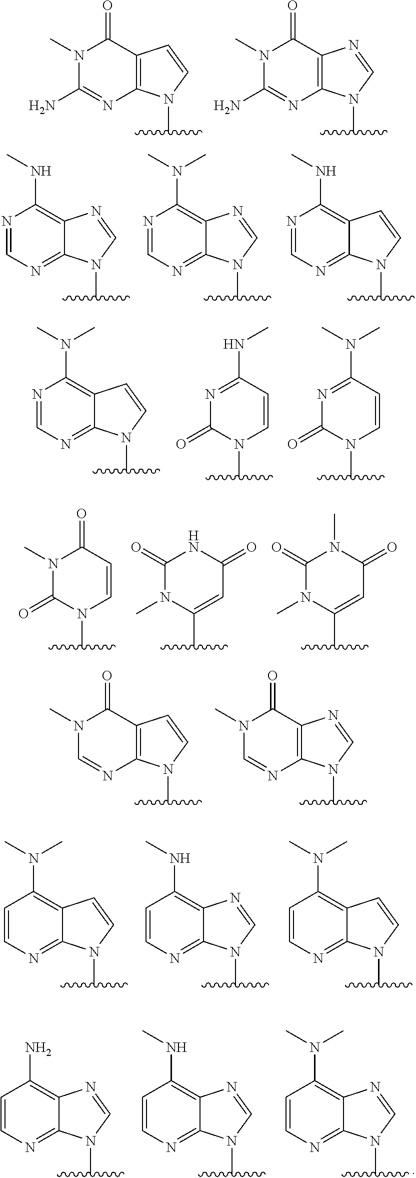

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

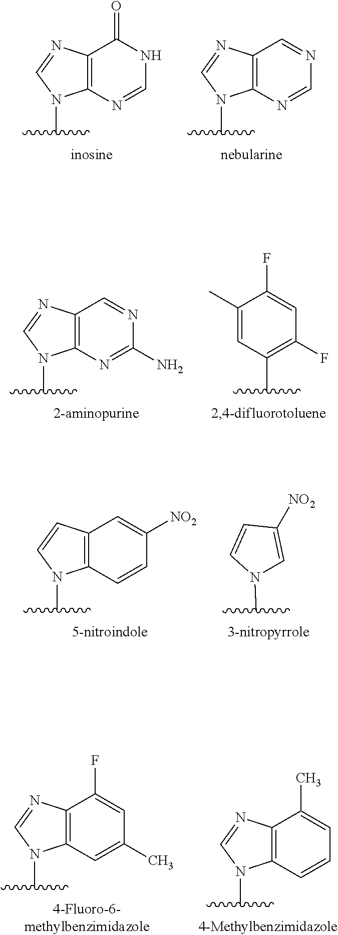

In some embodiments, the thermally destabilizing modification of the duplex in the seed region of the antisense strand includes one or more α-nucleotide complementary to the base on the target mRNA, such as:

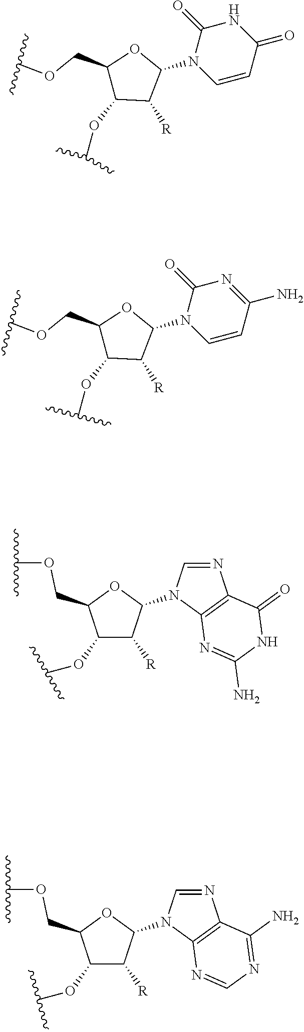

wherein R is H, OH, OCH₃, F, NH₂, NIMe, NMe₂ or O-alkyl

Exemplary phosphate modifications known to decrease the thermal stability of dsRNA duplexes compared to natural phosphodiester linkages are:

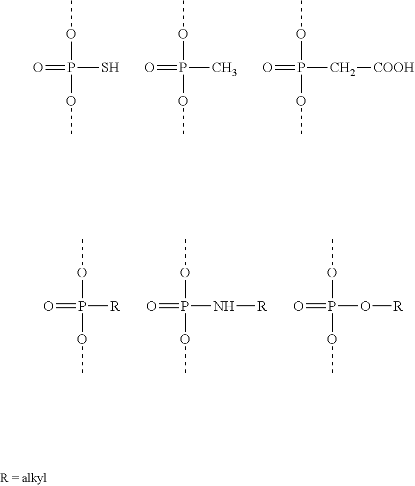

The alkyl for the R group can be a C₁-C₆alkyl. Specific alkyls for the R group include, but are not limited to methyl, ethyl, propyl, isopropyl, butyl, pentyl and hexyl.

In some embodiments of any one of the aspects described herein, the oligonucleotide can comprise one or more stabilizing modifications. For example, the oligonucleotide can comprise at least two (e.g., two, three, four, five, six, seven, eight, nine, ten or more) stabilizing modifications.

In some embodiments, the oligonucleotide comprises at least two (e.g., two, three, four, five, six, seven, eight, nine, ten or more) stabilizing modifications. Without limitations, a stabilizing modification in the oligonucleotide can be present at any positions. In some embodiments, the oligonucleotide comprises stabilizing modifications at positions 2, 6, 8, 9, 14 and 16, counting from the 5′-end. In some other embodiments, the oligonucleotide comprises stabilizing modifications at positions 2, 6, 14 and 16, counting from the 5′-end. In still some other embodiments, the oligonucleotide comprises stabilizing modifications at positions 2, 14 and 16, counting from the 5′-end. In some embodiments, the oligonucleotide comprises stabilizing modifications at positions 7, 10 and 11, counting from the 5′-end. In some other embodiments, the oligonucleotide comprises stabilizing modifications at positions 7, 9, 10 and 11, counting from the 5′-end.

In some embodiments, the oligonucleotide comprises at least one stabilizing modification adjacent to a destabilizing modification. For example, the stabilizing modification can be the nucleotide at the 5′-end or the 3′-end of the destabilizing modification, i.e., at position −1 or +1 from the position of the destabilizing modification. In some embodiments, the oligonucleotide comprises a stabilizing modification at each of the 5′-end and the 3′-end of the destabilizing modification, i.e., positions −1 and +1 from the position of the destabilizing modification.

In some embodiments, the oligonucleotide comprises at least two stabilizing modifications at the 3′-end of a destabilizing modification, i.e., at positions +1 and +2 from the position of the destabilizing modification.

Exemplary thermally stabilizing modifications include, but are not limited to 2′-fluoro modifications. Other thermally stabilizing modifications include, but are not limited to LNA.

### Double-Stranded RNAs

The skilled person is well aware that double-stranded RNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer double-stranded oligonucleotides can be effective as well.

Accordingly, in one aspect, provided herein is a double-stranded RNA (dsRNA) comprising a first strand (also referred to as an antisense strand or a guide strand) and a second strand (also referred to as a sense strand or passenger strand, wherein at least one of the first (i.e., the antisense strand) or the second strand (i.e., the sense strand) is an oligonucleotide described herein. In other words, at least one of the first (i.e., the antisense strand) or the second strand (i.e., the sense strand) comprises at least one nucleotide of Formula (I).

In some embodiments of any one of the aspects described herein, the sense strand is an oligonucleotide described herein. In other words, the sense strand comprises at least one nucleotide of Formula (I).

In some embodiments of any one of the aspects described herein, the antisense strand is an oligonucleotide described herein. In other words, the antisense strand comprises at least one nucleotide of Formula (I).

In some embodiments of the various aspects described herein, the antisense strand is substantially complementary to a target nucleic acid, e.g., a target gene or mRNA gene and the dsRNA is capable of inducing targeted cleavage of the target nucleic acid.

Each strand of the dsRNA molecule can range from 15-35 nucleotides in length. For example, each strand can be between, 17-35 nucleotides in length, 17-30 nucleotides in length, 25-35 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length. For example, the sense strand and the antisense strand independently have a length of 18, 19, 20, 21, 22, 23, 24 or 25 nucleotides.

In some embodiments, the antisense strand is of length 15-35 nucleotides. In some embodiments, the antisense strand is 15-35, 17-35, 17-30, 25-35, 27-30, 17-23, 17-21, 17-19, 19-25, 19-23, 19-21, 21-25, 21-25, or 21-23 nucleotides in length. For example, the antisense strand can be 15, 16, 17, 18, 19, 20, 21, 22, 23, 24, 25, 26, 27, 28, 29, 30, 31, 32, 33, 34 or 35 nucleotides in length. In some embodiments, the antisense strand is 19, 20, 21, 22, 23, 24 or 25 nucleotides in length. For example, the antisense strand is 21, 22, 23, 24 or 25 nucleotides in length. In some particular embodiments, the antisense strand is 22, 23 or 24 nucleotides in length. For example, the antisense strand is 23 nucleotides in length.

Similar to the antisense strand, the sense strand can be, in some embodiments, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length. For example, the sense strand can be 15, 16, 17, 18, 19, 20, 21, 22, 23, 24, 25, 26, 27, 28, 29, 30, 31, 32, 33, 34 or 35 nucleotides in length. In some embodiments, the sense strand is 17, 18, 19, 20, 21, 22, 23, 24 or 25 nucleotides in length. For example, the sense strand is 19, 20, 21, 22 or 23 nucleotides in length. In some particular embodiments, the sense strand is 20, 21 or 22 nucleotides in length. For example, the sense strand is 21nucleotides in length

In some embodiments, the sense strand can be 15-35 nucleotides in length, and the antisense strand can be independent from the sense strand, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length, and the antisense strand is independently 15-35, 17-35, 17-30, 25-35, 27-30, 17-23, 17-21, 17-19, 19-25, 19-23, 19-21, 21-25, 21-25, or 21-23 nucleotides in length. For example, the sense and the antisense strand can be independently 15, 16, 17, 18, 19, 20, 21, 22, 23, 24, 25, 26, 27, 28, 29, 30, 31, 32, 33, 34 or 35 nucleotides in length. In some embodiments, the sense strand and the antisense strand are independently 17, 18, 19, 20, 21, 22, 23, 24 or 25 nucleotides in length. For example, the sense strand is 19, 20, 21, 22 or 23 nucleotides in length and the antisense strand is 21, 22, 23, 24 or 25 nucleotides in length. In some particular embodiments, the sense strand is 20, 21 or 22 nucleotides in length and the antisense strand is 22, 23 or 24 nucleotides in length. For example, the sense strand is 21 nucleotides in length and the antisense strand is 23 nucleotides in length.

The sense strand and antisense strand typically form a double-stranded or duplex region. Without limitations, the duplex region of a dsRNA agent described herein can be 12-35 nucleotide (or base) pairs in length. For example, the duplex region can be between 14-35 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length. In some embodiments, the duplex region is 18, 19, 20, 21, 22, 23, 24 or 25 nucleotide pairs in length. For example, the duplex region is 19, 20, 21, 22 or 23 nucleotide pairs in length. In some embodiments, the the duplex region is 20, 21 or 22 nucleotide pairs in length. For example, the dsRNA molecule has a duplex region of 21 base pairs.

As described herein, the dsRNA molecule described herein can comprise at least one, e.g., 1, 2, 3, 4, 5, 6, 7, 8, 9, 10 or more of nucleotide of Formula (I). Without limitations, the nucleotides of Formula (I) all can be present in one strand. The nucleotide of Formula (I) may occur on any nucleotide of the sense strand or antisense strand or both in any position of the strand.

In some embodiments, the sense strand comprises 1, 2, 3, 4, 5, 6, 7, 8, 9, 10 or more nucleotides of Formula (I) described herein. The nucleotide of Formula (I) described herein can be present at any position of the sense strand. For example, the nucleotide of Formula (I) described herein can be present at a terminal region of the sense strand. For example, the nucleotide of Formula (I) described herein can be present at one or more of positions 1, 2, 3 and 4, counting from the 5′-end of the sense strand. In another non-limiting example, the nucleotide of Formula (I) described herein can be present at one or more of positions 1, 2, 3 and 4, counting from the 3′-end of the sense strand. In some embodiments, the nucleotide of Formula (I) can be present at one or more of positions 18, 19, 20 and 21, counting from 5′-end of the sense strand. The nucleotide of Formula (I) described herein can also be located at a central region of sense strand. For example, the nucleotide of Formula (I) described herein can be located at one or more of positions 6, 7, 8, 9, 10, 11, 12 and 13, counting from 5′-end of the sense strand. In some embodiments, the nucleotide of Formula (I) is at the 5-terminus of the sense strand.

In some embodiments, the antisense strand comprises 1, 2, 3, 4, 5, 6, 7, 8, 9, 10 or more of nucleotides of Formula (I) described herein. The nucleotide of Formula (I) described herein can be present at any position of the antisense strand. For example, the nucleotide of Formula (I) described herein can be present at a terminal region of the antisense strand. For example, the nucleotide of Formula (I) described herein can be present at one or more of positions 1, 2, 3 and 4, counting from the 5′-end of the antisense strand. In another non-limiting example, the nucleotide of Formula (I) described herein nucleotide can be present at one or more of positions 1, 2, 3, 4, 5 and 6, counting from the 3′-end of the antisense strand. In some embodiments, the nucleotide of Formula (I) described herein nucleotide can be present at one or more of positions 18, 19, 20, 21, 22 and 23, counting from 5′-end of the antisense strand. The nucleotide of Formula (I) described herein nucleotide can also be located at a central region of the antisense strand. For example, the nucleotide of Formula (I) described herein nucleotide can be located at one or more of positions 6, 7, 8, 9, 10, 11, 12 and 13, counting from 5′-end of the antisense strand. In some embodiments, the nucleotide of Formula (I) is at the 3′-termnus of the antisense strand.

As described herein, the dsRNA agent can comprise one or more, e.g., 1, 2, 3, 4, 5, 6, 7, 8, 9, 10 or more nucleotides comprising a modified sugar. Accordingly, in some embodiments, the dsRNA agent can comprise one or more, e.g., 1, 2, 3, 4, 5, 6, 7, 8, 9, 10 or more nucleotides independently selected from the group consisting of 2′-F, 2-OMe, acyclic nucleotides, locked nucleic acid (LNA), HNA, CeNA, 2′-methoxyethyl, 2′-O-allyl, 2′-C-allyl, 2′-O—N-methylacetamido (2′-O-NMA), a 2′-O-dimethylaminoethoxyethyl (2′-O-DMAEOE), 2′-O-aminopropyl (2′-O-AP), and 2′-ara-F. A nucleotide comprising modified sugar can be present anywhere in the dsRNA molecule. For example, a nucleotide comprising a modified sugar can be present in the sense strand or a nucleotide comprising a modified sugar can be present in the antisense strand. When two or more nucleotides comprising a modified sugar are present in the dsRNA molecule, they can all be in the sense strand, antisense strand or both in the sense and antisense strands.

As described herein, the dsRNA molecule described herein can comprise at least one, e.g., 1, 2, 3, 4, 5, 6, 7, 8, 9, 10 or more 2′-fluoro (2′-F) nucleotides. In some embodiments, the sense strand comprises 1, 2, 3, 4, 5, 6, 7, 8, 9, 10 or more 2′-fluoro nucleotides. The 2′-fluoro nucleotides can be located anywhere in the sense strand. For example, the sense strand comprises a 2′-fluoro nucleotide at position 10, counting from 5′-end of the sense strand. In some embodiments, the sense strand comprises a 2′-fluoro nucleotide at position 10, counting from 5′-end of the sense strand and the sense strand further comprises a 2′-fluoro nucleotide at position 8, 9, 11 or 12, counting from 5′-end of the sense strand. For example, the sense strand comprises a 2′-fluoro nucleotide at positions 9 10, counting from 5′-end of the sense strand. In another example, the sense strand comprises a 2′-fluoro nucleotide at positions 10 and 11, counting from 5′-end of the sense strand. In some embodiments, the sense strand comprises a 2′-fluoro nucleotide at positions 9, 10 and 11, counting from 5′-end of the sense strand. In some other embodiments, the sense strand comprises a 2′-fluoro nucleotide at positions 8, 9 and 10, counting from 5′-end of the sense strand. In yet some other embodiments, the sense strand comprises a 2′-fluoro nucleotide at positions 10, 11 and 12, counting from 5′-end of the sense strand.

In some embodiments, the antisense comprises 2′-fluoro nucleotides at positions 7, 10 and 11 from the 5′-end. In some other embodiments, the sense strand comprises 2′-fluoro nucleotides at positions 7, 9, 10 and 11 from the 5′-end. In some embodiments, the sense strand comprises 2′-fluoro nucleotides at positions opposite or complimentary to positions 11, 12 and 15 of the antisense strand, counting from the 5′-end of the antisense strand. In some other embodiments, the sense strand comprises 2′-fluoro nucleotides at positions opposite or complimentary to positions 11, 12, 13 and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complimentary to a thermally destabilizing modification of the duplex in the antisense strand.

In some embodiments, the antisense strand comprises 1, 2, 3, 4, 5, 6, 7, 8, 9, 10 or more 2′-fluoro nucleotides. The 2′-fluoro nucleotides can be located anywhere in the antisense strand. For example, the antisense strand can comprise a 2′-fluoro nucleotide at position 14, counting from 5′-end of the antisense strand. In some embodiments, the antisense comprises 2′-fluoro nucleotides at positions 2, 14 and 16, counting from the 5′-end of the antisense strand. In some other embodiments, the antisense comprises 2′-fluoro nucleotides at positions 2, 6, 14 and 16 from the 5′-end. In still some embodiments, the antisense comprises 2′-fluoro nucleotides at positions 2, 6, 8, 9, 14 and 16 from the 5′-end.

In some embodiments, the antisense strand comprises at least one 2′-fluoro nucleotide adjacent to a destabilizing modification. For example, the 2′-fluoro nucleotide can be the nucleotide at the 5′-end or the 3′-end of a destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification. In some embodiments, the antisense strand comprises at least two 2′-fluoro nucleotides at the 3′-end of the destabilizing modification, i.e., at positions +1 and +2 from the position of the destabilizing modification.

In some embodiments, both the sense and the antisense strands comprise at least one 2′-fluoro nucleotide. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

As described herein, the dsRNA molecule described herein can comprise at least one, e.g., 1, 2, 3, 4, 5, 6, 7, 8, 9, 10 or more 2′-OMe nucleotides. Without limitations, the 2′-OMe nucleotides all can be present in one strand. The 2′-OMe nucleotide may occur on any nucleotide of the sense strand or antisense strand or both in any position of the strand.

In some embodiments, the sense strand comprises 1, 2, 3, 4, 5, 6, 7, 8, 9, 10 or more 2′-OMe nucleotides. The 2′-OMe nucleotides can be located anywhere in the sense strand. In some embodiments, the antisense strand comprises 1, 2, 3, 4, 5, 6, 7, 8, 9, 10 or more 2′-OMe nucleotides. The 2′-OMe nucleotides can be located anywhere in the antisense strand.

As described herein, the dsRNA molecule described herein can comprise at least one, e.g., 1, 2, 3, 4, 5, 6, 7, 8, 9, 10 or more 2′-deoxy, e.g., 2′-H ribose nucleotides. For example, the dsRNA can comprise 1, 2, 3, 4, 5, 6, 7, 8, 9 or 10 2′-deoxy, e.g., 2′-H nucleotides. The 2′-deoxy nucleotide may occur on any nucleotide of the sense strand or antisense strand or both in any position of the strand.

As described herein, the dsRNA can comprise at least one, e.g., at least two, at least three, at least four, at least five, at least six, at least seven or more, 2′-deoxy modifications in a central region of the sense strand and/or the antisense strand. For example, at least one of the sense stand and the antisense can comprise at least one, e.g., at least two, at least three, at least four, at least five, at least six, at least seven or more, 2′-deoxy modification in positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the sense strand or the antisense strand.

In some embodiments, the antisense strand comprises 1, 2, 3, 4, 5 or 6 of 2′-deoxy nucleotides. For example, antisense strand can comprise 2, 3, 4, 5 or 6 of 2′-deoxy nucleotides. The 2′-deoxy nucleotides can be located anywhere in the antisense strand. For example, the antisense strand comprises a 2′-deoxy nucleotide at 1, 2, 3, 4, 5 or 6 of positions 2, 5, 7, 12, 14 and 16, counting from 5′-end of the antisense strand. In one non-limiting example, the antisense strand comprises a 2′-deoxy nucleotide at 1, 2, 3 or 4 of positions 2, 5, 7, and 12, counting from 5′-end of the antisense strand.

In some embodiments, the antisense comprises a 2′-deoxy nucleotide at positions 5 and 7, counting from 5′-end of the antisense strand. For example, the antisense strand comprises a 2′-deoxy nucleotide at positions 5, 7 and 12, counting from 5′-end of the antisense strand. In some embodiments, the antisense strand comprises a 2′-deoxy nucleotide at positions 2, 5 and 7, counting from 5′-end of the antisense strand. For example, the antisense strand comprises a 2′-deoxy nucleotide at positions 2, 5, 7 and 12, counting from 5′-end of the antisense strand. In some embodiments, the antisense strand comprises a 2′-deoxy nucleotide at positions 2, 5, 7, 12 and 14, counting, from 5′-end of the antisense strand. For example, the antisense strand comprises a 2′-deoxy nucleotide at positions 2, 5, 7, 12, 14 and 16, counting from 5′-end of the antisense strand

In some embodiments, the antisense comprises a 2′-deoxy nucleotide at position 2 or 12, counting from 5′-end of the antisense strand. For example, the antisense comprises a 2′-deoxy nucleotide at position 12, counting from 5′-end of the antisense strand.

In some embodiments, the dsRNA comprises at least three 2′-deoxy modifications, wherein the 2′-deoxy modifications are at positions 2 and 14 of the antisense strand, counting from 5′-end of the antisense strand, and at position 11 of the sense strand, counting from 5′-end of the sense strand.

In some embodiments, the dsRNA comprises at least five 2′-deoxy modifications, wherein the 2′-deoxy modifications are at positions 2, 12 and 14 of the antisense strand, counting from 5′-end of the antisense strand, and at positions 9 and 11 of the sense strand, counting from 5′-end of the sense strand.

In some embodiments, the dsRNA comprises at least seven 2′-deoxy modifications, wherein the 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In some embodiments, the antisense strand comprises at least five 2′-deoxy modifications at positions 2, 5, 7, 12 and 14, counting from 5′-end of the antisense strand.

In one non-limiting example, the sense strand does not comprise a 2′-deoxy nucleotide at position 11, counting from 5′-end of the sense strand.

In some embodiments, the dsRNA can comprise one or more, e.g., 1, 2, 3, 4, 5, 6, 7, 8, 9, 10 or more nucleotides comprising a non-natural nucleobase

A nucleotide comprising a non-natural nucleobase can be present anywhere in the dsRNA molecule. For example, a nucleotide comprising a non-natural nucleobase can be present in the sense strand or a nucleotide comprising a non-natural nucleobase can be present in the antisense strand. When two or more nucleotides comprising a non-natural nucleobase are present in the dsRNA molecule, they can all be in the sense strand, antisense strand or both in the sense and antisense strands.

The dsRNA molecule described herein can further comprise at least one phosphorothioate or methylphosphonate internucleoside linkage. The phosphorothioate or methylphosphonate internucleoside linkage modification may occur on any nucleotide of the sense strand or antisense strand or both in any position of the strand. For instance, the internucleoside linkage modification may occur on every nucleotide on the sense strand and/or antisense strand; each internucleoside linkage modification may occur in an alternating pattern on the sense strand or antisense strand; or the sense strand or antisense strand comprises both internucleoside linkage modifications in an alternating pattern. The alternating pattern of the internucleoside linkage modification on the sense strand may be the same or different from the antisense strand, and the alternating pattern of the internucleoside linkage modification on the sense strand may have a shift relative to the alternating pattern of the internucleoside linkage modification on the antisense strand.

In some embodiments, the dsRNA molecule comprises the phosphorothioate or methylphosphonate internucleoside linkage modification in the overhang region. For example, the overhang region comprises two nucleotides having a phosphorothioate or methylphosphonate internucleoside linkage between the two nucleotides. Internucleoside linkage modifications also may be made to link the overhang nucleotides with the terminal paired nucleotides within duplex region. For example, at least 2, 3, 4, or all the overhang nucleotides may be linked through phosphorothioate or methylphosphonate internucleoside linkage, and optionally, there may be additional phosphorothioate or methylphosphonate internucleoside linkages linking the overhang nucleotide with a paired nucleotide that is next to the overhang nucleotide. For instance, there may be at least two phosphorothioate internucleoside linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleoside linkages separated by 1, 2, 3, 4, 5, 6, 7, 8, 9, 10, 11, 12, 13, 14, 15 or 16 phosphate internucleoside linkages, wherein one of the phosphorothioate or methylphosphonate internucleoside linkages is placed at any position in the oligonucleotide sequence and the said sense strand is paired with an antisense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of two phosphorothioate or methylphosphonate internucleoside linkages separated by 1, 2, 3, 4, 5, 6, 7, 8, 9, 10, 11, 12, 13, 14, 15, 16, 17, or 18 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of three phosphorothioate or methylphosphonate internucleoside linkages separated by 1, 2, 3, 4, 5, 6, 7, 8, 9, 10, 11, 12, 13, 14, 15 or 16 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of four phosphorothioate or methylphosphonate internucleoside linkages separated by 1, 2, 3, 4, 5, 6, 7, 8, 9, 10, 11, 12, 13 or 14 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of five phosphorothioate or methylphosphonate internucleoside linkages separated by 1, 2, 3, 4, 5, 6, 7, 8, 9, 10, 11 or 12 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of six phosphorothioate or methylphosphonate internucleoside linkages separated by 1, 2, 3, 4, 5, 6, 7, 8, 9 or 10 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleoside linkages separated by 1, 2, 3, 4, 5, 6, 7 or 8 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleoside linkages separated by 1, 2, 3, 4, 5 or 6 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleoside linkages separated by 1, 2, 3 or 4 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the dsRNA molecule described herein further comprises one or more phosphorothioate or methylphosphonate internucleoside linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleoside linkage at one end or both ends of the sense and/or antisense strand.

In some embodiments, the dsRNA molecule described herein comprises one or more phosphorothioate or methylphosphonate internucleoside linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleoside linkage at position 8-16 of the duplex region counting from the 5′-end of the sense strand; the dsRNA molecule can optionally further comprise one or more phosphorothioate or methylphosphonate internucleoside linkage modification within 1-10 of the termini position(s).

In some embodiments, the dsRNA molecule described herein further comprises one to five phosphorothioate or methylphosphonate internucleoside linkage modification(s) within position 1-5 and one to five phosphorothioate or methylphosphonate internucleoside linkage modification(s) within the last 3 positions of the sense strand (counting from the 5′-end), and one to five phosphorothioate or methylphosphonate internucleoside linkage modification at positions 1 and 2 and one to five phosphorothioate or methylphosphonate internucleoside linkage modification within the last six positions of the antisense strand (counting from the 5′-end).

In some embodiments, the dsRNA molecule described herein further comprises one phosphorothioate internucleoside linkage modification within position 1-5 and one phosphorothioate or methylphosphonate internucleoside linkage modification within the last six positions of the sense strand (counting from the 5′-end), and one phosphorothioate internucleoside linkage modification at positions 1 and 2 and two phosphorothioate or methylphosphonate internucleoside linkage modifications within the last six the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and two phosphorothioate internucleoside linkage modifications within the last four positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and two phosphorothioate internucleoside linkage modifications within the last four positions of the sense strand (counting from the 5′-end), and one phosphorothioate internucleoside linkage modification at positions 1 and 2 and one phosphorothioate internucleoside linkage modification within the last six positions of the antisense strand (counting from the 5′-end).

In some embodiments, the dsRNA molecule described herein further comprises one phosphorothioate internucleoside linkage modification within position 1-5 and one phosphorothioate internucleoside linkage modification within the last four positions of the sense strand (counting from the 5′-end), and two phosphorothioate internucleoside linkage modifications at positions 1 and 2 and two phosphorothioate internucleoside linkage modifications within the last six positions of the antisense strand (counting from the 5′-end).

In some embodiments, the dsRNA molecule described herein further comprises one phosphorothioate internucleoside linkage modification within position 1-5 and one within the last six positions of the sense strand (counting from the 5′-end), and two phosphorothioate internucleoside linkage modification at positions 1 and 2 and one phosphorothioate internucleoside linkage modification within the last six positions of the antisense strand (counting from the 5′-end).

In some embodiments, the dsRNA molecule described herein further comprises one phosphorothioate internucleoside linkage modification within position 1-5 (counting from the 5′-end) of the sense strand, and two phosphorothioate internucleoside linkage modifications at positions 1 and 2 and one phosphorothioate internucleoside linkage modification within the last six positions of the antisense strand (counting from the 5′-end).

In some embodiments, the dsRNA molecule described herein further comprises two phosphorothioate internucleoside linkage modifications within position 1-5 (counting from the 5′-end) of the sense strand, and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and one within the last six positions of the sense strand (counting from the 5′-end), and two phosphorothioate internucleoside linkage modifications at positions 1 and 2 and one phosphorothioate internucleoside linkage modification within the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and two phosphorothioate internucleoside linkage modifications at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 20 and 21 of the sense strand (counting from the 5′-end), and one phosphorothioate internucleoside linkage modification at positions 1 and one at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 20 and 21 the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 21 and 22 of the sense strand (counting from the 5′-end), and one phosphorothioate internucleoside linkage modification at positions 1 and one phosphorothioate internucleoside linkage modification at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 21 and 22 the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 22 and 23 of the sense strand (counting from the 5′-end), and one phosphorothioate internucleoside linkage modification at positions 1 and one phosphorothioate internucleoside linkage modification at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 22 and 23 the antisense strand (counting from the 5′-end).

In some embodiments, the sense strand comprises at least two phosphorothioate internucleoside linkages between the first five nucleotides counting from the 5′ end of the sense strand. For example, the sense strand comprises phosphorothioate linkages between nucleotides 1 and 2, and between nucleotides 2 and 3, counting from 5′-end of the sense strand.

In some embodiments, the antisense strand comprises at least two phosphorothioate internucleoside linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand.

In some embodiments, the antisense strand comprises at least two phosphorothioate internucleoside linkages between the first five nucleotides counting from the 3′ end of the antisense strand. For example, the antisense strand comprises phosphorothioate linkages between nucleotides n and n−1, and between nucleotides n-1 and n-2, where n is length of the antisense strand, i.e, number of nucleotides in the antisense strand. In other words, the antisense strand comprises phosphorothioate linkages between nucleotides 1 and 2, and between nucleotides 2 and 3, counting from 3′-end of the antisense strand.

In some embodiments, the antisense strand comprises at least two phosphorothioate internucleoside linkages between the first five nucleotides counting from the 5′-end of the antisense strand and at least two phosphorothioate internucleoside linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand and between nucleotides 1 and 2, and between nucleotides 2 and 3, counting from 3′-end of the antisense strand.

In some embodiments, the sense strand comprises at least two phosphorothioate internucleoside linkages between the first five nucleotides counting from the 5′ end of the sense strand and the antisense strand comprises at least two phosphorothioate internucleoside linkages between the first five nucleotides counting from the 5′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 5′-end of the antisense strand.

In some embodiments, the sense strand comprises at least two phosphorothioate internucleoside linkages between the first five nucleotides counting from the 5′ end of the sense strand and the antisense strand comprises at least two phosphorothioate internucleoside linkages between the first five nucleotides counting from the 3′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 3′-end of the antisense strand.

In some embodiments, dsRNA molecule described herein comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, dsRNA molecule described herein comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, dsRNA molecule described herein comprises a 5′-block is an Sp block wherein each sugar moiety comprises a 2′-fluoro modification. In some embodiments, a 5′-block is an Sp block wherein each of internucleotidic linkage is a modified internucleotidic linkage and each sugar moiety comprises a 2′-fluoro modification. In some embodiments, a 5′-block is an Sp block wherein each of internucleoside linkage is a phosphorothioate linkage and each sugar moiety comprises a 2′-fluoro modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-fluoro modification. In some embodiments, a 3′-block is an Sp block wherein each of internucleotidic linkage is a modified internucleotidic linkage and each sugar moiety comprises a 2′-fluoro modification. In some embodiments, a 3′-block is an Sp block wherein each of internucleotidic linkage is a phosphorothioate linkage and each sugar moiety comprises a 2′-fluoro modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, dsRNA molecule described herein comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

Various publications describe multimeric siRNA which can all be used with the oligonucleotide and dsRNA of the invention. Such publications include WO2007/091269, U.S. Pat. No. 7,858,769, WO2010/141511, WO2007/117686, WO2009/014887 and WO2011/031520 which are hereby incorporated by their entirely.

In some embodiments, the dsRNA molecule described herein comprises one or more overhang regions and/or capping groups of dsRNA molecule at the 3′-end, or 5′-end or both ends of a strand. The overhang can be 1-10 nucleotides in length. For example, the overhang can be 1, 2, 3, 4, 5, 6, 7, 8, 9 or 10 nucleotides in length. In some embodiments, the overhang is 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target sequence or it can be complementary to the gene sequences being targeted or it can be the other sequence. The first and second strands can also be joined, e.g., by additional bases to form a hairpin, or by other non-base linkers.

In some embodiments, the nucleotides in the overhang region of the dsRNA molecule described herein can each independently be a modified or unmodified nucleotide including, but not limited to 2′-sugar modified, such as, 2′-Fluoro 2′-O-methyl, thymidine (T), 2′-O-methoxyethyl-5-methyluridine, 2′-O-methoxyethyladenosine, 2′-O-methoxyethyl-5-methylcytidine, GNA, SNA, hGNA, hhGNA, mGNA, TNA, h′GNA, and any combinations thereof. For example, dTdT can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA molecule described herein may be phosphorylated. In some embodiments, the overhang region contains two nucleotides having a phosphorothioate between the two nucleotides, where the two nucleotides can be the same or different. In some embodiments, the overhang is present at the 3′-end of the sense strand, antisense strand or both strands. In some embodiments, this 3′-overhang is present in the antisense strand. In some embodiments, this 3′-overhang is present in the sense strand.

The dsRNA molecule described herein may comprise only a single overhang, which can strengthen the interference activity of the dsRNA, without affecting its overall stability. For example, the single-stranded overhang is located at the 3-terminal end of the sense strand or, alternatively, at the 3-terminal end of the antisense strand. The dsRNA can also have a blunt end, located at the 5′-end of the antisense strand (or the 3′-end of the sense strand) or vice versa.

Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process. For example, the single overhang is at least one, two, three, four, five, six, seven, eight, nine, or ten nucleotides in length. In some embodiments, the dsRNA has a 2 nucleotide overhang on the 3′-end of the antisense strand and a blunt end at the 5′-end of the antisense strand.

The dsRNA described herein can comprise one or more modified nucleotides. For example, every nucleotide in the sense strand and antisense strand of the dsRNA molecule can be modified. Each nucleotide can be modified with the same or different modification which can include one or more alteration of one or both of the non-linking phosphate oxygens and/or of one or more of the linking phosphate oxygens; alteration of a constituent of the ribose sugar; replacement of the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a central region, may only occur at a non-terminal region, or may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, the dsRNA molecule described herein comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA molecule described herein comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

In some embodiments of any one of the aspects described herein, the oligonucleotides described herein or at least one e.g., both strand of a dsRNA described herein are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (e.g., R^{P}(OH)(O)—O-5′-, R=alkyl, e.g., methyl, ethyl, isopropyl, propyl, etc.), 5′-alkenylphosphonates (i.e. vinyl, substituted vinyl, e.g., OH)₂(O)P-5′—CH═ or (OH)₂(O)P-5′—CH₂—), 5′-alkyletherphosphonates (e.g., R(OH)(O)P—O-5′, R=alkylether, e.g., methoxymethyl (MeOCH2-), ethoxymethyl, etc.) Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃— and/or Se.

In some embodiments of any one of the aspects described herein, the oligonucleotide or at least one (e.g., both) strand of a dsRNA described herein comprises a 5′-vinylphosphonate group. For example, the oligonucleotide or at least one (e.g., both) strand of a dsRNA described herein comprises a 5′-E-vinyl or at least one (e.g., both) strand of a dsRNA described herein phosphonate group. In some other non-limiting example, the oligonucleotide comprises a 5′-Z-vinylphosphonate group.

In one example, the 5′-modification can be placed in the antisense strand of a double-stranded nucleic acid, e.g., dsRNA molecule. For example, the antisense comprises a 5′-E-vinylphosphonate. In some other non-limiting example, the antisense strand comprises a 5′-Z-vinylphosphonate group.

In some embodiments, the sense strand comprises a 5′-morpholino, a 5′-dimethylamino, a 5′-deoxy, an inverted abasic, or an inverted abasic locked nucleic acid modification at the 5′-end.

The dsRNA agents of the invention can comprise thermally destabilizing modifications in the seed region of the antisense strand (i.e., at positions 2-9 of the 5′-end of the antisense strand) to reduce or inhibit off-target gene silencing. Without wishing to be bound by a theory, dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, thermally destabilizing modification of the duplex is located in positions 2-9, or preferably positions 4-8, from the 5′-end of the antisense strand. In some further embodiments, the thermally destabilizing modification of the duplex is located at position 5, 6, 7 or 8 from the 5′-end of the antisense strand.

In still some further embodiments, the thermally destabilizing modification of the duplex is located at position 7 from the 5′-end of the antisense strand.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand and/or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14 and 16 from the 5′-end. In some other embodiments, the antisense comprises stabilizing modifications at positions 2, 6, 14 and 16 from the 5′-end. In still some other embodiments, the antisense comprises stabilizing modifications at positions 2, 14 and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification. In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10 and 11 from the 5′-end. In some other embodiments, the sense strand comprises stabilizing modifications at positions 7, 9, 10 and 11 from the 5′-end. In some embodiments, the sense strand comprises stabilizing modifications at positions opposite or complimentary to positions 11, 12 and 15 of the antisense strand, counting from the 5′-end of the antisense strand. In some other embodiments, the sense strand comprises stabilizing modifications at positions opposite or complimentary to positions 11, 12, 13 and 15 of the antisense strand, counting from the 5′-end of the antisense strand. In some embodiments, the sense strand comprises a block of two, three or four stabilizing modifications.

In some embodiments, the sense strand does not comprise a stabilizing modification in position opposite or complimentary to the thermally destabilizing modification of the duplex in the antisense strand.

It is noted a thermally stabilizing modification can replace a 2′-fluoro nucleotide in the sense and/or antisense strand. For example, a 2′-fluoro nucleotide at positions 8, 9, 10, 11 and/or 12, counting from 5′-end, of the sense strand, can be replaced with a thermally stabilizing modification. Similarly, a 2′-fluoro nucleotide at position 14, counting from 5′-end, of the antisense strand, can be replaced with a thermally stabilizing modification.

For the dsRNA molecules to be more effective in vivo, the antisense strand must have some metabolic stability. In other words, for the dsRNA molecules to be more effective in vivo, some amount of the antisense stand may need to be present in vivo after a period time after administration. Accordingly, in some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 5 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 6 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 7 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 8 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 9 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 10 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 11 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 12 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 13 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 14 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 15 after in vivo administration.

Uses of Oligonucleotides and dsRNAs

In some embodiments of any one of the aspects, the oligonucleotide described herein or the antisense strand of the dsRNA molecule described herein comprises a nucleotide sequence substantially complementary to a target nucleic acid, e.g., a target gene or mRNA.

Accordingly, in another aspect, the disclosure is directed to a use of an oligonucleotide and/or dsRNA molecule described herein for inhibiting expression of a target gene. In some embodiments, the present invention further relates to a use of an oligonucleotide and/or dsRNA molecule described herein for inhibiting expression of a target gene in vitro.

In another aspect, the disclosure is directed to a use of an oligonucleotide and/or dsRNA molecule described herein for use in inhibiting expression of a target gene in a subject. The subject may be any animal, such as a mammal, e.g., a mouse, a rat, a sheep, a cattle, a dog, a cat, or a human

In some embodiments, the oligonucleotide and/or dsRNA molecule described herein is administered in buffer.

In some embodiments, oligonucleotide and/or dsRNA molecule described herein described herein can be formulated for administration to a subject. A formulated oligonucleotide and/or dsRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the siRNA composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A oligonucleotide and/or dsRNA preparation can be formulated in combination with another agent, e.g., another therapeutic agent or an agent that stabilizes an oligonucleotide and/or dsRNA, e.g., a protein that complexes with oligonucleotide and/or dsRNA. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

In some embodiments, the oligonucleotide and/or dsRNA preparation includes another dsRNA compound, e.g., a second dsRNA that can mediate RNAi with respect to a second gene, or with respect to the same gene. Still other preparation can include at least 3, 5, ten, twenty, fifty, or a hundred or more different siRNA species. Such dsRNAs can mediate RNAi with respect to a similar number of different genes.

In some embodiments, the oligonucleotide and/or dsRNA preparation includes at least a second therapeutic agent (e.g., an agent other than a RNA or a DNA). For example, a oligonucleotide and/or dsRNA composition for the treatment of a viral disease, e.g., HIV, might include a known antiviral agent (e.g., a protease inhibitor or reverse transcriptase inhibitor). In another example, a dsRNA composition for the treatment of a cancer might further comprise a chemotherapeutic agent.

Exemplary formulations which can be used for administering the oligonucleotide and/or dsRNA according to the present invention are discussed below.

Liposomes. A oligonucleotide and/or dsRNA preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide and/or dsRNA composition. The lipophilic material isolates the aqueous interior from an aqueous exterior, which typically does not include the oligonucleotide and/or dsRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide and/or dsRNA are delivered into the cell where the dsRNA can specifically bind to a target RNA and can mediate RNAi. In some embodiments, the liposomes are also specifically targeted, e.g., to direct the oligonucleotide and/or dsRNA to particular cell types.

A liposome containing oligonucleotide and/or dsRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the siRNA and condense around the dsRNA to form a liposome. After condensation, the detergent is removed, e.g., by dialysis, to yield a liposomal preparation of oligonucleotide and/or dsRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging oligonucleotide and/or dsRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992)₂₆₉-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration. Liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with dsRNA described herein are useful for treating a dermatological disorder.

Liposomes that include oligonucleotide and/or dsRNA described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include oligonucleotide and/or dsRNA described herein can be delivered, for example, subcutaneously by infection in order to deliver dsRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Surfactants. The oligonucleotide and/or dsRNA compositions can include a surfactant. In some embodiments, the dsRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the oligonucleotide and/or dsRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method, a first micellar composition is prepared which contains the oligonucleotide and/or dsRNA composition and at least the alkali metal alkyl sulphate. The first micellar composition is then mixed with at least three micelle forming compounds to form a mixed micellar composition. In another method, the micellar composition is prepared by mixing the dsRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, dsRNA preparations can be incorporated into a particle, e.g., a microparticle. Microparticles can be produced by spray-drying, but may also be produced by other methods including lyophilization, evaporation, fluid bed drying, vacuum drying, or a combination of these techniques.

### Pharmaceutical Compositions

The oligonucleotide and/or dsRNA described herein can be formulated for pharmaceutical use. The present invention further relates to a pharmaceutical composition comprising the oligonucleotide and/or dsRNA described herein. Pharmaceutically acceptable compositions comprise a therapeutically-effective amount of one or more of the dsRNA molecules in any of the preceding embodiments, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a dsRNA molecule described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association an oligonucleotide and/or dsRNA with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The oligonucleotide and/or dsRNA described herein may be formulated for administration in any convenient way for use in human or veterinary medicine, by analogy with other pharmaceuticals.

The term “treatment” is intended to encompass therapy and cure. The patient receiving this treatment is any animal in need, including primates, in particular humans, and other mammals such as equines, cattle, swine and sheep; and poultry and pets in general.

The oligonucleotide and/or dsRNA described herein or a pharmaceutical composition comprising an oligonucleotide and/or dsRNA described herein can be administered to a subject using different routes of delivery. A composition that includes an oligonucleotide and/or dsRNA described herein described herein can be delivered to a subject by a variety of routes. Exemplary routes include: intravenous, subcutaneous, topical, rectal, anal, vaginal, nasal, pulmonary, ocular.

The oligonucleotide and/or dsRNA described herein may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the oligonucleotide and/or dsRNA described herein in aerosol form. The vascular endothelial cells could be targeted by coating a balloon catheter with the oligonucleotide and/or dsRNA described herein and mechanically introducing the oligonucleotide and/or dsRNA described herein.

In one aspect, provided herein is a method of administering an oligonucleotide and/or dsRNA described herein, to a subject (e.g., a human subject). In another aspect, the present invention relates to an oligonucleotide and/or dsRNA described herein for use in inhibiting expression of a target gene in a subject. The method or the medical use includes administering a unit dose of the oligonucleotide and/or dsRNA described herein. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of oligonucleotide and/or dsRNA described herein per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities.

In some embodiments, a subject is administered an initial dose and one or more maintenance doses. The maintenance dose or doses can be the same or lower than the initial dose, e.g., one-half less of the initial dose. A maintenance regimen can include treating the subject with a dose or doses ranging from 0.01 g to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA molecule species. In another embodiment, the dsRNA molecule species has sequences that are non-overlapping and non-adjacent to another species with respect to a naturally occurring target sequence. In another embodiment, the plurality of dsRNA molecule species is specific for different naturally occurring target genes. In another embodiment, the dsRNA molecule is allele specific.

The oligonucleotide and/or dsRNA described herein can be administered to mammals, particularly large mammals such as nonhuman primates or humans in a number of ways.

In some embodiments, the administration of the oligonucleotide and/or dsRNA composition described herein is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

The invention provides methods, compositions, and kits, for rectal administration or delivery of oligonucleotide and/or dsRNA composition described herein.

### Methods of Inhibiting Expression of a Target Gene

Aspects of the disclosure also relate to methods for inhibiting the expression of a target gene. The method comprises administering to the subject in an amount sufficient to inhibit expression of the target gene: (i) a double-stranded RNA described herein, where the wherein the first strand is complementary to a target gene; and/or (ii) an oligonucleotide described herein, wherein the oligonucleotide is complementary to a target gene.

The present disclosure further relates to a use of an oligonucleotide and/or dsRNA molecule described herein for inhibiting expression of a target gene in a target cell. The present disclosure further relates to a use of an oligonucleotide and/or dsRNA molecule described herein for inhibiting expression of a target gene in a target cell in vitro.

Another aspect the invention relates to a method of modulating the expression of a target gene in a cell, comprising administering to said cell an oligonucleotide and/or dsRNA molecule described herein. In some embodiments, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erkl/2 gene, PCNA(P21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the P73 gene, mutations in the P21(WAF1/CIP1) gene, mutations in the P27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, and mutations in the P53 tumor suppressor gene.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I Freshney, ed., 1987); “Methods in Enzymology” (Academic Press, Inc.); “Current Protocols in Molecular Biology” (F. Mi. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxy, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxy, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, —NHCH₂CH₂CH₃, and —NHCH(CH₃)₂. Exemplary dialkylamino includes, but is not limited to, —N(C₁-C₁₀alkyl)₂, such as N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxy” and “hydroxyl” mean the radical OH.

The terms “alkoxyl” or “alkoxy” as used herein refers to an alkyl group, as defined above, having an oxygen radical attached thereto, and can be represented by one of —O-alkyl, —O-alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical C(O)—. It is noted that the carbonyl radical can be further substituted with a variety of substituents to form different carbonyl groups including acids, acid halides, amides, esters, ketones, and the like.

As used herein, the term “oxo” means double bonded oxygen, i.e., ═O.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes —COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}, —N⁺(O⁻)═, —O—, —S— or —S(O)₂—, —OS(O)₂—, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl-group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)N₁₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a R^{C}(═O)O— group.

“Alkoyl” refers to a R^{C}(═O)— group.

As used herein, the terms “dsRNA”, “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9,10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

The term “off-target” and the phrase “off-target effects” refer to any instance in which an effector molecule against a given target causes an unintended affect by interacting either directly or indirectly with another target sequence, a DNA sequence or a cellular protein or other moiety. For example, an “off-target effect” may occur when there is a simultaneous degradation of other transcripts due to partial homology or complementarity between that other transcript and the sense and/or antisense strand of an siRNA.

As used herein, the term “nucleoside” means a glycosylamine comprising a nucleobase and a sugar. Nucleosides includes, but are not limited to, naturally occurring nucleosides, abasic nucleosides, modified nucleosides, and nucleosides having mimetic bases and/or sugar groups.

As used herein, the term “nucleotide” refers to a glycosomine comprising a nucleobase and a sugar having a phosphate group covalently linked to the sugar. Nucleotides may be modified with any of a variety of substituents.

As used herein, the term “locked nucleic acid” or “LNA” or “locked nucleoside” or “locked nucleotide” refers to a nucleoside or nucleotide wherein the furanose portion of the nucleoside includes a bridge connecting two carbon atoms on the furanose ring, thereby forming a bicyclic ring system. Locked nucleic acids are also referred to as bicyclic nucleic acids (BNA).

As used herein, unless otherwise indicated, the term “methyleneoxy LNA” alone refers to β-D-methyleneoxy LNA.

As used herein, the term “MOE” refers to a 2′-O-methoxyethyl substituent.

As used herein, the term “gapmer” refers to a chimeric oligomeric compound comprising a central region (a “gap”) and a region on either side of the central region (the “wings”), wherein the gap comprises at least one modification that is different from that of each wing. Such modifications include nucleobase, monomeric linkage, and sugar modifications as well as the absence of modification (unmodified). Thus, in certain embodiments, the nucleotide linkages in each of the wings are different than the nucleotide linkages in the gap. In certain embodiments, each wing comprises nucleotides with high affinity modifications and the gap comprises nucleotides that do not comprise that modification. In certain embodiments the nucleotides in the gap and the nucleotides in the wings all comprise high affinity modifications, but the high affinity modifications in the gap are different than the high affinity modifications in the wings. In certain embodiments, the modifications in the wings are the same as one another. In certain embodiments, the modifications in the wings are different from each other. In certain embodiments, nucleotides in the gap are unmodified and nucleotides in the wings are modified. In certain embodiments, the modification(s) in each wing are the same. In certain embodiments, the modification(s) in one wing are different from the modification(s) in the other wing. In certain embodiments, oligomeric compounds are gapmers having 2′-deoxynucleotides in the gap and nucleotides with high-affinity modifications in the wing.

The term ‘BNA’ refers to bridged nucleic acid, and is often referred as constrained or inaccessible RNA. BNA can contain a 5-, 6-membered, or even a 7-membered bridged structure with a “fixed” C₃′-endo sugar puckering. The bridge is typically incorporated at the 2′-, 4′-position of the ribose to afford a 2′, 4′-BNA nucleotide (e.g., LNA, or ENA). Examples of BNA nucleotides include the following nucleosides:

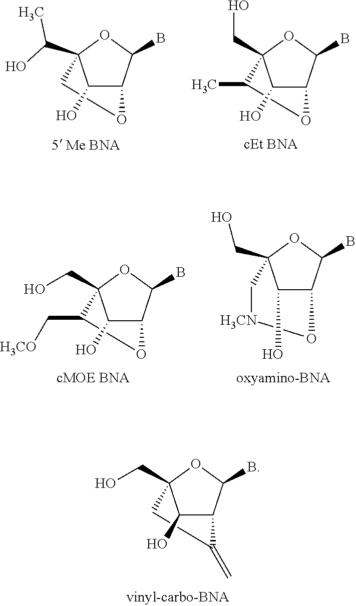

The term ‘LNA’ refers to locked nucleic acid, and is often referred as constrained or inaccessible RNA. LNA is a modified RNA nucleotide. The ribose moiety of an LNA nucleotide is modified with an extra bridge (e.g., a methylene bridge or an ethylene bridge) connecting the 2′ hydroxyl to the 4′ carbon of the same ribose sugar. For instance, the bridge can “lock” the ribose in the 3′-endo North) conformation:

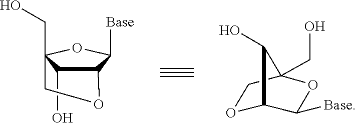

The term ‘ENA’ refers to ethylene-bridged nucleic acid, and is often referred as constrained or inaccessible RNA.

The “cleavage site” herein means the backbone linkage in the target gene or the sense strand that is cleaved by the RISC mechanism by utilizing the iRNA agent. And the target cleavage site region comprises at least one or at least two nucleotides on both side of the cleavage site. For the sense strand, the cleavage site is the backbone linkage in the sense strand that would get cleaved if the sense strand itself was the target to be cleaved by the RNAi mechanism. The cleavage site can be determined using methods known in the art, for example the 5′-RACE assay as detailed in Soutschek et al., Nature (2004) 432, 173-178, which is incorporated by reference in its entirety. As is well understood in the art, the cleavage site region for a conical double stranded RNAi agent comprising two 21-nucleotides long strands (wherein the strands form a double stranded region of 19 consecutive base pairs having 2-nucleotide single stranded overhangs at the 3′-ends), the cleavage site region corresponds to positions 9-12 from the 5′-end of the sense strand.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, a “terminal region” of a strand refers to positions 1-4, e.g., positions 1, 2, 3, and 4, counting from the nearest end of the strand. For example, a 5′-terminal region refers to positions 1-4, e.g., positions 1, 2, 3 and 4 counting from the 5′-end of the strand. Similarly, a 3′-terminal region refers to positions 1-4, e.g., positions 1, 2, 3 and 4 counting from the 3′-end of the strand.

For example, a 5′-terminal region for the antisense strand is positions 1, 2, 3 and 4 counting from the 5′-end of the antisense strand. A preferred 5′-terminal region for the antisense strand is positions 1, 2 and 3 counting from the 5′-end of the antisense strand. A 3′-terminal region for the antisense strand can be positions 1, 2, 3, and 4 counting from the 3′-end of the strand. A preferred 3′-terminal region for the antisense strand is positions 1, 2 and 3 counting from the 3′-end of the antisense strand.

Similarly, a 5′-terminal region for the sense strand is positions 1, 2, 3 and 4 counting from the 5′-end of the sense strand. A preferred 5′-terminal region for the sense strand is positions 1, 2 and 3 counting from the 5′-end of the sense strand. A 3′-terminal region for the sense strand can be positions 1, 2, 3, and 4 counting from the 3′-end of the strand. A preferred 3′-terminal region for the sense strand is positions 1, 2 and 3 counting from the 3′-end of the sense strand.

As used herein, a “central region” of a strand refers to positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the strand. For example, the central region of a strand means positions 5, 6, 7, 8, 9, 10, 11, 12, 13, 14, 15, 16 or 17 of the strand. A preferred central region for the sense strand is positions 6, 7, 8, 9, 10, 11, 12, 13, and 14, counting from the 5′-end of the sense strand. A more preferred central region for the sense strand is positions 7, 8, 9, 10, 11, 12 and 13, counting from the 5′-end of the sense strand. A preferred central region for the antisense strand is positions 9, 10, 11, 12, 13, 14, 15 16 and 17, counting from 5′-end of the antisense strand. A more preferred central region for the antisense strand is positions 10, 11, 12, 13, 14, 15 and 16, counting from 5′-end of the antisense strand.

As used herein, the term “in vitro” refers to events that occur in an artificial environment, e.g., in a test tube or reaction vessel, in cell culture, etc., rather than within an organism (e.g. animal or a plant). As used herein, the term “ex vivo” refers to cells which are removed from a living organism and cultured outside the organism (e.g., in a test tube). As used herein, the term “in vivo” refers to events that occur within an organism (e.g. animal, plant, and/or microbe).

As used herein, the term “subject” or “patient” refers to any organism to which a composition disclosed herein can be administered, e.g., for experimental, diagnostic, and/or therapeutic purposes. Typical subjects include animals (e.g., mammals such as mice, rats, rabbits, non-human primates, and humans) and/or plants. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to with domesticated animals and/or pets.

In some embodiments, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species. In some embodiments, the subject can be of European ancestry. In some embodiments, the subject can be of African American ancestry. In some embodiments, the subject can be of Asian ancestry.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

As used herein, the term “dosage unit” refers to a form in which a pharmaceutical agent is provided. In certain embodiments, a dosage unit is a vial comprising lyophilized antisense oligonucleotide. In certain embodiments, a dosage unit is a vial comprising reconstituted antisense oligonucleotide.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

Copper-assisted azide alkyne cycloadditions (CuAAC)¹⁻⁴ are versatile, and inventors have used this approach to functionalize a 1′-pentose-azide scaffold with the goal of modifying oligonucleotides with multiple ligands. The 1′ pentose-azide scaffold was reacted with various multivalent alkynes to produce scaffolds with mono-, bi-, tri-, and penta-valent ligation sites. As ligands, the inventors have used mono- and tri-valent carbohydrates, alkyls, cholesterol and other lipids, PEGs, peptides, polyamines, fluorophores, and biotin. The α- and β-anomers were separated after the CuAAC reaction to enable creation of a larger compound library from the same scaffold. Reactions were performed in solution as well as on solid supports. Regioisomeric conjugates can be derived using ruthenium-assisted click chemistry (RuAAC)⁵⁻⁷

Plethora of examples so far have demonstrated on copper (Cu)/ruthenium (Ru) assisted azide alkyne cycloadditions (CuAAC¹⁻⁴ and RuAAC⁵⁻⁷), metal-free cycloaddition⁸⁻⁹ (strained promoted cycloaddition (SPAAC¹⁰) and inverse electron-demand Diels-Alder chemistry (iEDDA¹¹⁻¹³)) and cycloaddition reactions assisted by other metal ions¹⁴⁻¹⁷ after the pioneering work by Sharpless, Meldal and Fokin¹⁸⁻²⁰. Due to the robust nature, versatility, and ease of application, the CuAAC is still the unparallel tool in this field encompassing broad-spectrum applications.

In this study, the inventors demonstrate approach to Click conjugation utilizing the pentose and proline scaffolds for CuAAC chemistry. The separation of α- and β-anomers pentose and hexose sugars after the CuAAC reaction with different multivalent alkynes allows preparation of larger compound library from the same scaffold. As the results presented herein show, CuAAC chemistry between anomeric azide with various multivalent alkynes produces mono-, bi-, tri- and penta-valent ligation sites which can be functionalized by CuAAC either on solid support^{19, 21} or in solution¹⁻⁴. The selection of azides depends on the targeting ligands/enzymes/active sites at the multiple clickable sites generated from sugars and proline scaffolds. The azides contain variety of functional groups viz., i) sugars scaffolds (e.g. MonoGalNAc, TriGalNAc), ii) simple alkyl (hexyl azide), iii) PEG groups (mPEG azide), iv) biomarkers (biotin azide), v) peptides (cRGD²²), vi) polyamine, vii) cholesterol and lipids, viii) fluorescent dyes, ix) adamentyl, cubane, x) DUPA etc. Ligands can be therapeutic agents, diagnostic agents, imaging agents, and/or targeting ligands. RuAAC chemistry can be combined with the CuAAC chemistry to widen the scope of the multiple click reactions demonstrated herein. CuAAC will generate the classical 1,4-regioisomer, RuAAC elicits the 1,5-disubstituted triazoles for azide-alkyne cycloaddition.

Various monomers for used in this study were synthesized following the methods shown in Schemes 1-7, 13 and 14.

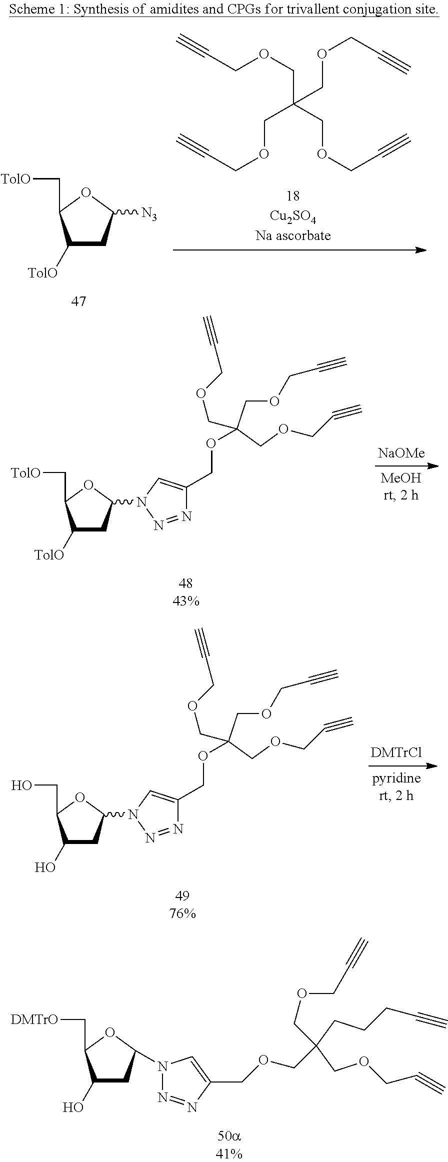

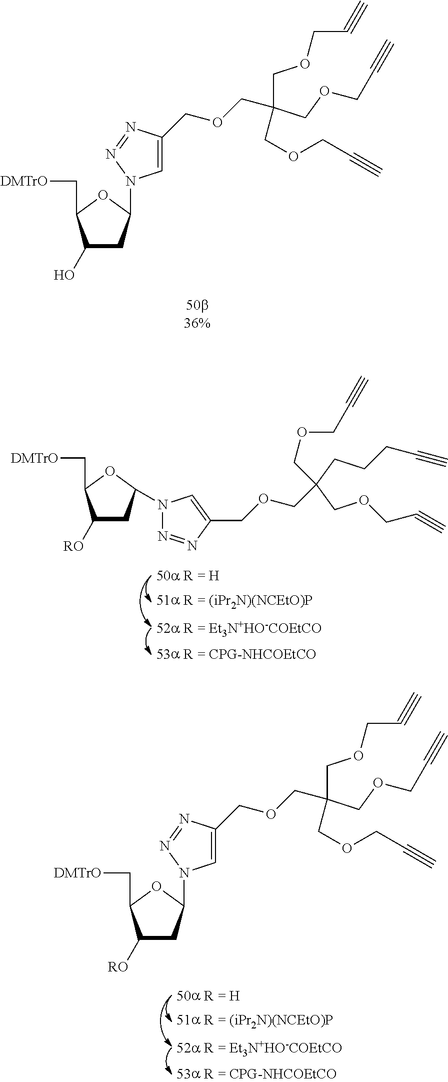

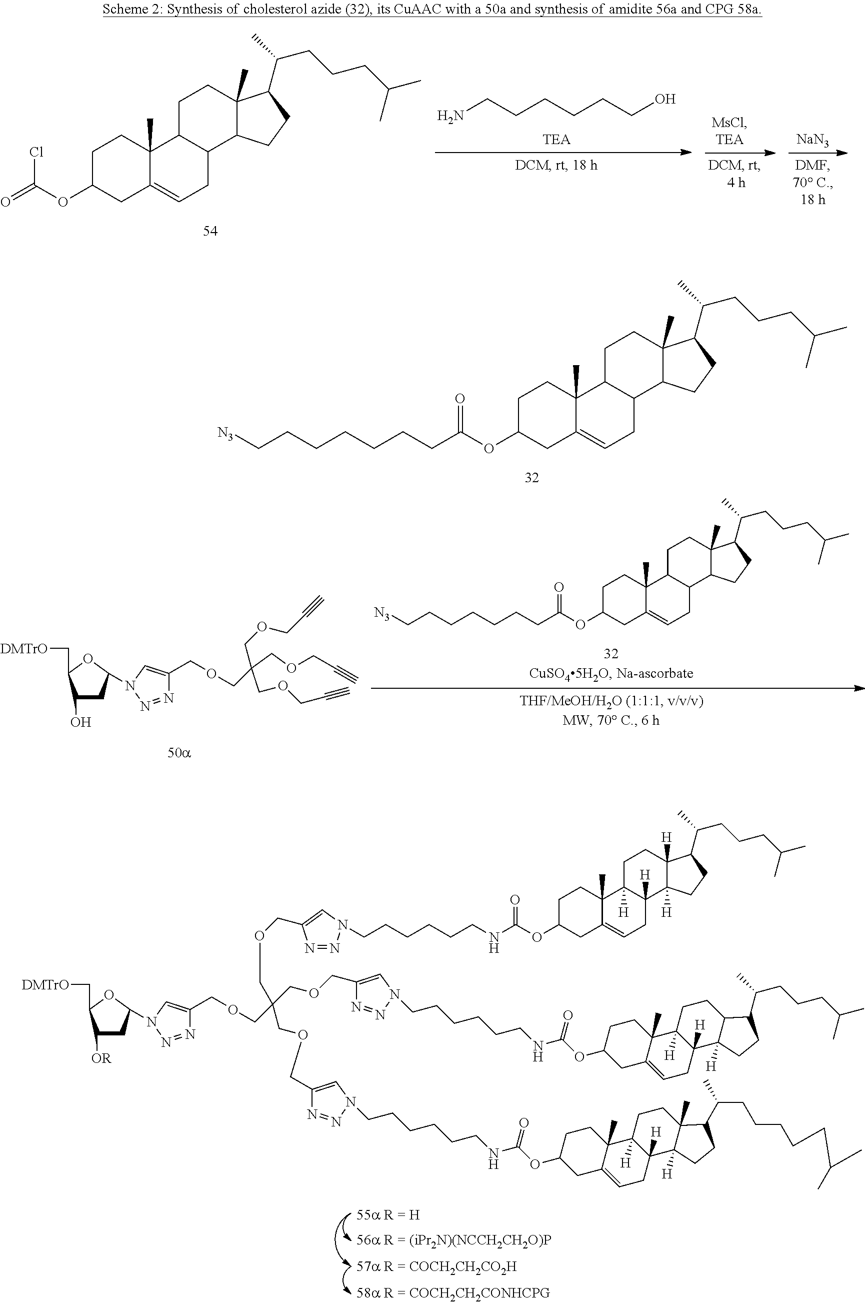

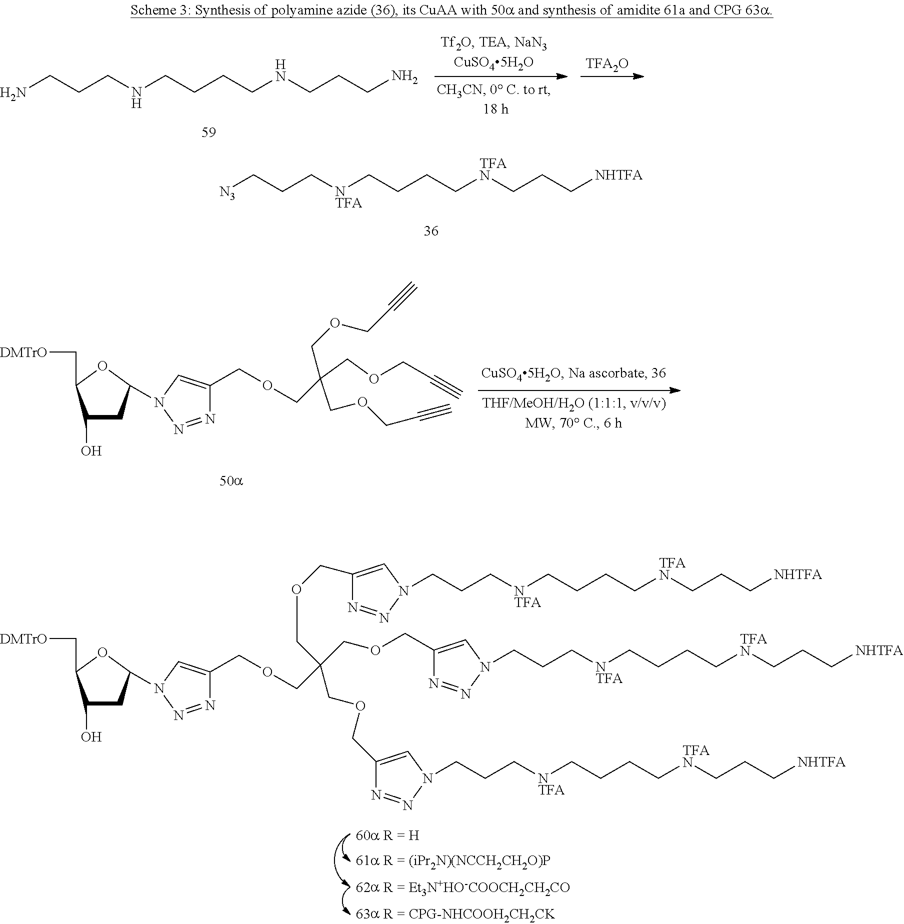

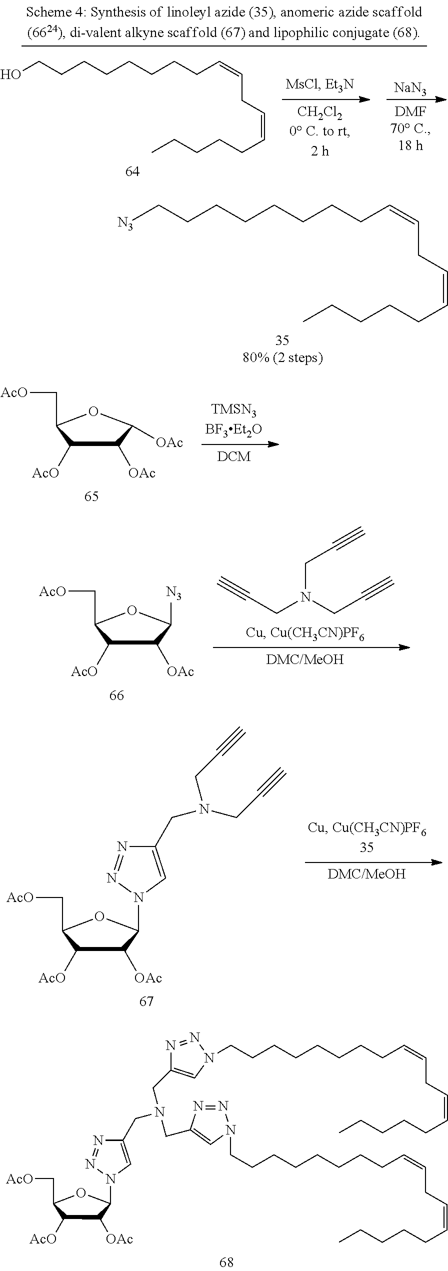

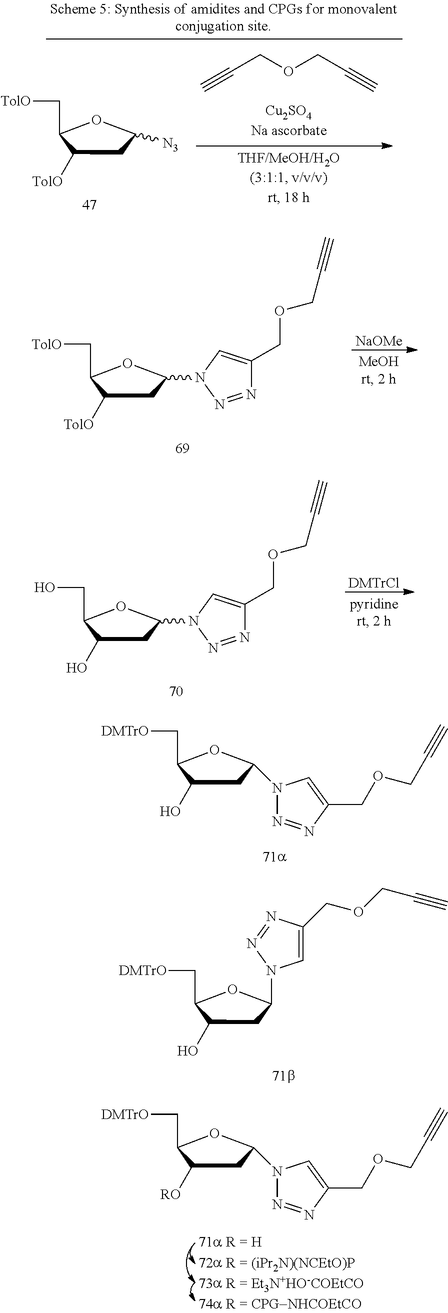

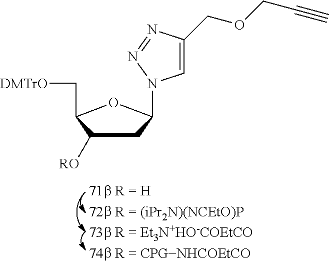

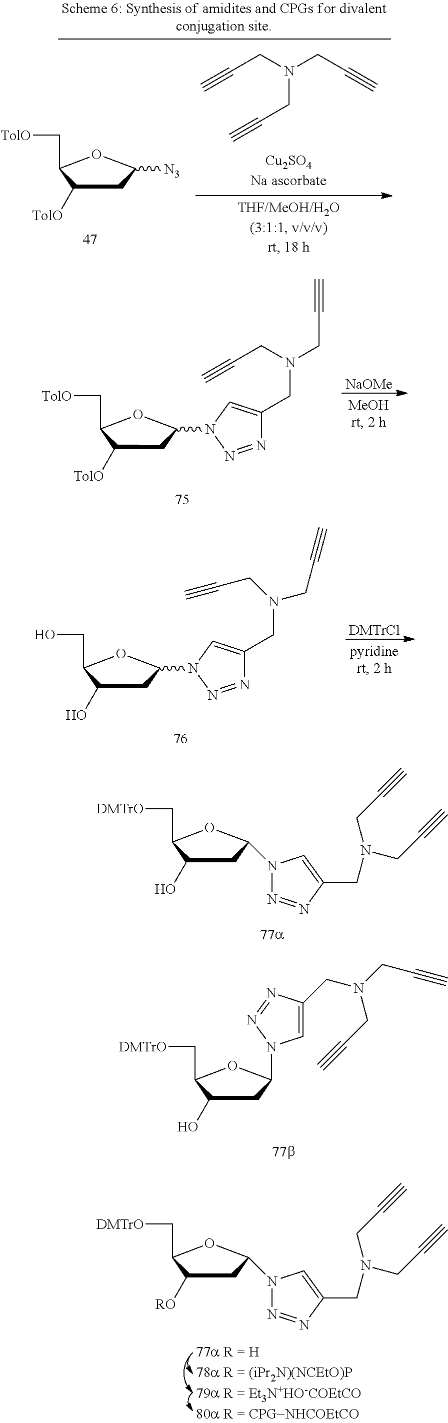

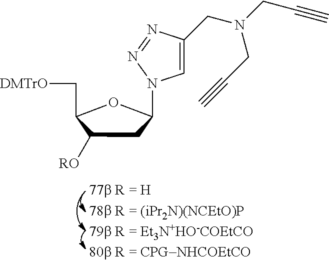

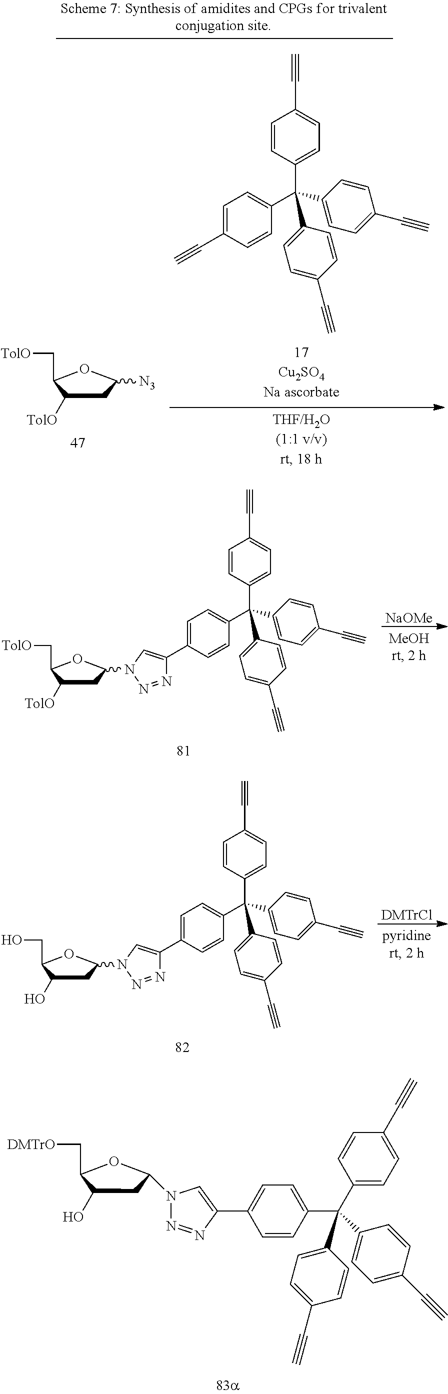

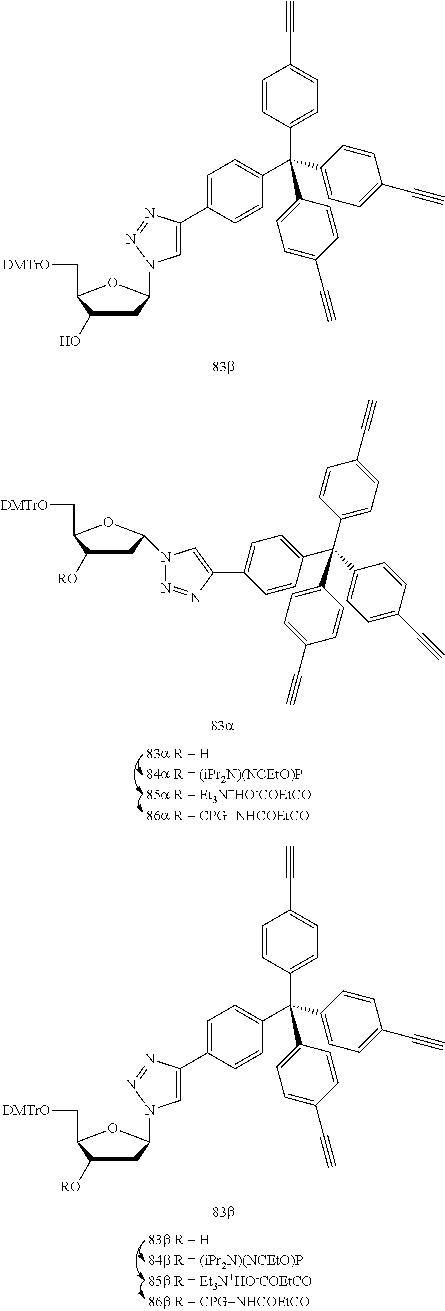

### Experimental Section

General condition for CuAAC: In solution phase: Azide (1 equiv.) in THF/MeOH/H₂O (3:1:1, v/v/v) was treated with alkyne (4 eqiv.), coppersulfate pentahydrate (0.02 equiv.) and sodium ascorbate (0.1 equiv.) at room temperature for 18 h. Then the whole solution was extracted with EtOAc/brine. The organic layer was dried with sodium sulfate and then concentrated under reduced pressure. The residue was chromatographed on silica gel. Eluted with DCM and MeOH mixture to afford pure compounds.

CuAAC⁷²⁻²⁸ and RuAAC²⁹⁻³⁰ on Solid Support:

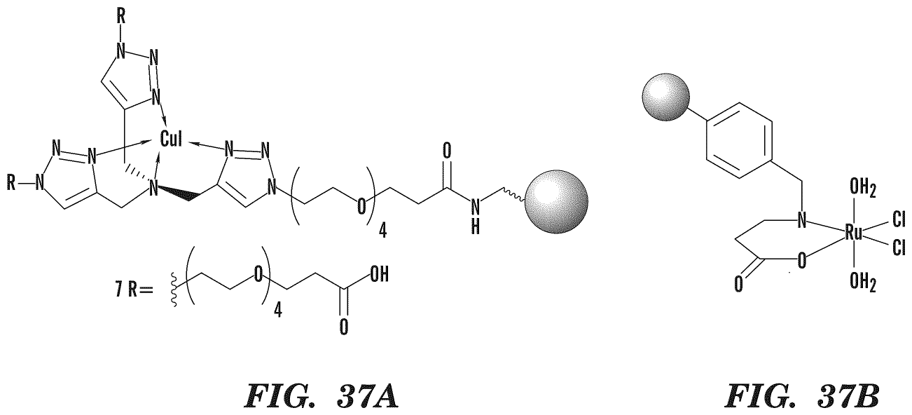

Preparation of solid-supported ligand-Cu (I) complex: To a solution of tetrakis(acetonitrile)copper(I) hexafluorophosphate [Cu(CH₃CN)₄PF₆] (0.23 mmol) in DMF was added chelating ligand (0.11 mmol). The suspension was gently shaken on a wrist action shaker at room temperature for 2 h. After filtration, the resin was washed with DCM (1 vol.), DCM/methanol (9:1, 2 vol.), DCM (1 vol.), and ether (2 vol.) and dried under vacuum to obtain the solid support 7 (0.507 g). Loading of the Cu(I) on the resin was analyzed by ICP/OES. Loading was 222 μmol/g, equivalent to ˜97% of the total amine content on the resin (FIG. 37A).

Click reaction: To a mixture of alkyne (1.0 eq) and azide (1.0 eq) in DMF/DCM (2:1) was added Cu-complex (0.1 eq), and the mixture was gently shaken on a wrist-action shaker overnight. Completion of the reaction was confirmed by TLC. The reaction mixture was filtered and washed with DCM (2 vol.). The filtrate was poured onto ice, and the product was extracted into DCM. The organic layer was washed with excess water, and then dried over anhydrous MgSO₄. The product was purified by flash silica gel column chromatography (eluent: DCM/MeOH to obtain pure compound.

General Procedure for Click Reaction on Solid Support.²⁸ To a 0.6 μmol solid-supported alkyne-RNA phosphotriester was added a mixture of an azide (3 equiv by alkyne, 1.8 μmol, 36 μL of a 50 mM solution in THF), freshly prepared CuSO₄ 5H₂O (0.4 equiv, 0.24 μmol, 5 μL of a 50 mM solution in H₂O), freshly prepared sodium ascorbate (3 equiv, 1.8 μmol, 37 μL of a 50 mM solution in H₂O), and TBTA (3 equiv, 1.8 μmol, 37 μL of a 50 mM solution in THF). Water/MeOH/THF (2:2:1 v/v) was added to obtain a total volume of 1200 μL. The resulting preparation was heated in a sealed glass tube in an Explorer-48 microwave synthesizer at a 100 W and a 30 s premixing time. The temperature was monitored with an internal infrared probe and held at 60° C. over 45 min. The solution was removed, and the CPG supports were washed with THF and MeOH then dried. The product on the CPG supports were cleaved and deprotected by treating the solid-support with 100 μL of methylamine solution (40 wt % in water) at 65° C. for 10 min. The mixture was cooled on dry ice for 5 min and the solid suspension was spun down. Of the supernatant, 80 μL was decanted into another microtube and heated with 120 μL of TEA 3HF at 65° C. for 12 min. The product was analyzed by LC-MS and by RP-HPLC(C₄ column, 150×3.9 μm i.d., 5 μm, 300 Å) using a linear gradient of 0 to 70% B over 24 min at 30° C. at a flow rate of 1 mL/min (buffer A: 50 mM TEAA, pH 7.0; buffer B: CH₃CN). Completion of the CuAAC reaction was estimated by comparing the purity of starting material alkyne-RNAs and their corresponding triazole products by RP-HPLC. After base and sugar deprotection, oligonucleotides were purified by RP-HPLC with C₄ column (300×7.8 mm i.d., 15 μm, 300 Å) using a linear gradient of 0 to 90% B in 40 min at room temperature at a flow rate of 3 mL/min (buffer A: 50 mM TEAA, pH 7.0; buffer B: CH₃CN).

General condition for RuAAC: A typical RuAAC procedure involves the reaction of an alkyne with an organic azide in the presence of catalytic amounts of a ruthenium(II) complex containing a [Cp*RuCl] unit in a nonprotic solvent. A variety of solvents can be used for the reaction, where the most used are aromatic solvents, like benzene or toluene, or ethers such as tetrahydrofuran (THF) and dioxane. Certain polar solvents can also be applied, i.e., dimethylformamide (DMF) and dimethylacetamide (DMA), while reactions using dimethyl sulfoxide (DMSO) have been reported to be problematic, which is most likely related to the ability of DMSO to act as ligand to ruthenium. Protic solvents are not suitable, giving low yields and a high degree of byproduct formation. Most reported RuAAC reactions employ either Cp*RuCl(PPh₃)₂ or Cp*RuCl(COD) as the catalyst, using between 1 and 5 μmol % catalyst, and both complexes are commercially available. Although heating is generally employed to shorten reaction times, reactions at ambient temperature are also possible, especially when using a catalyst with high reactivity such as Cp*RuCl(COD). A typical procedure for the RuAAC reaction has been described by Oakdale and Fokin.⁵ It is noted that Cp* is pentamethylcyclopentadienyl and COD is cyclooctadiene.

RuAAC on polymer support²⁹⁻³⁰: Solid-supported Ru catalyst has been described with employing a polymeric support³⁰. The polymer-bound ruthenium(III) catalyst 5 (FIG. 37B) was prepared by reaction of RuCl3 with a polystyrene-tethered β-alanine ligand, affording a supported catalyst containing 8.5 wt % ruthenium (determined by AAS). To test the catalyst in RuAAC, phenyl acetylene was mixed with sodium azide and benzyl bromide in the presence of varying amounts of the supported catalyst. Here, water provided the best medium for this transformation. Employing 2 μmol % catalyst and a reaction time of 3 h at 40° C. produced the 1,4-disubstituted 1,2,3-triazole in quantitative yield, after a simple workup involving only filtration, washing with ethanol, and concentration. The substitution pattern of the product is not surprising considering that the catalyst contains neither a Cp nor a Cp* ligand, and thus, this result is in accord with earlier studies by Jia, Fokin, and co-workers.³¹ The recyclability was investigated, and the supported catalyst could be employed up to six times without noticeable reduction in catalytic activity.

General conditions: TLC was performed on Merck silica gel 60 plates coated with F254. Compounds were visualized under UV light (254 nm) or after spraying with the p-anisaldehyde staining solution followed by heating. Flash column chromatography was performed using a Teledyne ISCO Combi Flash system with pre-packed RediSep Teledyne ISCO silica gel cartridges. All moisture-sensitive reactions were carried out under anhydrous conditions using dry glassware, anhydrous solvents, and argon atmosphere. All commercially available reagents and solvents were purchased from Sigma-Aldrich unless otherwise stated and were used as received. ESI-MS spectra were recorded on a Waters QTof Premier instrument using the direct flow injection mode. 1H NMR spectra were recorded at 300, 400 and 500 MHz. ¹³C NMR spectra were recorded at 75, 101, and 126 MHz. ³¹P NMR spectra were recorded at 162 and 202 MHz. Chemical shifts are given in ppm referenced to the solvent residual peak (DMSO-d₆-1H: δ at 2.50 ppm and ¹³C δ at 39.5 ppm; CDCl₃-¹H: δ at 7.26 ppm and ¹³C δ at 77.16 ppm). Coupling constants are given in Hertz. Signal splitting patterns are described as singlet (s), doublet (d), triplet (t), septet (sept), broad signal (brs), or multiplet (m).

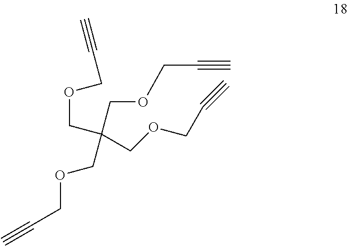 [SMILES: C#CCOCC(COCC#C)(COCC#C)COCC#C]

Compound 18. To a solution of pentaerythritol (4.00 g, 29.40 mmol) in anhydrous DMF (50 ml) was added sodium hydride (3.52 g, 146.98 mmol) at 0° C. The solution was vigorously stirred for 30 min at 0° C. Then propargyl bromide (17.48 g, 146.98 mmol) was added slowly at 0° C. The solution was then allowed to heat to 60° C. and then vigorously stirred for 18 h. TLC and LC-Mass analysis showed reaction completed (Rf=0.5 at Hexane/EtOAc=9:1, stained with PMA). After quenching with water, whole solution was extracted with Et₂O/sat NH₄Cl aq. The organic layer was dried with sodium sulfate and then concentrated under reduced pressure. Obtained yellow syrup was subjected to flash silica gel chromatography. Eluent with Hexane/EtOAc (9:1, v/v) gave 18 (7.70 g, 26.7 mmol, 90%) as a yellow foam. LC-Mass: Calcd for C₁₇H₂₁O₄=289.0, found=289.0. ¹H-NMR (400 MHz, DMSO-d₆) δ 3.32-3.42 (12H, m), 4.10-4.16 (8H, d).

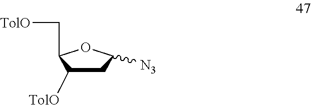

Compound 47¹⁵: To a suspension of [(2R,3S)-5-chloro-3-(4-methylbenzoyl)oxy-tetrahydrofuran-2-yl]methyl 4-methylbenzoate (Hoffer chloro sugar) (10.0 g, 25.72 mmol) in dimethylformamide (50 mL) was added sodium azide (2.6 g, 39.99 mmol, 1.41 mL) slowly in portions and stirred vigorously for 2.5 hr and TLC was checked. After completion of reaction, reaction mixture was diluted with EtOAc (100 mL) and water (50 mL). Organic layer was separated and washed with brine solution (3×100 mL). Ethyl acetate layer was separated, dried over anhydrous Na₂SO₄, filtered, and filtrate was evaporated to dryness. The crude brown oil was purified by flash column chromatography (gradient: 5-20% EtOAc in hexane) to afford 47 (8.5 g, 84% yield) as transparent oil (mixture of both anomers). ¹H NMR (600 MHz, CDCl₃) δ 8.07-7.77 (m, 4H), 7.25 (ddd, J=13.0, 8.0, 4.1 Hz, 4H), 5.75-5.70 (m, 1H), 5.63-5.41 (m, 1H), 4.65-4.50 (m, 3H), 2.63-2.15 (m, 8H) ppm. ¹³C NMR (151 MHz, CDCl₃) δ 166.43, 166.42, 166.28, 166.06, 144.44, 144.39, 144.16, 144.03, 129.95, 129.92, 129.88, 129.84, 129.82, 129.79, 129.34, 129.32, 129.28, 129.25, 127.01, 126.93, 126.74, 126.62, 92.22, 92.16, 83.71, 82.81, 75.00, 74.69, 64.31, 64.11, 38.96, 38.77, 21.86, 21.84, 21.82 ppm.

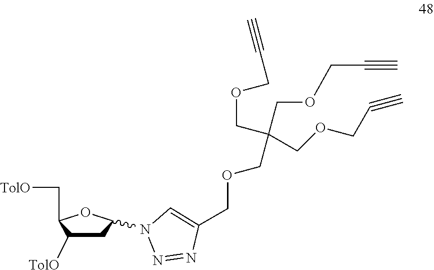

Compound 48. Compound 47 (10.34 g, 26.15 mmol) in THF/MeOH/H₂O (3:1:1, v/v/v) (200 ml) was treated with 18 (23.78 g, 82.4 mmol), copper sulfate pentahydrate (130 mg, 0.52 mmol) and sodium ascorbate (518 mg, 2.62 mmol) at room temperature for 18 h. Then the whole solution was extracted with EtOAc/brine. The organic layer was dried with sodium sulfate and then concentrated under reduced pressure. The residue was chromatographed on silica gel. Eluent with Hexane/EtOAc (8:2, v/v) gave 48 as a yellow gum (7.67 g, 11.19 mmol, 43%). ¹H NMR (600 MHz, CDCl₃) δ 8.02-7.73 (m, 4H), 7.66 (d, J=8.2 Hz, 1H), 7.29-7.18 (m, 5H), 6.47 (ddd, J=6.4, 3.8, 2.2 Hz, 1H), 5.81-5.58 (m, 1H), 4.82-4.51 (m, 5H), 4.15-4.06 (m, 7H), 3.56-3.44 (m, 9H), 3.28-3.18 (m, 1H), 3.05-2.77 (m, 1H), 2.46-2.36 (m, 9H) ppm. ¹³C NMR (151 MHz, CDCl₃) δ 166.27, 166.12, 165.99, 146.28, 145.97, 144.63, 144.59, 144.37, 144.23, 129.92, 129.85, 129.83, 129.45, 129.41, 129.39, 129.37, 126.84, 126.78, 126.54, 126.31, 121.36, 120.81, 89.95, 88.89, 84.75, 83.65, 80.20, 80.18, 80.16, 74.99, 74.63, 74.30, 74.21, 69.38, 69.35, 69.18, 69.03, 68.98, 65.38, 65.19, 64.11, 64.01, 60.53, 58.87, 58.82, 58.80, 53.56, 45.05, 45.04, 38.70, 38.25, 21.87, 21.85, 21.83 ppm. HRMS calc. for C₃₈H₄₂O₉N₃ [M+H]⁺ 684.2921, found 684.2913.

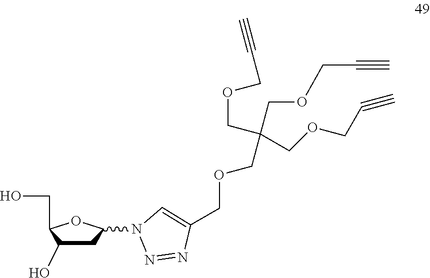

Compound 49. To a solution of 48 (6.00 g, 8.78 mmol) in methanol (200 ml) was added 0.5 M sodium methoxide in methanol (200 ml). After vigorous stirring for 2 h, the solution was concentrated under reduced pressure. The residue was extracted with EtOAc/sat NH₄Claq. The organic layer was dried with sodium sulfate and then concentrated under reduced pressure to give dark gum. The gum was subjected to silica gel chromatography. Eluent with 7% methanol in DCM gave 49 (3.14 g, 6.68 mmol, 76%) as yellow gum. ¹H NMR (600 MHz, DMSO) δ 8.28 (s, 2H), 8.24 (s, 2H), 7.86-7.81 (m, 1H), 7.32-7.27 (m, 1H), 6.39-6.33 (m, 3H), 5.47 (d, J=4.0 Hz, 2H), 5.35 (d, J=4.4 Hz, 2H), 4.93-4.84 (m, 3H), 4.49 (d, J=8.1 Hz, 7H), 4.37 (dq, J=6.0, 4.1 Hz, 2H), 4.28 (dq, J=7.1, 3.5 Hz, 2H), 4.15-4.02 (m, 22H), 3.86 (td, J=5.1, 3.4 Hz, 2H), 3.55-3.48 (m, 2H), 3.45-3.38 (m, 33H), 3.16 (d, J=5.2 Hz, 2H), 2.77 (dt, J=14.4, 7.3 Hz, 2H), 2.61 (dt, J=13.3, 6.1 Hz, 2H), 2.40-2.33 (m, 3H), 2.29 (dt, J=14.2, 3.1 Hz, 2H) ppm. ¹³C NMR (151 MHz, DMSO) δ 167.36, 144.37, 144.31, 143.10, 129.38, 129.19, 128.03, 122.50, 122.23, 88.67, 88.60, 88.28, 87.96, 80.37, 77.23, 70.53, 70.44, 68.90, 68.88, 68.64, 64.17, 64.10, 61.68, 61.41, 58.11, 48.65, 44.36, 21.19 ppm. HRMS calc. for C₂₂H₃₀N₃O₇ [M+H]⁺ 448.2084, found 448.2083.

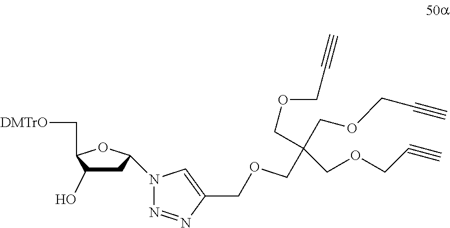

Compound 50α. Compound 49 (3.14 g, 7.02 mmol) in anhydrous pyridine (100 ml) was treated with DMTrCl (2.61 g, 7.72 mmol) for 2 h at ambient temperature. The solution was extracted with EtOAc/sat aqueous NaHCO₃. The organic layer was dried with sodium sulfate and then concentrated under reduced pressure. Then the residue was subjected to flash silica gel chromatography. Eluent with Hexane/EtOAc (4:6, v/v) gave 50α (2.18 g, 2.91 mmol, 41%). LC-Mass: Calcd for C₄₃H₄₇N₃O₉Na=772.2, found 772.2. ¹H-NMR (400 MHz, DMSO-d₆) δ 2.31-2.36 (1H, dd), 2.78-2.85 (1H, m) 2.97-3.02 (1H, dd), 3.13-3.17 (1H, dd), 3.33-3.40 (11H, m), 3.73 (6H, s), 4.05-4.09 (6H, d), 4.21-4.27 (2H, m), 4.52 (2H, s), 5.50-5.51 (1H, d, diminished with D₂O), 6.46-6.49 (1H, dd), 6.88-6.92 (4H, d), 7.20-7.40 (9H, m), 8.31 (1H, s). ¹³C-NMR (400 MHz, DMSO-d₆) δ 44.37, 55.02, 58.08, 63.70, 64.16, 68.63, 68.88, 70.83, 77.03, 80.29, 85.51, 86.63, 88.69, 113.21, 122.21, 126.66, 127.66, 127.84, 129.67, 135.43, 135.53, 144.37, 144.77, 158.07 ppm. HRMS calc. for C₄₃H₄₈N₃O₉ [M+H]⁺ 750.3391, found 750.3392.

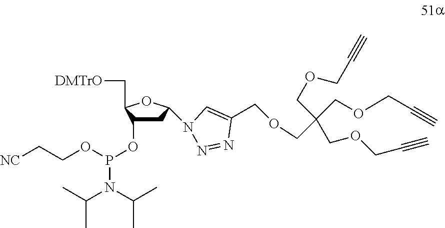

Compound 51α. Compound 50α (500 mg, 0.56 mmol) in anhydrous DCM (10 ml) was treated with diisopropylethylamine (290 ml, 1.67 mmol) and 2-cyanoethyl-N,N-diisopropylchlorophosphoramidite (185 ml, 0.83 mmol) at room temperature for 1 h. After quenched with methanol, the solution was extracted with DCM/sat aqueous NaHSO₄. The organic layer was dried with sodium sulfate and then concentrated under reduced pressure. The residue was then chromatographed on silica gel. Eluent with Hexane/EtOAc/TEA (60:40:0.5, v/v/v) gave 51a (538 mg, 0.56 mmol, 99%). LC-Mass: Calcd for C₅₂H₆₄N₅NaO₁₀P=972.2, found 972.2. ¹H-NMR (400 MHz, DMSO-d₆) δ 0.96-1.24 (12H, m), 2.58-2.72 (4H, m), 2.92-3.06 (2H, m), 3.22-3.30 (2H, m), 3.40 (8H, b), 3.45-3.66 (5H, m), 3.74-3.75 (6H, d), 4.08-4.09 (6H, d), 4.34-4.42 (1H, m), 4.50-4.51 (2H, d), 6.55-6.56 (1H, m), 6.89-6.92 (4H, m), 7.23-7.41 (9H, m), 8.23-8.24 (1H, d). ¹³C-NMR (400 MHz, DMSO-d₆) δ 19.77, 24.13, 24.21, 24.30, 42.51, 42.64, 44.35, 55.03, 58.08, 58.13, 62.69, 68.60, 77.11, 80.28, 85.63, 88.74, 88.81, 109.31, 113.22, 118.68, 118.88, 122.05, 122.09, 126.73, 127.60, 127.67, 127.88, 127.91, 129.69, 135.24, 135.36, 135.44, 144.19, 144.22, 144.68, 158.11. ³¹P-NMR (400 MHz, DMSO-d₆) δ 152.43, 152.84.

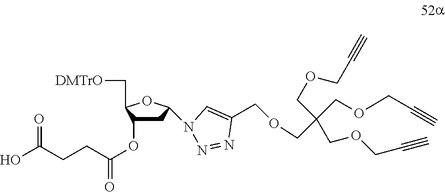

Compound 52α. Compound 50α (500 mg, 0.56 mmol) in anhydrous DCM (10 ml) was treated with N,N-dimethylaminopyridine (162 mg, 1.33 mmol) and succinic anhydride (339 mg, 1.00 mmol) for 4 h. The solution was chromatographed on silica gel without aqueous work-up. Eluent with DCM/methanol/TEA (90:5:5, v/v/v) gave 52a (630 mg, 0.66 mmol, 99%). LC-Mass: Calcd for C₄₇H₅₁N₃NaO₁₂=872.2, found=872.2. ¹H-NMR (400 MHz, DMSO-d₆) δ 0.95-0.97 (9H, dd), 2.33-2.41 (3H, m), 2.48-2.53 (8H, m), 2.93-3.04 (1H, m), 3.08-3.11 (1H, m), 3.21-3.25 (1H, m), 3.35-3.39 (11H, m), 3.73 (6H, s), 4.07-4.08 (6H, d), 4.44-4.47 (1H, dd), 4.51 (2H, s), 5.20-5.22 (1H, m), 6.61-6.63 (1H, dd), 6.89-6.91 (4H, m), 7.23-7.40 (9H, m), 8.20 (1H, s). ¹³C-NMR (400 MHz, DMSO-d₆) δ 11.01, 11.02, 11.08, 11.30, 11.35, 29.16, 29.30, 44.37, 45.55, 55.21, 58.09, 64.08, 68.62, 77.19, 80.31, 80.33, 85.76, 89.11, 113.39, 113.43, 113.45, 121.90, 122.12, 129.53, 129.85, 129.86, 129.90, 129.92, 135.27, 135.43, 144.23, 158.13, 172.05, 173.73.

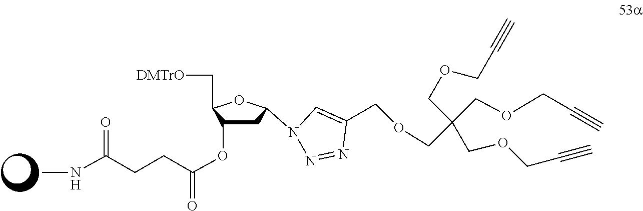

Compound 53α. Compound 52α (630 mg, 0.66 mmol) in DMF/DCM (2:1, v/v) (150 ml) was treated with N,N-diisopropylethylamine (462 ml, 2.65 mmol), HBTU (276 mg, 0.73 mmol) and CPG-NH₂ (Prime Synthesis CPG-500, NH₂ loading=140 mmol/g) (5.21 g) at ambient temperature for 4 h. The solid was collected by filtration, washed with DMF (100 ml), then 10% MeOH in DCM (200 ml) and dried under suction for 10 min. The residual amino groups were capped by shaking with Ac20/pyridine/TEA (75:25:5, v/v/v) (50 ml) for 30 min. Filtration and washing with 10% MeOH/DCM (300 ml) then drying overnight gave 53α (5.38 g, loading ratio=70 μmol/g).

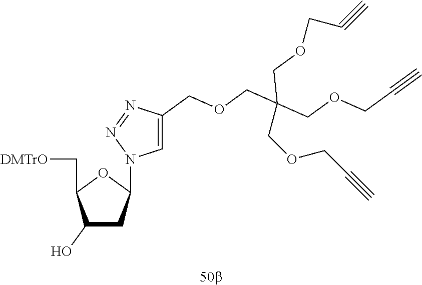

Compound 50β. Compound 50β was synthesized as an isomer of 50α. Compound 50β (1.99 g, 2.57 mmol, 36%) was eluted with Hexane/EtOAc (4:6, v/v) after 50a was completely eluted. LC-Mass: Calcd for C₄₃H₄₇N₃O₉Na=772.2, found 772.2. ¹H-NMR (400 MHz, DMSO-d₆) δ 2.36-2.42 (1H, m), 2.67-2.71 (1H, m), 3.04-3.10 (2H, m), 3.35-3.40 (11H, m), 3.73 (6H, s), 3.96-4.09 (7H, m), 4.39-4.42 (1H, m), 4.45 (2H, s), 5.40-5.42 (1H, d, diminished with D₂O), 6.37-6.40 (1H, dd), 6.82-6.92 (4H, m), 7.18-7.33 (9H, m), 8.16 (1H, s). ¹³C-NMR (400 MHz, DMSO-d₆) δ 44.34, 55.00, 58.06, 64.05, 68.55, 68.80, 70.34, 77.10, 80.29, 85.40, 86.02, 87.44, 113.11, 122.89, 126.59, 127.65, 127.76, 129.63, 129.70, 135.36, 135.60, 144.16, 144.72, 157.99 ppm.

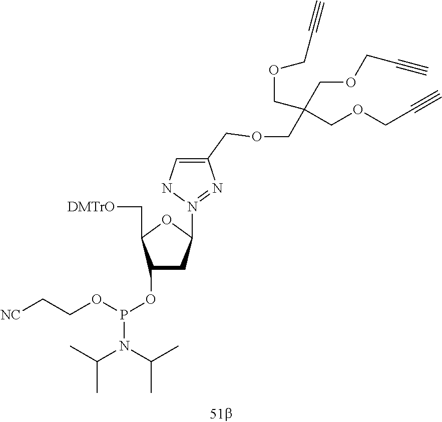

Compound 51β. Compound 50β (554 mg, 0.62 mmol) in anhydrous DCM (10 ml) was treated with diisopropylethylamine (320 ml, 1.85 mmol) and 2-cyanoethyl-N,N-diisopropylchlorophosphoramidite (210 ml, 0.83 mmol) at room temperature for 1 h. After quenched with methanol, the solution was extracted with DCM/sat NaHSO₄aq. The organic layer was dried with sodium sulfate and then concentrated under reduced pressure. The residue was then chromatographed on silica gel. Eluent with Hexane/EtOAc/TEA (60:40:0.5, v/v/v) gave 51β (552 mg, 0.58 mmol, 93%). LC-Mass: Calcd for C₅₂H₆₄N₅NaO₁₀P=972.2, found 972.2. ¹H-NMR (400 MHz, DMSO-d₆) δ 0.95-1.24 (12H, m), 2.53-2.65 (2H, m), 2.74-2.77 (1H, dd), 2.84-2.90 (1H, m), 3.03-3.19 (2H, m), 3.36-3.38 (11H, m), 3.47-3.62 (3H, m), 3.67-3.77 (7H, m), 3.97-4.14 (8H, m), 4.45 (2H, b), 4.63-4.71 (1H, m), 6.40-6.45 (1H, m), 6.81-6.85 (4H, m), 7.16-7.32 (9H, m), 8.21-8.22 (1H, d). ¹³C-NMR (400 MHz, DMSO-d₆) δ 14.1, 19.7, 19.8, 19.9, 20.8, 24.1, 24.2, 24.3, 24.3, 24.4, 42.5, 42.6, 44.3, 55.0, 58.1, 59.8, 64.1, 68.5, 77.1, 80.3, 85.5, 87.3, 113.1, 118.7, 119.0, 123.2, 126.6, 127.8, 129.7, 129.7, 135.4, 144.2, 144.6, 144.6, 158.0. ³¹P-NMR (400 MHz, DMSO-d₆) δ 152.36, 152.93 ppm.

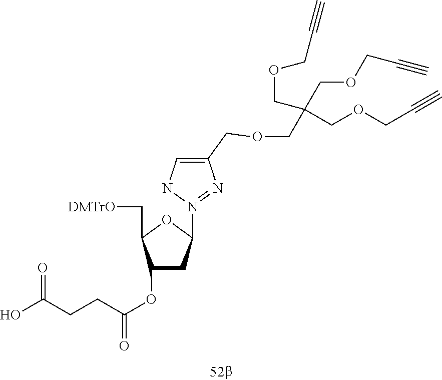

Compound 52P. Compound 50β (560 mg, 0.75 mmol) in anhydrous DCM (10 ml) was treated with N,N-dimethylaminopyridine (182 mg, 1.49 mmol) and succinic anhydride (379 mg, 1.12 mmol) for 4 h. The solution was chromatographed on silica gel without aqueous work-up. Eluent with DCM/methanol/TEA (90:5:5, v/v/v) gave 52P (680 mg, 0.71 mmol, 95%). LC-Mass: Calcd for C₄₇H₅₁N₃NaO₁₂=872.2, found=872.2. ¹H-NMR (400 MHz, DMSO-d₆) δ 0.98-1.22 (9H, m), 2.49-2.58 (8H, m), 2.97-3.03 (3H, m), 3.13-3.14 (3H, m), 3.36-3.37 (11H, m), 3.73 (6H, s), 4.04-4.05 (6H, d), 4.15-4.16 (1H, dd), 4.46 (2H, s), 5.36-5.37 (1H, m), 6.41-6.44 (4H, dd), 6.83-6.85 (4H, dd), 7.17-7.32 (9H, m), 8.19 (1H, s). ¹³C-NMR (400 MHz, DMSO-d₆) δ 28.7, 28.8, 44.4, 45.5, 58.1, 64.1, 68.6, 74.2, 74.5, 76.1, 77.1, 77.1, 77.2, 78.1, 80.3, 85.7, 113.1, 113.3, 113.3, 113.4, 113.5, 128.0, 130.0, 130.1, 135.3, 135.4, 144.4, 144.6, 158.1, 171.8, 173.4 ppm.

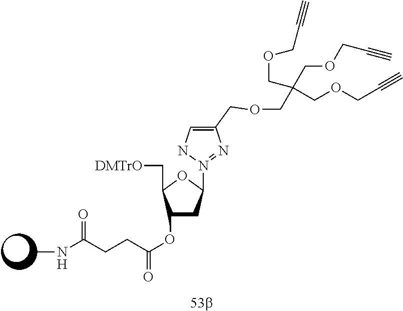

Compound 53β. Compound 52β (680 mg, 0.71 mmol) in DMF/DCM (2:1, v/v) (150 ml) was treated with N,N-diisopropylethylamine (498 ml, 2.86 mmol), HBTU (298 mg, 0.79 mmol) and CPG-NH₂ (Prime Synthesis CPG-500, NH₂ loading=140 mmol/g) (6.35 g) at ambient temperature for 4 h. The solid was collected by filtration, washed with DMF (100 ml), then 10% MeOH in DCM (200 ml) and dried under suction for 10 min. The residual amino groups were capped by shaking with Ac₂O/pyridine/TEA (75:25:5, v/v/v) (50 ml) for 30 min. Filtration and washing with 10% MeOH/DCM (300 ml) then drying overnight gave 53β (6.51 g, loading ratio=77 μmol/g).

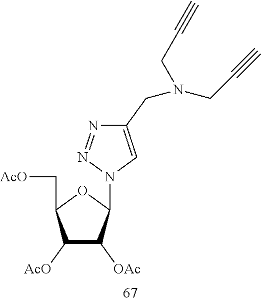

Compound 67. Compound 66 (800 mg, 2.66 mmol) in DCM/MeOH (4:1, 30 ml) was treated with cupper (33 mg, 0.53 mmol), tetrakis-acetonitrilecupper hexafluorophosphate (197 mg, 0.53 mmol) and tripropargylamine (1.90 ml, 13.28 mmol) at ambient temperature for 3 d. Then the solution was concentrated under reduced pressure and then silica gel chromatography gave 67 (668 mg, 1.54 mmol, 58%) as yellow gum. LC-MS calcd for [M+H]⁺ 433.1, found 433.1

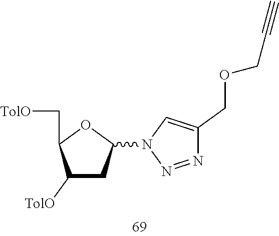

Compound 69: To a clear suspension of 47 (2.0 g, 5.06 mmol) in THE (30 mL) methanol (10 mL) and water (10 mL) was added copper (II) sulfate pentahydrate (25.26 mg, 101.16 μmol) and sodium ascorbate (100.21 mg, 505.81 μmol) in single portions. To this reaction mixture was added propargyl ether (1.43 g, 15.17 mmol, 1.56 mL) in single portion. Reaction mixture was stirred for 18 hr at 22° C. and TLC was checked and then diluted with EtOAc (40 mL). Organic layer was washed with water (30 mL) and brine (2×30 mL). Organic layer was separated, dried over anhydrous Na₂SO₄, filtered, and the filtrate was evaporated under high vacuum. Crude residue was purified by flash column chromatography (gradient: 10-70% EtOAc in hexane) to afford 69 (1.22 g, 49 yield) as transparent gum (R_{f}=0.5 in 25% EtOAc in hexane). ¹H NMR (600 MHz, CDCl₃) δ 8.00-7.74 (m, 4H), 7.70-7.60 (m, 1H), 7.28 (s, 1H), 7.22 (dd, J=15.1, 8.0 Hz, 2H), 6.58-6.42 (m, 1H), 5.87-5.58 (m, 1H), 4.82-4.49 (m, 5H), 4.18 (dd, J=7.6, 2.4 Hz, 2H), 3.19-3.12 (m, 1H), 3.07-2.77 (m, 1H), 2.46 (td, J=2.4, 1.4 Hz, 1H), 2.43 (d, J=2.5 Hz, 3H), 2.40 (d, J=6.7 Hz, 3H) ppm. ¹³C NMR (151 MHz, CDCl₃) δ 166.27, 166.25, 166.07, 165.99, 145.03, 144.68, 144.66, 144.59, 144.40, 144.27, 129.93, 129.84, 129.46, 129.43, 129.41, 126.81, 126.76, 126.50, 126.26, 121.61, 121.18, 90.15, 89.02, 85.04, 83.75, 79.40, 79.34, 75.10, 75.07, 74.93, 74.67, 64.05, 64.04, 63.12, 62.96, 57.63, 38.87, 38.51, 21.87, 21.85, 21.83 ppm. HRMS calc. for C₂₇H₂₇O₆N₃Na [M+Na]⁺ 512.1798, found 512.1804.

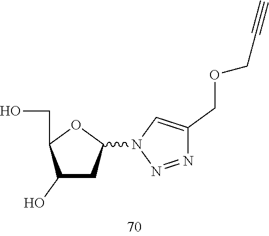

Compound 70: To a suspension of 69 (1.5 g, 3.06 mmol) in dry methanol (30 mL) was added sodium methoxide (397.27 mg, 7.35 mmol) at and stirred for 2 hr. TLC was checked which showed complete consumption of starting material. All the volatile matters were removed under high vacuum pump and the crude residue thus obtained, was purified by flash column chromatography (gradient: 0-5% MeOH in DCM) to afford 70 (0.69 g, 89% yield) as transparent gum. ¹H NMR (600 MHz, DMSO-d₆) δ 8.33 (s, 1H), 8.30 (s, 1H), 6.40-6.33 (m, 2H), 5.45 (d, J=3.9 Hz, 1H), 5.34 (d, J=4.4 Hz, 1H), 4.90 (t, J=5.5 Hz, 1H), 4.85 (t, J=5.7 Hz, 1H), 4.57 (d, J=7.3 Hz, 4H), 4.37 (dq, J=5.9, 4.1 Hz, 1H), 4.29 (dq, J=6.7, 3.3 Hz, 1H), 4.18 (dd, J=2.4, 1.1 Hz, 5H), 4.13-4.04 (m, 2H), 3.86 (td, J=4.9, 3.5 Hz, 1H), 3.55-3.49 (m, 1H), 3.48 (td, J=2.4, 1.4 Hz, 2H), 3.47-3.40 (m, 3H), 3.17 (d, J=5.2 Hz, 1H), 2.77 (ddd, J=14.4, 7.7, 6.8 Hz, 1H), 2.59 (dt, J=13.4, 6.0 Hz, 1H), 2.36 (ddd, J=13.4, 6.6, 4.3 Hz, 1H), 2.27 (dt, J=14.2, 2.8 Hz, 1H) ppm. ¹³C NMR (151 MHz, DMSO-d₆) δ 143.60, 143.58, 122.72, 122.55, 88.81, 88.77, 88.27, 88.04, 80.04, 77.53, 77.51, 70.49, 70.44, 62.11, 62.04, 61.60, 61.43, 56.76, 56.75, 48.62, 40.20, 40.06 ppm. HRMS calc. for C₁₁H₁₅O₄N₃Na [M+Na]⁺ 276.0960, found 276.0956.

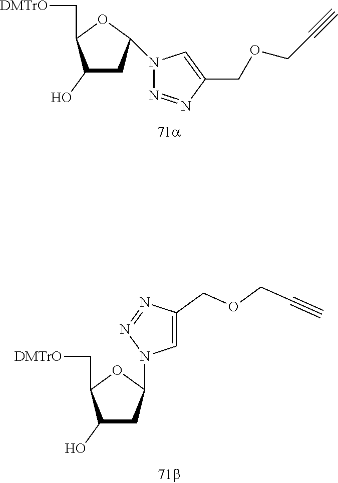

Compound 71α and 71β: To a clear solution of 70 (0.73 g, 2.88 mmol) (mixture of α- and β-isomers) in pyridine (10 mL) was added 4,4′-dimethoxytrityl chloride (1.17 g, 3.46 mmol) in two portions. Reaction mixture was stirred at for 10 hrs, diluted with DCM (20 mL) and then quenched with 10% NaHCO₃ (20 mL). Organic layer was washed with brine (2×20 mL), separated, dried over anhydrous Na₂SO₄ and filtered. Filtrate was evaporated under high vacuum pump and crude mass obtained, was purified by flash column chromatography (gradient: 20-70% EtOAc in hexane) to afford α-isomer (0.8 g) and ρ-isomer (0.36 g) as white foam (combined yield 72%).

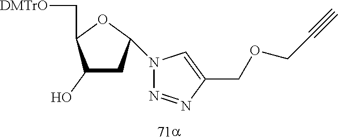

Data for 71α: ¹H NMR (600 MHz, DMSO-d₆) δ 8.37 (s, 1H), 7.41-7.36 (m, 2H), 7.32 (dd, J=8.5, 7.1 Hz, 2H), 7.28-7.19 (m, 5H), 6.95-6.88 (m, 4H), 6.48 (dd, J=7.6, 3.0 Hz, 1H), 5.51 (s, 1H), 4.59 (s, 2H), 4.28-4.25 (m, 1H), 4.24-4.22 (m, 1H), 4.19 (d, J=2.4 Hz, 2H), 3.74 (s, 7H), 3.49 (dd, J=2.7, 2.2 Hz, 1H), 3.15 (dd, J=10.2, 3.7 Hz, 1H), 3.00 (dd, J=10.2, 5.0 Hz, 1H), 2.82 (dt, J=14.4, 7.3 Hz, 1H), 2.33 (dt, J=14.2, 3.1 Hz, 1H) ppm. ¹³C NMR (151 MHz, DMSO-d₆) δ 158.09, 144.82, 143.62, 135.55, 135.44, 129.70, 127.90, 127.68, 126.71, 122.66, 113.25, 88.84, 86.78, 85.52, 80.03, 77.52, 70.87, 63.72, 62.09, 56.75, 55.08, 55.04, 40.22 ppm. HRMS calc. for C₃₂H₃₃N₃O₆Na [M+Na]⁺ 578.2267, found 578.2271.

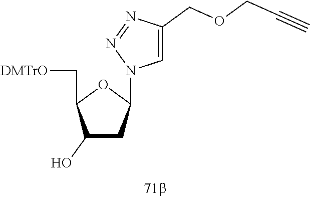

Data for 71β: ¹H NMR (600 MHz, DMSO-d₆) δ 8.24 (s, 1H), 7.35-7.30 (m, 2H), 7.29-7.23 (m, 2H), 7.23-7.17 (m, 5H), 6.87-6.81 (m, 4H), 6.39 (dd, J=6.8, 4.6 Hz, 1H), 5.41 (s, 1H), 4.53 (s, 2H), 4.42 (q, J=5.9 Hz, 1H), 4.14 (d, J=2.4 Hz, 2H), 3.99 (q, J=5.0 Hz, 1H), 3.73 (s, 6H), 3.49 (t, J=2.4 Hz, 1H), 3.08 (d, J=5.0 Hz, 2H), 2.70 (ddd, J=13.3, 6.5, 4.6 Hz, 1H), 2.40 (dt, J=13.1, 6.5 Hz, 1H) ppm. ¹³C NMR (151 MHz, DMSO-d₆) δ 158.02, 144.79, 143.45, 135.61, 135.37, 129.71, 129.66, 127.78, 127.66, 126.61, 123.23, 113.13, 87.52, 86.07, 85.41, 79.96, 77.49, 70.33, 64.06, 61.95, 56.70, 55.02 ppm. HRMS calc. for C₃₂H₃₃N₃O₆Na [M+Na]⁺ 578.2267, found 578.2289.

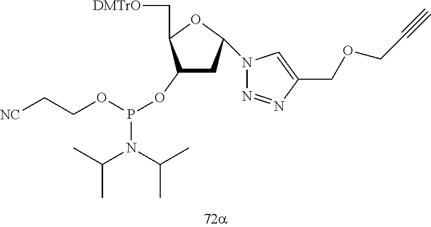

3-[[(2R,5R)-2-[[bis(4-methoxyphenyl)-phenyl-methoxy]methyl]-5-[4-(prop-2-ynoxymethyl) triazol-1-yl]tetrahydrofuran-3-yl]oxy-(diisopropylamino)phosphanyl]oxypropanenitrile Compound 72α: To a clear solution of 71a (0.57 g, 1.03 mmol) in dichloromethane (20 mL) was added N-methylimidazole (126.34 mg, 1.54 mmol, 122.66 μL) and diisopropylethylamine (662.92 mg, 5.13 mmol, 893.43 μL) in single portions. After stirring the reaction mixture for 5 minutes at 22° C., 2-cyanoethyl-N,N-diisopropylchlorophosphoramidite (485.61 mg, 2.05 mmol, 458.12 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-70% EtOAc in hexane) to afford 72a (0.64 g, 82% yield) as hygroscopic foam. ¹H NMR (600 MHz, CD₃CN) δ 8.13 (s, 1H), 7.48-7.43 (m, 2H), 7.36-7.29 (m, 6H), 7.27-7.21 (m, 1H), 6.91-6.85 (m, 4H), 6.52 (ddd, J=13.3, 7.6, 2.0 Hz, 1H), 4.63 (d, J=3.7 Hz, 2H), 4.56 (dddt, J=11.0, 7.0, 4.6, 2.3 Hz, 1H), 4.50-4.35 (m, 1H), 4.19 (t, J=2.4 Hz, 2H), 3.77 (dd, J=2.2, 0.5 Hz, 6H), 3.72-3.59 (m, 2H), 3.53 (dh, J=10.4, 6.8 Hz, 2H), 3.34-3.24 (m, 1H), 3.10 (ddd, J=10.4, 4.3, 2.8 Hz, 1H), 3.01-2.89 (m, 1H), 2.74 (td, J=2.4, 0.9 Hz, 1H), 2.68-2.45 (m, 3H), 1.26 (dd, J=11.1, 6.7 Hz, OH), 1.14 (dd, J=6.8, 1.7 Hz, 6H), 1.10-0.98 (m, 6H) ppm. ¹³C NMR (151 MHz, CD₃CN) δ 159.71, 146.04, 146.03, 145.02, 145.00, 136.88, 136.82, 136.72, 136.68, 131.02, 130.99, 128.98, 128.93, 128.89, 127.87, 122.95, 122.92, 119.49, 119.37, 114.11, 114.09, 90.93, 90.87, 88.15, 88.12, 88.03, 87.99, 87.17, 87.16, 80.63, 76.03, 76.02, 75.32, 75.20, 74.78, 74.67, 64.54, 63.50, 63.47, 60.96, 59.54, 59.50, 59.41, 59.38, 57.87, 57.86, 55.90, 44.04, 44.00, 43.95, 43.92, 41.07, 41.04, 40.89, 40.87, 24.90, 24.84, 24.79, 21.01, 20.97, 20.93 ppm. ³¹P NMR (243 MHz, CD₃CN) δ 148.67, 148.19 ppm. HRMS calc. for C₄₁H₅₁N₅O₇P [M+H]⁺ 756.3526, found 756.3532.

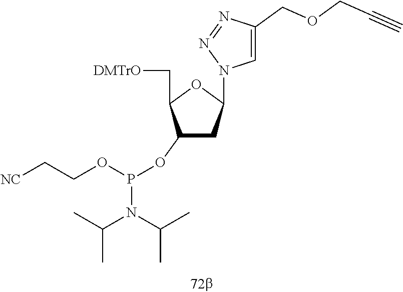

3-[[(2R,5R)-2-[[bis(4-methoxyphenyl)-phenyl-methoxy]methyl]-5-[4-(prop-2-ynoxymethyl) triazol-1-yl]tetrahydrofuran-3-yl]oxy-(diisopropylamino)phosphanyl]oxypropanenitrile Compound 72p: To a clear solution of 71β (0.77 g, 1.39 mmol) in dichloromethane (20 mL) was added N-methylimidazole (170.67 mg, 2.08 mmol, 165.70 μL) and diisopropylethylamine (895.53 mg, 6.93 mmol, 1.21 mL) in single portions. After stirring the reaction mixture for 5 minutes at 22° C., 2-cyanoethyl-N,N-diisopropylchlorophosphoramidite (656.00 mg, 2.77 mmol, 618.87 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-70% EtOAc in hexane) to afford 72β (0.87 g, 1.15 mmol, 83.06% yield) as white hygroscopic foam. ¹H NMR (600 MHz, CD₃CN) δ 7.91 (d, J=1.4 Hz, 1H), 7.39-7.34 (m, 2H), 7.30-7.18 (m, 7H), 6.82 (ddd, J=9.0, 6.8, 2.1 Hz, 4H), 6.35 (ddd, J=9.9, 6.7, 4.7 Hz, 1H), 4.82-4.69 (m, 1H), 4.57-4.52 (m, 2H), 4.22-4.14 (m, 1H), 4.10 (td, J=2.3, 0.8 Hz, 2H), 3.83-3.72 (m, 7H), 3.69-3.51 (m, 3H), 3.28-3.19 (m, 1H), 3.14 (dt, J=10.6, 5.4 Hz, 1H), 2.91 (dtd, J=13.3, 6.5, 4.7 Hz, 1H), 2.72 (td, J=2.4, 1.1 Hz, 1H), 2.67-2.55 (m, 2H), 2.52 (t, J=6.0 Hz, 1H), 1.19-1.11 (m, 9H), 1.05 (d, J=6.8 Hz, 3H) ppm. ¹³C NMR (151 MHz, CD₃CN) δ 159.63, 145.93, 145.08, 145.06, 136.79, 136.76, 136.70, 136.63, 131.01, 130.99, 130.95, 128.98, 128.93, 128.81, 127.80, 127.79, 123.70, 119.54, 119.37, 114.02, 89.18, 89.16, 87.06, 87.05, 86.77, 86.74, 86.59, 86.55, 80.55, 76.01, 74.19, 74.08, 73.60, 73.48, 64.46, 64.20, 63.23, 60.95, 59.62, 59.53, 59.50, 59.40, 57.76, 55.90, 55.88, 44.04, 43.96, 40.11, 40.09, 39.87, 39.84, 24.92, 24.90, 24.89, 24.87, 24.84, 24.80, 21.14, 21.05, 21.00, 20.96, 20.91 ppm. ³¹P NMR (243 MHz, CD₃CN) δ 148.29, 148.19 ppm.

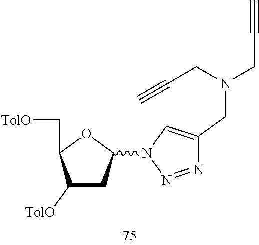

Compound 75: To a clear suspension of 47 (2 g, 5.06 mmol) in THF (30 mL) methanol (10 mL) and water (10 mL) was added copper (II) sulfate pentahydrate (25.26 mg, 101.16 μmol) and sodium ascorbate (100.21 mg, 505.81 μmol) in single portions. To this reaction mixture was added trispropargylamine (1.99 g, 15.17 mmol, 2.14 mL) in single portion. Reaction mixture was stirred for 18 hr at 22° C. and TLC was checked and then diluted with EtOAc (40 mL). Organic layer was washed with water (30 mL) and brine (2×30 mL). Organic layer was separated, dried over anhydrous Na₂SO₄, filtered, and the filtrate was evaporated under high vacuum. Crude residue was purified by flash column chromatography (gradient: 10-40% EtOAc in hexane) to afford 75 (1.54 g, 58% yield) as transparent gum. ¹H NMR (600 MHz, DMSO-d₆) δ 8.21 (d, J=8.5 Hz, 1H), 7.93 (dd, J=14.6, 8.2 Hz, 2H), 7.86-7.81 (m, 1H), 7.71-7.66 (m, 1H), 7.39-7.28 (m, 4H), 6.64-6.56 (m, 1H), 5.79-5.55 (m, 1H), 4.91-4.56 (m, 1H), 4.42 (dd, J=11.8, 5.8 Hz, 1H), 3.73-3.65 (m, 2H), 3.37-3.30 (m, 6H), 3.21-3.04 (m, 3H), 2.93-2.80 (m, 1H), 2.41-2.36 (m, 6H) ppm. ¹³C NMR (151 MHz, DMSO-d₆) δ 165.49, 165.41, 165.24, 165.18, 144.09, 144.00, 143.92, 143.81, 143.77, 143.50, 129.51, 129.40, 129.37, 129.31, 129.28, 129.26, 129.25, 126.56, 126.53, 126.46, 126.36, 123.34, 122.15, 89.11, 87.72, 83.47, 82.20, 78.86, 78.82, 75.96, 75.94, 74.66, 74.56, 64.02, 63.90, 59.75, 54.91, 47.25, 47.15, 41.19, 41.16, 37.81, 36.59, 21.19 ppm. HRMS calc. for C₃₀H₃₁O₅N₄ [M+H]⁺ 527.2294, found 527.2285.

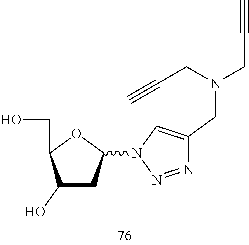

Compound 76: To a suspension of 75 (1.5 g, 2.85 mmol) in dry methanol (30 mL) was added sodium methoxide (1.48 g, 6.84 mmol) at and stirred for 3 hrs. TLC was checked which showed complete consumption of starting material. All the volatile matters were removed under high vacuum pump and the crude residue thus obtained, was purified by flash column chromatography (gradient: 0-5% MeOH in DCM) to afford 76 (0.75 g, 2.58 mmol, 90.69% yield) as transparent gum which turned into white solid. ¹H NMR (600 MHz, DMSO-d₆) δ 8.22 (s, 1H), 8.17 (s, 1H), 6.39-6.32 (m, 2H), 5.44 (d, J=4.0 Hz, 1H), 5.32 (d, J=4.4 Hz, 1H), 4.90-4.81 (m, 2H), 4.37 (dq, J=6.0, 4.1 Hz, 1H), 4.31-4.25 (m, 1H), 4.12-4.05 (m, 2H), 3.86 (td, J=4.9, 3.5 Hz, 1H), 3.70 (d, J=6.2 Hz, 3H), 3.56-3.40 (m, 3H), 3.36 (d, J=2.4 Hz, 7H), 3.21 (td, J=2.4, 0.9 Hz, 3H), 3.19-3.15 (m, 02H), 2.76 (ddd, J=14.4, 7.7, 6.9 Hz, 1H), 2.60 (dt, J=13.4, 6.0 Hz, 1H), 2.36 (ddd, J=13.4, 6.6, 4.3 Hz, 1H), 2.27 (dt, J=14.2, 2.9 Hz, 1H) ppm. ¹³C NMR (151 MHz, DMSO-d₆) δ 143.64, 143.61, 122.38, 122.23, 88.71, 88.66, 88.21, 87.97, 78.87, 75.98, 70.48, 70.42, 61.56, 61.43, 48.60, 47.24, 47.21, 41.18, 41.16, 40.17, 40.03 ppm. HRMS calc. for C₁₄H₁₉O₃N₄ [M+H]⁺ 291.1457, found 291.1466.

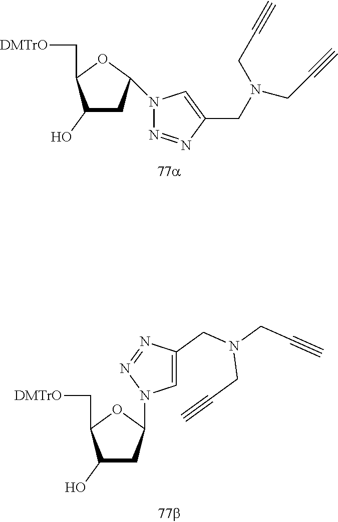

To a clear solution of 76 (0.62 g, 2.14 mmol) (mixture of α- and β-isomers) in pyridine (20 mL) was added 4,4′-dimethoxytrityl chloride (868.33 mg, 2.56 mmol) in two portions. Reaction mixture was stirred at for 10 hrs, diluted with DCM (20 mL) and then quenched with 10% NaHCO₃ (20 mL). Organic layer was washed with brine (2×20 mL), separated, dried over anhydrous Na₂SO₄ and filtered. Filtrate was evaporated under high vacuum pump and crude mass obtained, was purified by flash column chromatography (gradient: 20-70% EtOAc in hexane) to afford α-isomer (0.48 g, fast moving faction) and β-isomer (0.30 g, slow moving fraction) as white foam (combined yield 62%).

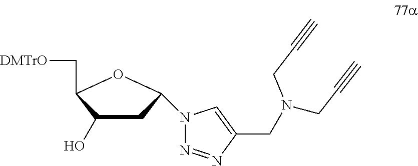

Data for (2R,3R,5S)-2-((bis(4-methoxyphenyl)(phenyl)methoxy)methyl)-5-(4-((di(prop-2-yn-1-yl)amino)methyl)-1H-1,2,3-triazol-1-yl)tetrahydrofuran-3-ol 77α: ¹H NMR (600 MHz, DMSO-d₆) δ 8.26 (s, 1H), 7.41-7.37 (m, 2H), 7.32 (dd, J=8.5, 7.1 Hz, 2H), 7.26-7.20 (m, 3H), 6.95-6.88 (m, 4H), 6.47 (dd, J=7.7, 3.0 Hz, 1H), 5.50 (d, J=4.1 Hz, 1H), 4.29-4.21 (m, 2H), 3.74 (s, 6H), 3.72 (s, 2H), 3.37 (d, J=2.5 Hz, 4H), 3.22 (t, J=2.4 Hz, 2H), 3.14 (dd, J=10.2, 3.7 Hz, 1H), 3.00 (dd, J=10.2, 4.9 Hz, 1H), 2.81 (p, J=7.3 Hz, 1H), 2.33 (dt, J=14.2, 3.1 Hz, 1H) ppm. ¹³C NMR (151 MHz, DMSO-d₆) δ 158.09, 144.82, 143.70, 135.55, 135.44, 129.71, 129.70, 127.90, 127.68, 126.71, 122.35, 113.25, 88.80, 86.74, 85.52, 78.88, 76.00, 70.90, 63.71, 59.77, 55.04, 47.22, 41.17, 40.22, 40.06 ppm.

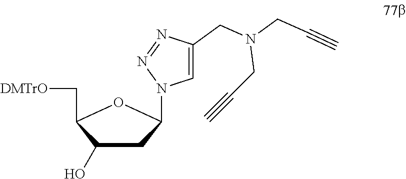

Data for (2R,3S,5R)-2-((bis(4-methoxyphenyl)(phenyl)methoxy)methyl)-5-(4-((di(prop-2-yn-1-yl)amino)methyl)-1H-1,2,3-triazol-1-yl)tetrahydrofuran-3-ol 77β: ¹H NMR (600 MHz, DMSO-d₆) δ 8.09 (s, 1H), 7.34-7.30 (m, 2H), 7.28-7.22 (m, 2H), 7.22-7.17 (m, 5H), 6.87-6.80 (m, 4H), 6.37 (dd, J=6.8, 4.6 Hz, 1H), 5.40 (d, J=4.8 Hz, 1H), 4.43 (dd, J=8.9, 4.9 Hz, 1H), 3.98 (q, J=4.9 Hz, 1H), 3.73 (d, J=1.3 Hz, 6H), 3.64 (s, 2H), 3.30 (d, J=2.4 Hz, 4H), 3.20 (t, J=2.4 Hz, 2H), 3.07 (d, J=4.9 Hz, 2H), 2.70 (ddd, J=13.3, 6.5, 4.6 Hz, 1H), 2.38 (dt, J=13.1, 6.5 Hz, 1H) ppm. ¹³C NMR (151 MHz, DMSO-d₆) δ 158.01, 144.76, 143.49, 135.64, 135.34, 129.72, 129.64, 127.78, 127.65, 126.60, 122.89, 113.13, 87.49, 86.05, 85.41, 78.79, 75.96, 70.36, 64.04, 59.76, 55.02, 47.20, 41.12, 40.06 ppm.

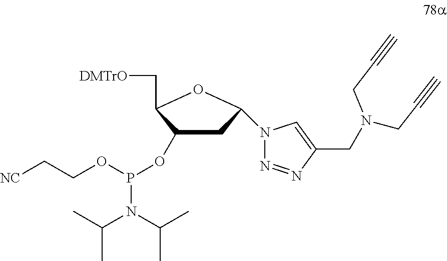

3-[[(2R,5R)-2-[[bis(4-methoxyphenyl)-phenyl-methoxy]methyl]-5-[4-[[bis(prop-2-ynyl)amino]methyl]triazol-1-yl]tetrahydrofuran-3-yl]oxy-(diisopropylamino)phosphanyl]oxypropanenitrile 78α: To a clear solution of 77α (0.33 g, 556.79 μmol) in dichloromethane (30 mL) was added N-methylimidazole (68.57 mg, 835.19 μmol, 66.57 μL) and diisopropylethylamine (359.80 mg, 2.78 mmol, 484.90 μL) in single portions. After stirring the reaction mixture for 5 minutes at 22° C., 2-cyanoethyl N,N-diisopropylchlorophosphoramidite (263.56 mg, 1.11 mmol, 248.64 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-70% EtOAc in hexane) to afford 78α (0.37 g, 84% yield) as white foam. ¹H NMR (600 MHz, CD₃CN) δ 8.05 (d, J=2.5 Hz, 1H), 7.48-7.42 (m, 2H), 7.32 (tdd, J=8.0, 4.5, 2.6 Hz, 7H), 7.27-7.20 (m, 1H), 6.91-6.85 (m, 5H), 6.50 (ddd, J=9.5, 7.6, 2.0 Hz, 1H), 4.61-4.52 (m, 1H), 4.48-4.33 (m, 1H), 3.79-3.74 (m, 9H), 3.74-3.61 (m, 2H), 3.54 (dpd, J=10.6, 6.8, 5.1 Hz, 2H), 3.41 (dd, J=2.5, 1.4 Hz, 5H), 3.33-3.24 (m, 1H), 3.10 (ddd, J=10.4, 5.1, 4.4 Hz, 1H), 2.94 (tq, J=14.7, 7.3 Hz, 1H), 2.66-2.57 (m, 2H), 2.55-2.48 (m, 4H), 1.26 (dd, J=11.2, 6.8 Hz, OH), 1.14 (dd, J=6.8, 2.1 Hz, 7H), 1.11-1.02 (m, 7H) ppm. ¹³C NMR (151 MHz, CD₃CN) δ 159.71, 146.05, 146.03, 145.25, 145.23, 136.89, 136.82, 136.74, 136.68, 131.02, 130.99, 128.99, 128.93, 128.89, 127.86, 122.77, 122.75, 119.49, 119.38, 114.11, 114.09, 90.81, 90.77, 88.02, 87.99, 87.90, 87.86, 87.16, 87.15, 79.65, 79.64, 75.34, 75.22, 74.71, 74.65, 74.63, 74.60, 64.54, 64.52, 59.59, 59.55, 59.46, 59.42, 55.90, 48.62, 48.56, 44.05, 44.01, 43.97, 43.93, 42.29, 42.27, 40.96, 40.93, 40.83, 40.81, 24.91, 24.89, 24.86, 24.85, 24.80, 21.05, 21.00, 20.96 ppm. ³¹P NMR (243 MHz, CD₃CN) δ 148.89, 148.21 ppm.

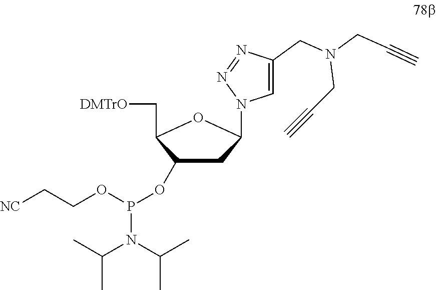

3-[[(2R,5R)-2-[[bis(4-methoxyphenyl)-phenyl-methoxy]methyl]-5-[4-[[bis(prop-2-ynyl)amino]methyl]triazol-1-yl]tetrahydrofuran-3-yl]oxy-(diisopropylamino)phosphanyl]oxypropanenitrile 78ρ: To a clear solution of 77β (0.62 g, 1.05 mmol) in dichloromethane (30 mL) was added N-methylimidazole (128.83 mg, 1.57 mmol, 125.07 μL) and diisopropylethylamine (675.98 mg, 5.23 mmol, 911.03 μL) in single portions. After stirring the reaction mixture for 5 minutes at 22° C., 2-cyanoethyl-N,N-diisopropylchlorophosphoramidite (495.18 mg, 2.09 mmol, 467.15 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-70% EtOAc in hexane) to afford 78β (0.64 g, 77% yield) as transparent gum. ¹H NMR (600 MHz, CD₃CN) δ 7.82 (d, J=2.6 Hz, 1H), 7.47-7.13 (m, 11H), 6.91-6.79 (m, 5H), 6.33 (ddd, J=10.9, 6.7, 4.8 Hz, 1H), 4.91-4.61 (m, 1H), 4.24 (dtd, J=8.2, 6.0, 1.9 Hz, 1H), 4.18 (dq, J=13.0, 4.5 Hz, 1H), 3.85-3.71 (m, 9H), 3.71-3.51 (m, 6H), 3.32 (t, J=2.1 Hz, 5H), 3.28-3.19 (m, 1H), 3.14 (ddd, J=10.5, 6.8, 5.2 Hz, 1H), 2.91 (dtd, J=13.7, 6.9, 4.9 Hz, 1H), 2.82 (td, J=5.9, 1.0 Hz, 1H), 2.67-2.55 (m, 3H), 2.54-2.47 (m, 3H), 1.26 (dd, J=11.2, 6.8 Hz, 4H), 1.19-1.12 (m, 11H), 1.06 (d, J=6.8 Hz, 4H) ppm. ¹³C NMR (151 MHz, CD₃CN) δ 159.63, 145.92, 145.19, 145.17, 136.83, 136.79, 136.71, 136.63, 131.03, 131.01, 130.99, 130.96, 129.00, 128.94, 128.89, 128.83, 127.81, 127.80, 123.50, 123.48, 119.54, 119.38, 114.03, 89.17, 89.13, 87.07, 87.06, 86.73, 86.70, 86.57, 86.53, 79.58, 74.64, 74.61, 74.20, 74.09, 73.58, 73.46, 64.43, 64.14, 62.83, 62.79, 59.64, 59.55, 59.52, 59.42, 55.91, 55.90, 55.33, 48.41, 48.37, 48.33, 44.05, 43.96, 42.27, 42.21, 40.17, 40.15, 39.92, 39.90, 24.93, 24.91, 24.90, 24.88, 24.85, 24.80, 22.78, 22.76, 21.34, 21.33, 21.06, 21.01, 20.97, 20.92, 20.12, 20.06 ppm. ³¹P NMR (243 MHz, CD₃CN) δ 148.24, 148.16 ppm.

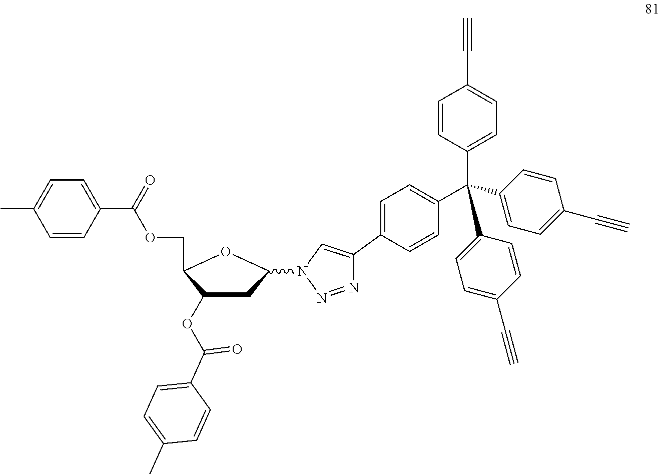

Compound 81: To a clear suspension of 47 (0.5 g, 1.26 mmol) in THE (30 mL) methanol (10 mL) and water (10 mL) was added copper (II) sulfate pentahydrate (15.79 mg, 63.23 μmol) and sodium ascorbate (125.26 mg, 632.26 μmol) in single portions. Reaction mixture was stirred for 18 hr at 22° C. and TLC was checked and then diluted with EtOAc (40 mL). Organic layer was washed with water (30 mL) and brine (2×30 mL). Organic layer was separated, dried over anhydrous Na₂SO₄, filtered, and the filtrate was evaporated under high vacuum. Crude residue was purified by flash column chromatography (gradient: 10-40% EtOAc in hexane) to afford 81 (0.9 g, 88% yield) as yellow foam (combining both anomers).

¹H NMR (600 MHz, DMSO-d₆) δ 8.82 (d, J=5.2 Hz, 1H), 8.71 (d, J=4.1 Hz, 1H), 7.97-7.90 (m, 4H), 7.83-7.76 (m, 4H), 7.74-7.67 (m, 2H), 7.63-7.58 (m, 2H), 7.49-7.41 (m, 9H), 7.39-7.34 (m, 4H), 7.25-7.12 (m, 13H), 7.08-6.98 (m, 2H), 6.74-6.70 (m, 1H), 6.64 (td, J=6.2, 2.2 Hz, 1H), 5.81 (dt, J=7.3, 4.1 Hz, 1H), 5.62 (dt, J=6.6, 1.7 Hz, 1H), 4.99 (p, J=2.3 Hz, 1H), 4.64 (td, J=5.0, 3.5 Hz, 1H), 4.59-4.48 (m, 3H), 4.41 (dd, J=11.9, 5.2 Hz, 1H), 4.26 (t, J=7.1 Hz, OH), 4.21 (s, 4H), 3.24-3.17 (m, 1H), 3.13-3.06 (m, 1H), 2.91 (ddd, J=13.9, 6.5, 4.2 Hz, OH), 2.85 (d, J=15.1 Hz, 1H), 2.40 (d, J=5.4 Hz, 6H), 2.28 (d, J=6.0 Hz, 3H), 2.22 (d, J=15.5 Hz, 3H) ppm. ¹³C NMR (151 MHz, DMSO) δ 165.54, 165.45, 165.30, 165.02, 146.33, 146.28, 145.76, 144.93, 144.72, 144.18, 144.01, 143.81, 131.57, 131.48, 130.79, 130.71, 130.59, 130.57, 129.58, 129.47, 129.44, 129.38, 129.36, 129.33, 129.10, 128.78, 128.45, 126.58, 126.52, 126.46, 126.40, 125.04, 120.66, 120.13, 119.80, 119.73, 89.63, 88.05, 84.00, 83.09, 82.34, 81.19, 74.66, 74.50, 68.31, 64.29, 64.24, 64.12, 63.86, 59.81, 54.98, 38.44, 36.78, 27.46, 21.79, 21.27, 21.25, 21.16, 20.82 ppm. HRMS calc. for C₅₄H₄₂N₃O₅ [M+H]⁺ 812.3124, found 812.3123.

### Azide Ligands:

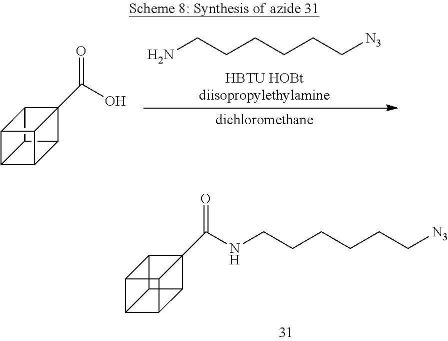 [SMILES: O=C(O)C12C3C4C5C3C1C5C42 ; [N-]=[N+]=NCCCCCCN ; [N-]=[N+]=NCCCCCCNC(=O)C12C3C4C5C3C1C5C42]

Compound 31: To a clear solution of cubane-1-carboxylic acid (0.5 g, 3.37 mmol) in dichloromethane (20 mL) was added HBTU (1.28 g, 3.37 mmol) followed by HOBt (456.00 mg, 3.37 mmol) and diisopropylethylamine (872.31 mg, 6.75 mmol, 1.18 mL). Reaction mixture was stirred for 5 minutes and to the resulting solution was added 6-azidohexan-1-amine (527.88 mg, 3.71 mmol) in single portion. Reaction mixture was stirred for 16 hr at 22° C. and then all the volatile matters were removed under high vacuum pump. Crude mass thus obtained was purified by flash chromatography (gradient: 20-60% EtOAc in hexane) to afford 31 (0.78 g, 85% yield) as white solid. ¹H NMR (600 MHz, CDCl₃) δ 5.52 (t, J=6.1 Hz, 1H), 4.19 (ddd, J=5.9, 3.9, 2.3 Hz, 3H), 4.00 (tt, J=7.1, 2.7 Hz, 4H), 3.28 (dt, J=8.0, 6.2 Hz, 4H), 1.61 (p, J=7.0 Hz, 2H), 1.53 (p, J=7.3 Hz, 2H), 1.45-1.31 (m, 4H) ppm. ¹³C NMR (151 MHz, CDCl₃) δ 172.33, 57.73, 51.45, 49.42, 47.97, 44.80, 39.11, 29.78, 28.83, 26.55, 26.51 ppm. HRMS calc. for C₁₅H₂₁N₄O [M+H]⁺ 273.1715, found 273.1719.

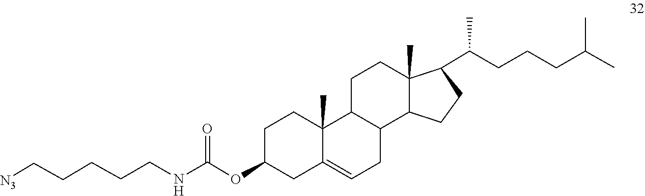

Compound 32. 1-amino-6-hexanol (5.00 g, 42.67 mmol) in anhydrous dichloromethane was treated with triethylamine (11.9 ml, 85.33 mmol) and cholesterol chloroformate 54 (19.16 g, 42.67 mmol) at room temperature for 4 h. The solution was concentrated under reduced pressure after it was quenched with methanol. Then the residue was extracted with DCM/brine. The organic layer was dried with sodium sulfate and then concentrated under reduce pressure. Then the residue (15.0 g, 28.31 mmol) was treated with triethylamine (7.9 ml, 56.62 mmol) and methanesulfonylchloride (2.4 ml, 31.14 mmol) at room temperature for 4 h. Then the solution was extracted with DCM/brine. Then the organic layer was concentrated under reduced pressure to give crude mesylate. The crude in anhydrous DMF (300 ml) was treated with sodium azide at 60° C. for 18 h. Then the solution was extracted with EtOAc/brine. The organic layer was dried on sodium sulfate and concentrated under reduced pressure. The residue was crystallized at ambient temperature in EtOAc. The crystal was washed with water. Filtration and drying on suction funnel and then vacuum gave 32 as a yellow crystal (13.24 g, 23.87 mmol, 84% (3 steps)). ¹H-NMR (400 MHz, CDCl₃) δ 0.67 (3H, s), 0.85-2.02 (45H, m), 2.26-2.37 (2H, m), 3.13-3.27 (2H, m), 4.46-4.60 (2H, m), 5.36-5.37 (1H, m) ppm.

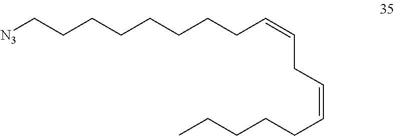 [SMILES: CCCCC/C=C\C/C=C\CCCCCCCCN=[N+]=[N-]]

Compound 35: Linoleyl alcohol 64 (10 ml, 32.3 mmol) in anhydrous DCM (80 ml) was treated with methanesulfonylchloride (2.75 ml, 35.5 mmol) and triethylamine (5.8 ml, 42.0 mmol) at room temperature for 2 h. Extraction with DCM/water, drying organic layer with sodium sulfate, and then evaporation gave crude mesylate. This crude was directly used for next step. The crude in anhydrous DMF (300 ml) was treated with sodium azide (10.5 g, 161.50 mmol) at 60° C. for 18 h. Extraction with EtOAc/water, drying organic layer with sodium sulfate, and then evaporation gave dark oil. The oil was chromatographed on silica gel. Eluent with hexane gave linoleyl azide 35 as clear oil (7.51 g, 25.67 mmol, 80% (2 steps).

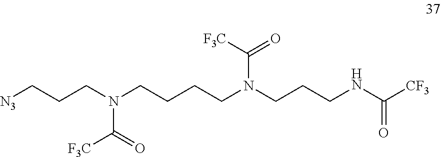 [SMILES: [N-]=[N+]=NCCCN(CCCCN(CCCNC(=O)C(F)(F)F)C(=O)C(F)(F)F)C(=O)C(F)(F)F]

Compound 37. Trifluoromethanesulfonic anhydride (6.0 mL, 35.3 mmol) was slowly added by a dropping funnel to the suspension of sodium azide (4.36 g, 67.07 mmol) in CH₃CN (80 mL) that was prepared previously in an ice bath. During the dropping, the internal temperature was kept under 10° C. After vigorous stirring for 2 h in an ice bath, the solution was filtrated. The filtrated was slowly added by a dropping funnel to the solution of spermine (7.16 mmol, 35.3 mmol), triethylamine (15.5 ml, 107.31 mmol) and copper sulfate pentahydrate (87 mg, 0.35 mmol) in CH₃CN (50 ml) in an ice bath that was prepared previously. During the dropping, internal temperature was kept under 10° C. After vigorous stirring for 18 h at 0° C. to ambient temperature, triethylamine (96.0 ml, 670.67 mmol) was added to the solution. Then trifluoroacetic anhydride (46.0 ml, 335.33 mmol) was added slowly by a dropping funnel to the solution in an ice bath. During the dropping, internal temperature was kept under 10° C. After vigorous stirring for 18 h at 0° C. to ambient temperature, the reaction was quenched with water. The solution was extracted with EtOAc/sat NaHCO₃aq without concentration of the reaction solution. The organic layer was dried on sodium sulfate and then concentrated under reduced pressure. The residue was subjected to flash silica gel chromatography. Eluent with 5% MeOH in DCM gave 37 (9.20 g, 17.8 mmol, 50%, R_{f}=0.15) as a yellow gum. LC-Mass: Calcd for C₁₆H₂₁F₉N₆O₃=534.0, found=534.0.

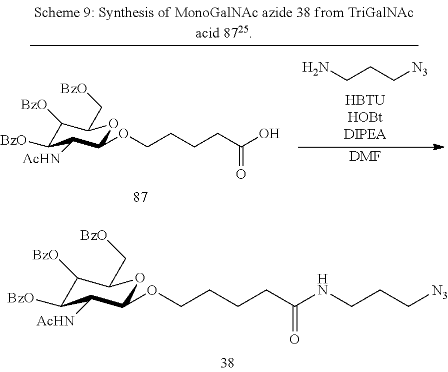

Compound 38: To a clear solution of 87 (3.0 g, 4.73 mmol) in dry dimethylformamide (23.19 mL) was added HBTU (2.02 g, 5.21 mmol), 1-hydroxybenzotriazole hydrate (813.83 mg, 5.21 mmol) and diisopropylethylamine (1.85 g, 14.20 mmol, 2.50 mL) in single portions. Reaction mixture was stirred for 5 minutes and then was added 3-azidopropan-1-amine (711.05 mg, 7.10 mmol) slowly. Resulting mixture was stirred at 22° C. for 16 hr and then all volatile matters were removed under high vacuum pump. Residue was diluted with DCM (70 mL) and washed with NH₄Cl solution (3×30 mL), NaHCO₃ solution (3×40 mL), water (50 mL) and brine (2×50 mL). Organic layer was separated, dried over anhydrous Na₂SO₄, filtered and the filtrate was evaporated to dryness. Solid residue thus obtained, was evaporated with DCM (20 mL) and kept for drying overnight at 22° C. to afford 38 (2.71 g, 80% yield) as yellow solid. ¹H NMR (600 MHz, DMSO-d₆) δ 7.99 (d, J=9.3 Hz, 1H), 7.93 (ddt, J=8.3, 6.9, 1.4 Hz, 4H), 7.82 (t, J=5.7 Hz, 1H), 7.74-7.68 (m, 3H), 7.68-7.62 (m, 1H), 7.58 (ddd, J=8.4, 7.4, 6.3 Hz, 3H), 7.53-7.47 (m, 2H), 7.42-7.36 (m, 2H), 5.76 (d, J=3.4 Hz, 1H), 5.38 (dd, J=11.1, 3.4 Hz, 1H), 4.74 (d, J=8.5 Hz, 1H), 4.50-4.43 (m, 2H), 4.38-4.33 (m, 1H), 4.28 (dt, J=11.0, 8.9 Hz, 1H), 3.84-3.77 (m, 1H), 3.64-3.47 (m, 1H), 3.35-3.31 (m, 3H), 3.17-3.07 (m, 2H), 2.07 (t, J=7.1 Hz, 2H), 1.71 (s, 3H), 1.64 (p, J=6.8 Hz, 2H), 1.53 (tdd, J=12.0, 7.4, 4.4 Hz, 4H), 1.25 (dd, J=8.3, 6.7 Hz, 3H) ppm. ¹³C NMR (151 MHz, DMSO-d₆) δ 172.03, 169.42, 165.23, 165.18, 164.89, 133.80, 133.53, 133.50, 129.23, 129.21, 129.19, 129.06, 129.04, 128.99, 128.73, 128.61, 100.91, 71.86, 70.00, 68.76, 67.94, 62.05, 53.60, 49.76, 48.41, 41.85, 38.23, 35.75, 35.02, 28.59, 28.49, 22.71, 21.83, 18.07, 16.72, 12.48 ppm. HRMS calc. for C₃₇H₄₁N₅O₁₀Na [M+Na]⁺ 738.2751, found 738.2747.

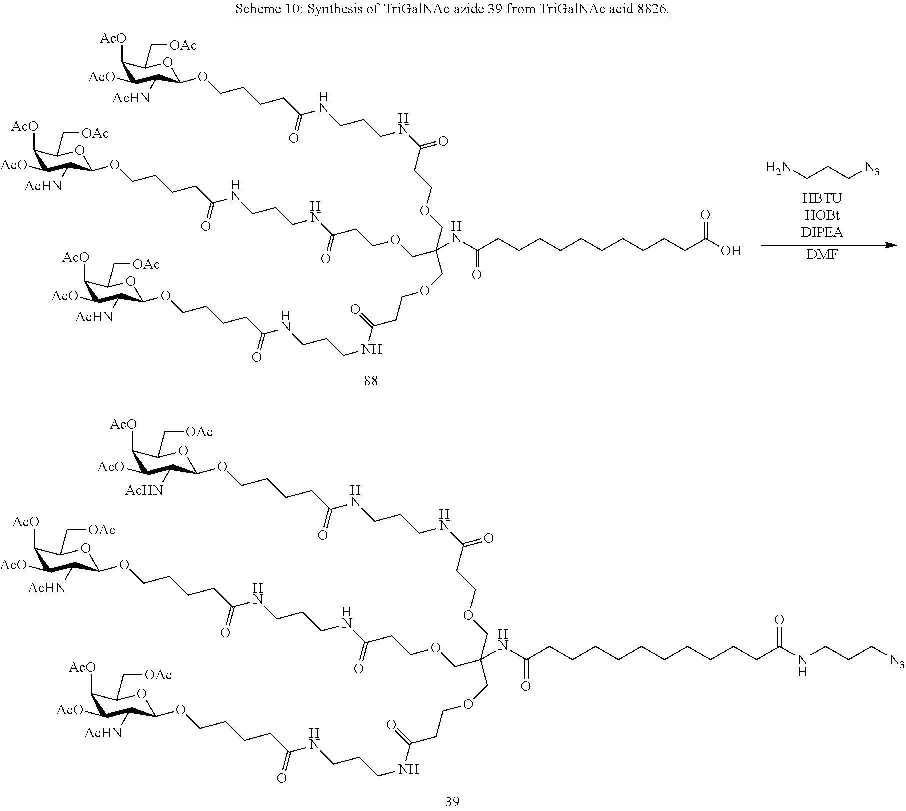

Compound 39: To a clear solution of 88 (3.0 g, 1.50 mmol) in dry dimethylformamide (22.36 mL) was added HBTU (636.55 mg, 1.64 mmol), 1-hydroxybenzotriazole hydrate (257.04 mg, 1.64 mmol) and diisopropylethylamine (585.64 mg, 4.49 mmol, 789.28 μL) in single portions. Reaction mixture was stirred for 5 minutes and then was added 3-azidopropan-1-amine (299.44 mg, 2.99 mmol) slowly. Resulting mixture was stirred at 22° C. for 16 hr and then all volatile matters were removed under high vacuum pump. Residue was diluted with DCM (70 mL) and washed with NH₄Cl solution (3×30 mL), NaHCO₃ solution (3×40 mL), water (50 mL) and brine (2×50 mL). Organic layer was separated, dried over anhydrous Na₂SO₄, filtered and the filtrate was evaporated to dryness. Solid residue thus obtained, was co-evaporated with DCM (20 mL) and kept for drying overnight at 22° C. to afford 39 (2.95 g, 95% yield) as yellow solid. ¹H NMR (600 MHz, DMSO-d₆) δ 7.85-7.79 (m, 7H), 7.73 (t, J=5.7 Hz, 3H), 6.98 (s, 1H), 5.21 (d, J=3.4 Hz, 3H), 4.96 (dd, J=11.2, 3.4 Hz, 3H), 4.48 (d, J=8.5 Hz, 3H), 4.07-3.98 (m, 9H), 3.87 (dt, J=11.2, 8.8 Hz, 3H), 3.70 (dt, J=9.6, 5.9 Hz, 3H), 3.53 (dd, J=12.3, 5.8 Hz, 12H), 3.41 (tt, J=9.6, 6.3 Hz, 3H), 3.11-2.99 (m, 14H), 2.27 (t, J=6.4 Hz, 6H), 2.10 (s, 9H), 2.04 (td, J=7.4, 4.3 Hz, 10H), 1.99 (s, 9H), 1.89 (s, 8H), 1.77 (s, 9H), 1.63 (p, J=6.8 Hz, 2H), 1.54-1.41 (m, 22H), 1.22 (s, 12H) ppm. ¹³C NMR (151 MHz, DMSO-d₆) δ 172.49, 172.18, 171.93, 170.08, 170.00, 169.91, 169.63, 169.35, 162.30, 100.97, 70.46, 69.82, 68.65, 68.25, 67.32, 66.69, 61.42, 59.46, 49.36, 48.42, 38.24, 36.35, 36.26, 36.01, 35.89, 35.78, 35.72, 35.40, 35.04, 30.76, 29.34, 28.95, 28.83, 28.78, 28.67, 28.62, 28.58, 28.46, 25.31, 25.27, 22.75, 21.83, 20.51, 20.45, 20.43 ppm. MALDI mass calc. for C₉₄H₁₅₄O₃₈N₁₄Na [M+Na]⁺ 2110.0446, found. 2113.793

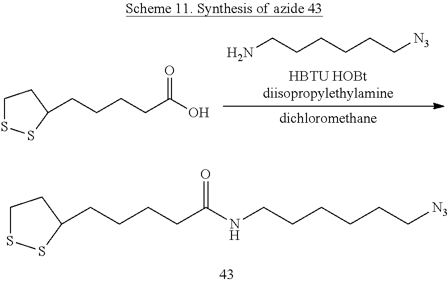 [SMILES: [N-]=[N+]=NCCCCCCN ; O=C(O)CCCCC1CCSS1 ; [N-]=[N+]=NCCCCCCNC(=O)CCCCC1CCSS1]

Compound 43: To a clear solution of 5-(dithiolan-3-yl)pentanoic acid (1.0 g, 4.85 mmol) in dichloromethane (20 mL) was added HBTU (1.84 g, 4.85 mmol) followed by HOBt (654.89 mg, 4.85 mmol) and diisopropylethylamine (1.25 g, 9.69 mmol, 1.69 mL). Reaction mixture was stirred for 5 minutes and to the resulting solution was added 6-azidohexan-1-amine (689.21 mg, 4.85 mmol) in single portion. Reaction mixture was stirred for 16 hr at 22° C. and then all the volatile matters were removed under high vacuum pump. Crude mass thus obtained was purified twice by flash chromatography (gradient: 20-60% EtOAc in hexane) to afford 46 (1.53 g, 96% yield) as yellow oil. ¹H NMR (600 MHz, CDCl₃) δ 5.51 (d, J=6.9 Hz, 1H), 3.57 (dq, J=8.8, 6.3 Hz, 1H), 3.31-3.22 (m, 4H), 3.18 (ddd, J=11.0, 7.1, 5.3 Hz, 1H), 3.12 (dt, J=11.1, 6.9 Hz, 1H), 2.80 (d, J=0.7 Hz, 3H), 2.46 (dtd, J=13.1, 6.6, 5.4 Hz, 1H), 2.21-2.13 (m, 2H), 1.94-1.86 (m, 1H), 1.74-1.57 (m, 4H), 1.53-1.31 (m, 6H) ppm. ¹³C NMR (151 MHz, CDCl₃) δ 172.78, 56.57, 51.45, 40.37, 39.45, 38.73, 38.58, 36.66, 34.73, 29.71, 29.02, 28.85, 26.56, 26.51, 25.55 ppm. HRMS calc. for C₁₄H₂₇N₄O₁S₂ [M+H]⁺ 331.1626, found 331.1628.

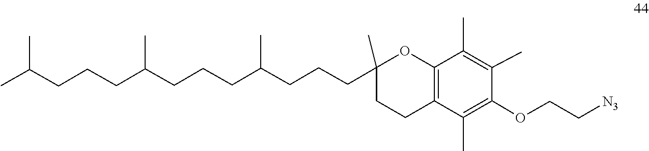 [SMILES: Cc1c(C)c2c(c(C)c1OCCN=[N+]=[N-])CCC(C)(CCCC(C)CCCC(C)CCCC(C)C)O2]

Compound 44: 2,5,7,8-tetramethyl-2-(4,8,12-trimethyltridecyl)chroman-6-ol (α-tocopherol) (5 g, 11.61 mmol), 2-bromoethanol (1.81 g, 14.51 mmol) and sodium hydroxide (696.53 mg, 17.41 mmol, 327.01 μL) were mixed in anhydrous dimethylformamide (25 mL), and stirred at 90° C. for 16 hr. The mixture was cooled to room temperature, then poured into water (100 mL). The solution was extracted with methyl tert-butyl ether (3×50 mL). The organic layers were combined and evaporated in vacuum to leave the residue that was further purified by flash column chromatography (gradient: 5-25% EtOAc in hexane) to afford 2-[2,5,7,8-tetramethyl-2-(4,8,12-trimethyltridecyl)chroman-6-yl]oxyethanol (tocopherol alcohol³²⁻³³)(4.92 g, 89% yield) as brown oil. ¹H NMR (600 MHz, CDCl₃) δ 3.96-3.90 (m, 2H), 3.81-3.76 (m, 2H), 2.57 (t, J=6.8 Hz, 2H), 2.37 (t, J=6.2 Hz, 1H), 2.18 (s, 3H), 2.14 (s, 3H), 2.08 (s, 3H), 1.85-1.71 (m, 2H), 1.63-1.48 (m, 3H), 1.47-1.33 (m, 2H), 1.33-1.19 (m, 9H), 1.18-1.01 (m, 4H), 0.89-0.83 (m, 13H) ppm. ¹³C NMR (151 MHz, CDCl₃) δ 148.10, 147.73, 127.82, 125.85, 123.11, 117.74, 74.96, 73.81, 62.55, 40.23, 40.19, 39.50, 37.71, 37.69, 37.62, 37.59, 37.54, 37.52, 37.49, 37.47, 37.42, 32.93, 32.90, 32.83, 32.80, 31.37, 31.32, 28.11, 24.95, 24.94, 24.57, 24.00, 22.86, 22.76, 21.18, 21.16, 20.78, 19.88, 19.82, 19.79, 19.75, 19.73, 12.85, 11.99, 11.92 ppm.

To a clear solution of tocopherol alcohol (3.48 g, 7.33 mmol) in dichloromethane (50 mL) was added triethylamine (2.97 g, 29.32 mmol, 4.09 mL) and cooed to 0° C. To this reaction mixture was added methanesulfonyl chloride (1.68 g, 14.66 mmol, 1.13 mL) drop-wise for 5 minutes. Reaction mixture was kept for stirring at 0° C. for 10 minutes and then stirred at 22° C. After 1 hr the reaction mixture was quenched with water (40 mL). Organic layer was separated, and aqueous layer was washed with DCM (30 mL). Combined organic layer was dried over anhydrous Na₂SO₄, filtered and filtrate was evaporated under high vacuum to afford a brown oil. The crude product obtained was dissolved in dimethylformamide (30 mL) and to the reaction mixture was added sodium azide (2.86 g, 43.98 mmol). Reaction mixture was stirred at 65° C. for 10 hrs and then cooled. Methyl-tert-butyl ether (40 mL) was added to the reaction mixture and the resulting organic layer was washed with water (2×40 mL) and brine (3×40 mL) respectively. Organic layer was dried over anhydrous Na₂SO₄, filtered and filtrate was evaporated to dryness. Crude compound thus obtained was purified by flash column chromatography (gradient: 0-10% EtOAc in hexane) to afford 44 (2.89 g, 79% yield in two steps) as a yellow transparent gum. ¹H NMR (600 MHz, CDCl₃) δ 3.82 (dd, J=5.5, 4.6 Hz, 2H), 3.58 (dd, J=5.5, 4.5 Hz, 2H), 2.57 (t, J=6.8 Hz, 2H), 2.19 (s, 3H), 2.15 (s, 3H), 2.08 (s, 3H), 1.85-1.71 (m, 2H), 1.60-1.45 (m, 3H), 1.43-1.34 (m, 2H), 1.33-1.19 (m, 9H), 1.18-1.01 (m, 3H), 0.88-0.83 (m, 12H) ppm. ¹³C NMR (151 MHz, CDCl₃) δ 148.20, 147.70, 127.85, 125.90, 123.17, 117.79, 75.00, 71.30, 51.37, 40.18, 40.13, 39.51, 37.71, 37.69, 37.62, 37.60, 37.55, 37.53, 37.50, 37.47, 37.43, 32.94, 32.92, 32.84, 32.82, 31.39, 31.34, 28.12, 24.96, 24.95, 24.58, 24.01, 22.87, 22.77, 21.18, 21.16, 20.79, 19.89, 19.83, 19.80, 19.76, 19.74, 12.77, 11.93, 11.90 ppm. HRMS calc. for C₃₁H₅₃N₃O₂ [M]⁺ 499.4138, found 499.4159.

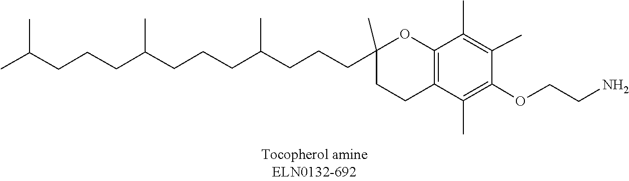 [SMILES: Cc1c(C)c2c(c(C)c1OCCN)CCC(C)(CCCC(C)CCCC(C)CCCC(C)C)O2]

2-[2,5,7,8-tetramethyl-2-(4,8,12-trimethyltridecyl)chroman-6-yl]oxyethanamine Tocopherol amine: To a clear solution of 44 (1.27 g, 2.54 mmol) in THE (20 mL) was added triphenylphosphine (1.33 g, 5.08 mmol) in single portion. To this reaction mixture was added water (5 mL) dropwise until the effervescence ceased. Additional portion of water (4 mL) was then added to the mixture while it became milky. Reaction mixture kept on stirring for 12 hr. All the volatile matters were removed under high vacuum pump and CHCl₃ (30 mL) was added to it. Organic layer was separated, washed with brine (20 mL), dried over anhydrous Na₂SO₄, and filtered. Filtrate was evaporated to dryness to afford the crude product which was then purified by flash column chromatography (gradient: 0-10% MeOH in DCM) to afford Tocopherol amine (1.11 g, 92% yield) as colorless oil. ¹H NMR (600 MHz, CDCl₃) δ 3.69 (t, J=5.2 Hz, 2H), 3.07 (t, J=5.2 Hz, 2H), 2.57 (t, J=6.8 Hz, 2H), 2.18 (s, 3H), 2.14 (s, 3H), 2.08 (s, 3H), 1.81 (dt, J=14.0, 7.1 Hz, 1H), 1.75 (dt, J=13.3, 6.5 Hz, 1H), 1.62-1.45 (m, 5H), 1.45-1.31 (m, 2H), 1.32-1.18 (m, 8H), 1.19-1.01 (m, 3H), 0.91-0.80 (m, 12H) ppm. ¹³C NMR (151 MHz, CDCl₃) δ 148.01, 147.91, 127.83, 125.83, 123.00, 117.65, 74.92, 42.72, 40.19, 40.15, 39.48, 37.68, 37.67, 37.59, 37.57, 37.52, 37.49, 37.47, 37.44, 37.39, 32.90, 32.88, 32.80, 32.77, 31.37, 31.32, 28.09, 24.93, 24.91, 24.55, 23.99, 22.84, 22.74, 21.15, 21.13, 20.76, 19.86, 19.80, 19.77, 19.73, 19.71, 12.87, 12.00, 11.90 ppm.

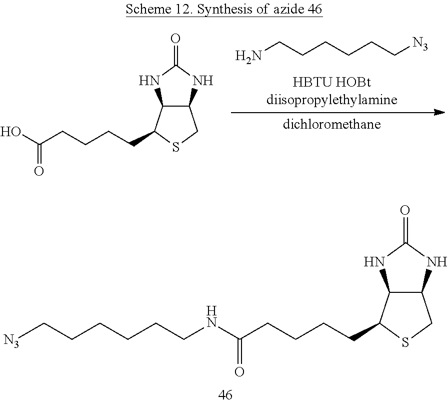 [SMILES: O=C(O)CCCC[C@@H]1SC[C@@H]2NC(=O)N[C@@H]21 ; [N-]=[N+]=NCCCCCCN ; [N-]=[N+]=NCCCCCCNC(=O)CCCC[C@@H]1SC[C@@H]2NC(=O)N[C@@H]21]

5-[(3aS,4S,6aR)-2-oxo-1,3,3a,4,6,6a-hexahydrothieno[3,4-d]imidazol-4-yl]-N-(6-azidohexyl)pentanamide Compound 46: To a clear solution of 5-(2-oxo-1,3,3a,4,6,6a-hexahydrothieno[3,4-d]imidazol-4-yl)pentanoic acid (1.0 g, 4.09 mmol) in dichloromethane (20 mL) was added HBTU (1.55 g, 4.09 mmol) followed by HOBt (553.07 mg, 4.09 mmol) and diisopropylethylamine (1.06 g, 8.19 mmol, 1.43 mL). Reaction mixture was stirred for 5 minutes and to the resulting solution was added 6-azidohexan-1-amine (640.26 mg, 4.50 mmol) in single portion. Reaction mixture was stirred for 16 hr at 22° C. and then all the volatile matters were removed under high vacuum pump to afford Compound 46 (1.44 g, 96% yield) as white hygroscopic solid. ¹H NMR (600 MHz, DMSO-d₆) δ 7.83-7.49 (m, 1H), 6.45 (d, J=5.8 Hz, 1H), 6.38 (s, 1H), 4.30 (dd, J=7.8, 5.0 Hz, 1H), 4.12 (ddd, J=7.7, 4.5, 1.8 Hz, 1H), 3.62 (pd, J=6.6, 3.9 Hz, 1H), 3.32 (dt, J=12.4, 6.9 Hz, 2H), 3.18-3.09 (m, 2H), 3.01 (q, J=6.7 Hz, 2H), 2.92-2.86 (m, 4H), 2.82 (ddd, J=12.4, 5.1, 3.0 Hz, 1H), 2.57 (d, J=12.4 Hz, 1H), 2.22-1.97 (m, 2H), 1.69-1.43 (m, 6H), 1.44-1.19 (m, 13H) ppm. ¹³C NMR (151 MHz, DMSO-d₆) δ 174.51, 171.82, 162.74, 160.87, 61.07, 59.20, 55.51, 55.45, 53.58, 50.60, 50.53, 44.34, 41.85, 40.06, 38.28, 35.24, 29.11, 29.08, 28.27, 28.25, 28.16, 28.13, 28.09, 25.98, 25.92, 25.77, 25.64, 25.39, 24.58, 18.10, 16.75, 12.54 ppm.

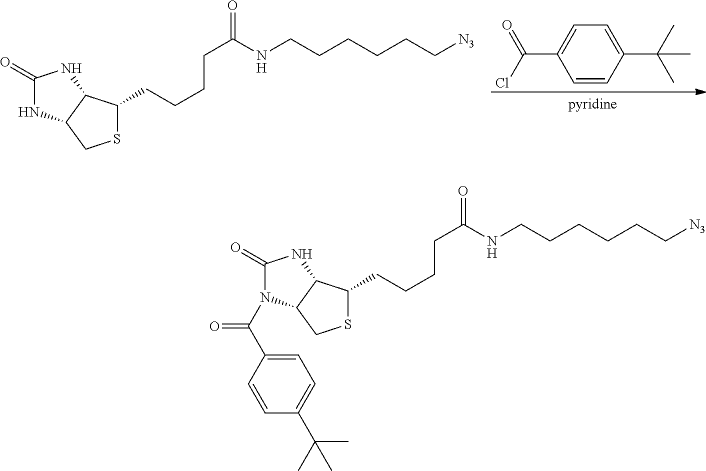

5-[(3aR,6S,6aS)-3-(4-tert-butylbenzoyl)-2-oxo-3a,4,6,6a-tetrahydro-1H-thieno[3,4-d]imidazol-6-yl]-N-(6-azidohexyl)pentanamide: To a suspension of 46 (1.25 g, 3.39 mmol) in pyridine (20 mL) was added 4-tert-butylbenzoyl chloride (800.58 mg, 4.07 mmol, 792.65 μL) slowly. Turbid reaction mixture turned clear after 4 hrs. Stirring was continued for 12 hr ar room temperature then quenched with saturated NaHCO₃ solution (30 mL) and aqueous layer was extracted with DCM (3×20 mL). Combined organic layer was dried over anhydrous Na₂SO₄, filtered and filtrate was evaporated to dryness. Crude compound was purified by flash column chromatography (gradient: 0-5% MeOH in DCM) to afford 5-[(3aR,6S,6aS)-3-(4-tert-butylbenzoyl)-2-oxo-3a,4,6,6a-tetrahydro-1H-thieno[3,4-d]imidazol-6-yl]-N-(6-azidohexyl)pentanamide (1.35 g, 75% yield) as yellowish white foam. 1H NMR (600 MHz, DMSO) δ 7.91 (d, J=1.7 Hz, 1H), 7.75 (t, J=5.6 Hz, 1H), 7.41 (d, J=0.8 Hz, 5H), 5.06 (ddd, J=7.8, 5.2, 1.0 Hz, 1H), 4.21 (ddd, J=8.0, 4.5, 1.6 Hz, 1H), 3.31 (t, J=6.9 Hz, 1H), 3.26 (ddd, J=8.9, 6.0, 4.5 Hz, 1H), 3.03 (p, J=6.2 Hz, 3H), 2.88 (d, J=13.1 Hz, 1H), 2.06 (t, J=7.4 Hz, 2H), 1.74-1.64 (m, 1H), 1.60-1.46 (m, 6H), 1.38 (p, J=7.1 Hz, 2H), 1.29 (d, J=0.8 Hz, 10H) ppm. 13C NMR (151 MHz, DMSO) δ 171.77, 168.90, 155.48, 153.58, 132.59, 128.47, 124.13, 61.74, 57.31, 54.84, 50.58, 38.27, 38.25, 37.43, 35.21, 34.63, 30.96, 29.05, 28.27, 28.21, 28.19, 27.93, 25.95, 25.92, 25.89, 25.86, 25.29 ppm.

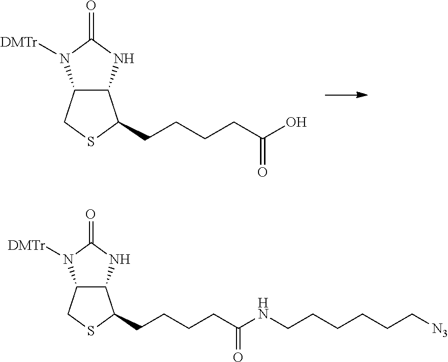

5-[(3aR,6aS)-3-[bis(4-methoxyphenyl)-phenyl-methyl]-2-oxo-3a,4,6,6a-tetrahydro-1H-thieno[3,4-d]imidazol-6-yl]-N-(6-azidohexyl)pentanamide: To a clear solution of 5-[(3aR,6aS)-3-[bis(4-methoxyphenyl)-phenyl-methyl]-2-oxo-3a,4,6,6a-tetrahydro-1H-thieno[3,4-d]imidazol-6-yl]pentanoic acid (0.8 g, 1.46 mmol) in DMF (15 mL) was added HBTU (554.98 mg, 1.46 mmol) followed by HOBt (197.73 mg, 1.46 mmol) and diisopropylethylamine (378.26 mg, 2.93 mmol, 509.78 μL). Reaction mixture was stirred for 5 minutes and to the resulting solution was added 6-azidohexan-1-amine (228.91 mg, 1.61 mmol) in single portion. Reaction mixture was stirred for 16 hr at 22° C. and then all the volatile matters were removed under high vacuum pump. The residue was dissolved in DCM (30 mL) and organic layer was washed with saturated NaHCO₃ (20 mL) solution, 10% NH₄Cl solution (20 mL), and brine (2×20 mL). Organic layer was separated, dried over anhydrous Na₂SO₄, filtered and the filtrate was evaporated to dryness. Crude compound was purified by flash column chromatography (gradient: 0-5% MeOH in DCM) to afford 5-[(3aR,6aS)-3-[bis(4-methoxyphenyl)-phenyl-methyl]-2-oxo-3a,4,6,6a-tetrahydro-1H-thieno[3,4-d]imidazol-6-yl]-N-(6-azidohexyl)pentanamide (0.59 g, 60% yield) as white foam. 1H NMR (600 MHz, DMSO) δ 7.72 (t, J=5.6 Hz, 1H), 7.33-7.26 (m, 2H), 7.28-7.17 (m, 4H), 7.12-7.01 (m, 5H), 6.91-6.81 (m, 5H), 6.75 (dd, J=8.5, 1.9 Hz, 1H), 4.33 (ddd, J=7.7, 4.4, 3.2 Hz, 1H), 4.28 (dt, J=5.6, 2.5 Hz, 1H), 3.74 (s, 6H), 3.30 (t, J=6.9 Hz, 2H), 3.12 (ddd, J=8.9, 6.1, 4.1 Hz, 1H), 3.01 (td, J=6.9, 5.6 Hz, 2H), 2.23-2.17 (m, 3H), 2.03 (t, J=7.4 Hz, 2H), 1.68-1.58 (m, 1H), 1.56-1.21 (m, 11H) ppm. 13C NMR (151 MHz, DMSO) δ 171.77, 160.79, 157.78, 144.10, 135.93, 135.91, 131.03, 129.40, 127.23, 126.48, 112.61, 112.58, 71.74, 64.49, 59.29, 55.02, 54.34, 50.58, 38.25, 35.23, 29.04, 28.45, 28.21, 28.17, 25.94, 25.88, 25.32 ppm.

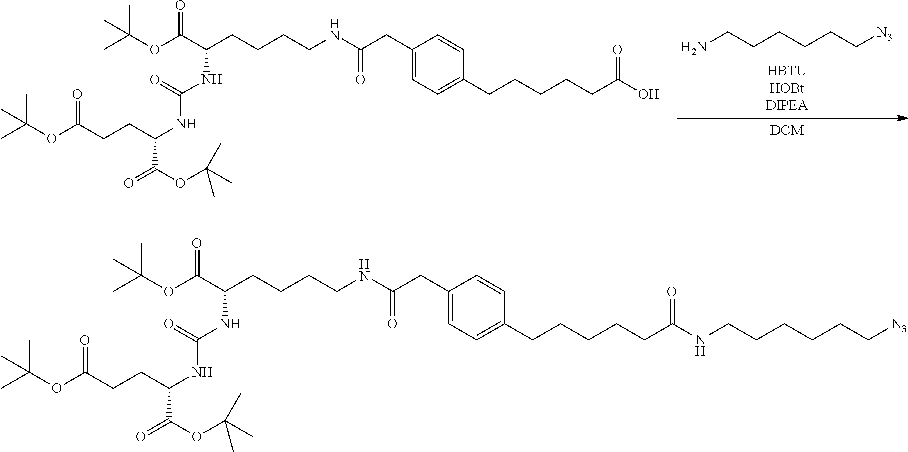 [SMILES: CC(C)(C)OC(=O)CC[C@H](NC(=O)N[C@@H](CCCCNC(=O)Cc1ccc(CCCCCC(=O)O)cc1)C(=O)OC(C)(C)C)C(=O)OC(C)(C)C ; [N-]=[N+]=NCCCCCCN ; CC(C)(C)OC(=O)CC[C@H](NC(=O)N[C@@H](CCCCNC(=O)Cc1ccc(CCCCCC(=O)NCCCCCCN=[N+]=[N-])cc1)C(=O)OC(C)(C)C)C(=O)OC(C)(C)C]

Ditert-butyl(2S)-2-[[(1S)-5-[[2-[4-[6-(6-azidohexylamino)-6-oxo-hexyl]phenyl]acetyl]amino]-1-tert-butoxycarbonyl-pentyl]carbamoylamino]pentanedioate Compound 45: To a clear solution of 6-[4-[2-[[(5S)-6-tert-butoxy-5-[[(1S)-4-tert-butoxy-1-tert-butoxycarbonyl-4-oxo-butyl]carbamoylamino]-6-oxo-hexyl]amino]-2-oxo-ethyl]phenyl]hexanoic acid (0.41 g, 569.52 μmol) in DCM (20 mL) was added HBTU (215.99 mg, 569.52 μmol) followed by HOBt (76.95 mg, 569.52 μmol) and DIPEA (147.21 mg, 1.14 mmol, 198.40 μL). Reaction mixture was stirred for 5 minutes and to the resulting solution was added 6-azidohexan-1-amine (105.28 mg, 740.38 μmol) in single portion. Reaction mixture was stirred for 18 hr at 22° C. and then all the volatile matters were removed under high vacuum pump. Crude mass thus obtained was purified by flash chromatography (gradient: 0-5% MeOH in DCM) to afford 45 (0.41 g, 85% yield) as white hygroscopic foam.

¹H NMR (600 MHz, CDCl₃) δ 7.22-7.04 (m, 4H), 5.75 (s, 1H), 5.51 (s, 1H), 5.22 (d, J=16.7 Hz, 1H), 4.32 (td, J=8.2, 4.9 Hz, 1H), 4.26 (td, J=7.9, 4.6 Hz, 1H), 3.62-3.43 (m, 2H), 3.30-3.20 (m, 5H), 3.17-3.09 (m, 1H), 2.60 (t, J=7.7 Hz, 2H), 2.38-2.24 (m, 2H), 2.14 (t, J=7.6 Hz, 2H), 2.07 (ddt, J=15.6, 9.3, 5.3 Hz, 1H), 1.85 (dddd, J=14.1, 9.4, 8.3, 6.1 Hz, 1H), 1.79-1.69 (m, 1H), 1.66-1.57 (m, 10H), 1.52-1.46 (m, 2H), 1.45 (d, J=0.9 Hz, 19H), 1.43 (s, 10H), 1.41-1.32 (m, 5H) ppm. ¹³C NMR (151 MHz, CDCl₃) δ 173.12, 172.61, 172.52, 172.37, 171.70, 157.11, 141.68, 132.52, 129.50, 129.14, 82.16, 81.82, 80.71, 53.38, 53.14, 51.48, 43.49, 39.45, 39.15, 36.88, 35.45, 32.47, 31.75, 31.19, 29.73, 28.98, 28.94, 28.88, 28.50, 28.22, 28.17, 28.15, 26.58, 26.54, 25.76, 22.28 ppm.

### Conjugates

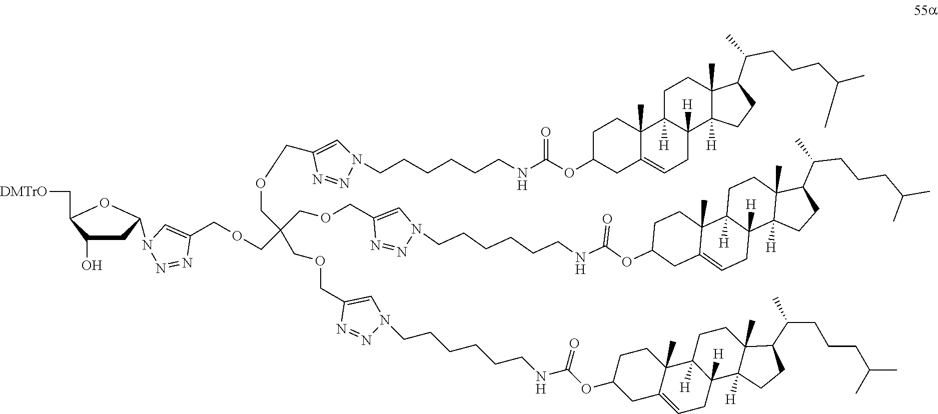

Compound 55a. Compound 50α (300 mg, 0.40 mmol) and 32 (3.00 mmol, 1.20 mmol) were dissolved in THF/MeOH (1:1, v/v) (4 ml). To the solution were added H₂O (2 ml), copper sulfate pentahydrate (2 mg, 0.01 mmol) and sodium ascorbate (8 g, 0.04 mmol). The heterogeneous solution was irradiated by microwave at 70° C. for 6 h. After the irradiation, the heterogeneous solution became two phase solution. Whole solution was extracted with DCM/brine. Then organic layer was dried on sodium sulfate and concentrated under reduced pressure. The residue was subjected to flash silica gel chromatography. Eluent with 3% MeOH in DCM gave 55α (710 mg, 0.29 mmol, 73%, R_{f}=0.5) as a white foam.

¹H-NMR (400 MHz, DMSO-d₆) δ 0.65 (9H, s), 0.84-1.54, 1.76-1.98, 2.19-2.36 (6H, m), 2.91-2.95 (6H, m), 3.37 (6H, s), 3.40 (2H, s), 3.74 (6H, s), 4.01-4.07 (1H, m), 4.22-4.32 (12H, m), 4.44 (6H, s), 4.47 (2H, s), 5.31-5.34 (4H, m, 1H diminished with D₂O), 6.41-6.44 (1H, dd), 6.69-6.77 (1H, b), 6.87-6.89 (4H, d), 7.19-7.39 (10H, m), 7.92 (3H, s), 8.23 (1H, s). ¹³C-NMR (400 MHz, DMSO-d₆, at 60° C.) d 11.65, 18.57, 18.94, 20.61, 22.31, 22.52, 23.27, 23.82, 25.52, 25.56, 27.31, 27.67, 27.91, 29.16, 29.52, 31.35, 31.49, 35.13, 35.73, 36.12, 36.64, 41.92, 45.04, 49.23, 49.63, 55.07, 55.77, 56.20, 63.80, 64.39, 68.81, 70.98, 72.86, 85.67, 86.79, 88.76, 113.25, 121.64, 123.17, 126.63, 127.72, 127.76, 129.65, 135.59, 135.66, 139.90, 144.11, 144.49, 144.73, 155.61, 158.19 ppm. ¹H NMR (600 MHz, (CDCl₃) δ 8.15 (s, 1H), 7.57 (s, 3H), 7.46-7.41 (m, 2H), 7.36-7.26 (m, 8H), 7.25-7.19 (m, 1H), 6.86-6.81 (m, 4H), 6.64-6.54 (m, 1H), 5.47 (s, 1H), 5.36 (dt, J=5.1, 2.2 Hz, 3H), 4.80 (t, J=6.0 Hz, 3H), 4.63-4.57 (m, 5H), 4.54 (s, 7H), 4.52-4.43 (m, 3H), 4.32 (t, J=7.2 Hz, 7H), 3.79 (d, J=0.9 Hz, 6H), 3.45 (s, 8H), 3.32 (dd, J=10.2, 3.9 Hz, 1H). 3.15-3.09 (m, 7H), 2.98 (ddd, J=14.4, 7.9, (0.5 Hz, 1H), 2.49 (d, J=14.7 Hz, 1H). 2.37-2.31 (m, 3H), 2.30-2.21 (m, 3H), 2.03-1.95 (m, 4H), 1.95-1.72 (m, 19H), 1.62-1.20 (m, 56H), 1.18-1.07 (m, 13H), 1.07-0.88 (m, 29H). 0.86 (dd, J=6.6, 2.9 Hz, 19H), 0.67 (s, 91.1) ppm LC-MS calcd for [M+H]⁺ 2413.71, found 2413.70.

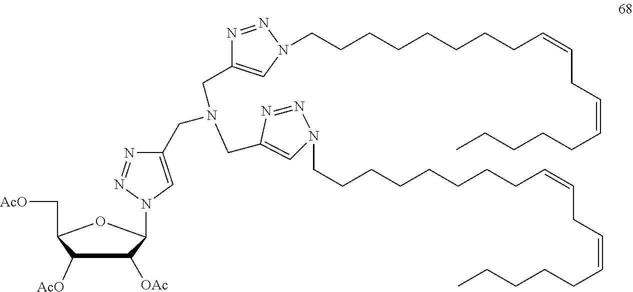

Compound 68. Compound 67 (640 mg, 1.48 mmol) was treated with cupper (18 mg, 0.30 mmol), tetrakis-acetonitrilecupper hexafluorophosphate (110 mg, 0.30 mmol) and 35 (950 mg, 3.26 mmol) at ambient temperature overnight. Then the solution was concentrated under reduced pressure and then chromatography gave crude 68. LC-MS calcd for [M+H]⁺ 1015.4, found 1015.4.

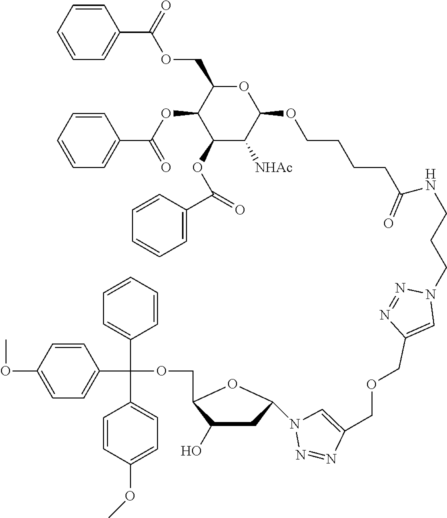 [SMILES: COc1ccc(C(OC[C@H]2O[C@H](n3cc(COCc4cn(CCCNC(=O)CCCCO[C@@H]5O[C@H](COC(=O)c6ccccc6)[C@H](OC(=O)c6ccccc6)[C@H](OC(=O)c6ccccc6)[C@H]5NC(C)=O)nn4)nn3)C[C@H]2O)(c2ccccc2)c2ccc(OC)cc2)cc1]

[(2R,3R,4R,5R,6R)-5-acetamido-3,4-dibenzoyloxy-6-[5-[3-[4-[[1-[(2S,5R)-5-[[bis(4-methoxyphenyl)-phenyl-methoxy]methyl]-4-hydroxy-tetrahydrofuran-2-yl]triazol-4-yl]methoxymethyl]triazol-1-yl]propylamino]-5-oxo-pentoxy]tetrahydropyran-2-yl]methyl benzoate ELN0132-573: To a clear suspension of 71a (0.8 g, 1.44 mmol) in THE (30 mL) methanol (10 mL) and water (10 mL) was added 38 (1.03 g, 1.44 mmol), copper (II) sulfate pentahydrate (17.98 mg, 71.99 μmol) and sodium ascorbate (142.62 mg, 719.92 μmol) in single portions. Reaction mixture was stirred for 16 hr at 22° C. and TLC was checked and then diluted with EtOAc (40 mL). Organic layer was washed with water (30 mL) and brine (2×30 mL). Organic layer was separated, dried over anhydrous Na₂SO₄, filtered, and the filtrate was evaporated under high vacuum. Crude residue was purified by flash column chromatography (gradient: 10-70% EtOAc in hexane) to afford ELN0132-573 (1.16 g, 63% yield) as white foam. ¹H NMR (600 MHz, DMSO) δ 8.40 (s, 1H), 8.18 (s, 1H), 7.98 (d, J=9.3 Hz, 1H), 7.95-7.85 (m, 5H), 7.70 (ddt, J=10.4, 7.7, 1.3 Hz, 3H), 7.64 (ddt, J=8.8, 7.1, 1.3 Hz, 1H), 7.57 (dddd, J=10.0, 8.4, 7.4, 1.5 Hz, 3H), 7.52-7.46 (m, 2H), 7.42-7.36 (m, 4H), 7.32 (dd, J=8.5, 7.1 Hz, 2H), 7.29-7.23 (m, 4H), 6.94-6.87 (m, 4H), 6.49 (dd, J=7.6, 3.0 Hz, 1H), 5.75 (d, J=3.9 Hz, 1H), 5.51 (d, J=4.0 Hz, 1H), 5.36 (dd, J=11.1, 3.4 Hz, 1H), 4.74 (d, J=8.5 Hz, 1H), 4.58 (d, J=7.2 Hz, 4H), 4.50-4.43 (m, 2H), 4.40-4.32 (m, 3H), 4.30-4.19 (m, 3H), 3.83-3.75 (m, 1H), 3.73 (s, 6H), 3.51 (dt, J=10.0, 6.2 Hz, 1H), 3.14 (dd, J=10.2, 3.6 Hz, 1H), 3.07-2.97 (m, 3H), 2.82 (p, J=7.2 Hz, 1H), 2.33 (dt, J=14.2, 3.0 Hz, 1H), 2.08 (t, J=7.1 Hz, 2H), 1.94 (p, J=6.9 Hz, 2H), 1.70 (s, 3H), 1.57-1.48 (m, 4H) ppm. ¹³C NMR (151 MHz, DMSO) δ 172.16, 169.42, 165.22, 165.17, 164.87, 158.07, 144.79, 144.05, 143.62, 135.52, 135.41, 133.80, 133.53, 133.51, 129.68, 129.20, 129.18, 129.04, 129.01, 128.99, 128.72, 128.61, 127.88, 127.66, 126.69, 124.33, 122.72, 113.23, 100.90, 88.90, 86.80, 85.51, 71.84, 70.84, 69.97, 68.75, 67.92, 63.69, 62.37, 62.02, 55.02, 54.91, 49.73, 47.26, 35.63, 34.99, 29.94, 28.59, 22.70, 21.79 ppm.

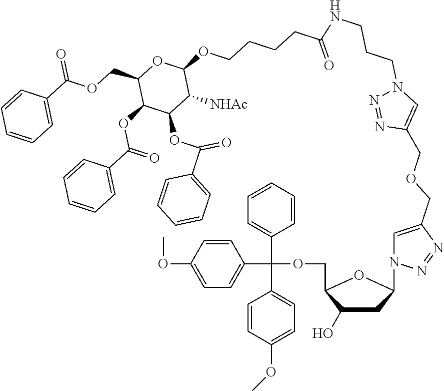 [SMILES: COc1ccc(C(OC[C@H]2O[C@@H](n3cc(COCc4cn(CCCNC(=O)CCCCO[C@@H]5O[C@H](COC(=O)c6ccccc6)[C@H](OC(=O)c6ccccc6)[C@H](OC(=O)c6ccccc6)[C@H]5NC(C)=O)nn4)nn3)C[C@H]2O)(c2ccccc2)c2ccc(OC)cc2)cc1]

[(2R,3R,4R,5R,6R)-5-acetamido-3,4-dibenzoyloxy-6-[5-[3-[4-[[1-[(2S,5R)-5-[[bis(4-methoxyphenyl)-phenyl-methoxy]methyl]-4-hydroxy-tetrahydrofuran-2-yl]triazol-4-yl]methoxymethyl]triazol-1-yl]propylamino]-5-oxo-pentoxy]tetrahydropyran-2-yl]methyl benzoate ELN0132-574: To a clear suspension of 71β (0.4 g, 719.92 μmol) and 38 (515.28 mg, 719.92 μmol) in THE (30 mL) and water (10 mL) was added copper (II) sulfate pentahydrate (179.76 mg, 719.92 μmol) and sodium ascorbate (71.31 mg, 359.96 μmol) in single portions. Reaction mixture was stirred for 16 hr at 22° C. and TLC was checked and then diluted with EtOAc (40 mL). Organic layer was washed with water (30 mL) and brine (2×30 mL). Organic layer was separated, dried over anhydrous Na₂SO₄, filtered, and the filtrate was evaporated under high vacuum. Crude residue was purified by flash column chromatography (gradient: 10-70% EtOAc in hexane) to afford ELN0132-574 (0.48 g, 52% yield) as white foam. ¹H NMR (600 MHz, DMSO) δ 8.24 (s, 1H), 8.04 (s, 1H), 7.98 (d, J=9.3 Hz, 1H), 7.92 (tt, J=7.9, 1.4 Hz, 4H), 7.87 (t, J=5.7 Hz, 1H), 7.70 (ddt, J=9.8, 7.3, 1.3 Hz, 3H), 7.67-7.61 (m, 1H), 7.60-7.53 (m, 3H), 7.52-7.46 (m, 2H), 7.42-7.36 (m, 2H), 7.33-7.27 (m, 2H), 7.27-7.20 (m, 2H), 7.17 (ddd, J=9.9, 4.9, 2.1 Hz, 5H), 6.87-6.79 (m, 4H), 6.38 (dd, J=6.8, 4.6 Hz, 1H), 5.75 (d, J=5.1 Hz, 2H), 5.36 (dd, J=11.1, 3.4 Hz, 1H), 4.73 (d, J=8.5 Hz, 1H), 4.51 (d, J=5.8 Hz, 4H), 4.48-4.39 (m, 3H), 4.38-4.23 (m, 4H), 3.97 (q, J=5.2 Hz, 1H), 3.80 (dq, J=9.3, 5.1 Hz, 1H), 3.70 (d, J=1.0 Hz, 6H), 3.51 (dt, J=9.7, 5.9 Hz, 1H), 3.11-3.00 (m, 4H), 2.69 (ddd, J=13.3, 6.4, 4.5 Hz, 1H), 2.39 (dt, J=13.1, 6.5 Hz, 1H), 2.10-2.05 (m, 2H), 1.91 (p, J=7.0 Hz, 2H), 1.70 (s, 3H), 1.55-1.49 (m, 5H) ppm. ¹³C NMR (151 MHz, DMSO) δ 172.14, 169.40, 165.21, 165.16, 164.86, 157.97, 144.75, 143.90, 135.58, 135.34, 133.79, 133.53, 133.50, 129.68, 129.61, 129.19, 129.17, 129.03, 128.99, 128.89, 128.72, 128.60, 127.74, 127.61, 127.40, 126.55, 124.05, 123.16, 113.09, 112.74, 100.88, 87.46, 86.02, 85.37, 71.83, 70.27, 69.96, 68.74, 67.90, 63.95, 62.71, 62.54, 62.01, 54.98, 54.96, 54.90, 49.72, 47.15, 35.62, 34.98, 29.98, 28.58, 22.69, 21.78 ppm.

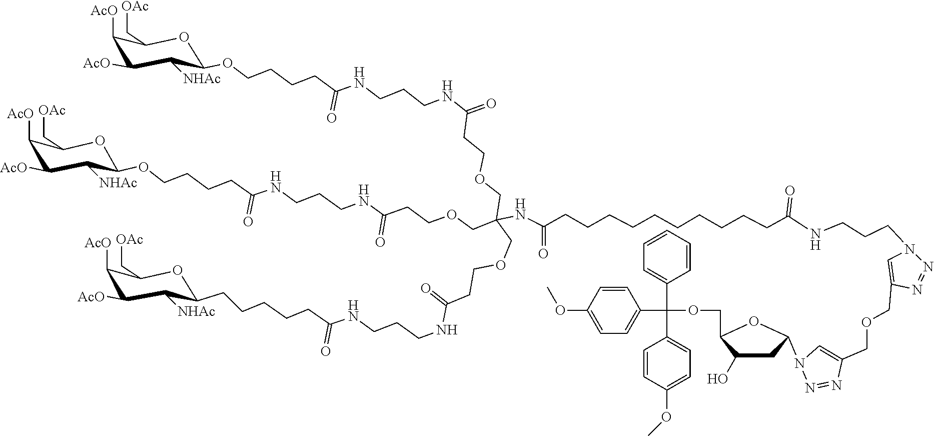

[(2R,3R,4R,5R,6R)-5-acetamido-6-[5-[3-[3-[3-[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]-2-[[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]methyl]-2-[[12-[3-[4-[[1-[(2S,5R)-5-[[bis(4-methoxyphenyl)-phenyl-methoxy]methyl]-4-hydroxy-tetrahydrofuran-2-yl]triazol-4-yl]methoxymethyl]triazol-1-yl]propylamino]-12-oxo-dodecanoyl]amino]propoxy]propanoylamino]propylamino]-5-oxo-pentoxy]-3,4-diacetoxy-tetrahydropyran-2-yl]methyl acetate ELN0132-580: To a clear suspension of 71a and 39 (1.20 g, 575.93 μmol) in THE (30 mL) and water (10 mL) was added copper (II) sulfate pentahydrate (7.19 mg, 28.80 μmol) and sodium ascorbate (57.05 mg, 287.97 μmol) in single portions. Reaction mixture was stirred for 16 hr at 22° C. and TLC was checked and then diluted with EtOAc (40 mL). Organic layer was washed with water (30 mL) and brine (2×30 mL). Organic layer was separated, dried over anhydrous Na₂SO₄, filtered, and the filtrate was evaporated under high vacuum. Crude residue was purified by trituration (1:1 EtOAc in hexane) to afford ELN0132-580 (1.21 g, 79% yield) as yellowish white foam. ¹H NMR (600 MHz, DMSO) δ 8.37 (s, 1H), 8.15 (s, 1H), 7.92-7.84 (m, 11H), 7.78 (t, J=5.7 Hz, 5H), 7.38 (dt, J=8.6, 1.8 Hz, 3H), 7.35-7.28 (m, 4H), 7.28-7.20 (m, 7H), 7.00 (s, 2H), 6.93-6.86 (m, 6H), 6.48 (dd, J=7.6, 3.1 Hz, 1H), 5.53 (d, J=4.1 Hz, 1H), 5.21 (d, J=3.4 Hz, 5H), 4.96 (dd, J=11.3, 3.4 Hz, 5H), 4.61-4.55 (m, 5H), 4.49 (d, J=8.5 Hz, 5H), 4.34 (t, J=7.0 Hz, 2H), 4.29-4.19 (m, 3H), 4.02 (qt, J=8.4, 4.4 Hz, 20H), 3.87 (dt, J=11.2, 8.8 Hz, 5H), 3.75-3.67 (m, 14H), 3.53 (dd, J=12.0, 5.7 Hz, 22H), 3.40 (dt, J=9.6, 6.3 Hz, 5H), 3.03 (p, J=6.6 Hz, 25H), 2.94-2.77 (m, 3H), 2.28 (t, J=6.4 Hz, 11H), 2.10 (d, J=1.8 Hz, 14H), 2.08 (s, 8H), 1.99 (s, 9H), 1.89 (d, J=1.4 Hz, 16H), 1.77 (d, J=0.9 Hz, 15H), 1.54-1.40 (m, 42H), 1.25-1.19 (m, 21H) ppm.

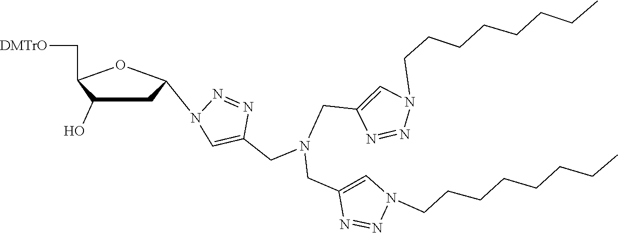

(2R,5S)-2-[[bis(4-methoxyphenyl)-phenyl-methoxy]methyl]-5-[4-[[bis[(1-octyltriazol-4-yl)methyl]amino]methyl]triazol-1-yl]tetrahydrofuran-3-ol ELN0132-591: To a clear suspension of 77α (0.8 g, 1.35 mmol) and 1-azidooctane (1.26 g, 8.1 mmol) in THF (30 mL) and water (20 mL) was added copper (II) sulfate pentahydrate (33.70 mg, 134.98 mol) and sodium ascorbate (267.41 mg, 1.35 mmol) in single portions. Reaction mixture was stirred for 16 hr at 22° C. and TLC was checked and then diluted with EtOAc (40 mL). Organic layer was washed with water (30 mL) and brine (2×30 mL). Organic layer was separated, dried over anhydrous Na₂SO₄, filtered, and the filtrate was evaporated under high vacuum. Crude residue was purified by flash column chromatography (gradient: 0-5% MeOH in DCM) to afford ELN0132-591 (0.71 g, 58% yield) as white foam. ¹H NMR (600 MHz, DMSO) δ 8.36 (s, 1H), 8.12-8.09 (m, 2H), 7.41-7.37 (m, 2H), 7.31 (t, J=7.7 Hz, 2H), 7.28-7.20 (m, 5H), 6.94-6.87 (m, 4H), 6.50-6.46 (m, 1H), 5.52 (d, J=4.0 Hz, 1H), 4.33 (t, J=7.0 Hz, 4H), 4.26 (dt, J=15.6, 3.7 Hz, 2H), 3.73 (s, 6H), 3.68 (s, 5H), 3.01 (dd, J=10.3, 4.9 Hz, 1H), 2.83 (d, J=6.6 Hz, OH), 2.34 (d, J=14.2 Hz, 1H), 1.79 (t, J=7.8 Hz, 4H), 1.30-1.14 (m, 17H), 0.81 (t, J=7.0 Hz, 6H) ppm. ¹³C NMR (151 MHz, DMSO) δ 158.09, 144.79, 135.53, 135.42, 129.69, 127.88, 127.67, 126.70, 113.23, 86.87, 85.52, 70.87, 63.71, 55.03, 49.48, 31.13, 29.59, 28.49, 28.30, 25.81, 22.03, 13.91 ppm.

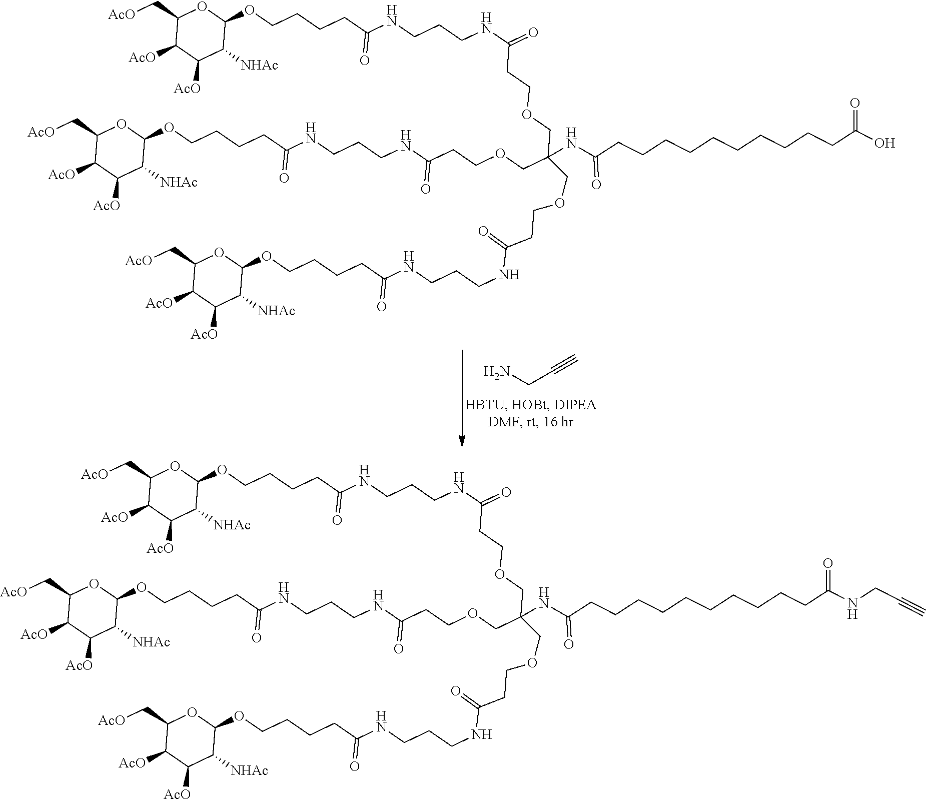

[(2R,3R,4R,5R,6R)-5-acetamido-6-[5-[3-[3-[3-[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]-2-[[3-[3-[5-[(2R,3R,4R,5R,6R)-3-acetamido-4,5-diacetoxy-6-(acetoxymethyl) tetrahydropyran-2-yl]oxypentanoylamino]propylamino]-3-oxo-propoxy]methyl]-2-[[12-oxo-12-(prop-2-ynylamino)dodecanoyl]amino]propoxy]propanoylamino]propylamino]-5-oxo-pentoxy]-3,4-diacetoxy-tetrahydropyran-2-yl]methyl acetate (ELN0132-288)³⁴⁻³⁵: To a clear solution of TriGalNac acid (6.4 g, 3.19 mmol) in dry dimethylformamide (25 mL) was added HBTU (1.36 g, 3.51 mmol), 1-Hydroxybenzotriazole hydrate (548.36 mg, 3.51 mmol) and diisopropylethylamine (1.25 g, 9.57 mmol, 1.68 mL) in single portions. Reaction mixture was stirred for 5 minutes and then was added prop-2-yn-1-amine (537.88 mg, 9.57 mmol, 625.44 μL) slowly. Resulting mixture was stirred at 22° C. for 16 hr and then all volatile matters were removed under high vacuum pump. Residue was diluted with DCM (70 mL) and washed with NH₄Cl solution (3×30 mL), NaHCO₃ solution (3×40 mL), water (50 mL) and brine (2×50 mL). Organic layer was separated, dried over anhydrous Na₂SO₄, filtered and the filtrate was evaporated to dryness. Solid residue thus obtained, was co-evaporated with DCM (20 mL) and kept for drying overnight at 22° C. to afford ELN0132-288 (6.08 g, 93% yield) as yellow solid. ¹H NMR (400 MHz, DMSO-d₆) δ 8.19 (s, OH), 7.72 (t, J=5.6 Hz, 3H), 6.97 (s, 1H), 5.21 (d, J=3.4 Hz, 3H), 4.97 (dd, J=11.2, 3.4 Hz, 3H), 4.48 (d, J=8.4 Hz, 3H), 4.01 (d, J=5.1 Hz, 8H), 3.93-3.79 (m, 4H), 3.70 (dt, J=10.2, 5.5 Hz, 3H), 3.53 (d, J=7.3 Hz, 12H), 3.41 (dt, J=9.5, 6.0 Hz, 3H), 3.08-2.98 (m, 13H), 2.69 (s, 3H), 2.27 (t, J=6.3 Hz, 6H), 2.10 (s, 9H), 2.04 (t, J=6.9 Hz, 10H), 1.99 (s, 9H), 1.89 (s, 9H), 1.77 (s, 9H), 1.56-1.39 (m, 23H), 1.22 (s, 13H) ppm. ¹³C NMR (126 MHz, DMSO-d₆) δ 172.47, 171.91, 171.89, 170.06, 169.97, 169.89, 169.60, 169.33, 100.96, 81.35, 79.15, 72.67, 70.44, 69.81, 68.63, 68.26, 67.31, 66.68, 61.41, 59.45, 49.36, 38.22, 36.34, 36.25, 36.00, 35.89, 35.03, 29.33, 28.92, 28.81, 28.75, 28.61, 28.57, 27.67, 25.29, 25.12, 22.73, 21.81, 20.49, 20.43, 20.41 ppm. MALDI calc. for C₉₄H₁₅₁N₁₁O₃₈Na [M+Na]⁺ 2066.27; found 2068.86.

### Synthesis of Hexaose-Azides

Exemplary hexose-azides are prepared with synthetic protocols shown in Schemes 13 and 14.

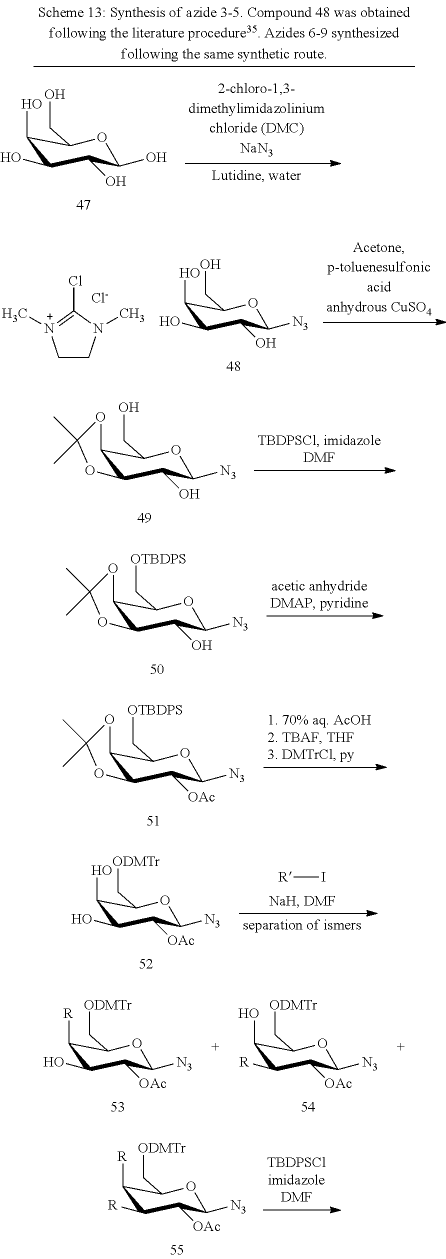

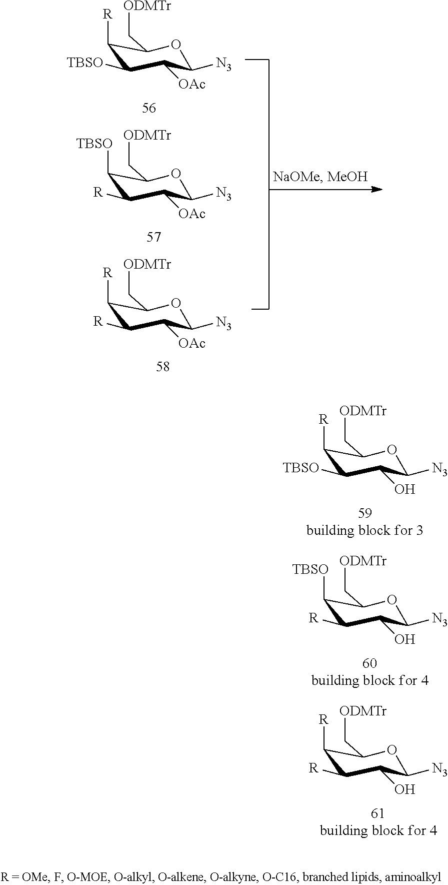

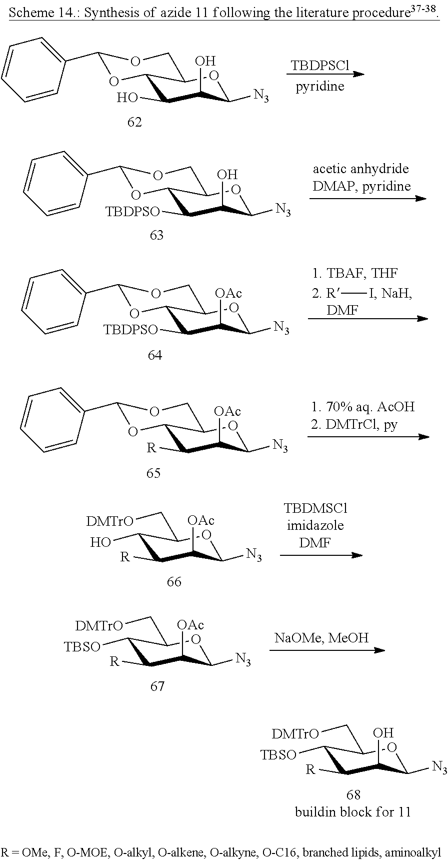

### Oligonucleotide Synthesis:

Oligonucleotides used for the exonuclease assay were synthesized on an ABI-394 and those used for in vitro efficacy assays were synthesized on a MerMade 192 synthesizer on 1-μmol scale using universal or custom supports. A solution of 0.25 M 5-(S-ethylthio)-1H-tetrazole in acetonitrile (CH₃CN) was used as the activator. The solutions of commercially available phosphoramidites and synthesized 5′-(R)—C-methyl-guanosine phosphoramidities were 0.15 M in anhydrous CH₃CN or ACN/DMF (9:1, v/v). The 5′-(S)—C-methyl-guanosine phosphoramidities were 0.15 M in anhydrous 15% DCM in CH₃CN. The oxidizing reagent was 0.02 M I₂ in THF/pyridine/H₂O. The detritylation reagent was 3% dichloroacetic acid in CH₂Cl₂. After completion of the automated synthesis, the oligonucleotide was manually released from support and deprotected using a mixture of aqueous MeNH₃ (40% wt) at 60° C. for 12 min. After filtration through a 0.45-μm nylon filter, oligonucleotides were either purified or, for oligonucleotides containing ribose sugars, the 2′ hydroxyl was deprotected by treatment with Et₃N·3HF at 60° C. for 30 min. Oligonucleotides were purified using IEX-HPLC using an appropriate gradient of mobile phase (buffer A: 0.15 M NaCl, 10% CH₃CN; buffer B 1.0 M NaBr, 10% MeCN) and desalted using size-exclusion chromatography with water as an eluent. Oligonucleotides were then quantified by measuring the absorbance at 260 nm. Extinction coefficients were calculated using the following extinction coefficients for each residue: A, 13.86; T/U, 7.92; C, 6.57; and G, 10.53 M⁻¹ cm⁻¹. The purity and identity of modified ONs were verified by analytical anion exchange chromatography and mass spectrometry, respectively.

After the trityl-off synthesis using the MerMade 192, columns were incubated with 150 μL of 40% aqueous methylamine for 30 min at room temperature, and solutions were drained via vacuum into a 96-well plate. After repeating the incubation and draining with a fresh portion of aqueous methylamine, the plate containing the crude oligonucleotides was sealed and shaken at room temperature for 60 min to completely remove all protecting groups. In the case of RNA, the 2′ hydroxyl was deprotected by treating with Et₃N·3HF at 60° C. for 60 min. Precipitation of the crude oligonucleotides was accomplished via the addition of 1.2 mL of ACN/EtOH (9:1, v/v) to each well, followed by centrifugation at 3000 rpm for 45 min at 4° C. The supernatant was removed from each well, and the pellets were resuspended in 950 μL of 20 mM aqueous NaOAc. Oligonucleotides were desalted over a GE Hi-Trap desalting column (Sephadex G25 Superfine) using water as an eluant. The identities and purities of all oligonucleotides were confirmed using ESI-MS and IEX-HPLC, respectively.

For oligonucleotides synthesized using the ABI 394, the manufacturer's standard protocols were used for cleavage and deprotection. Crude oligonucleotides were purified using strong anion exchange with phosphate buffers (pH 8.5) containing NaBr. The identities and purities of all oligonucleotides were confirmed using ESI-LC/MS and IEX-HPLC respectively.

### Exemplary Results

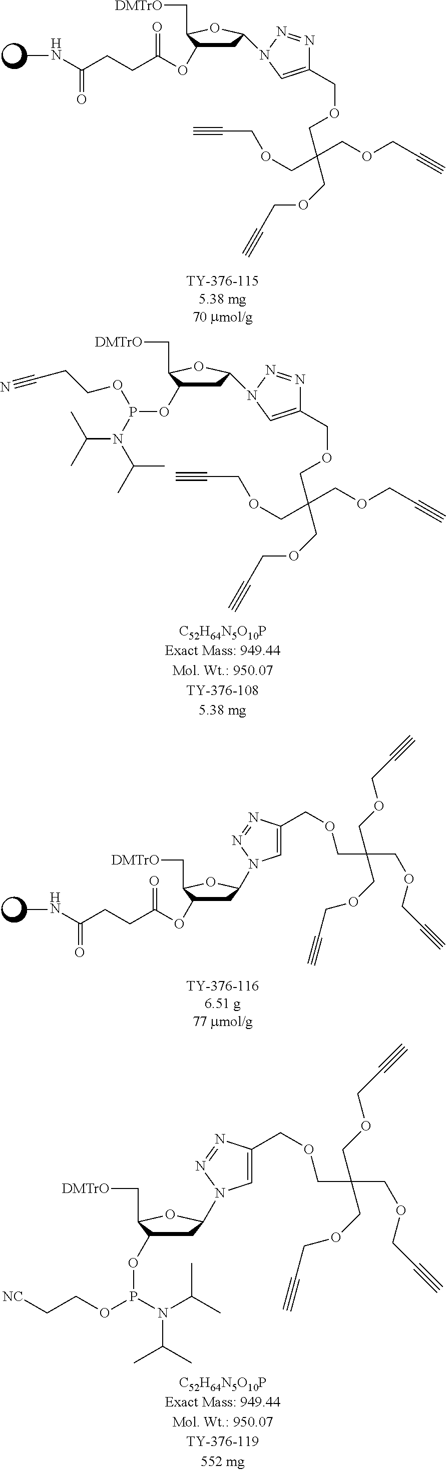

Some exemplary target sequences are show in Table 1.

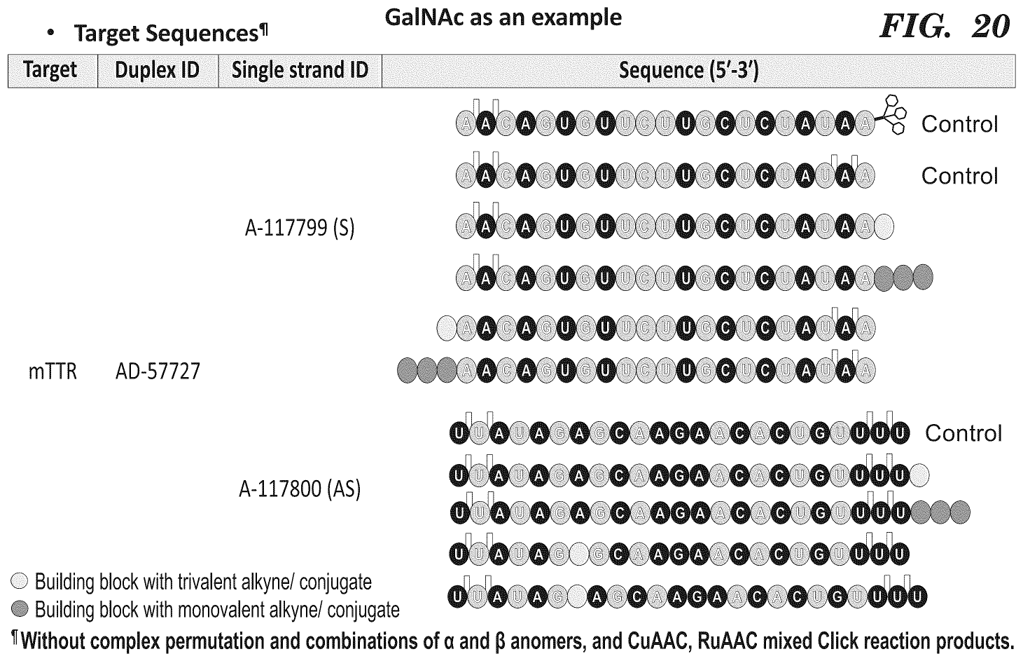

TABLE 1
Exemplary target sequences
Target Sequences (FIG. 20)

| A-117799 | Control-1 | AfsasCfaGfuGfuUfCfUfuGfcUfcUfaUfaAfL96 (SEQ ID NO. 1) |
|---|---|---|
| (Sense) | Control-2 | AfsasCfaGfuGfuUfCfUfuGfcUfcUfaUfsasAf (SEQ ID NO. 2) |
| | 1 | AfsasCfaGfuGfuUfCfUfuGfcUfcUfaUfaAf(M1) (SEQ ID NO. 1) |
| | 2 | AfsasCfaGfuGfuUfCfUfuGfcUfcUfaUfaAf(M2)(M2)(M2) (SEQ ID NO. 1) |
| | 3 | (M1)AfaCfaGfuGfuUfCfUfuGfcUfcUfaUfsasAf (SEQ ID NO. 48) |
| | 4 | (M2)(M2)(M2)AfaCfaGfuGfuUfCfUfuGfcUfcUfaUfsasAf (SEQ ID NO. 48) |
| A-117800 | Control | usUfsaUfaGfaGfcAfagaAfcAfcUfgUfususu (SEQ ID NO. 3) |
| (Anti- | 1 | usUfsaUfaGfaGfcAfagaAfcAfcUfgUfususu(M1) (SEQ ID NO. 3) |
| sense) | 2 | usUfsaUfaGfaGfcAfagaAfcAfcUfgUfususu(M2)(M2)(M2) (SEQ ID NO. 3) |
| | 3 | usUfsaUfaGf(M1)GfcAfagaAfcAfcUfgUfususu (SEQ ID NO. 4) |
| | 4 | usUfsaUfaGf(M1)aGfcAfagaAfcAfcUfgUfususu(SEQ ID NO. 4) |

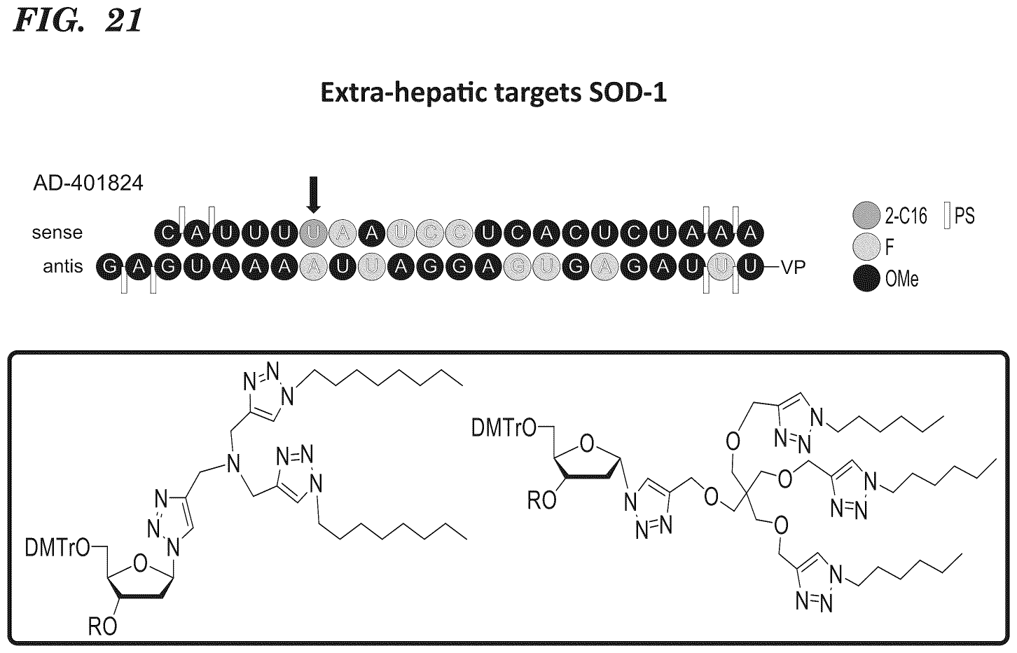

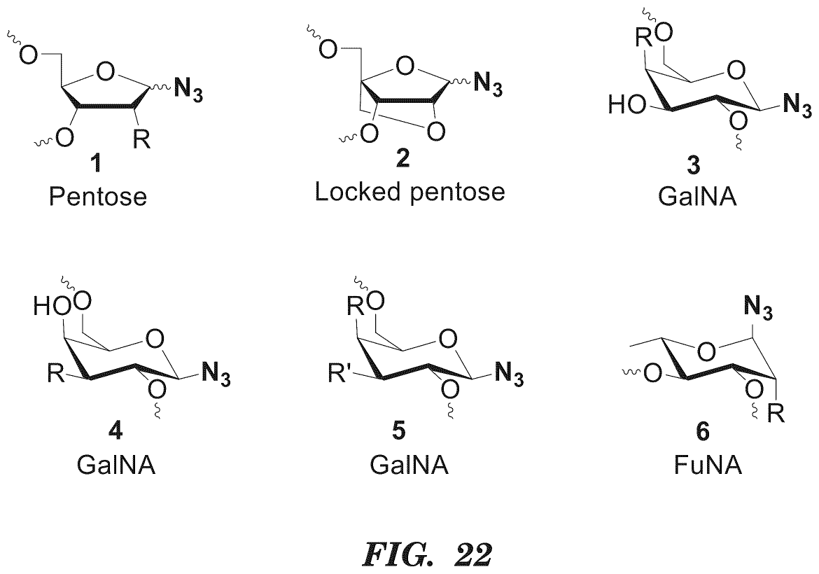

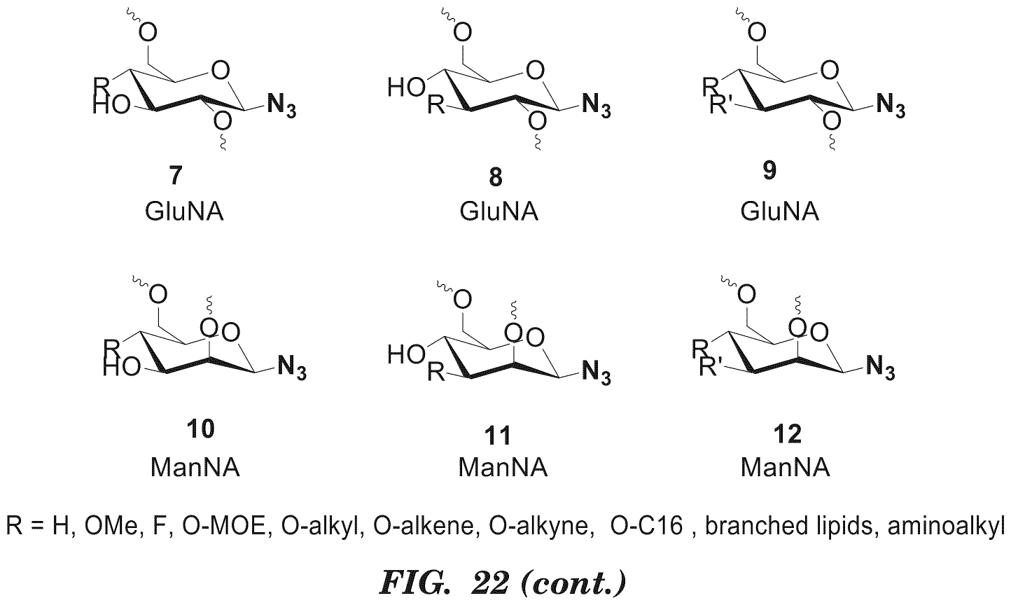

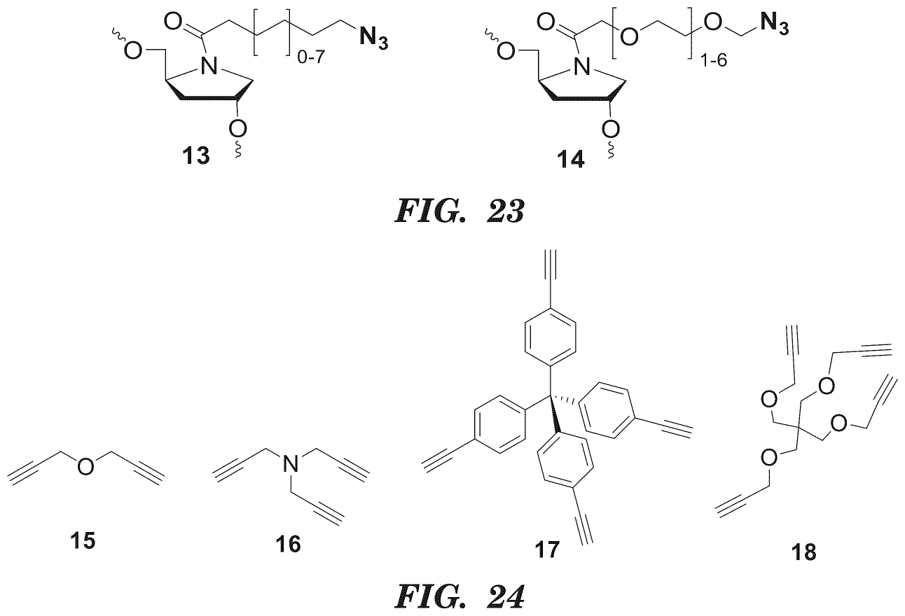

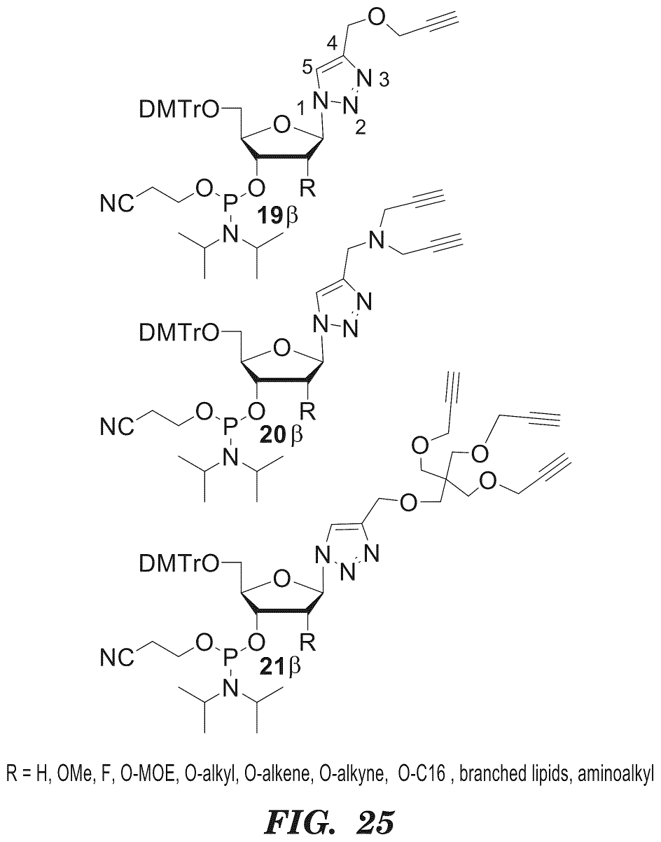

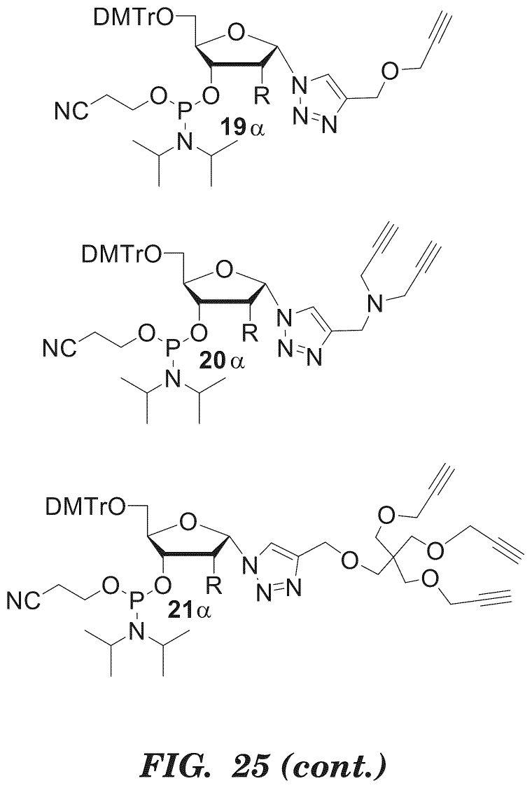

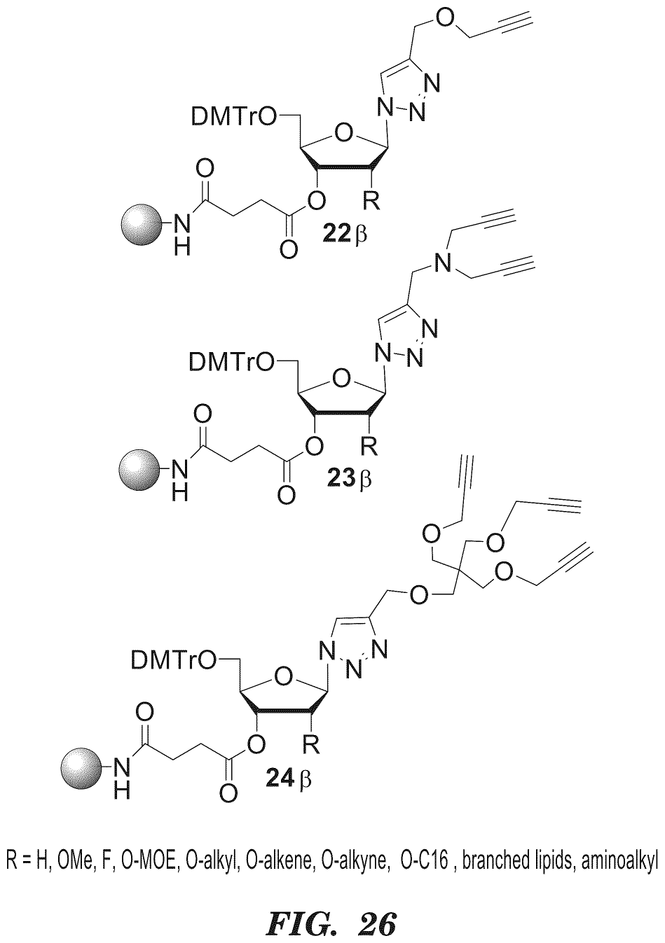

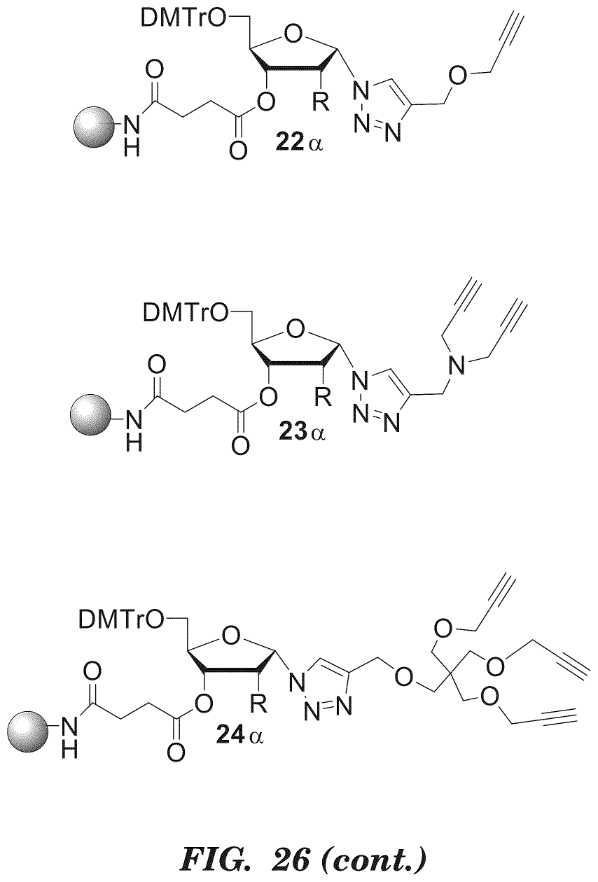

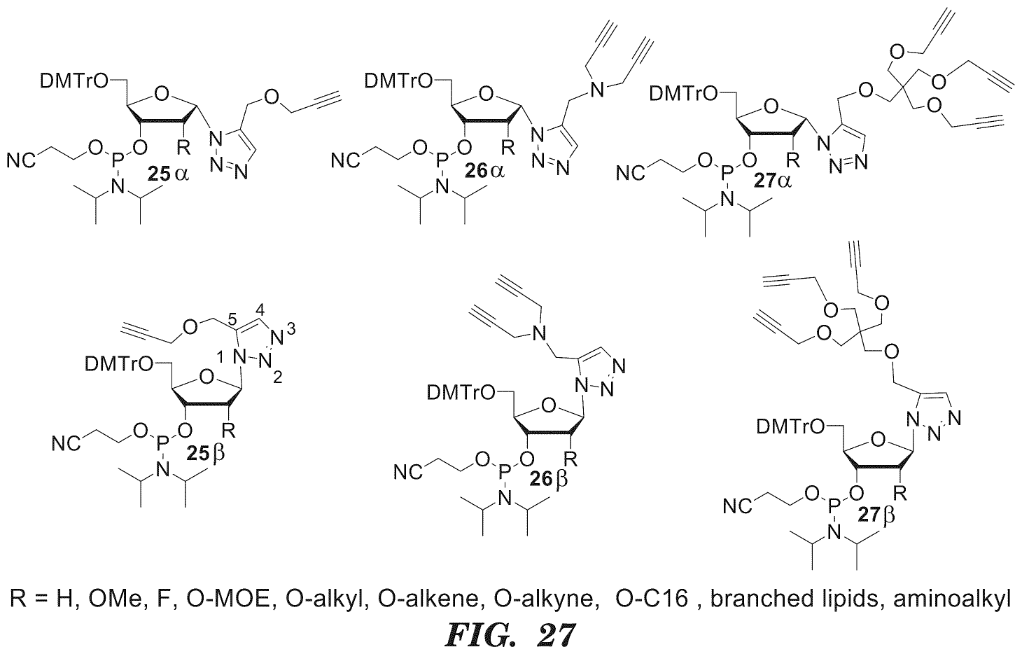

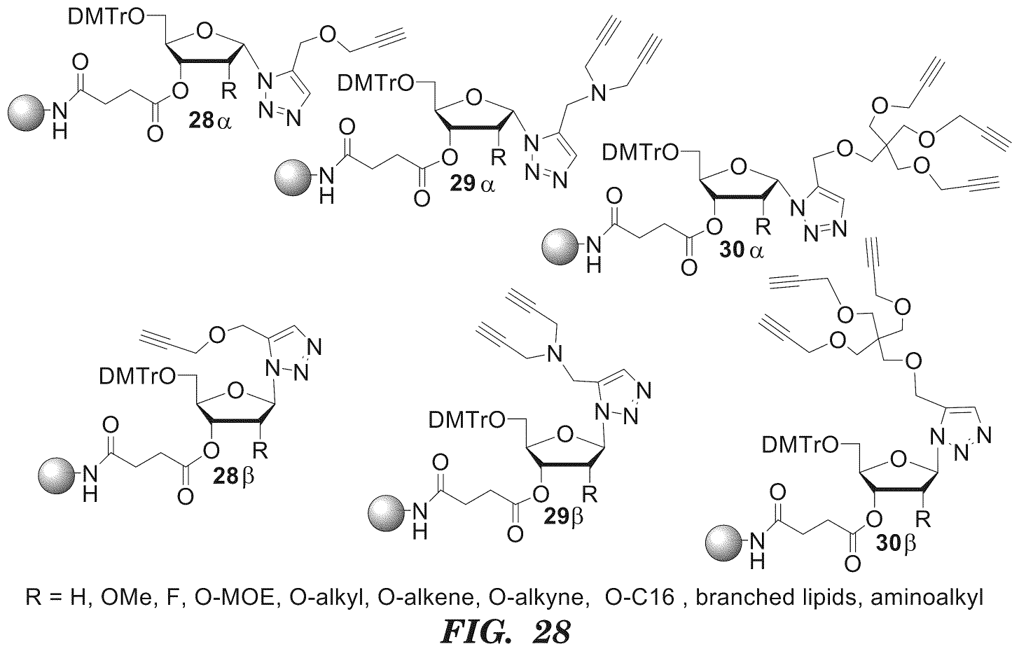

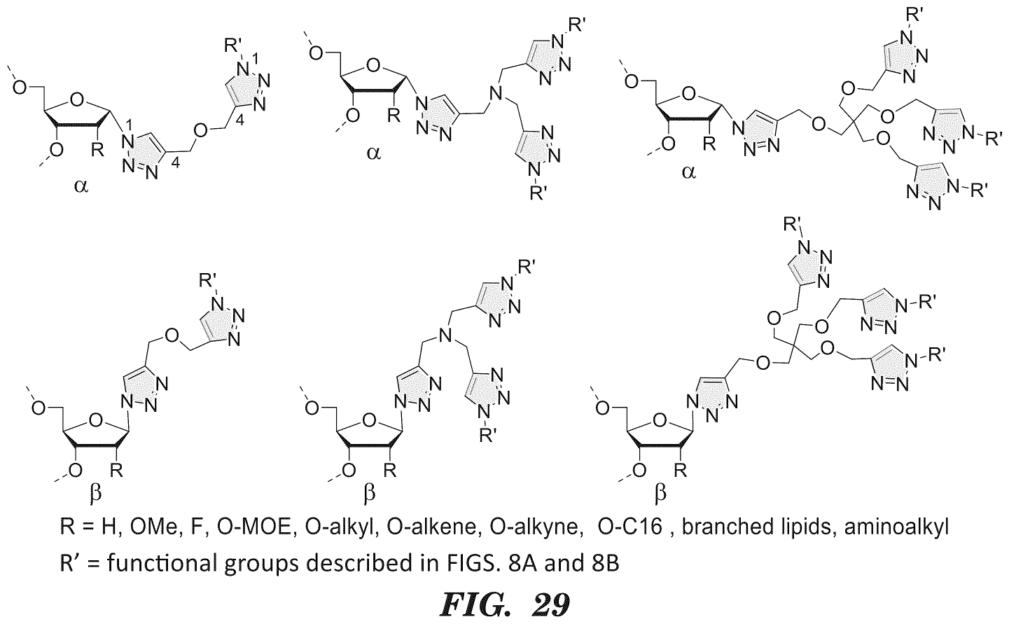

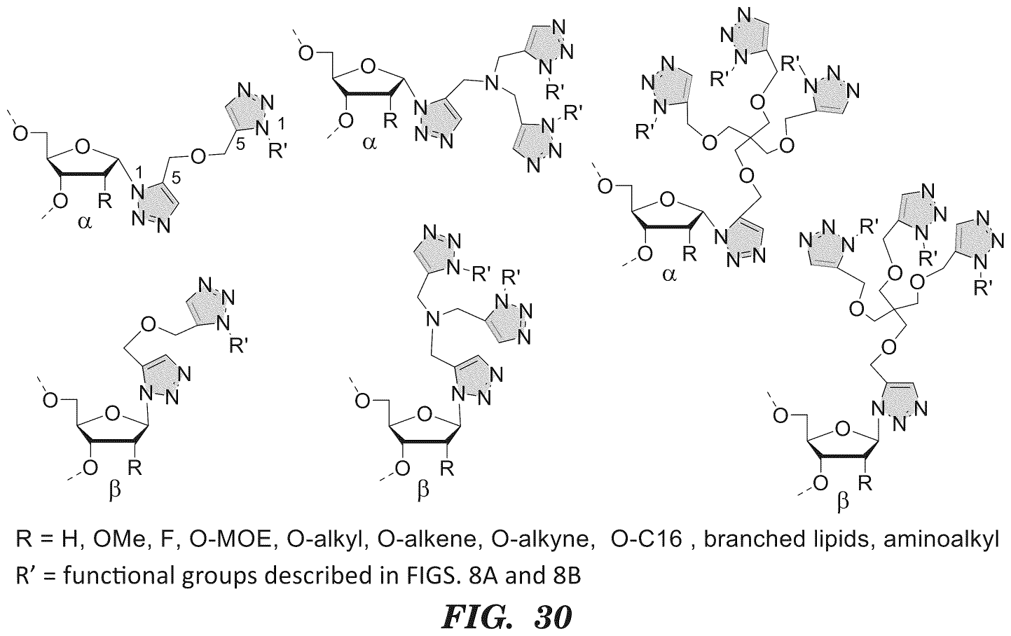

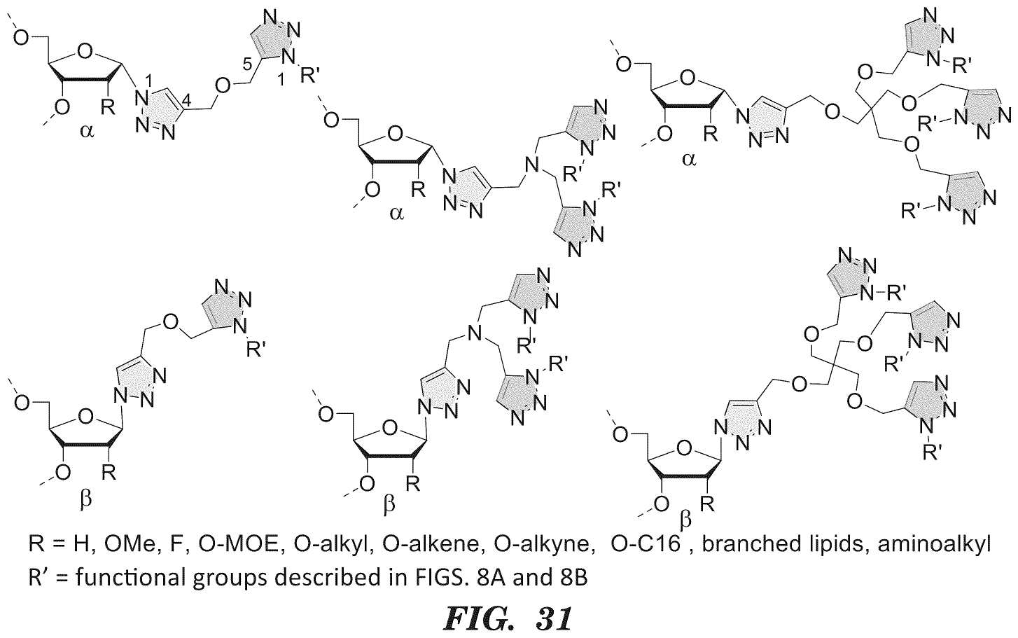

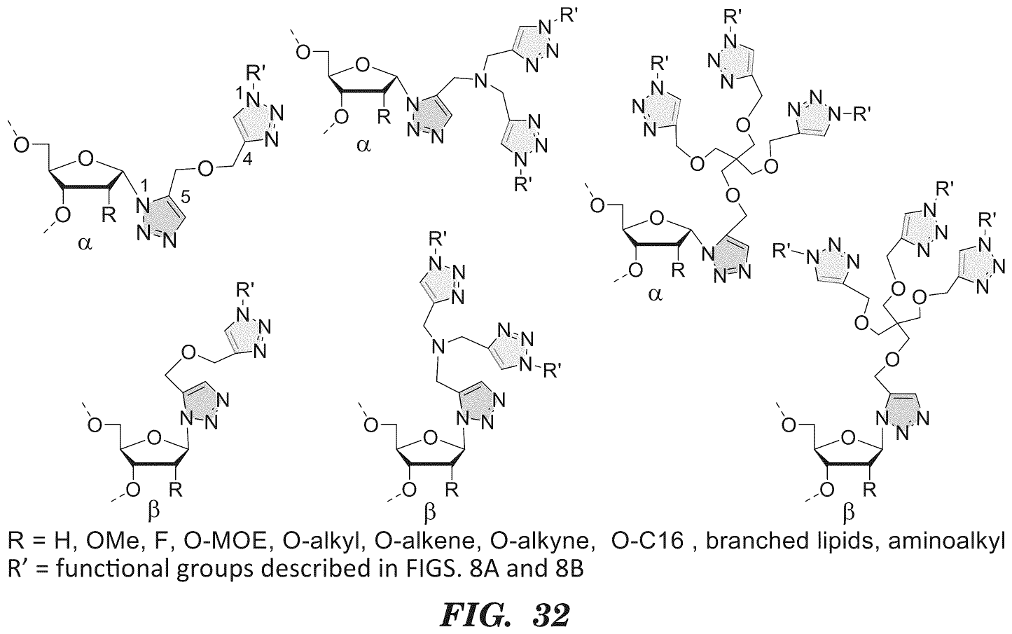

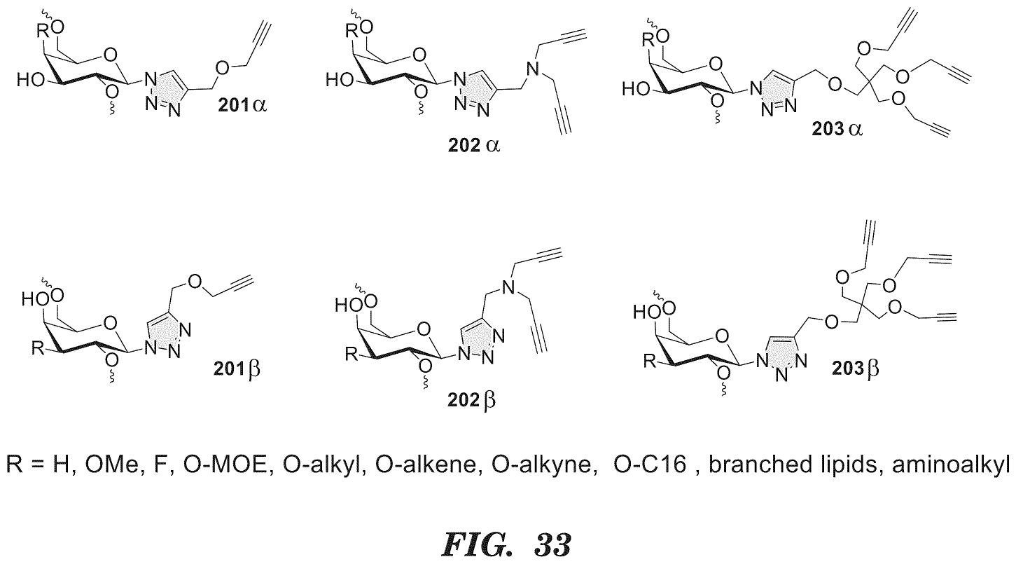

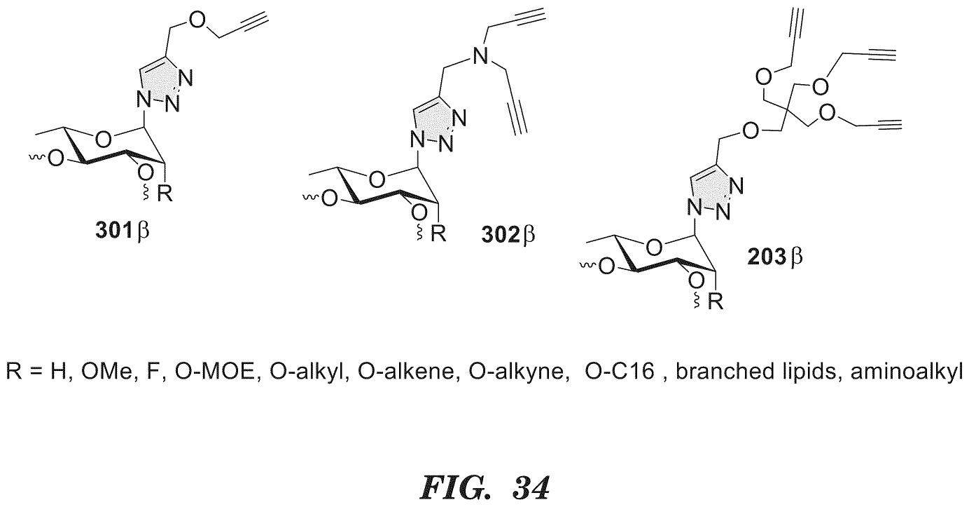

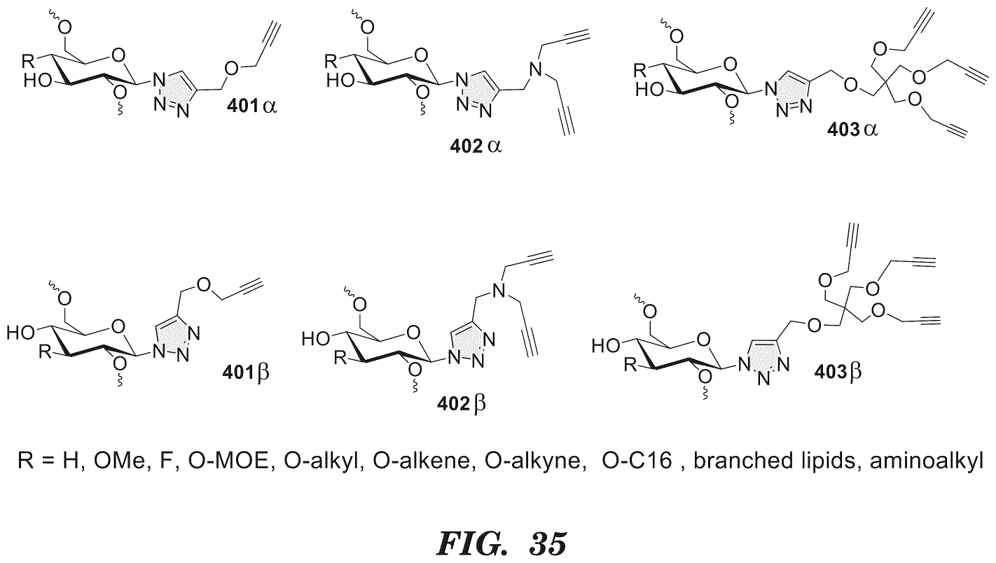

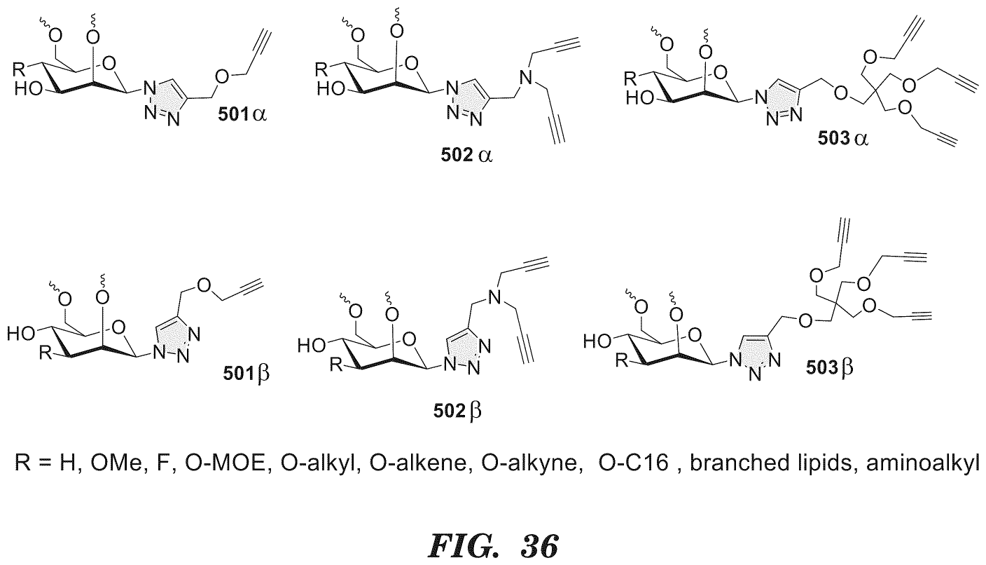

Extra-hepatic targets SOD-1 (FIG. 21)

| | | |
|---|---|---|
| Sense | Control | csasuuu(Uhd)AfaUfCfCfucacucuasasa (SEQ ID NO. 5) |
| | 1 | csasuuu(M3)AfaUfCfCfucacucuasasa (SEQ ID NO. 6) |
| | 2 | (M3)csasuuuAfaUfCfCfucacucuasasa(SEQ ID NO. 6) |
| | 3 | csasuuuAfaUfCfCfucacucuasasa(M3) (SEQ ID NO. 6) |
| Antisense | 1 | VPusUfsuagAfgUfGfaggaUfuAfaaaugsasg (SEQ ID NO. 7) |
| | 2 | VPusUfsuagAfgUfGfaggaUfuAfaaaugsasg(M3) (SEQ ID NO. 7) |
| | 3 | VPusUfsuagAf(M3)UfGfaggaUfuAfaaaugsasg (SEQ ID NO. 8) |

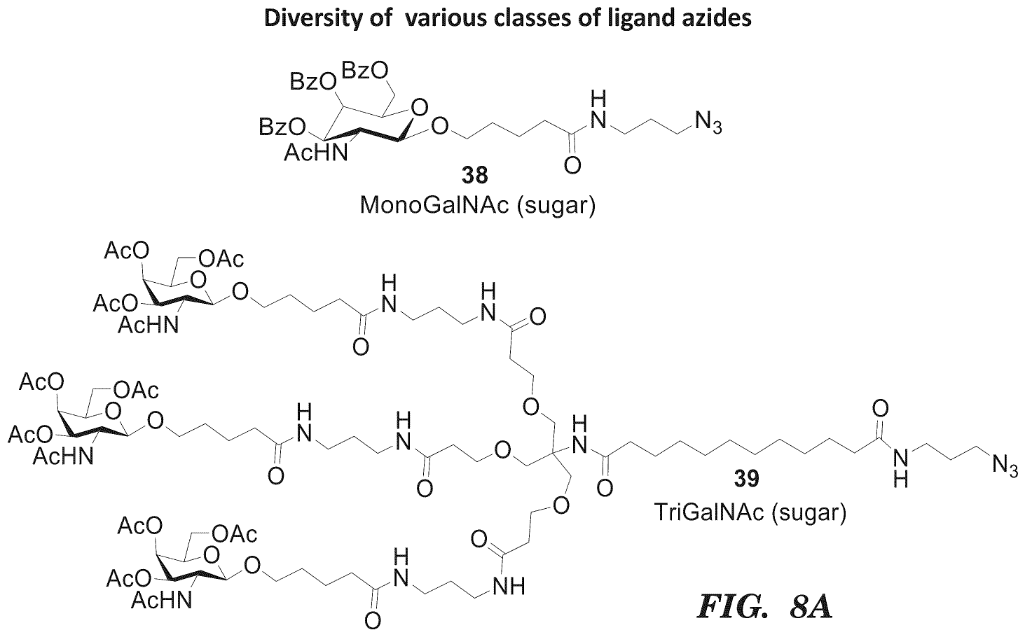

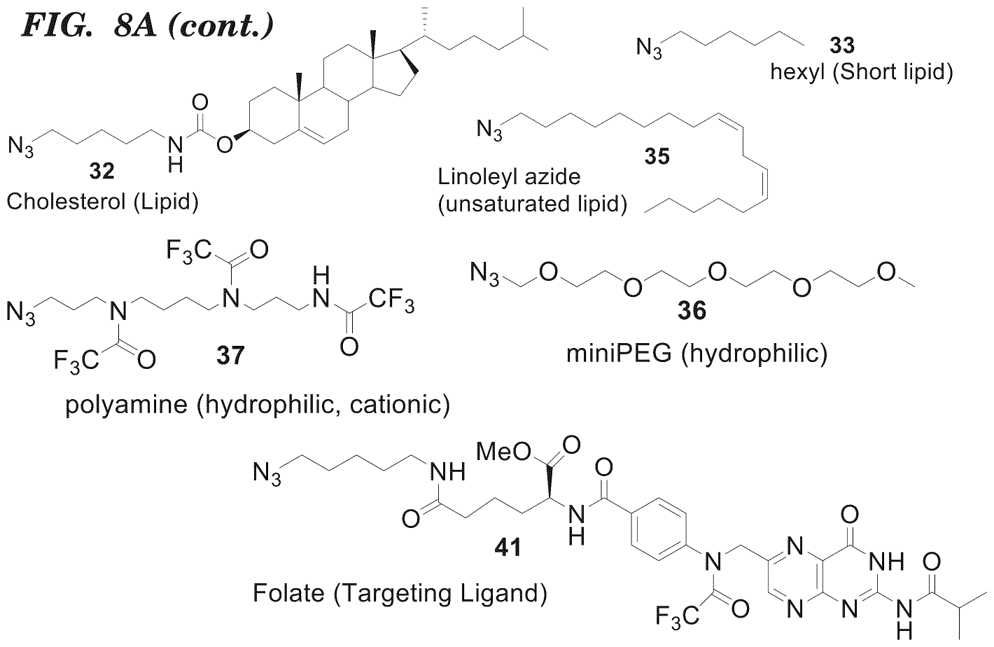

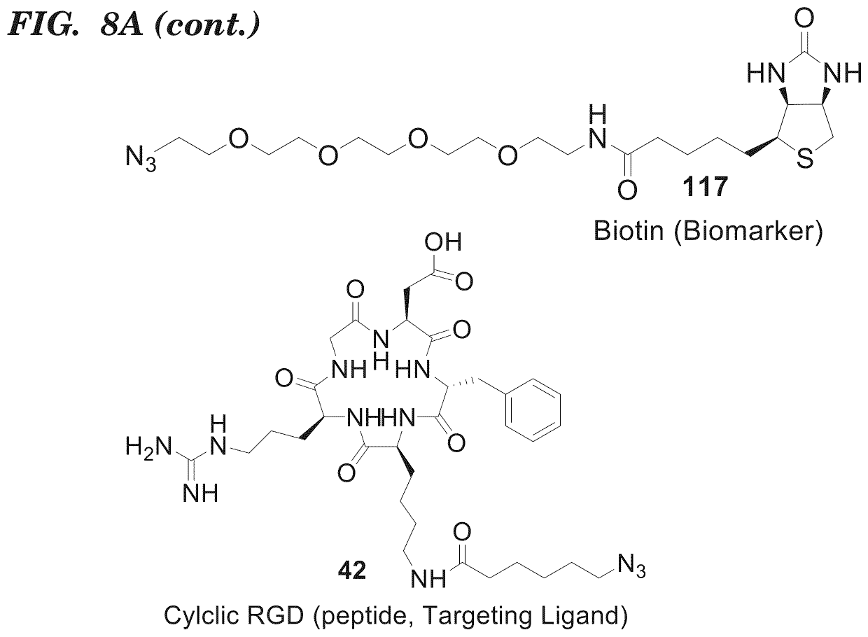

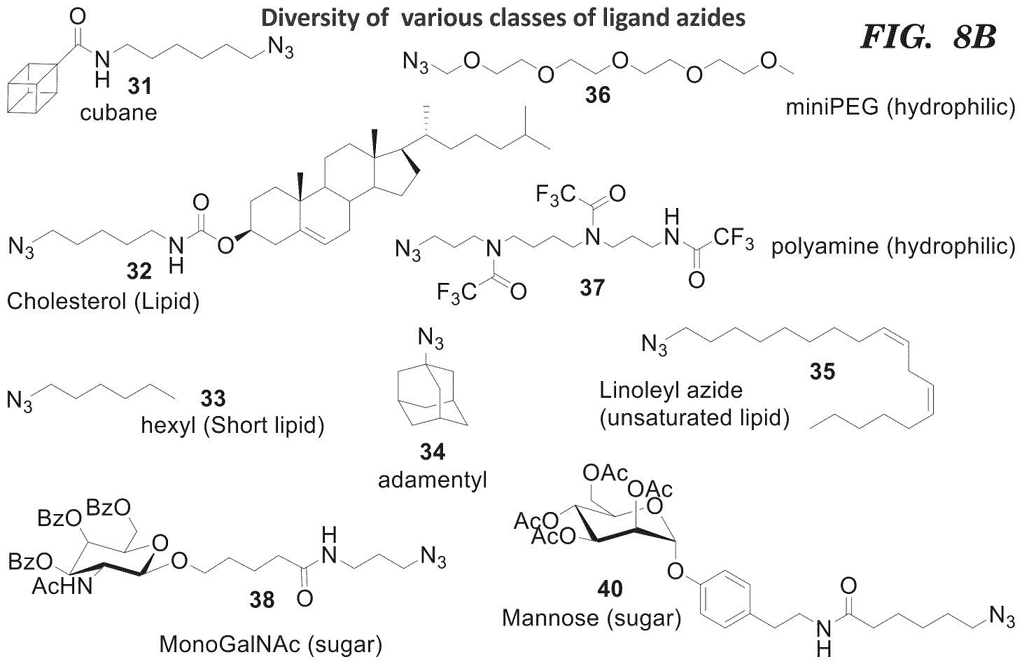

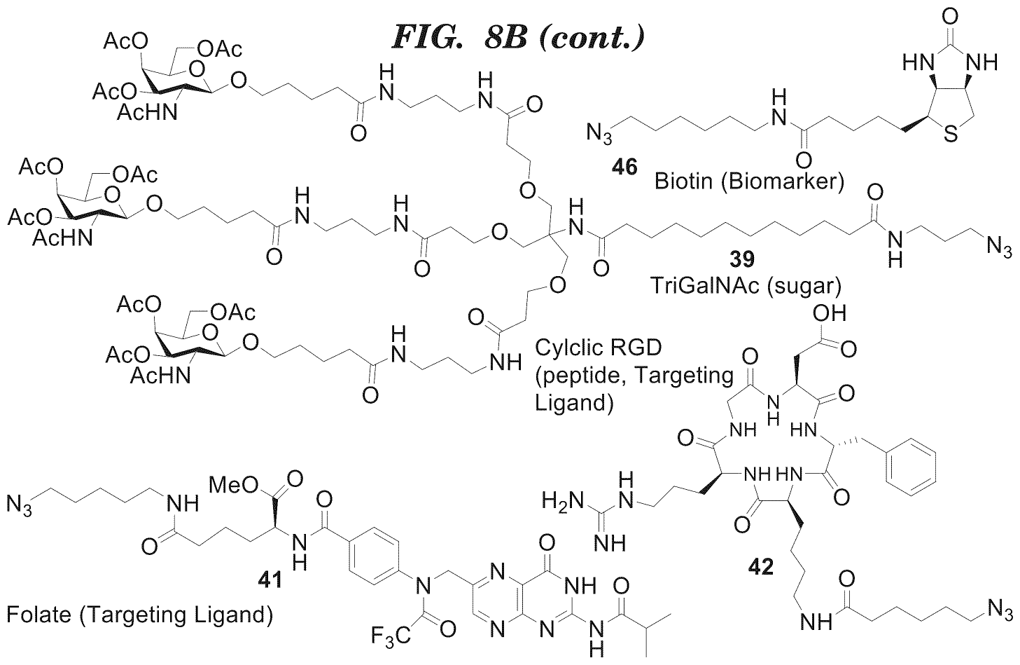

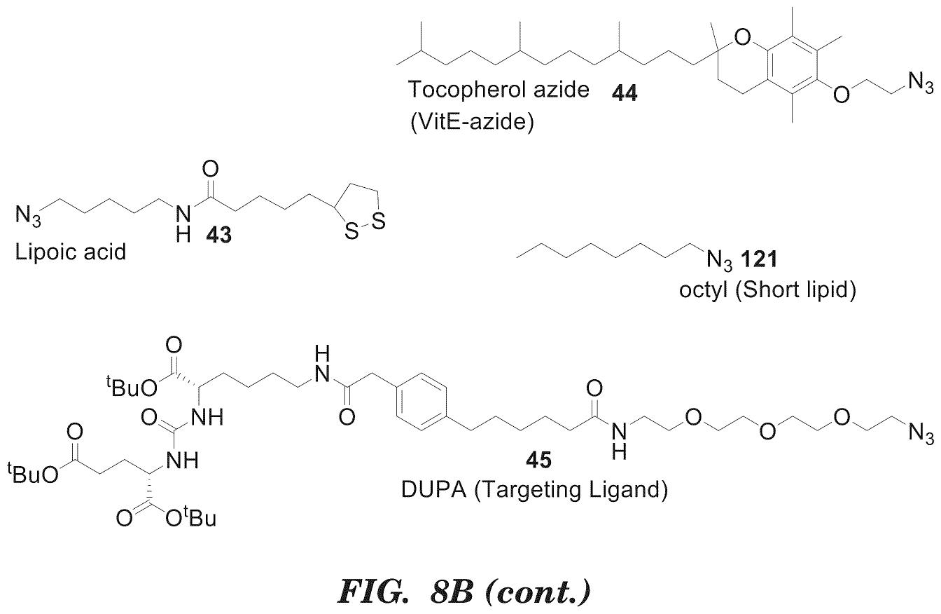

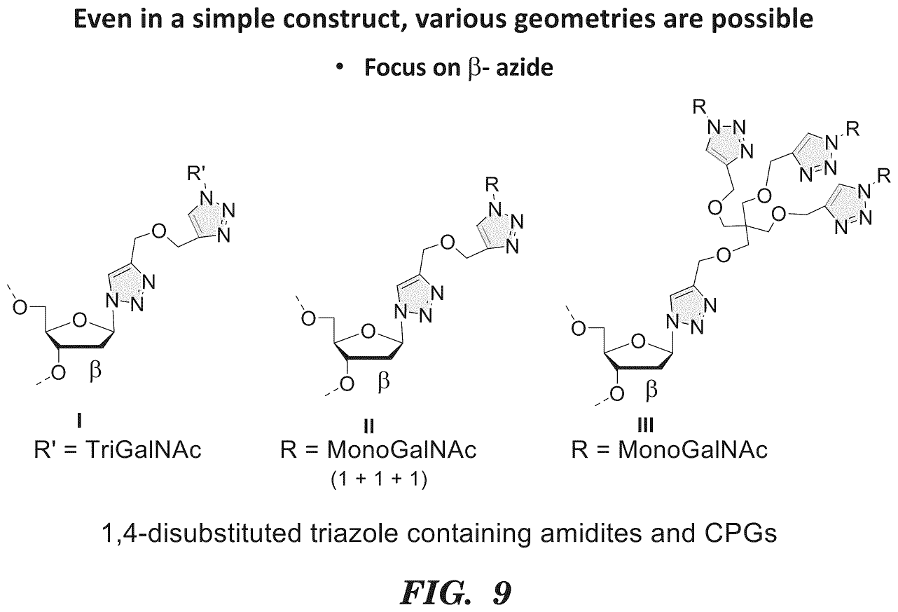

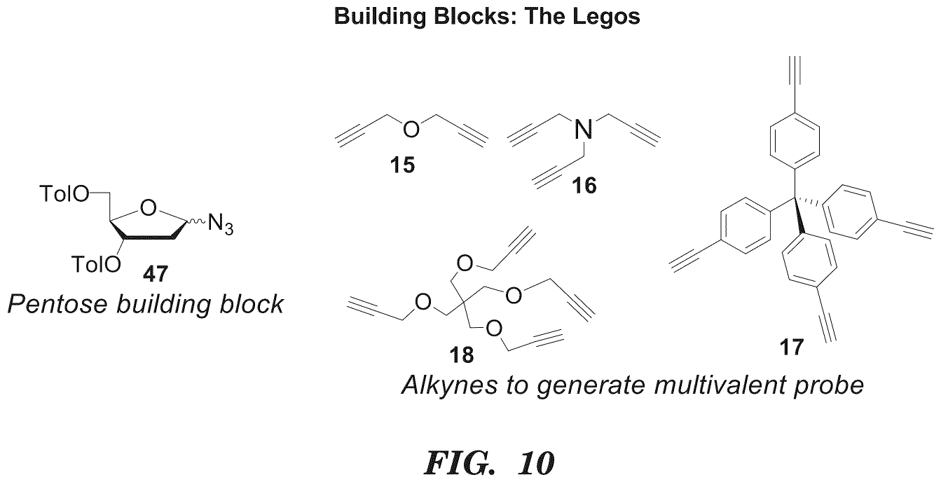

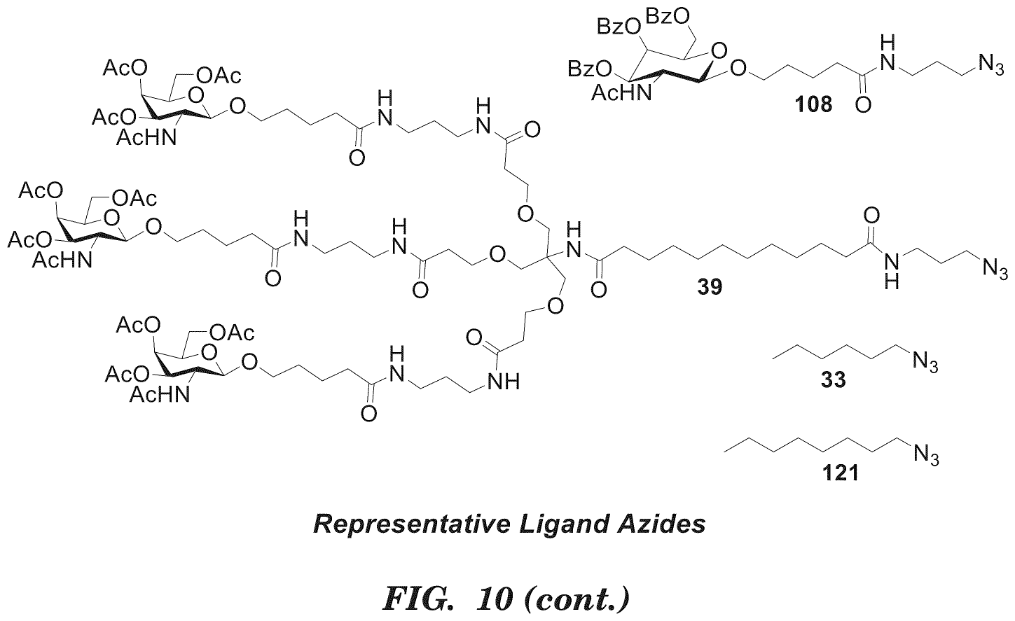

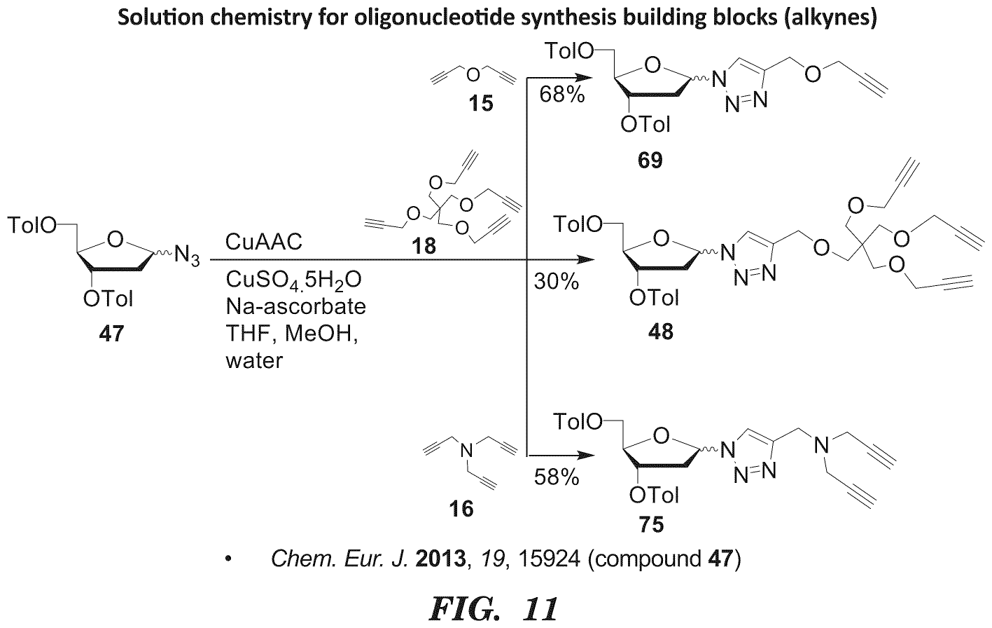

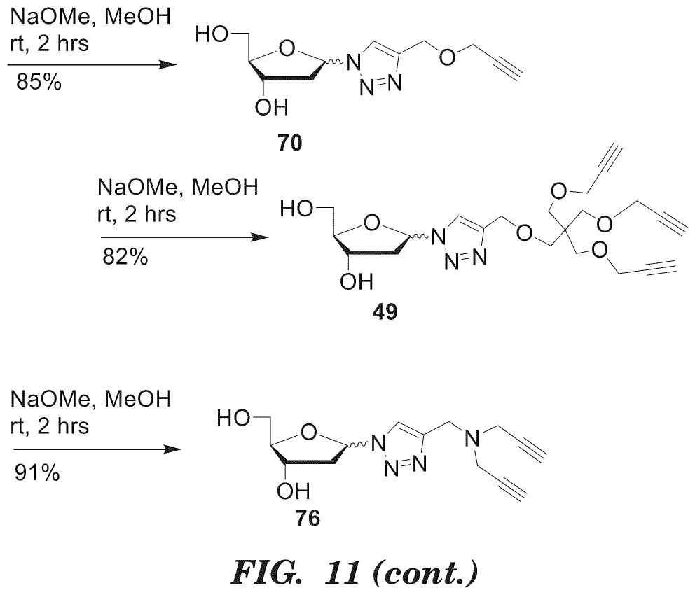

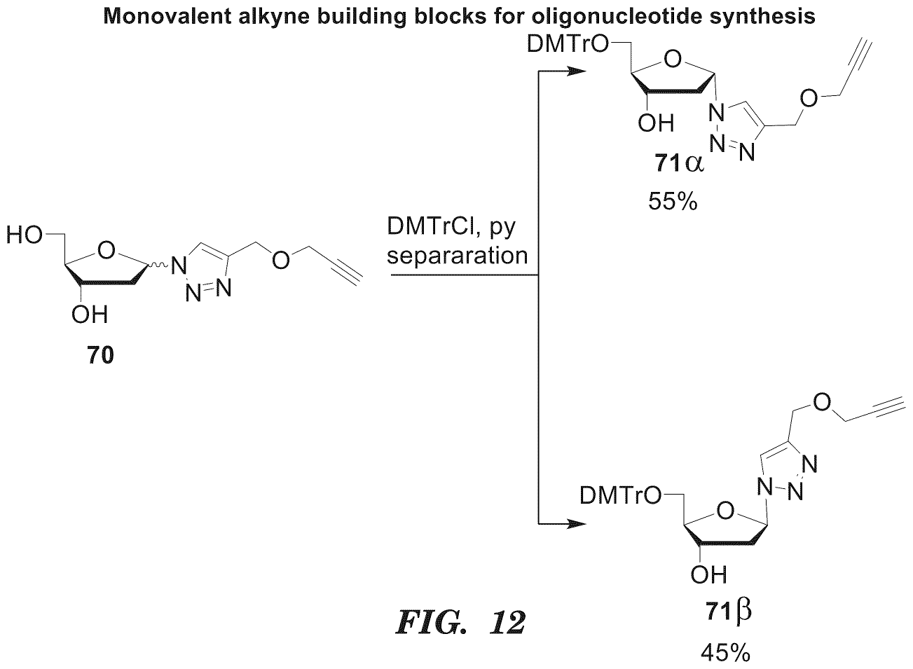

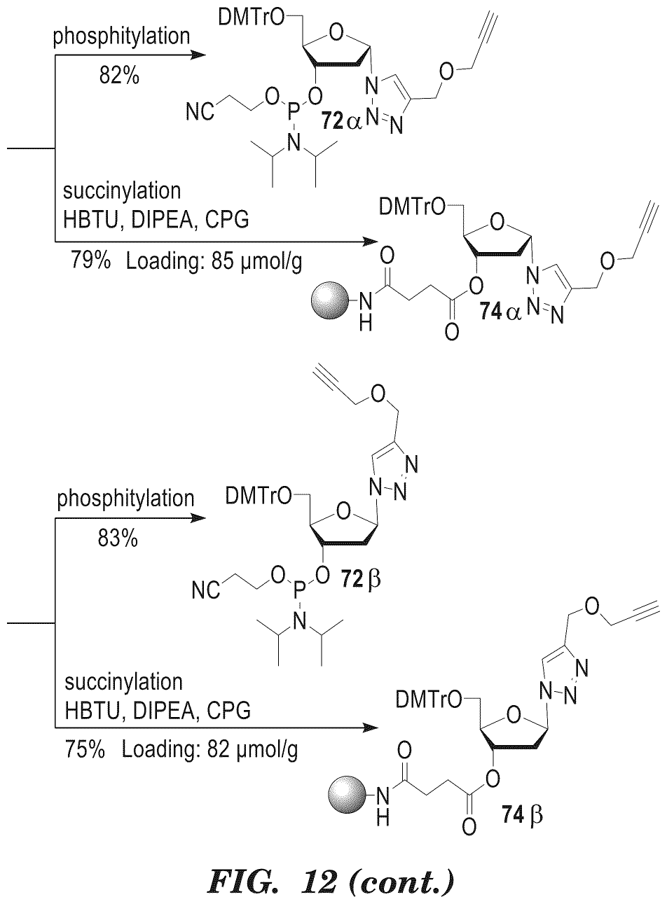

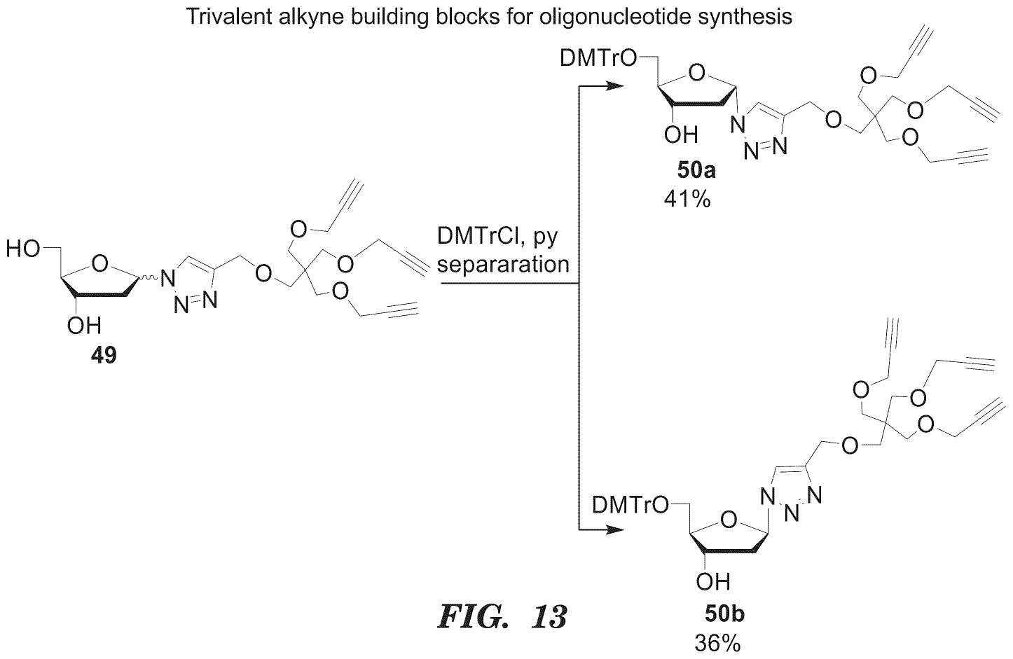

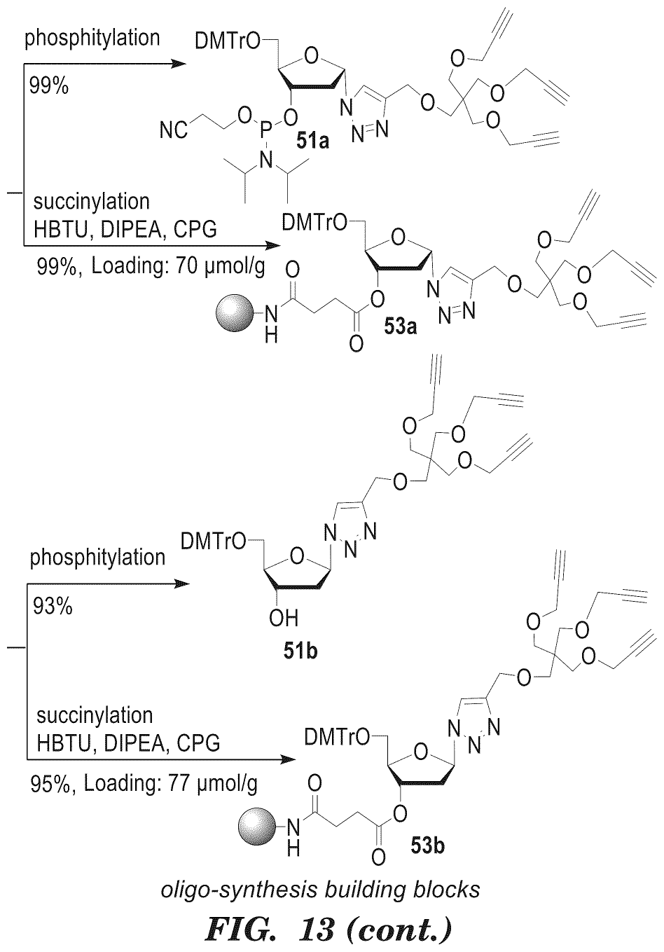

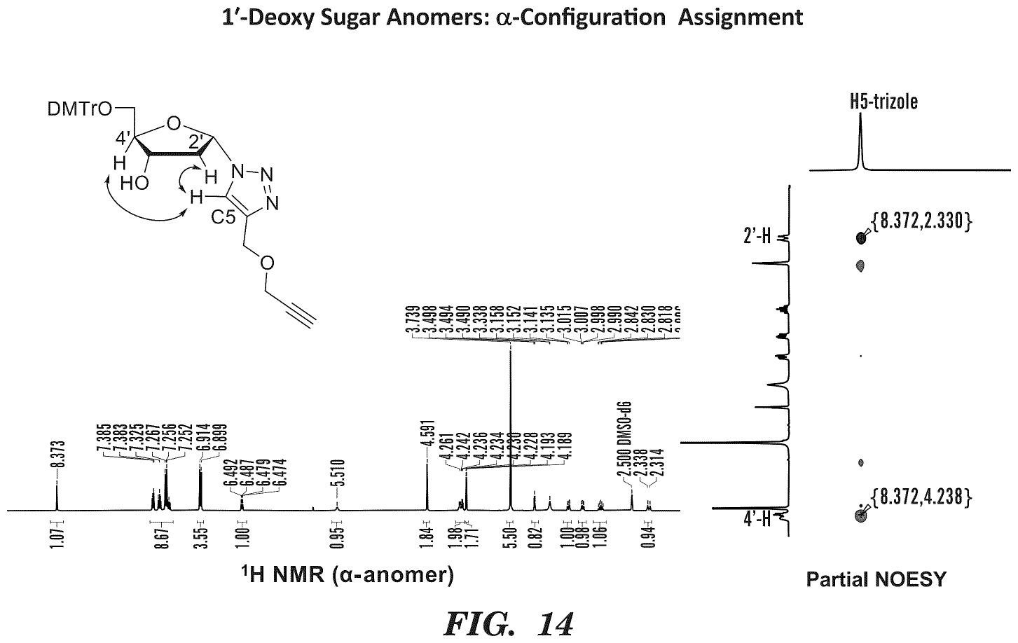

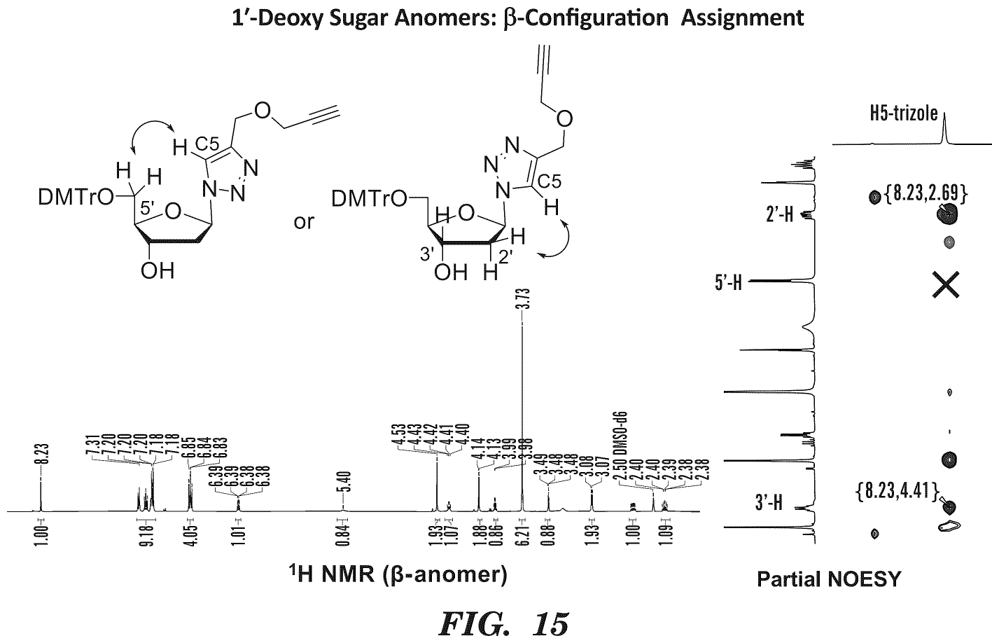

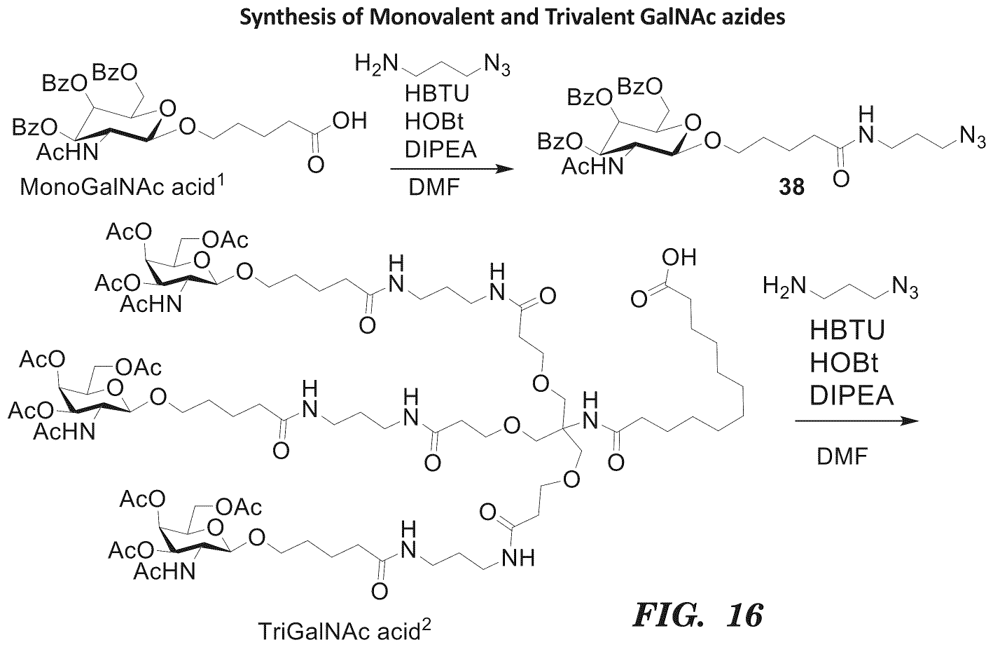

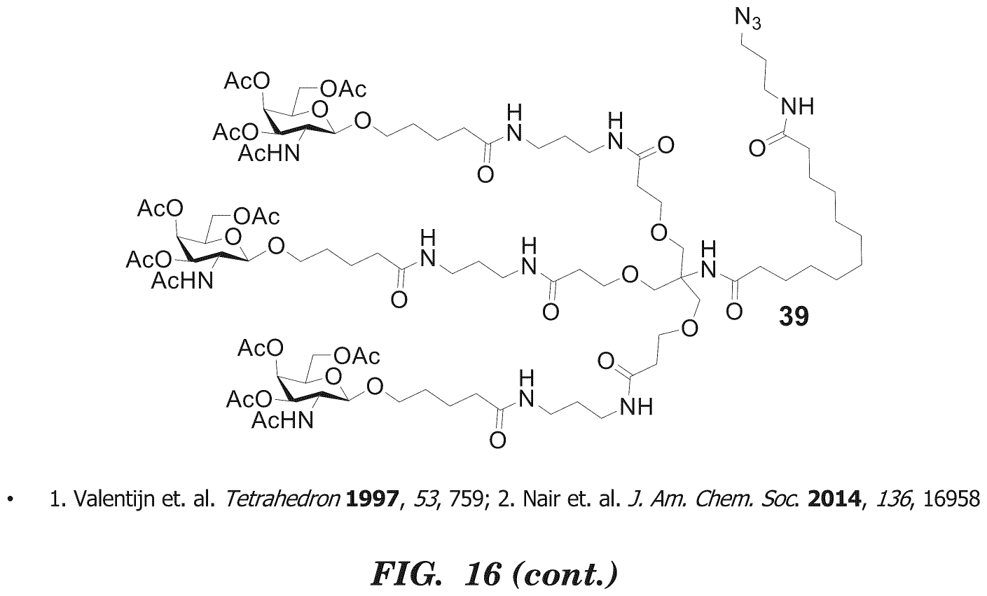

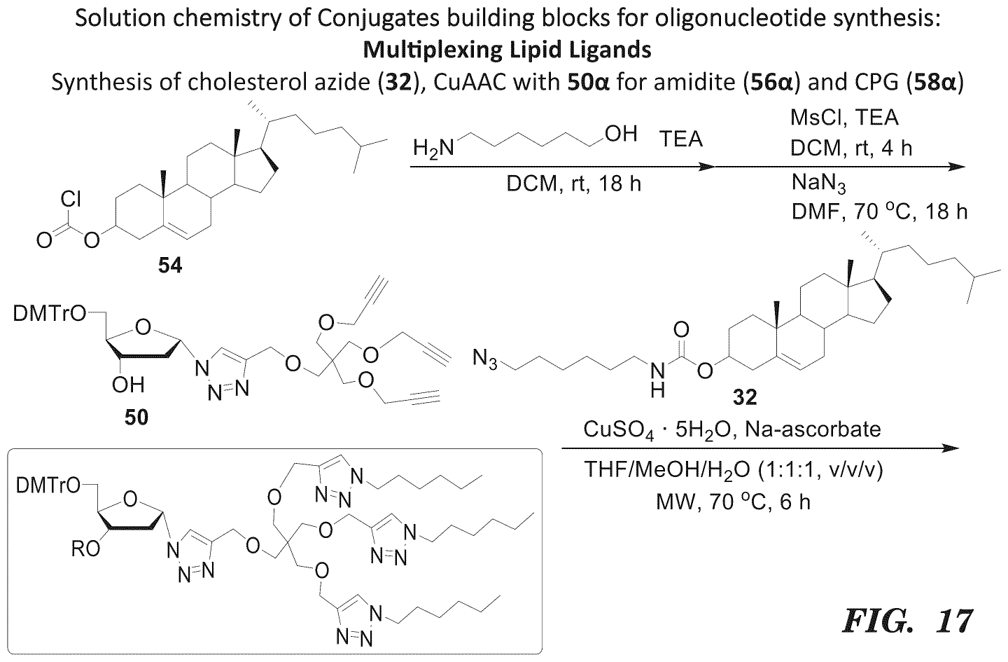

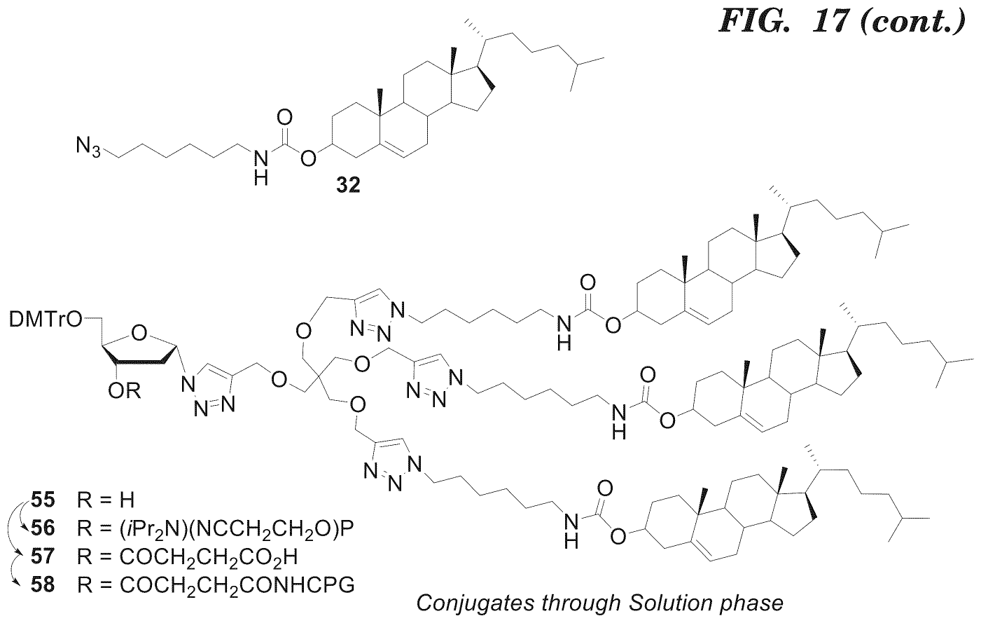

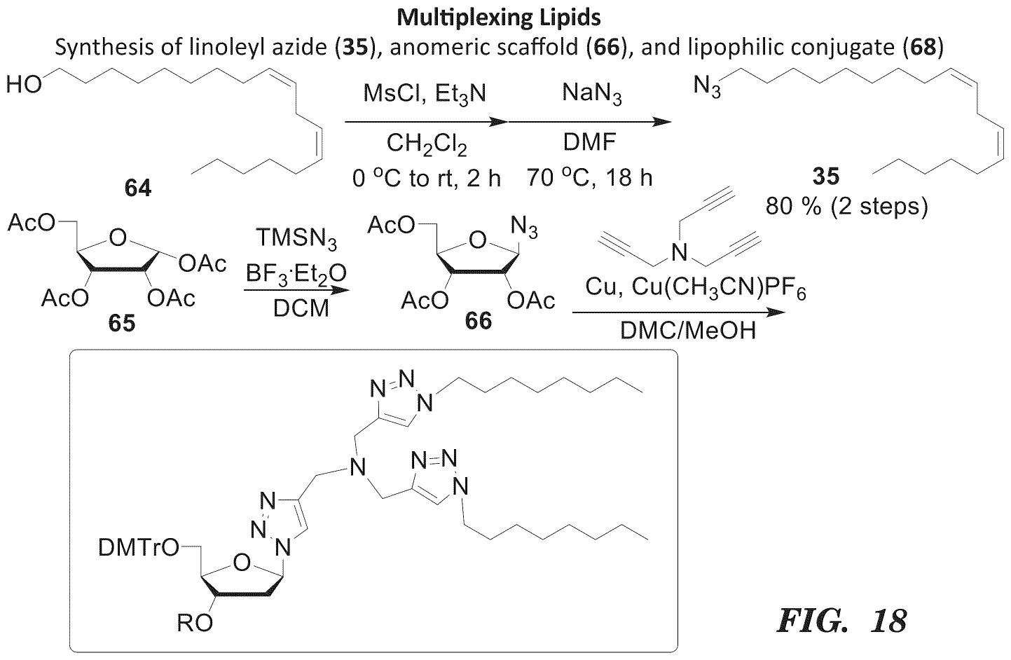

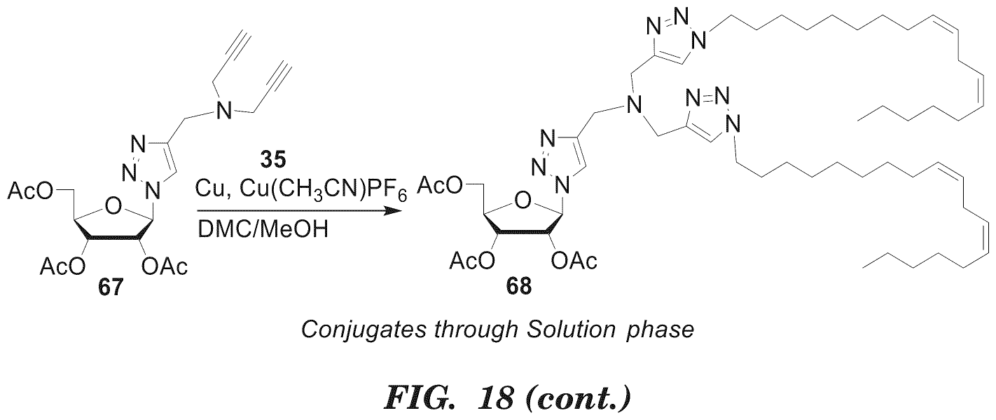

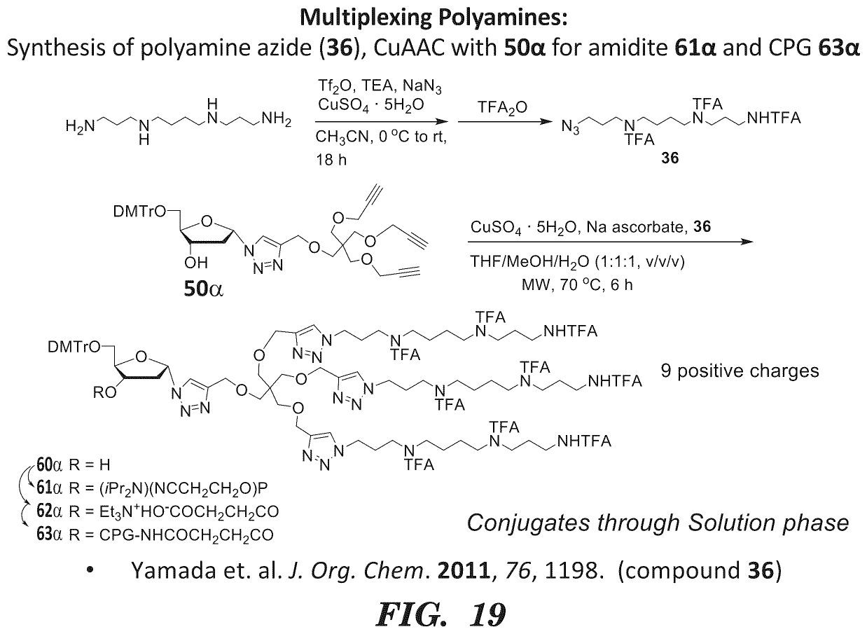

Monomers

| | |
|---|---|
| (M1) | Building block with trivalent alkyne (e.g., compound 49, 50α, 50β, 82, 83α or 83β) or conjugate |
| | (e.g., compound 55α, 60, or compound III of FIG. 9- see FIG. 8A for MonoGalNAc and azide-precursor) |
| (M2) | Building block with monovalent alkyne (e.g., compound 70, 71α or 71β) or conjugate (e.g., compound I or II of FIG. 9- see FIG. 8A for |
| | MonoGalNAc and TriGalNAc azide-precursors) |
| (M3) | Building block with multivalent lipophilic conjugate (e.g., compound 55α, 68, or the examples shown in FIG. 21) |
| L96 | N-[tris(GalNAc-alkyl)-amido-dodecanoyl)]-4-hydroxyprolinol “[Hyp-(GalNAc-alky1)3]” |
| | 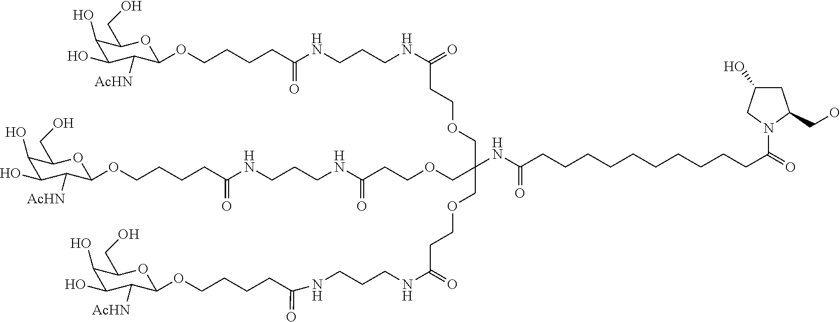 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| (Uhd) | 2′-O-hexadecyl-uridine-3′-phosphate |
| Af | 2′-deoxy-2′-fluoroadenosine-3′-phosphate |
| Afs | 2′-deoxy-2′-fluoroadenosine-3′-phosphorothioate |
| Cf | 2′-deoxy-2′-fluorocytidine-3′-phosphate |
| Cfs | 2′-deoxy-2′-fluorocytidine-3′-phosphorothioate |
| Gf | 2′-deoxy-2′-fluoroguanosine-3′-phosphate |
| Gfs | 2′-deoxy-2′-fluoroguanosine-3′-phosphorothioate |
| Uf | 2′-deoxy-2′-fluorouridine-3′-phosphate |
| Ufs | 2′-deoxy-2′-fluorouridine-3′-phosphorothioate |
| a | 2′-O-methyladenosine-3′-phosphate |
| as | 2′-O-methyladenosine-3′-phosphorothioate |
| c | 2′-O-methylcytidine-3′-phosphate |
| CS | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| S | phosphorothioate linkage |
| VP | 5′-E-vinylphosphonate |

### REFERENCES

1. Fantoni, N. Z.; El-Sagheer, A. H.; Brown, T., A Hitchhiker's Guide to Click-Chemistry with Nucleic Acids. Chem. Rev. (Washington, DC, U. S.) 2021, 121 (12), 7122-7154.
2. Agrahari, A. K.; Bose, P.; Jaiswal, M. K.; Rajkhowa, S.; Singh, A. S.; Hotha, S.; Mishra, N.; Tiwari, V. K., Cu(I)—Catalyzed Click Chemistry in Glycoscience and Their Diverse Applications. Chem. Rev. (Washington, DC, U. S.) 2021, 121 (13), 7638-7956.
3. Perrone, D.; Marchesi, E.; Preti, L.; Navacchia, M. L., Modified nucleosides, nucleotides and nucleic acids via click azide-alkyne cycloaddition for pharmacological applications. Molecules 2021, 26 (11), 3100.
4. Salta, J.; Arp, F. F.; Kuehne, C.; Reissig, H.-U., Multivalent 1,2,3-Triazole-Linked Carbohydrate Mimetics by Huisgen-Meldal-Sharpless Cycloadditions of an Azidopyran. Eur. J. Org. Chem. 2020, 2020 (47), 7333-7342.
5. Johansson, J. R.; Beke-Somfai, T.; Said Staalsmeden, A.; Kann, N., Ruthenium-Catalyzed Azide Alkyne Cycloaddition Reaction: Scope, Mechanism, and Applications. Chem. Rev. (Washington, DC, U. S.) 2016, 116 (23), 14726-14768.
6. Boren, B. C.; Narayan, S.; Rasmussen, L. K.; Zhang, L.; Zhao, H.; Lin, Z.; Jia, G.; Fokin, V. V., Ruthenium-Catalyzed Azide-Alkyne Cycloaddition: Scope and Mechanism. Journal of the American Chemical Society 2008, 130 (28), 8923-8930.
7. Engholm, E.; Stuhr-Hansen, N.; Blixt, O., Facile solid-phase ruthenium assisted azide-alkyne cycloaddition (RuAAC) utilizing the Cp*RuCl(COD)-catalyst. Tetrahedron Letters 2017, 58 (23), 2272-2275.
8. Kim, E.; Koo, H., Biomedical applications of copper-free click chemistry: in vitro, in vivo, and ex vivo. Chemical Science 2019, 10 (34), 7835-7851.
9. Ganz, D.; Harijan, D.; Wagenknecht, H.-A., Labelling of DNA and RNA in the cellular environment by means of bioorthogonal cycloaddition chemistry. RSC Chemical Biology 2020, 1 (3), 86-97.
10. Agard, N. J.; Prescher, J. A.; Bertozzi, C. R., A Strain-Promoted [3+2] Azide-Alkyne Cycloaddition for Covalent Modification of Biomolecules in Living Systems. Journal of the American Chemical Society 2004, 126 (46), 15046-15047.
11. Haun, J. B.; Devaraj, N. K.; Hilderbrand, S. A.; Lee, H.; Weissleder, R., Bioorthogonal chemistry amplifies nanoparticle binding and enhances the sensitivity of cell detection. Nature Nanotechnology 2010, 5 (9), 660-665.
12. Oliveira, B. L.; Guo, Z.; Bernardes, G. J. L., Inverse electron demand Diels-Alder reactions in chemical biology. Chemical Society Reviews 2017, 46 (16), 4895-4950.
13. Kubota, M.; Nainar, S.; Parker, S. M.; England, W.; Furche, F.; Spitale, R. C., Expanding the Scope of RNA Metabolic Labeling with Vinyl Nucleosides and Inverse Electron-Demand Diels-Alder Chemistry. ACS Chemical Biology 2019, 14 (8), 1698-1707.
14. Ding, S.; Jia, G.; Sun, J., Iridium-Catalyzed Intermolecular Azide-Alkyne Cycloaddition of Internal Thioalkynes under Mild Conditions. Angew. Chem., Int. Ed. 2014, 53 (7), 1877-1880.
15. Maity, A.; Choi, J.-S.; Teets, T. S.; Deligonul, N.; Berdis, A. J.; Gray, T. G., Cyclometalated Iridium(III) Complexes with Deoxyribose Substituents. Chem.-Eur. J. 2013, 19 (47), 15924-15932.
16. Powers, A. R.; Yang, X.; Del Castillo, T. J.; Ghiviriga, I.; Abboud, K. A.; Veige, A. S., Inorganic click (iClick) synthesis of heterotrinuclear PtII/AuI2 complexes. Dalton Trans. 2013, 42 (42), 14963-14966.
17. Sultana, J.; Sarma, D., Ag-catalyzed azide-alkyne cycloaddition: copper free approaches for synthesis of 1,4-disubstituted 1,2,3-triazoles. Catal. Rev.: Sci. Eng. 2020, 62 (1), 96-117.
18. Kolb, H. C.; Finn, M. G.; Sharpless, K. B., Click Chemistry: Diverse Chemical Function from a Few Good Reactions. Angewandte Chemie International Edition 2001, 40 (11), 2004-2021.
19. Tornoe, C. W.; Christensen, C.; Meldal, M., Peptidotriazoles on Solid Phase: [1,2,3]-Triazoles by Regiospecific Copper(I)-Catalyzed 1,3-Dipolar Cycloadditions of Terminal Alkynes to Azides. The Journal of Organic Chemistry 2002, 67 (9), 3057-3064.
20. Rostovtsev, V. V.; Green, L. G.; Fokin, V. V.; Sharpless, K. B., A Stepwise Huisgen Cycloaddition Process: Copper(I)-Catalyzed Regioselective “Ligation” of Azides and Terminal Alkynes. Angewandte Chemie International Edition 2002, 41 (14), 2596-2599.
21. Eltepu, L.; Jayaraman, M.; Rajeev, K. G.; Manoharan, M., An immobilized and reusable Cu(i) catalyst for metal ion-free conjugation of ligands to fully deprotected oligonucleotides through click reaction. Chemical Communications 2013, 49 (2), 184-186.
22. Alam, M. R.; Ming, X.; Fisher, M.; Lackey, J. G.; Rajeev, K. G.; Manoharan, M.; Juliano, R. L., Multivalent Cyclic RGD Conjugates for Targeted Delivery of Small Interfering RNA. Bioconjugate Chem. 2011, 22 (8), 1673-1681.
23. Stuhr-Hansen, N.; Risinger, C.; Engholm, E.; Blixt, O., Synthesis of oligo-(alkyne-triplet)peptide constructs. Org. Lett. 2017, 19 (24), 6522-6525.
24. Maksimovic, I.; Zheng, Q.; Trujillo, M. N.; Galligan, J. J.; David, Y., An Azidoribose Probe to Track Ketoamine Adducts in Histone Ribose Glycation. J. Am. Chem. Soc. 2020, 142 (22), 9999-10007.
25. Valentijn, A. R. P. M.; van der Marel, G. A.; Sliedregt, L. A. J. M.; van Berkel, T. J. C.; Biessen, E. A. L.; van Boom, J. H., Solid-phase synthesis of lysine-based cluster galactosides with high affinity for the asialoglycoprotein receptor. Tetrahedron 1997, 53 (2), 759-770.
26. Nair, J. K.; Willoughby, J. L. S.; Chan, A.; Charisse, K.; Alam, M. R.; Wang, Q.; Hoekstra, M.; Kandasamy, P.; Kel'in, A. V.; Milstein, S.; Taneja, N.; O'Shea, J.; Shaikh, S.; Zhang, L.; van der Sluis, R. J.; Jung, M. E.; Akinc, A.; Hutabarat, R.; Kuchimanchi, S.; Fitzgerald, K.; Zimmermann, T.; van Berkel, T. J. C.; Maier, M. A.; Rajeev, K. G.; Manoharan, M., Multivalent N-Acetylgalactosamine-Conjugated siRNA Localizes in Hepatocytes and Elicits Robust RNAi-Mediated Gene Silencing. J. Am. Chem. Soc. 2014, 136 (49), 16958-16961.
27. Jayaprakash, K. N.; Peng, C.-G.; Butler, D.; Varghese, J. P.; Maier, M. A.; Rajeev, K. G.; Manoharan, M., Non-Nucleoside Building Blocks for Copper-Assisted and Copper-Free Click Chemistry for the Efficient Synthesis of RNA Conjugates. Org. Lett. 2010, 12 (23), 5410-5413.
28. Yamada, T.; Peng, C. G.; Matsuda, S.; Addepalli, H.; Jayaprakash, K. N.; Alam, M. R.; Mills, K.; Maier, M. A.; Charisse, K.; Sekine, M.; Manoharan, M.; Rajeev, K. G., Versatile Site-Specific Conjugation of Small Molecules to siRNA Using Click Chemistry. The Journal of Organic Chemistry 2011, 76 (5), 1198-1211.
29. Johansson, J. R.; Beke-Somfai, T.; Said Stilsmeden, A.; Kann, N., Ruthenium-Catalyzed Azide Alkyne Cycloaddition Reaction: Scope, Mechanism, and Applications. Chemical Reviews 2016, 116 (23), 14726-14768.
30. Molla, R. A.; Roy, A. S.; Ghosh, K.; Salam, N.; Iqubal, M. A.; Tuhina, K.; Islam, S. M., Polymer anchored ruthenium complex: A highly active and recyclable catalyst for one-pot azide-alkyne cycloaddition and transfer-hydrogenation of ketones under mild conditions. Journal of Organometallic Chemistry 2015, 776, 170-179.
31. Zhang, L.; Chen, X.; Xue, P.; Sun, H. H. Y.; Williams, I. D.; Sharpless, K. B.; Fokin, V. V.; Jia, G., Ruthenium-Catalyzed Cycloaddition of Alkynes and Organic Azides. Journal of the American Chemical Society 2005, 127 (46), 15998-15999.
32. Chen, W.; Park, S. K.; Yu, W.; Xiong, A.; Sanders, B. G.; Kline, K., Synthesis and screening of novel vitamin E derivatives for anticancer functions. Eur. J. Med. Chem. 2012, 58, 72-83.
33. Liu, J.; Feng, M.; Liang, D.; Yang, J.; Tang, X., Vitamin E-Labeled Polyethylenimine for in vitro and in vivo Gene Delivery. Biomacromolecules 2016, 17 (10), 3153-3161.
34. Farzan, V. M.; Ulashchik, E. A.; Martynenko-Makaev, Y. V.; Kvach, M. V.; Aparin, I. O.; Brylev, V. A.; Prikazchikova, T. A.; Maklakova, S. Y.; Majouga, A. G.; Ustinov, A. V.; Shipulin, G. A.; Shmanai, V. V.; Korshun, V. A.; Zatsepin, T. S., Automated Solid-Phase Click Synthesis of Oligonucleotide Conjugates: From Small Molecules to Diverse N-Acetylgalactosamine Clusters. Bioconjugate Chem. 2017, 28 (10), 2599-2607.
35. Hofmeister, A.; Jahn-Hofmann, K.; Brunner, B.; Helms, M. W.; Metz-Weidmann, C.; Krack, A.; Kurz, M.; Li, Z.; Weitzenberg, M. M.; Pflimlin, E.; Plettenburg, O.; Scheidler, S., Syntheses of Morpholine-Based Nucleotide Analogs for Hepatic siRNA Targeting and Stabilization. J. Med. Chem. 2021, 64 (10), 6838-6855.
36. Tanaka, T.; Nagai, H.; Noguchi, M.; Kobayashi, A.; Shoda, S.-i., One-step conversion of unprotected sugars to β-glycosyl azides using 2-chloroimidazolinium salt in aqueous solution. Chem. Commun. (Cambridge, U. K)₂₀₀₉, (23), 3378-3379.
37. Fuchs, P.; Vana, P.; Zhang, K., Copper(I)-catalyzed azide-alkyne cycloaddition-assisted polymerization of linear glucose-derived co/polymers. Journal of Polymer Science 2020, 58 (11), 1535-1543.
38. Banerjee, A.; Senthilkumar, S.; Baskaran, S., Benzylidene Acetal Protecting Group as Carboxylic Acid Surrogate: Synthesis of Functionalized Uronic Acids and Sugar Amino Acids. Chemistry—A European Journal 2016, 22 (3), 902-906.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. A compound of Formula (III), (IV), (VI), (VII), (VIII) or (IX): 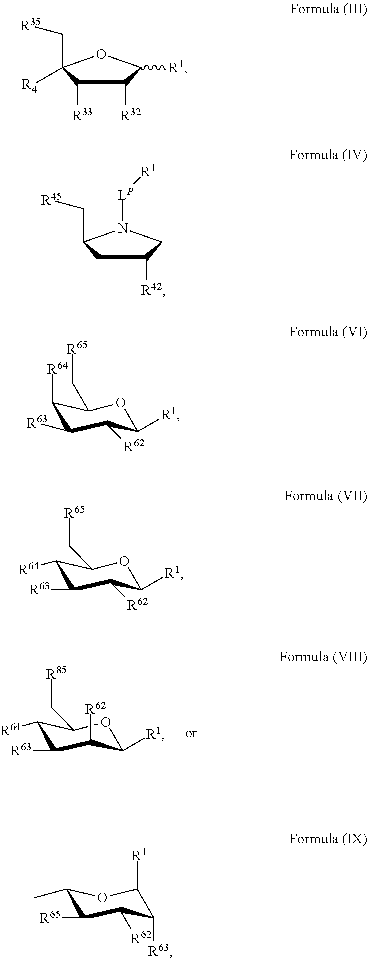
wherein:
L^{P} is absent or a linker;
R¹ is N₃ or 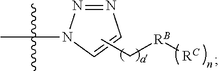
wherein:
a′ is 0 or 1;
n is 1, 2, 3, 4, or 5;
R^{B} is O, N, S, a heteroalkyl, a branched alkyl, a cycloalkyl, heterocyclyl, aryl (e.g., phenyl), or heteroaryl;
each R^{C} independently is 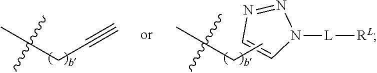
wherein:
each b′ is independently 0 or 1;
each L independently is absent or linker;
each R^{L} is a ligand, (e.g., selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, and polyethylene glycols (PEGs));
R³² is hydrogen, hydroxy, halogen, protected hydroxy, phosphate group, reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R³³ is hydrogen, hydroxy, halogen, protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker or a linker covalently bonded (e.g., —C(O)CH₂CH₂C(O)—) to a solid support, and optionally, only one of R³² and R³³ is a phosphate group, a reactive phosphorous group, a solid support, a linker, or a linker covalently bonded (e.g., —C(O)CH₂CH₂C(O)—) to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R³² taken together are 4′—C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
v is 1, 2 or 3;
or R⁴ and R³³ taken together with the atoms to which they are attached form an optionally substituted C₃₋₈cycloalkyl, optionally substituted C₃₋₈cycloalkenyl, or optionally substituted 3-8 membered heterocyclyl;
R³⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10);
R⁴² is hydroxy, halogen, protected hydroxy, phosphate group, reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R⁴⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10);
R⁶² is hydroxy, protected hydroxy, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —C(O)CH₂CH₂C(O)—) to a solid support;
R⁶³ and R⁶⁴ independently are hydrogen, hydroxy, halogen, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), or —O-lipid;
R⁶⁵ is hydroxy, protected hydroxy, phosphate group, optionally substituted C₁₋₃₀ alkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10); and
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl.

2. (canceled)

3. The compound of claim 1, wherein R¹ is

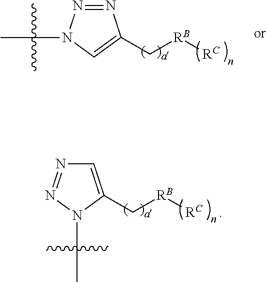

4. The compound of claim 1, wherein R^{c} is

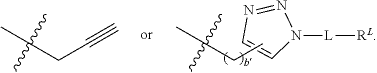

5. The compound of claim 4, wherein R^{c} is

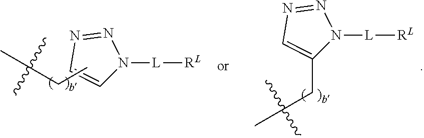

6. (canceled)

7. The compound of claim 1, wherein at least one R^{L} is selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof.

8. (canceled)

9. The compound of claim 1, wherein n is 1, 2, 3 or 5.

10. The compound of claim 1 wherein R^{B} is O, N, C(CH₂O—)₄, benzyl or

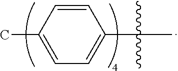

11.-52. (canceled)

53. The compound of claim 1, wherein the compound is of Formula (IIIc): 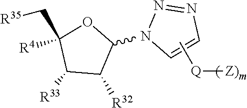
wherein
Q, Z, and m are defined as one of sets (i), (ii) or (iii), wherein
(i) Q is optionally substituted aryl (e.g., phenyl) or optionally substituted heteroaryl;
m is an integer selected from 1 to the maximum number of substituents for Q (e.g., when Q is phenyl, then m is 1, 2, 3, 4 or 5, such as 1, 2 or 3; or 1 or 2);
and each Z is —Z¹, —Z², or —C(R^{C})₃, wherein
R^{C} is aryl (e.g., phenyl or naphthyl) or heteroaryl, each substituted with one or more Z¹ or Z² groups (e.g., 1, 2, or 3);
each Z¹ is selected from the group consisting of 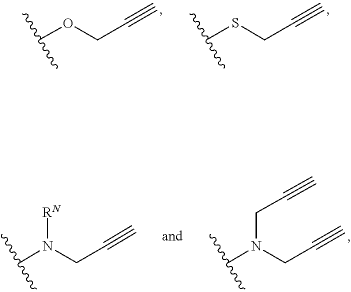
wherein R^{N} is hydrogen or C₁₋₆ alkyl; and
Z² is 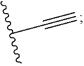 [SMILES: *C#C]
or
(ii) m is 1;
Q is —CH₂O—, —CH₂S—, or —CH₂N(R^{N})—, wherein the N, O, or S is bonded to Z;
and Z is 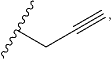 [SMILES: *CC#C]
—(CH₂)₀₋₁—Y—(Z³)ₚ, —C(H)(CH₂Z¹)₂, —CH₂C(H)(CH₂Z¹)₂, or —CH₂C(CH₂Z¹)₃, wherein
Y is optionally substituted aryl or optionally substituted heteroaryl;
each Z³ is Z¹ or Z²; and
p is an integer selected from 1 to the maximum number of substituents for Y (e.g., when Y is phenyl, then p is 1, 2, 3, 4 or 5, such as 1, 2, or 3; or 1 or 2);
or
(iii) Q is —CH₂N—;
m is 2;
and each Z is 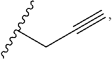 [SMILES: *CC#C]
—(CH₂)₀₋₁—Y—(Z³)ₚ, or —CH₂C(CH₂Z¹)₃.

54.-56. (canceled)

57. The compound of claim 1, wherein the compound is of Formula (IVb): 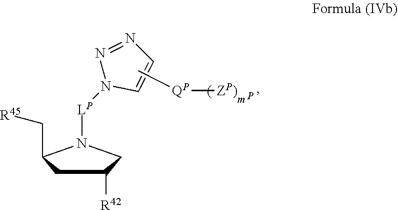
wherein:
Q^{P}, Z^{P}, and m^{P} are defined as one of sets (i), (ii) or (iii), wherein
(i) Q^{P} is optionally substituted aryl (e.g., phenyl) or optionally substituted heteroaryl;
m^{P} is an integer selected from 1 to the maximum number of substituents for Q^{P} (e.g., when Q^{P} is phenyl, then m is 1, 2, 3, 4 or 5, such as 1, 2 or 3; or 1 or 2);
and each Z^{P} is —Z^{P1}, —Z^{P2}, or —C(R^{PC})₃, wherein
R^{PC} is aryl (e.g., phenyl or naphthyl) or heteroaryl, each substituted with one or more Z^{P1} or Z^{P2} groups (e.g., 1, 2, or 3);
each Z^{P} is selected from the group consisting of 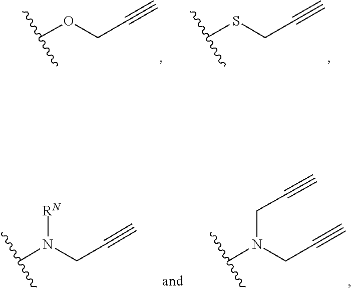
wherein R^{N} is hydrogen or C₁₋₆ alkyl; and
Z^{P2} is 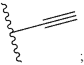 [SMILES: *C#C]
(ii) m^{P} is 1;
Q^{P} is —CH₂O—, —CH₂S—, or —CH₂N(R^{N})—, wherein the N, O, or S is bonded to Z^{P};
and Z^{P} is 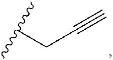 [SMILES: *CC#C]
—(CH₂)₀₋₁—Y—(Z³)ₚₚ, —C(H)(CH₂Z^{P1})₂, —CH₂C(H)(CH₂Z^{P1})₂, or —CH₂C(CH₂Z^{P1})₃, wherein
Y^{P} is optionally substituted aryl or optionally substituted heteroaryl;
each Z^{P3} is Z^{P1} or Z^{P2}; and
pp is an integer selected from 1 to the maximum number of substituents for Y^{P} (e.g., when Y^{P} is phenyl, then pp is 1, 2, 3, 4 or 5, such as 1, 2, or 3; or 1 or 2);
or
(iii) Q^{P} is —CH₂N—;
m^{P} is 2;
and each Z^{P} is 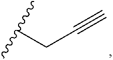 [SMILES: *CC#C]
—(CH₂)₀₋₁—Y—(Z^{P3})ₚₚ, or —CH₂C(CH₂Z^{P1})₃.

58. (canceled)

59. The compound of claim 1, wherein the compound is of Formula VIb, VIIb, VIIIb or IXb: 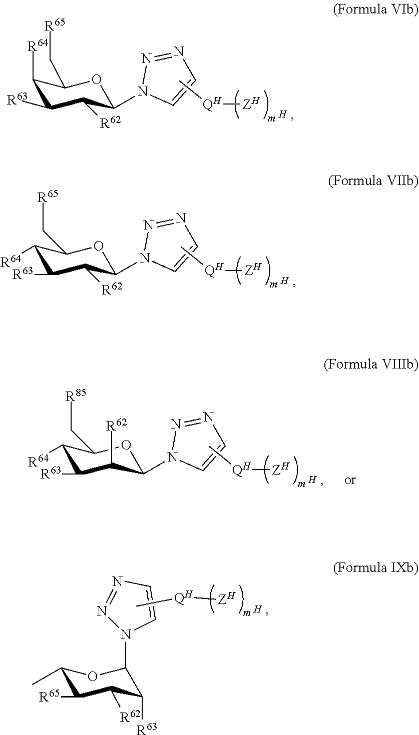
wherein:
Q^{H}, Z^{H}, andm^{H} are defined as one of sets (i), (ii) or (iii), wherein
(i) Q^{H} is optionally substituted aryl (e.g., phenyl) or optionally substituted heteroaryl;
m^{H} is an integer selected from 1 to the maximum number of substituents for Q^{H} (e.g., when Q^{H} is phenyl, then m is 1, 2, 3, 4 or 5, such as 1, 2 or 3; or 1 or 2);
and each Z^{H} is —Z^{H1}, —Z^{H2}, or —C(R^{HC})₃, wherein
R^{HC} is aryl (e.g., phenyl or naphthyl) or heteroaryl, each substituted with one or more Z^{H1} or Z^{H2} groups (e.g., 1, 2, or 3);
each Z^{H1} is selected from the group consisting of 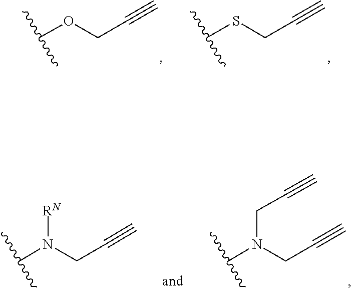
wherein R^{N} is hydrogen or C₁₋₆ alkyl; and
Z^{H2} is 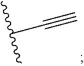 [SMILES: *C#C]
(ii) m^{H} is 1;
Q^{H} is —CH₂O—, —CH₂S—, or —CH₂N(R^{N})—, wherein the N, O, or S is bonded to Z^{H};
and Z^{H} is 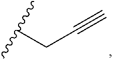 [SMILES: *CC#C]
—(CH₂)₀₋₁—Y—(Z^{H3})ₕₚ, —C(H)(CH₂Z^{P1})₂, —CH₂C(H)(CH₂Z^{H1})₂, or —CH₂C(CH₂Z^{H1})₃, wherein
Y^{H} is optionally substituted aryl or optionally substituted heteroaryl;
each Z^{H3} is ZHl or Z^{H2}; and
hp is an integer selected from 1 to the maximum number of substituents for Y^{H} (e.g., when Y^{H} is phenyl, then hp is 1, 2, 3, 4 or 5, such as 1, 2, or 3; or 1 or 2);
or
(iii) Q^{H} is —CH₂N—;
m^{H} is 2;
and each Z^{H} is 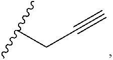 [SMILES: *CC#C]
—(CH₂)₀₋₁—Y—(Z^{H3})ₕₚ, or —CH₂C(CH₂Z^{H1})₃.

60. (canceled)

61. A composition that is an azide-alkyne cycloaddition (AAC) reaction product of a first compound of claim 53 and a second compound of the formula R^{L}-L-N₃, wherein L is a linker and R^{L} is a ligand.

62.-64. (canceled)

65. An oligonucleotide comprising a nucleoside of Formula (I), (V), (VIx), (VIIx), (VIIIx) or (IXx): 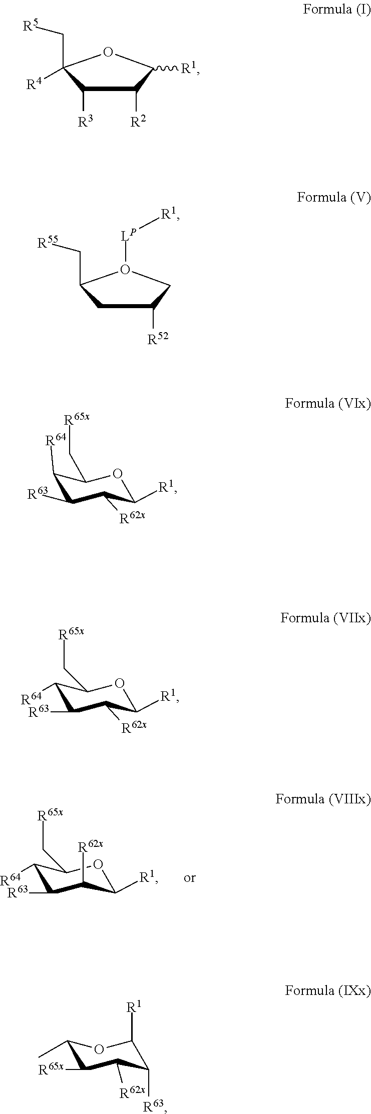
wherein:
L^{P} is absent or a linker;
R¹ is N₃ or 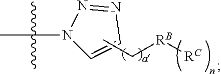
wherein:
a′ is 0 or 1;
n is 1, 2, 3, 4, or 5;
R^{B} is O, N, S, a heteroalkyl, a cycloalkyl, heterocyclyl, aryl or heteroaryl;
each R^{C} independently is 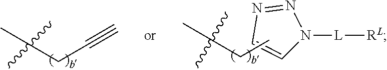
wherein:
each b′ is 0 or 1;
each L independently is absent or linker;
each R^{L} is selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, and polyethylene glycols (PEGs);
R² is hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a bond to an internucleotide linkage to a subsequent nucleotide, a 3′-oligonuclotide capping group, a ligand, a linker covalently bonded to one or more ligands, a solid support, a linker or a linker covalently bonded a solid support;
R³, R⁵² and R^{63x} are independently a bond to an internucleotide linkage to a subsequent nucleotide, hydrogen, hydroxy, protected hydroxy, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a 3′-oligonuclotide capping group, a ligand, a linker covalently bonded to one or more ligands, a solid support, a linker or a linker covalently bonded to a solid support;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R² taken together are 4′—C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R^{a13})C(O)—, or —C(O)N(R¹²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group;
v is 1, 2 or 3;
or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl;
R⁵, R⁵⁵ and R^{65x} represent independently a bond to an internucleotide linkage to a preceding nucleotide, hydrogen, hydroxy, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy, optionally substituted 3-8 membered heterocyclyl (e.g., morpholin-1-yl, piperidin-1-yl, or pyrrolidin-1-yl), halogen, alkoxyalkyl (e.g., 2-methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆ cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates [(R)(OH)(O)P—O-5′, R^{P} is optionally substituted C₁₋₃₀ alkyl, e.g., methyl, ethyl, isopropyl, or propyl)], alkyletherphosphonates [(R^{P1})(OH)(O)P—O-5′, RP¹ is alkoxyalkyl, e.g., methoxymethyl (CH₂OMe) or ethoxymethyl], (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein
X is O or S;
a and b are each independently 1-10;
R⁶³ and R⁶⁴ independently are hydrogen, hydroxy, halogen, protected hydroxy, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), or —O-lipid;
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl,
provided that,
(i) no more than one of R² and R³ is a bond to an internucleotide linkage to a subsequent nucleotide;
(ii) when both of R² and R³ are not a bond to an internucleotide linkage to a subsequent nucleotide, then R⁵ is a bond to an internucleotide linkage to a preceding nucleotide;
(iii) when R² is not a bond to an internucleotide linkage to a subsequent nucleotide, then R is a bond to an internucleotide linkage to a preceding nucleotide; and
(iv) when R⁵⁵ is not a bond to an internucleotide linkage to a preceding nucleotide, then R⁵² is a bond to an internucleotide linkage to a subsequent nucleotide.

66. (canceled)

67. The oligonucleotide of claim 65, wherein R¹ is

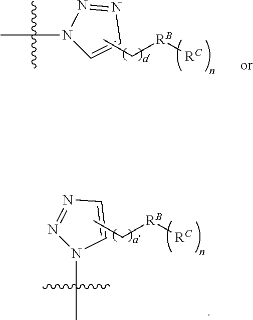

68. The oligonucleotide of claim 65, wherein R^{C} is

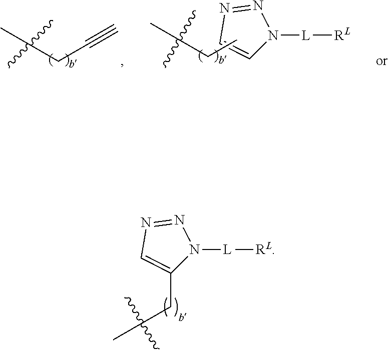

69. The oligonucleotide of claim 65 wherein R¹ is

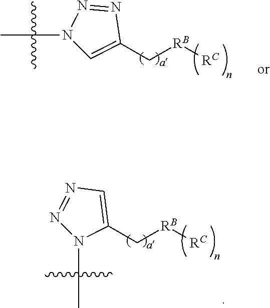

70. (canceled)

71. The oligonucleotide of claim 65, wherein at least one R^{L} is selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof.

72. (canceled)

73. The oligonucleotide of claim 65, wherein n is 1, 2, 3 or 5.

74. The oligonucleotide of claim 65, wherein R^{B} is O, N, C(CH₂O—)₄, benzyl, or

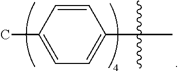

75.-109. (canceled)

110. A double-stranded nucleic acid comprising a first oligonucleotide strand and a second oligonucleotide strand substantially complementary to the first strand, wherein the first or second strand is an oligonucleotide of claim 65.

111.-115. (canceled)

116. A method of reducing the expression of a target gene in a subject, comprising administering to the subject:
an oligonucleotide of claim 65, wherein the oligonucleotide is complementary to a target gene.


SEQUENCE LISTING (48 sequences)

SEQ ID NO: 1 - RNA, 21 nt, synthetic construct
  source 1..21: mol_type = other RNA
  modified_base 1: mod_base = OTHER; note = 2-prime-deoxy-2-prime-fluoroadenosine-3-prime-phosp horothioate
  modified_base 2: mod_base = OTHER; note = 2-prime-O-methyladenosine-3-prime- phosphorothioate
  modified_base order(3,10): mod_base = OTHER; note = 2-prime-deoxy-2-prime-fluorocytidine-3-prime-phosph ate
  modified_base order(4,18,20): mod_base = OTHER; note = 2-prime-O-methyladenosine-3-prime-phosphate
  modified_base order(5,13): mod_base = OTHER; note = 2-prime-deoxy-2-prime-fluoroguanosine-3-prime-phosp hate
  modified_base order(6,12): mod_base = OTHER; note = 2-prime-O-methyluridine-3-prime-phosphate
  modified_base 7: mod_base = OTHER; note = 2-prime-deoxy-2-prime-fluoroguanosine-3-prime-phosp hate
  modified_base 8: mod_base = OTHER; note = 2-prime-O-methyluridine-3-prime-phosphate
  modified_base order(9,11,15,17,19): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base 21: mod_base = OTHER; note = 2-prime-deoxy-2-prime-fluoroadenosine-3-prime-phosp hate
aacagtgttcttgctctataa

SEQ ID NO: 2 - RNA, 21 nt, synthetic construct
  source 1..21: mol_type = other RNA
  modified_base order(1,21): mod_base = OTHER; note = 2-prime-deoxy-2-prime-fluoroadenosine-3-prime-phosp hate
  modified_base order(2,4,18): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17): mod_base = OTHER; note = 2-prime-deoxy-2-prime-fluorouridine-3-prime-phospha te
  modified_base order(14,16): mod_base = OTHER; note = 2-prime-O-methylcytidine-3-prime-phosphate
  modified_base 19: mod_base = OTHER; note = 2-prime-deoxy-2-prime-fluorouridine -3-prime-phosphorothioate
  modified_base 20: mod_base = OTHER; note = 2-prime-O-methyladenosine-3-prime- phosphorothioate
aacagtgttcttgctctataa

SEQ ID NO: 3 - RNA, 23 nt, synthetic construct
  source 1..23: mol_type = other RNA
  modified_base order(1,21..22): mod_base = OTHER; note = 2-prime-O-methyluridine-3-prime-phosphorothioate
  modified_base 2: mod_base = OTHER; note = 2-prime-deoxy-2-prime-fluorouridine -3-prime-phosphorothioate
  modified_base order(3,5,7,11,13): mod_base = OTHER; note = 2-prime-O-methyladenosine-3-prime-phosphate
  modified_base order(4,18,20): mod_base = OTHER; note = 2-prime-deoxy-2-prime-fluorouridine-3-prime-phospha te
  modified_base order(6,8): mod_base = OTHER; note = 2-prime-deoxy-2-prime-fluoroguanosine-3-prime-phosp hate
  modified_base order(9,15,17): mod_base = OTHER; note = 2-prime-O-methylcytidine-3-prime-phosphate
  modified_base order(10,14,16): mod_base = OTHER; note = 2-prime-deoxy-2-prime-fluoroadenosine-3-prime-phosp hate
  modified_base order(12,19): mod_base = OTHER; note = 2-prime-O-methylguanosine-3-prime-phosphate
  modified_base 23: mod_base = OTHER; note = 2-prime-O-methyluridine-3-prime-phosphate
ttatagagcaagaacactgtttt

SEQ ID NO: 4 - RNA, 22 nt, synthetic construct
  source 1..22: mol_type = other RNA
  modified_base order(1,20..21): mod_base = OTHER; note = 2-prime-O-methyluridine-3-prime-phosphorothioate
  modified_base 2: mod_base = OTHER; note = 2-prime-deoxy-2-prime-fluorouridine -3-prime-phosphorothioate
  modified_base order(3,5,10,12): mod_base = OTHER; note = 2-prime-O-methyladenosine-3-prime-phosphate
  modified_base order(4,17,19): mod_base = OTHER; note = 2-prime-deoxy-2-prime-fluorouridine-3-prime-phospha te
  modified_base order(6,7): mod_base = OTHER; note = 2-prime-deoxy-2-prime-fluoroguanosine-3-prime-phosp hate
  modified_base order(8,14,16): mod_base = OTHER; note = 2-prime-O-methylcytidine-3-prime-phosphate
  modified_base order(9,13,15): mod_base = OTHER; note = 2-prime-deoxy-2-prime-fluoroadenosine-3-prime-phosp hate
  modified_base order(11,18): mod_base = OTHER; note = 2-prime-O-methylguanosine-3-prime-phosphate
  modified_base 22: mod_base = OTHER; note = 2-prime-O-methyluridine-3-prime-phosphate
ttataggcaagaacactgtttt

SEQ ID NO: 5 - RNA, 21 nt, synthetic construct
  source 1..21: mol_type = other RNA
  modified_base 1: mod_base = OTHER; note = 2-prime-O-methylcytidine-3-prime-phosphorothioate
  modified_base order(2,19..20): mod_base = OTHER; note = 2-prime-O-methyladenosine-3-prime- phosphorothioate
  modified_base order(3,4,5,12,16,18): mod_base = OTHER; note = 2-prime-O-methyluridine-3-prime-phosphate
  modified_base 6: mod_base = OTHER; note = 2-prime-O-hexadecyl-uridine-3-prime-phosphate
  modified_base 7: mod_base = OTHER; note = 2-prime-deoxy-2-prime-fluoroadenosine-3-prime-phosp hate
  modified_base order(8,14,21): mod_base = OTHER; note = 2-prime-O-methyladenosine-3-prime-phosphate
  modified_base 9: mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base order(13,15,17): mod_base = OTHER; note = 2-prime-O-methylcytidine-3-prime-phosphate
cattttaatcctcactctaaa

SEQ ID NO: 6 - RNA, 20 nt, synthetic construct
  source 1..20: mol_type = other RNA
  modified_base 1: mod_base = OTHER; note = 2-prime-O-methylcytidine-3-prime- phosphorothioate
  modified_base order(2,18..19): mod_base = OTHER; note = 2-prime-O-methyladenosine-3-prime- phosphorothioate
  modified_base order(3,4,5,11,15,17): mod_base = OTHER; note = 2-prime-O-methyluridine-3-prime-phosphate
  modified_base 6: mod_base = OTHER; note = 2-prime-deoxy-2-prime-fluoroadenosine-3-prime-phosp hate
  modified_base order(7,13,20): mod_base = OTHER; note = 2-prime-O-methyladenosine-3-prime-phosphate
  modified_base 8: mod_base = OTHER; note = 2-prime-deoxy-2-prime-fluorouridine-3-prime-phospha te
  modified_base order(9,10): mod_base = OTHER; note = 2-prime-deoxy-2-prime-fluorocytidine-3-prime-phosph ate
  modified_base order(12,14,16): mod_base = OTHER; note = 2-prime-O-methylcytidine-3-prime-phosphate
catttaatcctcactctaaa

SEQ ID NO: 7 - RNA, 23 nt, synthetic construct
  source 1..23: mol_type = other RNA
  modified_base 1: mod_base = OTHER; note = 5-prime-E-vinylphosphonate 2-prime-O-methyluridine-3-prime-phosphorothioate
  modified_base 2: mod_base = OTHER; note = 2-prime-deoxy-2-prime-fluorouridine -3-prime-phosphorothioate
  modified_base order(3,15,20): mod_base = OTHER; note = 2-prime-O-methyluridine-3-prime-phosphate
  modified_base order(4,10,13,17..19): mod_base = OTHER; note = 2-prime-O-methyladenosine-3-prime-phosphate
  modified_base order(5,7,11,12,23): mod_base = OTHER; note = 2-prime-O-methylguanosine-3-prime-phosphate
  modified_base order(6,16): mod_base = OTHER; note = 2-prime-deoxy-2-prime-fluoroadenosine-3-prime-phosp hate
  modified_base order(8,14): mod_base = OTHER; note = 2-prime-deoxy-2-prime-fluorouridine-3-prime-phospha te
  modified_base 9: mod_base = OTHER; note = 2-prime-deoxy-2-prime-fluoroguanosine-3-prime-phosp hate
  modified_base 21: mod_base = OTHER; note = 2-prime-O-methylguanosine-3-prime- phosphorothioate
  modified_base 22: mod_base = OTHER; note = 2-prime-O-methyladenosine-3-prime- phosphorothioate
tttagagtgaggattaaaatgag

SEQ ID NO: 8 - RNA, 22 nt, synthetic construct
  source 1..22: mol_type = other RNA
  modified_base 1: mod_base = OTHER; note = 5-prime-E-vinylphosphonate 2-prime-O-methyluridine-3-prime-phosphorothioate
  modified_base 2: mod_base = OTHER; note = 2-prime-deoxy-2-prime-fluorouridine -3-prime-phosphorothioate
  modified_base order(3,14,19): mod_base = OTHER; note = 2-prime-O-methyluridine-3-prime-phosphate
  modified_base order(4,9,12,16..18): mod_base = OTHER; note = 2-prime-O-methyladenosine-3-prime-phosphate
  modified_base order(5,10,11,22): mod_base = OTHER; note = 2-prime-O-methylguanosine-3-prime-phosphate
  modified_base order(6,15): mod_base = OTHER; note = 2-prime-deoxy-2-prime-fluoroadenosine-3-prime-phosp hate
  modified_base order(7,13): mod_base = OTHER; note = 2-prime-deoxy-2-prime-fluorouridine-3-prime-phospha te
  modified_base 8: mod_base = OTHER; note = 2-prime-deoxy-2-prime-fluoroguanosine-3-prime-phosp hate
  modified_base 20: mod_base = OTHER; note = 2-prime-O-methylguanosine-3-prime- phosphorothioate
  modified_base 21: mod_base = OTHER; note = 2-prime-O-methyladenosine-3-prime- phosphorothioate
tttagatgaggattaaaatgag

SEQ ID NO: 9 - protein, 29 aa, synthetic construct
AALEALAEALEALAEALEALAEAAAAGGC

SEQ ID NO: 10 - protein, 30 aa, synthetic construct
AALAEALAEALAEALAEALAEALAAAAGGC

SEQ ID NO: 11 - protein, 15 aa, synthetic construct
ALEALAEALEALAEA

SEQ ID NO: 12 - protein, 22 aa, synthetic construct
GLFEAIEGFIENGWEGMIWDYG

SEQ ID NO: 13 - protein, 23 aa, synthetic construct
GLFGAIAGFIENGWEGMIDGWYG

SEQ ID NO: 14 - protein, 48 aa, synthetic construct
GLFEAIEGFIENGWEGMIDGWYGCGLFEAIEGFIENGWEGMIDGWYGC

SEQ ID NO: 15 - protein, 44 aa, synthetic construct
GLFEAIEGFIENGWEGMIDGGCGLFEAIEGFIENGWEGMIDGGC

SEQ ID NO: 16 - protein, 35 aa, synthetic construct
GLFGALAEALAEALAEHLAEALAEALEALAAGGSC

SEQ ID NO: 17 - protein, 34 aa, synthetic construct
GLFEAIEGFIENGWEGLAEALAEALEALAAGGSC

SEQ ID NO: 18 - protein, 41 aa, synthetic construct
  SITE 17: note = Nle
  SITE 38: note = Nle
GLFEAIEGFIENGWEGXIDGKGLFEAIEGFIENGWEGXIDG

SEQ ID NO: 19 - protein, 19 aa, synthetic construct
LFEALLELLESLWELLLEA

SEQ ID NO: 20 - protein, 20 aa, synthetic construct
GLFKALLKLLKSLWKLLLKA

SEQ ID NO: 21 - protein, 20 aa, synthetic construct
GLFRALLRLLRSLWRLLLRA

SEQ ID NO: 22 - protein, 30 aa, synthetic construct
WEAKLAKALAKALAKHLAKALAKALKACEA

SEQ ID NO: 23 - protein, 22 aa, synthetic construct
GLFFEAIAEFIEGGWEGLIEGC

SEQ ID NO: 24 - protein, 26 aa, synthetic construct
GIGAVLKVLTTGLPALISWIKRKRQQ

SEQ ID NO: 25 - protein, 8 aa, synthetic construct
HHHHHWYG

SEQ ID NO: 26 - protein, 10 aa, synthetic construct
CHKKKKKKHC

SEQ ID NO: 27 - protein, 16 aa, synthetic construct
RQIKIWFQNRRMKWKK

SEQ ID NO: 28 - protein, 14 aa, synthetic construct
GRKKRRQRRRPPQC

SEQ ID NO: 29 - protein, 27 aa, synthetic construct
GALFLGWLGAAGSTMGAWSQPKKKRKV

SEQ ID NO: 30 - protein, 18 aa, synthetic construct
LLIILRRRIRKQAHAHSK

SEQ ID NO: 31 - protein, 26 aa, synthetic construct
GWTLNSAGYLLKINLKALAALAKKIL

SEQ ID NO: 32 - protein, 18 aa, synthetic construct
KLALKLALKALKAALKLA

SEQ ID NO: 33 - protein, 9 aa, synthetic construct
RRRRRRRRR

SEQ ID NO: 34 - protein, 10 aa, synthetic construct
KFFKFFKFFK

SEQ ID NO: 35 - protein, 37 aa, synthetic construct
LLGDFFRKSKEKIGKEFKRIVQRIKDFLRNLVPRTES

SEQ ID NO: 36 - protein, 31 aa, synthetic construct
SWLSKTAKKLENSAKKRISEGIAIAIQGGPR

SEQ ID NO: 37 - protein, 30 aa, synthetic construct
ACYCRIPACIAGERRYGTCIYQGRLWAFCC

SEQ ID NO: 38 - protein, 36 aa, synthetic construct
DHYNCVSSGGQCLYSACPIFTKIQGTCYRGKAKCCK

SEQ ID NO: 39 - protein, 42 aa, synthetic construct
RRRPRPPYLPRPRPPPFFPPRLPPRIPPGFPPRFPPRFPGKR

SEQ ID NO: 40 - protein, 13 aa, synthetic construct
ILPWKWPWWPWRR

SEQ ID NO: 41 - protein, 16 aa, synthetic construct
AAVALLPAVLLALLAP

SEQ ID NO: 42 - protein, 11 aa, synthetic construct
AALLPVLLAAP

SEQ ID NO: 43 - protein, 12 aa, synthetic construct
RKCRIVVIRVCR

SEQ ID NO: 44 - RNA, 21 nt, synthetic construct
  source 1..21: mol_type = other RNA
  modified_base order(1,21): mod_base = OTHER; note = 2-prime-deoxy-2-prime-fluoroadenosine-3-prime-phosp hate
  modified_base order(5,7,13): mod_base = OTHER; note = 2-prime-deoxy-2-prime-fluoroguanosine-3-prime-phosp hate
  modified_base order(9,11,15,17,19): mod_base = OTHER; note = 2-prime-deoxy-2-prime-fluorouridine-3-prime-phospha te
  modified_base order(3,10): mod_base = OTHER; note = 2-prime-deoxy-2-prime-fluorocytidine-3-prime-phosph ate
  modified_base order(2,4,18,20): mod_base = OTHER; note = 2-prime-O-methyladenosine-3-prime-phosphate
  modified_base 14: mod_base = OTHER; note = 2-prime-O-methylcytidine-3-prime-phosphate
  modified_base order(6,8,12,16): mod_base = OTHER; note = 2-prime-O-methyluridine-3-prime-phosphate
aacagtgttcttgctctataa

SEQ ID NO: 45 - RNA, 23 nt, synthetic construct
  source 1..23: mol_type = other RNA
  modified_base order(10,14,16): mod_base = OTHER; note = 2-prime-deoxy-2-prime-fluoroadenosine-3-prime-phosp hate
  modified_base order(6,8): mod_base = OTHER; note = 2-prime-deoxy-2-prime-fluoroguanosine-3-prime-phosp hate
  modified_base order(2,4,18,20): mod_base = OTHER; note = 2-prime-deoxy-2-prime-fluorouridine-3-prime-phospha te
  modified_base order(3,5,7,11,13): mod_base = OTHER; note = 2-prime-O-methyladenosine-3-prime-phosphate
  modified_base order(12,19): mod_base = OTHER; note = 2-prime-O-methylguanosine-3-prime-phosphate
  modified_base order(9,15,17): mod_base = OTHER; note = 2-prime-O-methylcytidine-3-prime-phosphate
  modified_base order(1,21..23): mod_base = OTHER; note = 2-prime-O-methyluridine-3-prime-phosphate
ttatagagcaagaacactgtttt

SEQ ID NO: 46 - RNA, 21 nt, synthetic construct
  source 1..21: mol_type = other RNA
  modified_base order(1,13,15,17): mod_base = OTHER; note = 2-prime-O-methylcytidine-3-prime-phosphate
  modified_base order(2,8,14,19,20,21): mod_base = OTHER; note = 2-prime-O-methyladenosine-3-prime-phosphate
  modified_base order(3,4,5,12,16,18): mod_base = OTHER; note = 2-prime-O-methyluridine-3-prime-phosphate
  modified_base 6: mod_base = OTHER; note = 2 C-16
  modified_base 7: mod_base = OTHER; note = 2-prime-deoxy-2-prime-fluoroadenosine-3-prime-phosp hate
  modified_base 9: mod_base = OTHER; note = 2-prime-deoxy-2-prime-fluorouridine-3-prime-phospha te
  modified_base order(10,11): mod_base = OTHER; note = 2-prime-deoxy-2-prime-fluorocytidine-3-prime-phosph ate
  modified_base order(1^2,2^3,19^20,20^21): mod_base = OTHER; note = phosphorothioate linkage
cattttaatcctcactctaaa

SEQ ID NO: 47 - RNA, 23 nt, synthetic construct
  source 1..23: mol_type = other RNA
  modified_base order(1,3,12,13,17,19): mod_base = OTHER; note = 2-prime-O-methylguanosine-3-prime-phosphate
  modified_base order(2,5,6,7,11,14,20): mod_base = OTHER; note = 2-prime-O-methyladenosine-3-prime-phosphate
  modified_base order(4,9,21,23): mod_base = OTHER; note = 2-prime-O-methyluridine-3-prime-phosphate
  modified_base order(8,18): mod_base = OTHER; note = 2-prime-deoxy-2-prime-fluoroadenosine-3-prime-phosp hate
  modified_base order(10,16,22): mod_base = OTHER; note = 2-prime-deoxy-2-prime-fluorouridine-3-prime-phospha te
  modified_base 15: mod_base = OTHER; note = 2-prime-deoxy-2-prime-fluoroguanosine-3-prime-phosp hate
  modified_base order(1^2,2^3,21^22,22^23): mod_base = OTHER; note = phosphorothioate linkage
gagtaaaattaggagtgagattt

SEQ ID NO: 48 - RNA, 21 nt, synthetic construct
  source 1..21: mol_type = other RNA
  modified_base order(1,21): mod_base = OTHER; note = 2-prime-deoxy-2-prime-fluoroadenosine-3-prime-phosp hate
  modified_base order(2,4,18): mod_base = OTHER; note = 2-prime-O-methyladenosine-3-prime-phosphate
  modified_base order(3,10): mod_base = OTHER; note = 2-prime-deoxy-2-prime-fluorocytidine-3-prime-phosph ate
  modified_base order(5,7,13): mod_base = OTHER; note = 2-prime-deoxy-2-prime-fluoroguanosine-3-prime-phosp hate
  modified_base order(6,8,12): mod_base = OTHER; note = 2-prime-O-methyluridine-3-prime-phosphate
  modified_base order(9,11,15,17): mod_base = OTHER; note = 2-prime-deoxy-2-prime-fluorouridine-3-prime-phospha te
  modified_base 19: mod_base = OTHER; note = 2-prime-deoxy-2-prime-fluorouridine -3-prime-phosphorothioate
  modified_base order(14,16): mod_base = OTHER; note = 2-prime-O-methylcytidine-3-prime-phosphate
  modified_base 20: mod_base = OTHER; note = 2-prime-O-methyladenosine-3-prime- phosphorothioate
aacagtgttcttgctctataa